(19) United States
(12) Patent Application Publication
(10) Pub. No.: US 2022/0153766 A1
(43) Pub. Date: May 19, 2022

(54) CONDENSED TRICYCLIC COMPOUND USED AS KINASE INHIBITOR

(71) Applicant: Hangzhou Innogate Pharma Co., Ltd., Zhejiang (CN)

(72) Inventors: Hancheng ZHANG, Zhejiang (CN); Xin CHENG, Zhejiang (CN)

(21) Appl. No.: 17/439,548

(22) PCT Filed: Mar. 16, 2020
(86) PCT No.: PCT/IB2020/052388

§ 371 Date: Sep. 15, 2021

(30) Foreign Application Priority Data
Mar. 15, 2019 (CN) 2019101995274
Jun. 21, 2019 (CN) 2019105444177
Jan. 9, 2020 (CN) 2020100237991

(51) Int. Cl. C07F 9/6561

(52) U.S. Cl. CPC C07F 9/6561

(57) ABSTRACT

The present invention provides a class of compounds containing tricyclic heteroaryl groups. Specifically, the present invention provides compounds of the structure represented by the following formula (I) (the definition of each group is as described in the specification), pharmaceutical compositions containing the compounds of formula (I), as well as isotopic derivatives of these compounds, chiral isomers, allosteric forms, different salts, prodrugs, formulations, etc. The compounds of formula (I) can effectively inhibit protein kinases (including EGFR, EGFR (C797S), ALK, and HPK1, etc.), thereby playing a role in the treatment of various tumors.

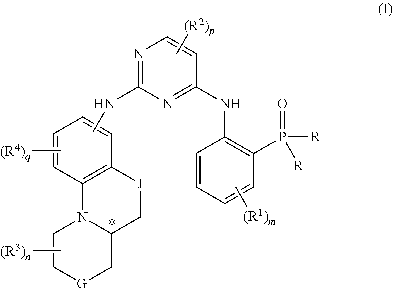

### TECHNICAL FIELD

The present invention relates to the field of medicinal chemistry; specifically, the present invention relates to a new type of derivatives containing tricyclic heteroaryl, its synthetic method and its use as the inhibitor of one or more protein kinases in the preparation of drugs for the treatment of tumors and other related diseases.

### BACKGROUND OF THE INVENTION

Cancer, also known as malignant tumor, is one of the diseases with the highest morbidity and mortality in the world. It is characterized by abnormal cell proliferation and metastasis, which spreads and metastasizes in a short or relatively short time after the onset of disease. Traditional treatment options include resection (if the conditions for resection are met), radiotherapy and chemotherapy. The targeted therapy developed in recent years has the advantages of reducing toxicity and side effects on patients, as well as improving survival rate. However, after using targeted drugs for a period of time, drug resistance will develop, after which the growth and spread of cancer cells will be extremely rapid. Common cancers are: hematological cancer, lung cancer, liver cancer, bladder cancer, rectal cancer, stomach cancer, and so on.

Among all cancers, the incidence and mortality of lung cancer account for the top few of all malignant tumors. Among them, non-small cell lung cancer (NSCLC) accounts for about 80% of all lung cancers.

At present, the most effective method for non-small cell lung cancer is individualized targeted therapy. Common targets are C-met, ALK and EGFR.

Epidermal growth factor receptor tyrosine kinase (EGFR) is composed of 1186 amino acids and encodes a transmembrane glycoprotein with a molecular weight of 170-kDa. EGFR can mediate multiple signal transduction pathways, transmit extracellular signals into the cell, and play an important role in regulating the proliferation, differentiation and apoptosis of normal cells and tumor cells. EGFR is a constitutively expressed component of many normal epithelial tissues (such as skin and hair follicles), while in most solid tumors, EGFR is overexpressed or highly expressed. For example, in lung cancer, the expression rate of EGFR reaches 40˜80%. Therefore, selectively inhibiting EGFR and interfering with its mediated signal transduction pathway can achieve the purpose of treating lung cancer, opening up a feasible way for targeted therapy of lung cancer.

Anaplastic lymphoma kinase (ALK) is a transmembrane receptor tyrosine kinase, which can be mutated in a variety of malignant tumors or fused with other oncogenes. It is the oncogenic driving gene of tumors. ALK inhibitors can be used to treat lung cancer.

Studies have shown that EGFR mutations are the most common mutations in patients with non-small cell lung cancer, especially after using epidermal growth factor receptor (EGFR) inhibitor drugs. About 30% to 40% Asian NSCLC patients are diagnosed carrying EGFR mutations, especially middle-aged women without smoking history. Therefore, the development of a new generation of EGFR inhibitors to combat cancer mutations is a difficult problem for scientists to overcome, and it is also one of the current research hotspots in the field of biomedicine.

There are currently three generations of EGFR inhibitors on the market, among which the representative inhibitors of the first generation are gefitinib (Iressa®), erlotinib (Tarceva®) and icotinib (Conmana). The representative inhibitor of the second generation is afatinib, and the representative inhibitor of the third generation is osimertinib (AZD9291). Osimertinib (AZD9291) is a third generation orally-available small molecule EGFR-TKI. It is the first lung cancer drug to target the EGFR T790M mutation. It can target the EGFR gene mutations of NSCLC (including exon 18, 19, 21 mutations) and EGFR-TKI acquired resistance mutation (T790M), its advent has brought good survival benefits to more lung cancer patients. AZD9291 can significantly prolong the survival period by about one year. However, the development of resistance is very rapid afterwards, usually within 9-13 months. In August 2016, the world's top academic journal “Nature” published a blockbuster article, announcing a new generation of targeted drug compound EA1045 that can overcome the resistance of AZD9291, which can be used for first-generation drug-resistant patients with T790M mutations, or for patients who are resistant to AZD9291 and have C797S mutations, that is, the fourth-generation inhibitors are mainly for patients with L858R/T790M double mutations and C797S mutations.

Hematopoietic progenitor kinase 1 (HPK1, also known as MAP4K1) is a member of the germinal center kinase family of serine/threonine kinases. It is mainly expressed by hematopoietic cells and is an intracellular negative regulator for T cell proliferation and signal transduction. It plays an important role in the activation of dendritic cells and is a new anti-cancer immunotherapy target. Therefore, the use of small molecule inhibitors to inhibit HPK1 through single drugs or in combination with other drugs has the potential to treat cancer and other diseases.

### SUMMARY OF THE INVENTION

The objective of the present invention is to provide a of novel protein kinase inhibitor.

In the first aspect of the present invention, a compound of formula (I), or the optical isomers (including racemates, single enantiomers, and possible diastereomers), pharmaceutically acceptable salts, prodrugs, deuterated derivatives, hydrates, or solvates thereof is provided:

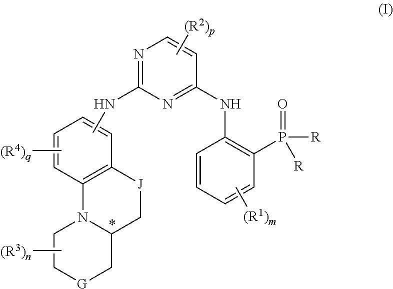

wherein in the formula (I):

“*” indicates a chiral center;

each R is independently C₁₋₄ alkyl;

each R¹ is independently hydrogen, deuterium, halogen, C₁₋₄ alkyl, C₂₋₄ alkenyl, C₂₋₄ alkynyl, C₃₋₆ cycloalkyl, 3- to 8-membered heterocyclic, aryl, heteroaryl, OR^{h}, or CN;

each R² is independently hydrogen, deuterium, halogen, C₁₋₄ alkyl, C₂₋₄ alkenyl, C₂₋₄ alkynyl, C₃₋₆ cycloalkyl, 3- to 8-membered heterocyclic, aryl, heteroaryl or CN;

each R³ is independently hydrogen, deuterium, or C₁₋₄ alkyl; or when two R³ are simultaneously attached to the same carbon atom, the two R³ and the carbon atom to which they are attached may optionally form a carbonyl group (C═O);

each R⁴ is independently hydrogen, deuterium, halogen, C₁₋₄ alkyl, C₁₋₄ haloalkyl, C₂₋₄ alkenyl, C₂₋₄ alkynyl, OR^{h}, SR^{h}, NR^{h}R^{h}, CN, C(O)R^{e}, C(O)OR^{h}, C(O)NR^{h}R^{h}, OC(O)R^{e}, NR^{h}C(O)R^{e}, or S(O)₂R^{e};

J and G are each independently NR^{f}, O, S, S(O), S(O)₂ or CR^{g}R^{g};

m is 0, 1, 2, 3, or 4;

n is 0, 1, 2, or 3;

p is 0, 1, or 2;

q is 0, 1, 2, or 3;

R^{f} is hydrogen, C₁₋₈ alkyl, C₁₋₈ haloalkyl, C₂₋₈ alkenyl, C₂₋₈ alkynyl, C₃₋₈ cycloalkyl, 3- to 12-membered heterocyclic, aryl, heteroaryl, C(O)R^{e}, C(O)OR^{h}, C(O)NR^{h}R^{h}, S(O)₂R^{e}, or S(O)₂NR^{h}R^{h}; wherein, each of the above groups is unsubstituted or substituted with 1-3 R^{e};

each R^{e} is independently selected from the group consisting of hydrogen, C₁₋₄ alkyl, C₁₋₄ haloalkyl, C₁₋₄ alkoxy substituted C₁₋₄ alkyl, C₂₋₄ alkenyl, C₂₋₄ haloalkenyl, C₁₋₄ alkoxy substituted C₂₋₄ alkenyl, hydroxyl substituted C₂₋₄ alkenyl, di(C₁₋₄ alkyl)amine substituted C₂₋₄ alkenyl, C₃₋₈ cycloalkyl substituted C₂₋₄ alkenyl, 3- to 8-membered heterocyclic group substituted C₂₋₄ alkenyl, aryl substituted C₂₋₄ alkenyl, heteroaryl substituted C₂₋₄ alkenyl, C₂₋₄ alkynyl, C₂₋₄ haloalkynyl, C₁₋₄ alkoxy substituted C₂₋₄ alkynyl, hydroxyl substituted C₂₋₄ alkynyl, di(C₁₋₄ alkyl)amine substituted C₂₋₄ alkynyl, C₃₋₈ cycloalkyl substituted C₂₋₄ alkynyl, 3- to 8-membered heterocyclic substituted C₂₋₄ alkynyl, aryl substituted C₂₋₄ alkynyl, heteroaryl substituted C₂₋₄ alkynyl, C₃₋₈ cycloalkyl, 3- to 8-membered heterocyclic group, aryl, or heteroaryl;

each R^{g} is independently selected from the group consisting of hydrogen, halogen, or C₁₋₄ alkyl; or two R^{g} together with the carbon atom to which they are attached form a carbonyl group (C═O); or two R^{g} together with the same carbon atom to which they attached form a 3- to 8-membered cyclic structure which optionally comprises 0, 1 or 2 heteroatoms selected from N, O, S;

each R^{h} is independently hydrogen or C₁₋₄ alkyl; or two R^{h} together with the nitrogen atom to which they are attached form a 3- to 8-membered cyclic structure, which comprises 1 or 2 N atom and 0 or 1 heteroatom selected from O and S;

wherein each of the above alkyl, alkenyl, alkynyl, cycloalkyl, heterocyclic, aryl and heteroaryl is optionally and independently substituted by 1 to 3 substituents independently selected from the group consisting halogen, C₁₋₄ alkyl, C₁₋₄ haloalkyl, C₂₋₄ alkenyl, C₂₋₄ alkynyl, C₃₋₈ cycloalkyl, 3- to 8-membered heterocyclic group, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, or S(O)₂R^{e}, provided that the chemical structure formed is stable and meaningful; wherein R^{e} and R^{h} are defined as above;

unless otherwise specified, the aryl is aromatic groups having 6 to 12 carbon atoms; the heteroaryl is 5- to 15-membered heteroaromatic groups; and the cyclic structure is saturated or unsaturated cyclic groups with or without heteroatoms.

In another preferred embodiment, the formula (I) is:

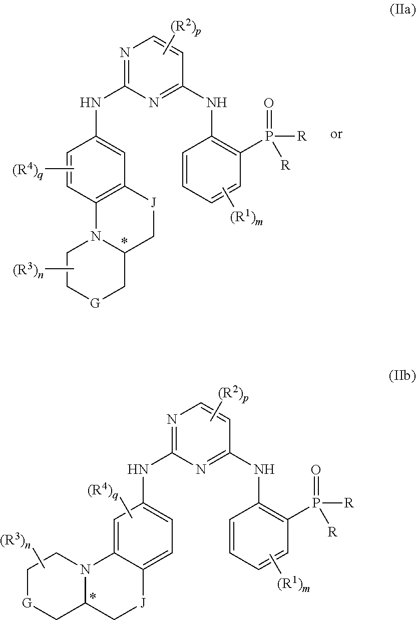

wherein each group is defined as in Claim 1.

In another preferred embodiment, each R is independently C₁₋₂ alkyl;

each R¹ is independently hydrogen, deuterium, halogen, or C₁₋₂ alkyl;

each R² is independently hydrogen, deuterium, halogen, or C₁₋₂ alkyl;

each of the R³ is independently hydrogen or C₁₋₄ alkyl; or when two R³ are simultaneously attached to the same carbon atom, the two R³ and the carbon atom to which they are attached form a carbonyl group (C═O);

each R⁴ is independently hydrogen, deuterated, halogen, C₁₋₄ alkyl, NR^{h}R^{h}, or NR^{h}C(O)R^{e};

m is 0, 1, or 2;

n is 0, 1, or 2;

p is 0, 1, or 2;

q is 0, 1, or 2;

wherein R^{e} and R^{h} are defined as in Claim 1.

In another preferred embodiment, the formula (I) is:

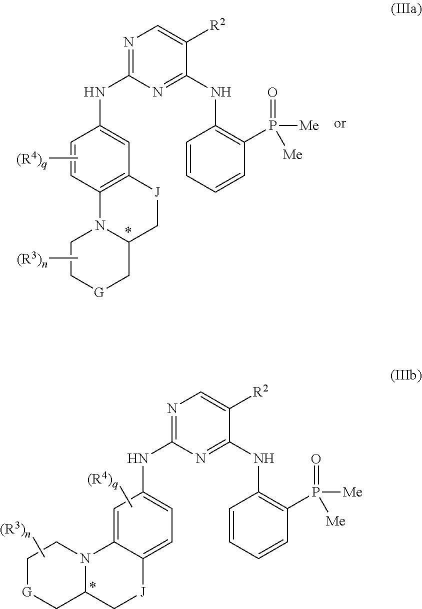

wherein R² is F, Cl, or Br; each R³ is independently hydrogen or C₁₋₄ alkyl; or when two R³ are simultaneously connected to the same carbon atom, the two R³ and the carbon atom to which they connected form a carbonyl group (C═O); each R⁴ is independently hydrogen, deuterium, halogen, C₁₋₄ alkyl, NR^{h}R^{h}, or NR^{h}C(O)R^{e}; n is 0, 1, or 2; q is 0, 1, or 2; wherein J, G, R^{e} and R^{h} are as defined in Claim 1.

In another preferred embodiment, the

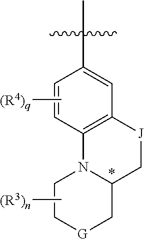

in formula (IIIa) is selected from:

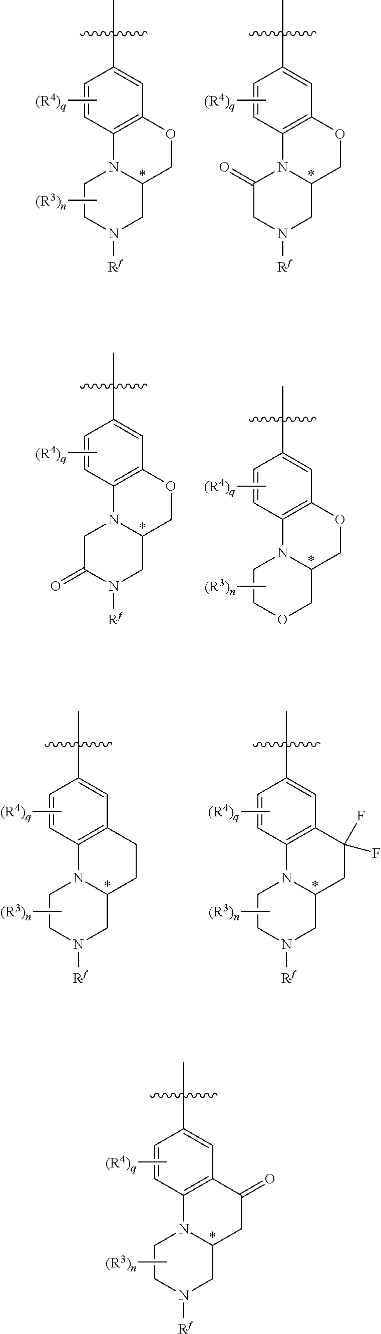

[symbol] means the connection site of the above structural fragment to other part in formula (IIIA);

wherein, each R³ is independently hydrogen or C₁₋₄ alkyl; when two R³ are simultaneously attached to the same carbon atom, the two R³ and the carbon atom to which they are attached form a carbonyl group (C═O);

each R⁴ is independently hydrogen, deuterated, halogen, C₁₋₂ alkyl, NR^{h}R^{h}, or NR^{h}C(O)R^{e};

n is 0, 1, or 2; q is 0 or 1;

R^{f} is hydrogen, C₁₋₄ alkyl, C₁₋₄ haloalkyl, C₂₋₄ alkenyl, C₂₋₄ alkynyl, C₃₋₆ cycloalkyl, 3- to 9-membered heterocyclic group, aryl, heteroaryl, C(O)R^{e}, C(O)OR^{h}, C(O)NR^{h}R^{h}, S(O)₂R^{e}, or S(O)₂NR^{h}R^{h}; wherein each alkyl, alkenyl, alkynyl, cycloalkyl, heterocyclic group, aryl and heteroaryl is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, S(O)₂R^{e}, or S(O)₂NR^{h}R^{h}; wherein R^{e} and R^{h} are as described in Claim 1.

In another preferred embodiment, the formula (I) is:

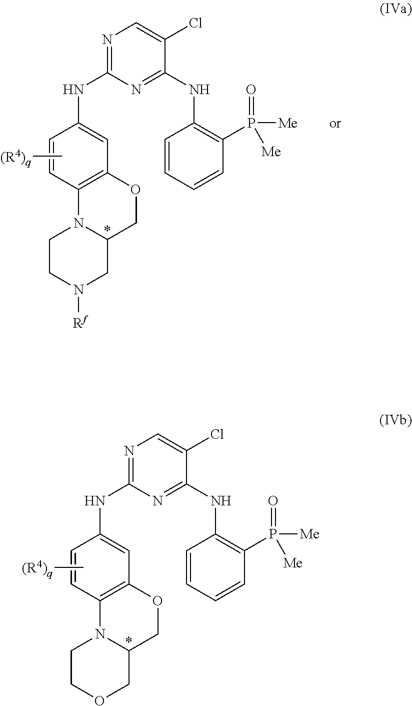

wherein each R⁴ is independently hydrogen, deuterium, halogen, C₁₋₂ alkyl, NR^{h}R^{h}, or NR^{h}C(O)R^{e}; q is 0 or 1;

R^{f} is hydrogen, C₁₋₄ alkyl, C₁₋₄ haloalkyl, C₂₋₄ alkenyl, C₂₋₄ alkynyl, C₃₋₆ cycloalkyl, 3- to 9-membered heterocyclic group, aryl, heteroaryl, C(O)R^{e}, C(O)OR^{h}, C(O)NR^{h}R^{h}, S(O)₂R^{e}, or S(O)₂NR^{h}R^{h}; wherein each alkyl, alkenyl, alkynyl, cycloalkyl, heterocyclic group, aryl, and heteroaryl is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, S(O)₂R^{e}, or S(O)₂NR^{h}R^{h}; wherein the definitions of R^{e} and R^{h} are as described in Claim 1.

In another preferred embodiment, R^{f} is selected from the group consisting of hydrogen, C₁₋₄ alkyl, C₁₋₄ haloalkyl, C₂₋₄ alkenyl, C₂₋₄ alkynyl, C₃₋₆ cycloalkyl, 3- to 9-membered heterocyclic group, aryl, heteroaryl, C(O)R^{e}, or S(O)R^{e}; wherein each alkyl, alkenyl, alkynyl, cycloalkyl, heterocyclic group, aryl, and heteroaryl is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{e}, SR^{e}, NR^{e}R^{e}, C(O)R^{e}, C(O)OR^{e}, C(O)NR^{e}R^{e}, NR^{e}C(O)R^{e}, S(O)₂R^{e}, or S(O)₂NR^{h}R^{h}; wherein the definitions of R^{e} and R^{h} are as described above.

In another preferred embodiment, the formula (I) is:

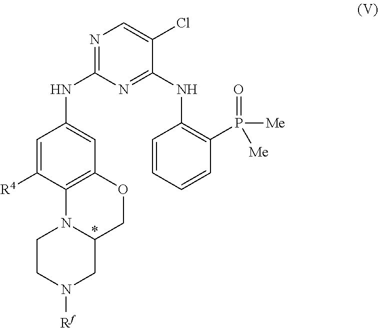

wherein, R⁴ is hydrogen, halogen, C₁₋₂ alkyl, NR^{h}R^{h}, or NR^{h}C(O)R^{e};

R^{f} is hydrogen, C₁₋₄ alkyl, C₁₋₄ haloalkyl, C₃₋₆ cycloalkyl, 3- to 9-membered heterocyclic group, aryl, heteroaryl, C(O)R^{e}, or S(O)R^{e}; wherein each alkyl, cycloalkyl, heterocyclic group, aryl and heteroaryl is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, S(O)₂R^{e}, or S(O)₂NR^{h}R^{h}; wherein the definitions of R^{e} and R^{h} are as described in Claim 1.

In another preferred embodiment, the formula (I) is:

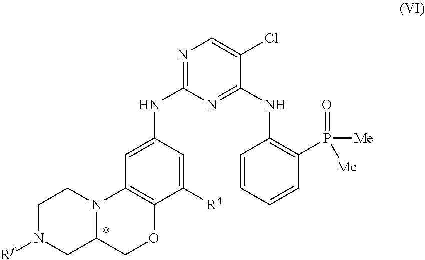

wherein, R⁴ is hydrogen, halogen, C₁₋₂ alkyl, NR^{h}R^{h}, or NR^{h}C(O)R^{e};

R^{f} is hydrogen, C₁₋₄ alkyl, C₁₋₄ haloalkyl, C₃₋₆ cycloalkyl, 3- to 9-membered heterocyclic group, aryl, heteroaryl, C(O)R^{e}, or S(O)R^{e}; wherein each alkyl, cycloalkyl, heterocyclic group, aryl and heteroaryl is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, S(O)₂R^{e}, or S(O)₂NR^{h}R^{h}; wherein the definitions of R^{e} and R^{h} are as described in the first aspect of the present invention.

In another preferred embodiment, the formula (I) is:

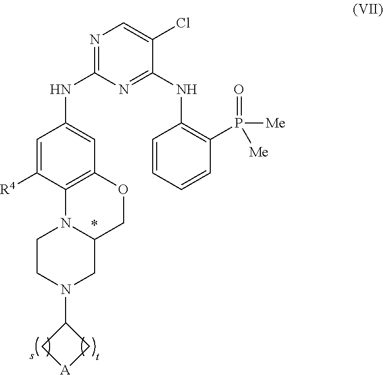

wherein, R⁴ is hydrogen, halogen or C₁₋₂ alkyl;

s and t are each independently 1, 2, or 3;

A is NR^{k}, O, or CR^{g}R^{g}; wherein R^{k} is hydrogen, C₁₋₄ alkyl, C₁₋₄ haloalkyl, hydroxy substituted C₁₋₄ alkyl, C₁₋₄ alkoxy substituted C₁₋₄ alkyl, di(C₁₋₄ alkyl) amine substituted C₁₋₄ alkyl, C₃₋₆ cycloalkyl, 3- to 9-membered heterocyclic group, aryl, heteroaryl, C(O)R^{e}, C(O)OR^{h}, C(O)NR^{h}R^{h}, S(O)₂R^{e}, or S(O)₂NR^{h}R^{h}; wherein each R^{e} is independently selected from the group consisting of hydrogen, C₁₋₄ alkyl, C₁₋₄ haloalkyl, C₂₋₄ alkenyl, C₁₋₄ alkoxy substituted C₂₋₄ alkenyl, di(C₁₋₄ alkyl)amine substituted C₂₋₄ alkenyl, C₂₋₄ alkynyl, C₃₋₆ cycloalkyl, 3- to 8-membered heterocyclic, aryl, or heteroaryl; the definitions of R^{g} and R^{h} are as described in the first aspect of the invention.

In another preferred embodiment, each R^{e} is independently selected from the group consisting of hydrogen, C₁₋₄ alkyl, C₁₋₄ haloalkyl, C₁₋₄ alkoxy substituted C₁₋₄ alkyl, C₂₋₄ alkenyl, C₁₋₄ alkoxy substituted C₂₋₄ alkenyl, hydroxyl substituted C₂₋₄ alkenyl, di(C₁₋₄ alkyl)amine substituted C₂₋₄ alkenyl, 3- to 6-membered heterocyclic substituted C₂₋₄ alkenyl, aryl substituted C₂₋₄ alkenyl, heteroaryl substituted C₂₋₄ alkenyl, C₂₋₄ alkynyl, C₃₋₈ cycloalkyl, 3- to 8-membered heterocyclic group, aryl, or heteroaryl.

In another preferred embodiment, each R⁴ is independently hydrogen, deuterium, halogen, C₁₋₂ alkyl, or NHC(O)CH═CH₂;

In another preferred embodiment, the compound is selected from the group consisting of:

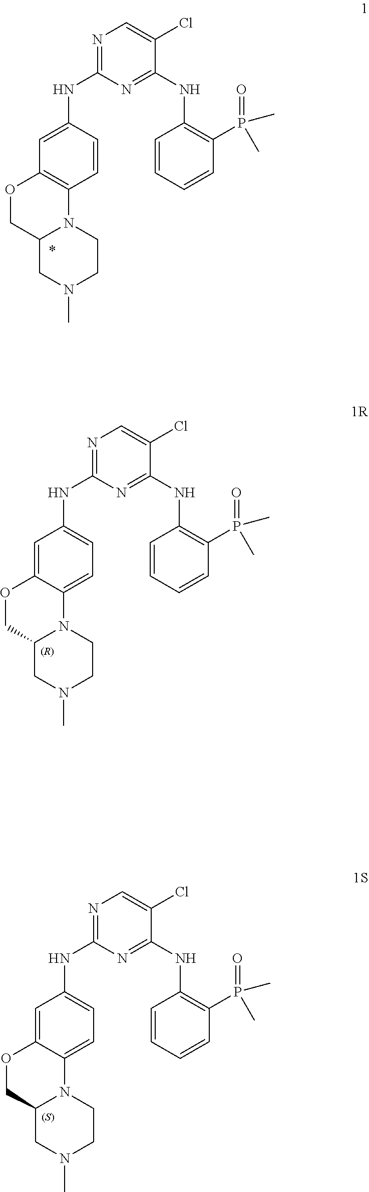

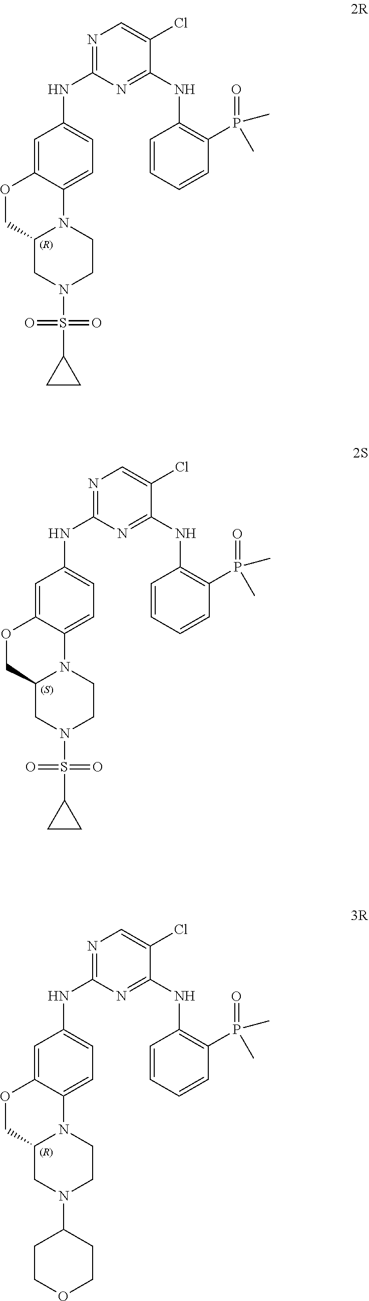

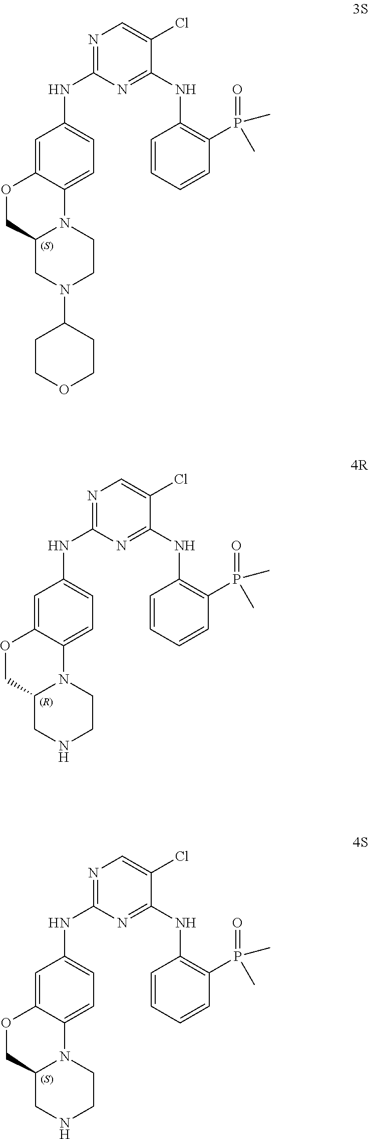

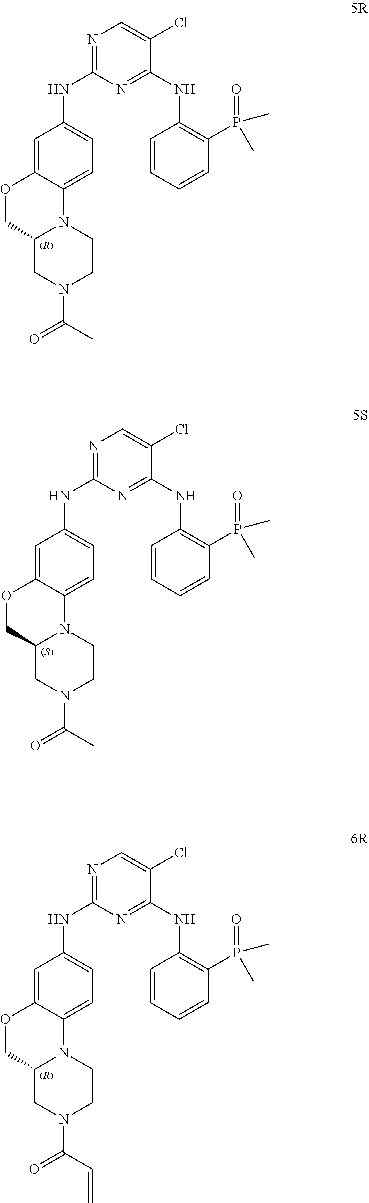

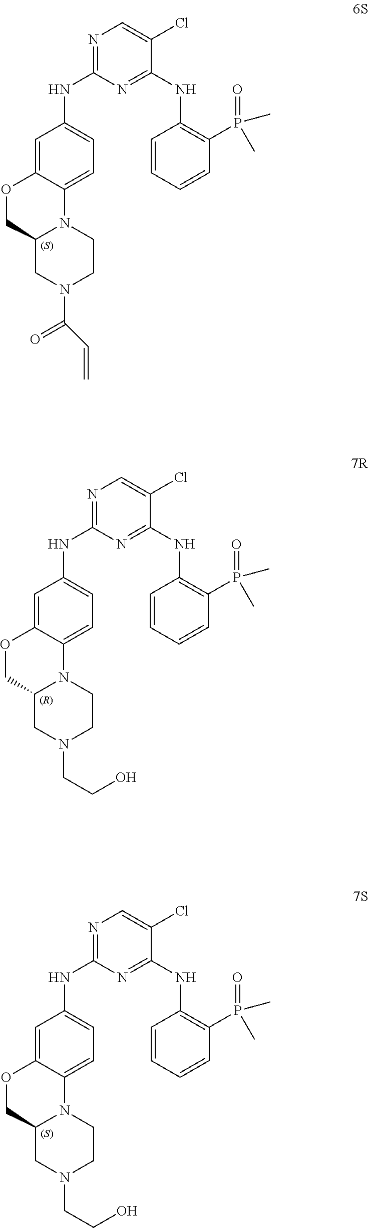

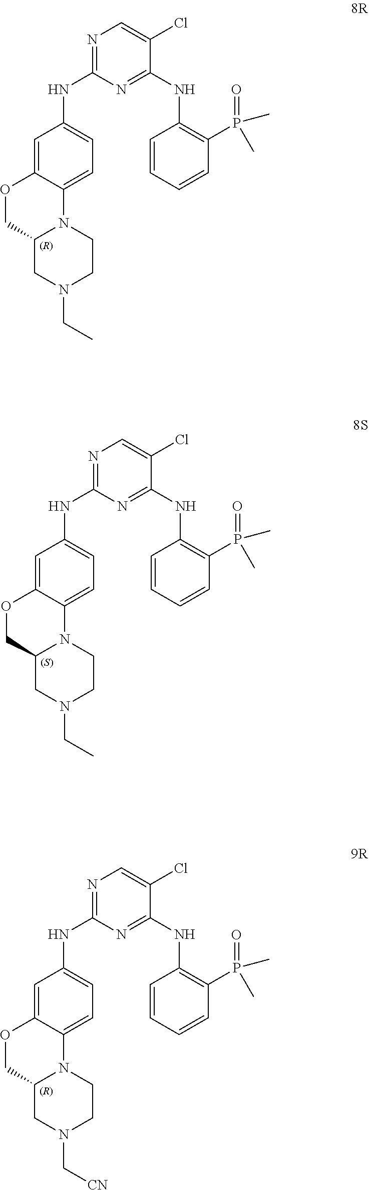

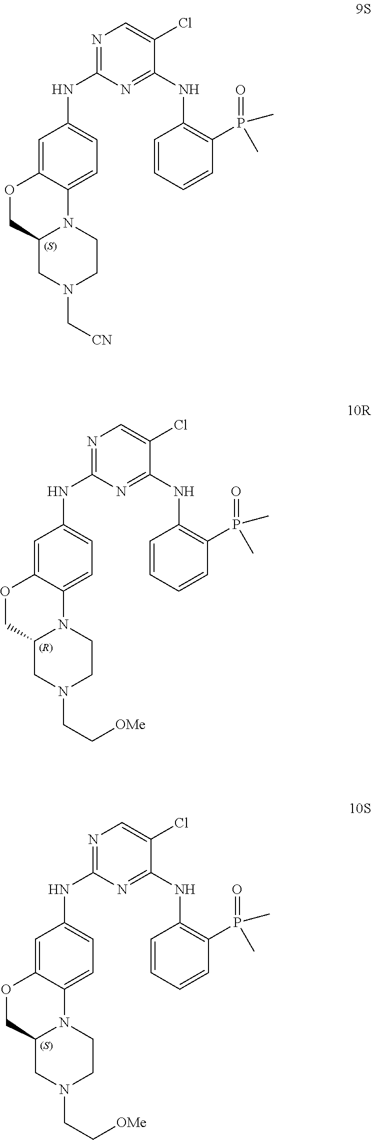

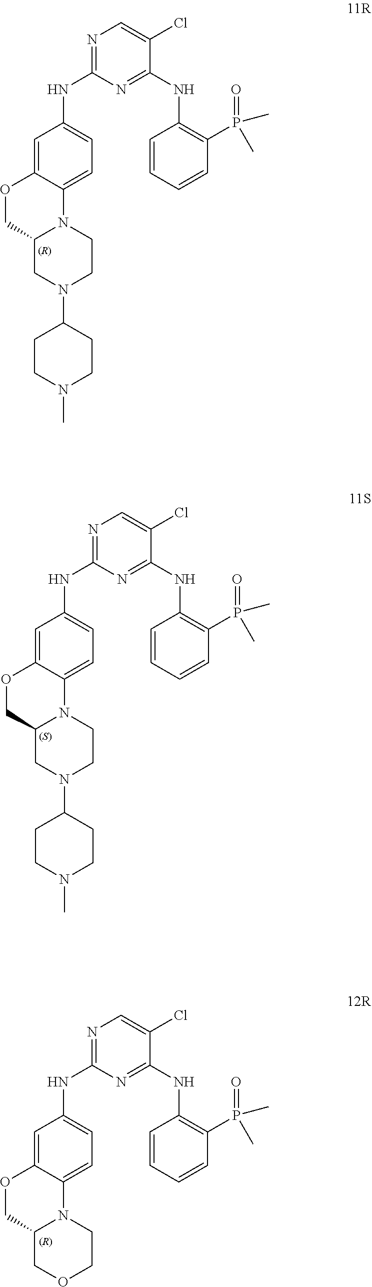

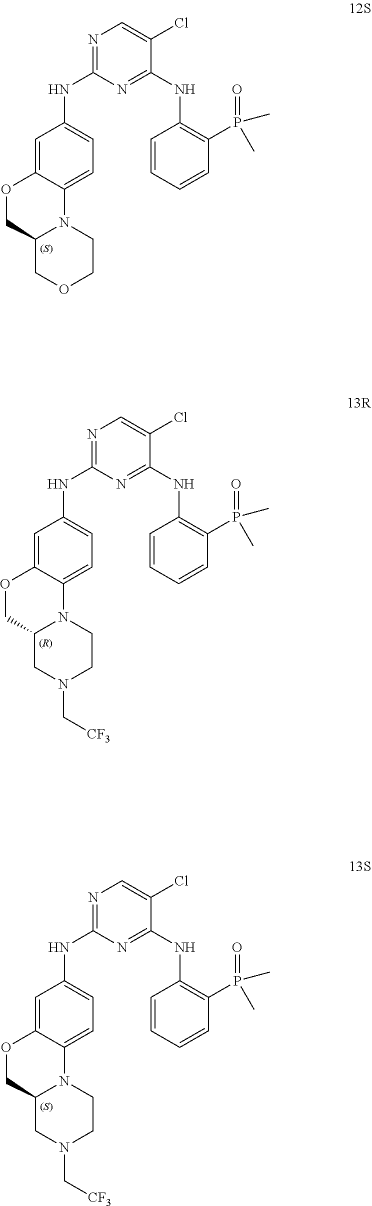

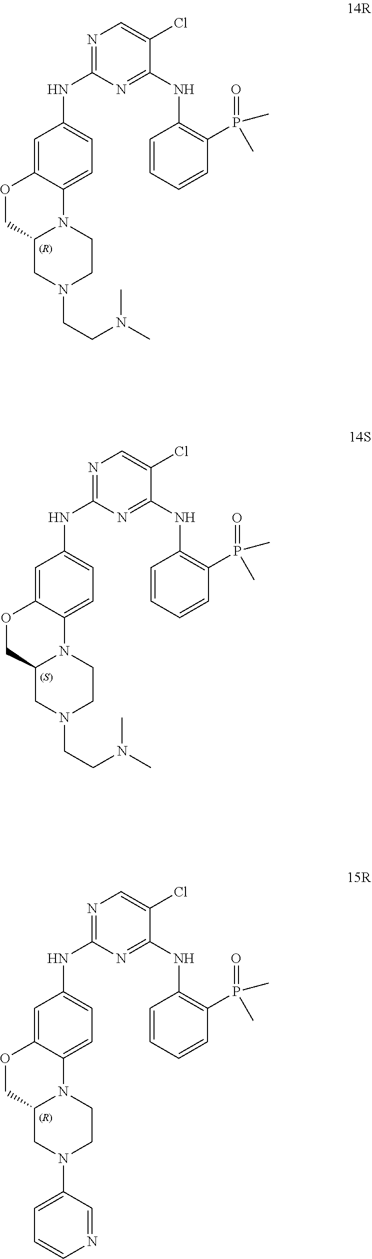

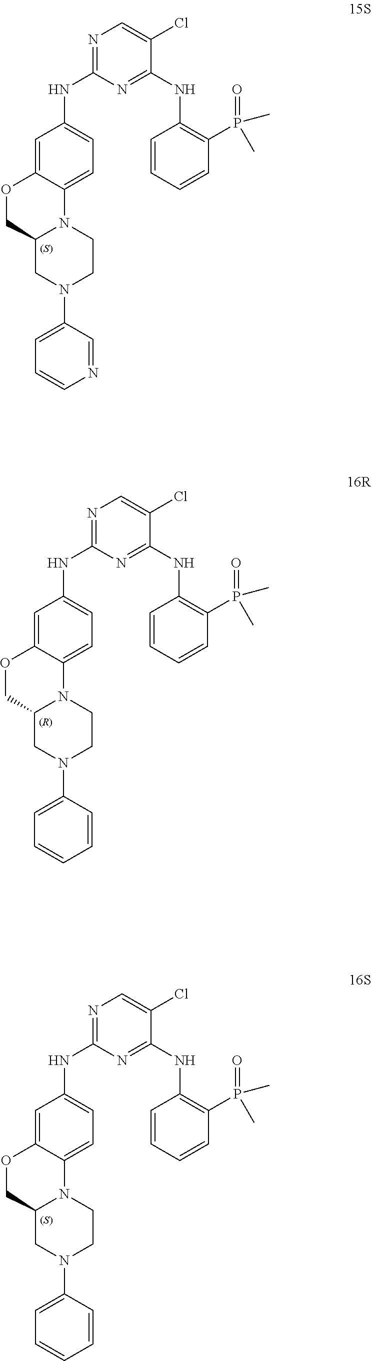

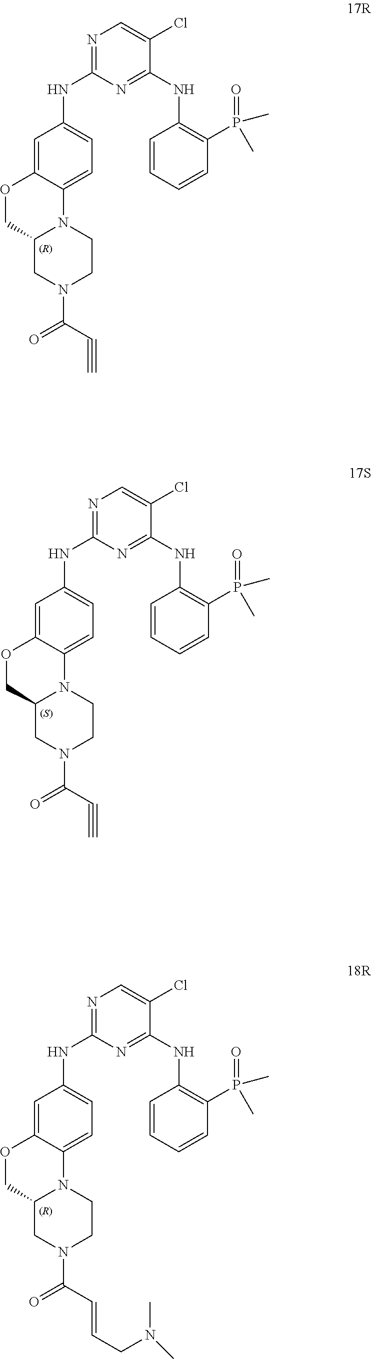

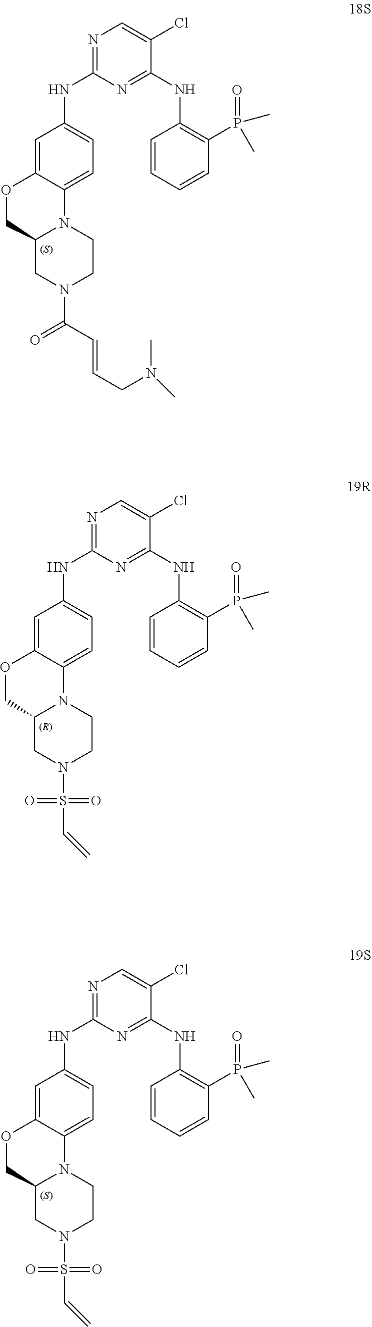

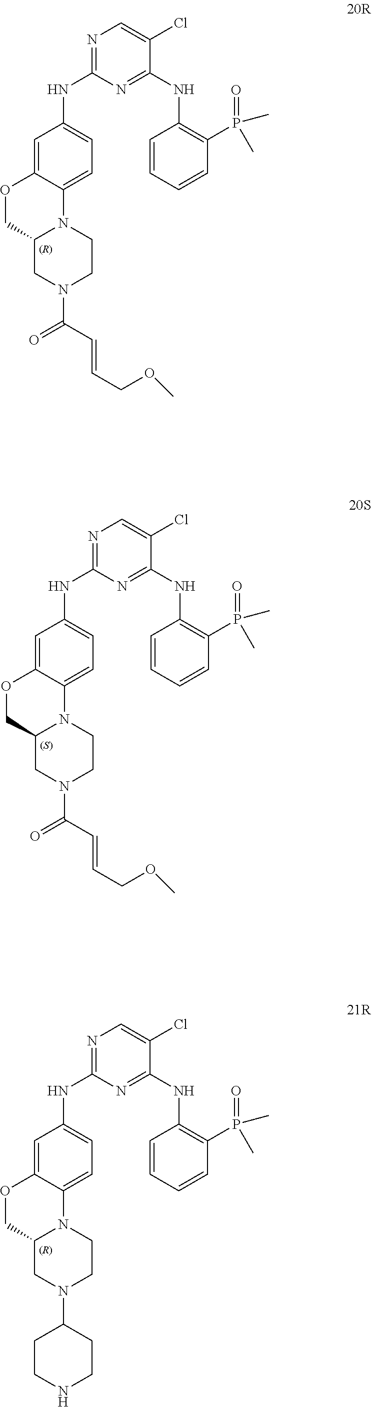

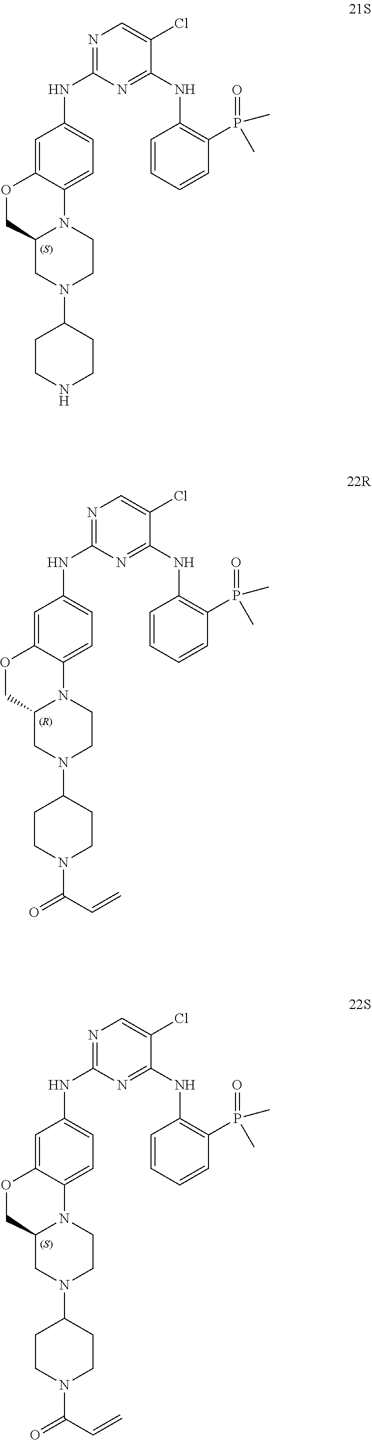

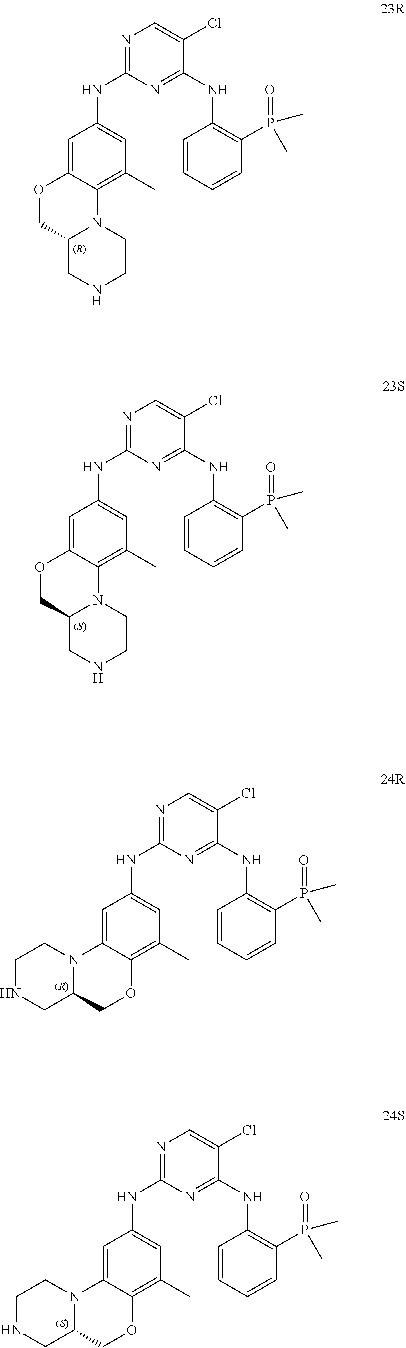

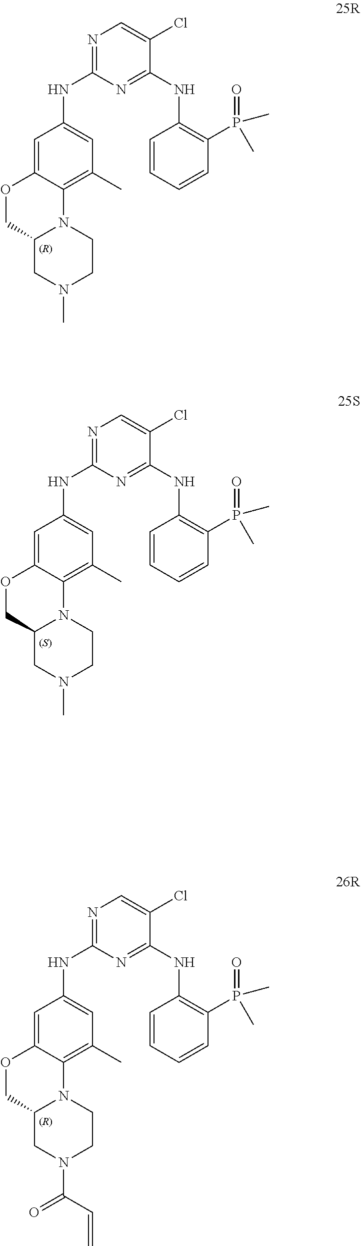

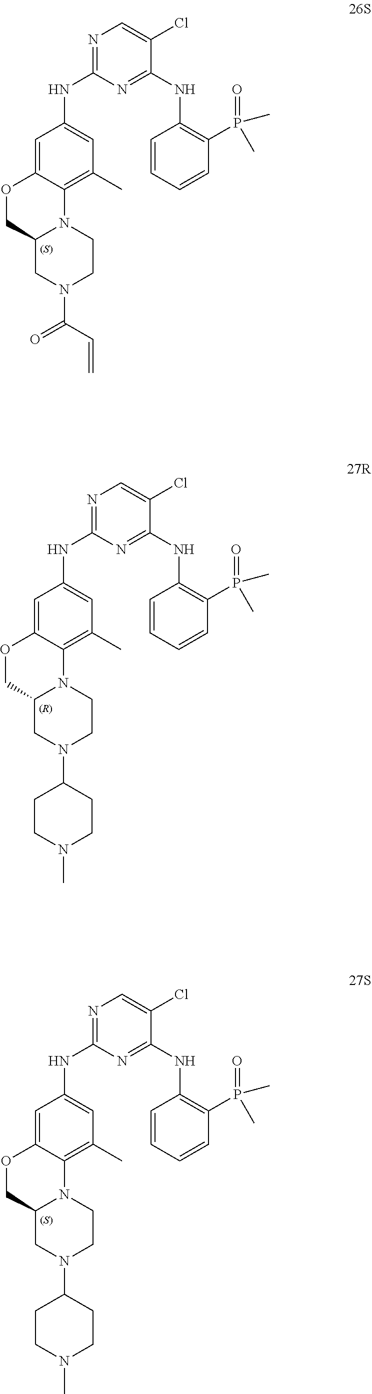

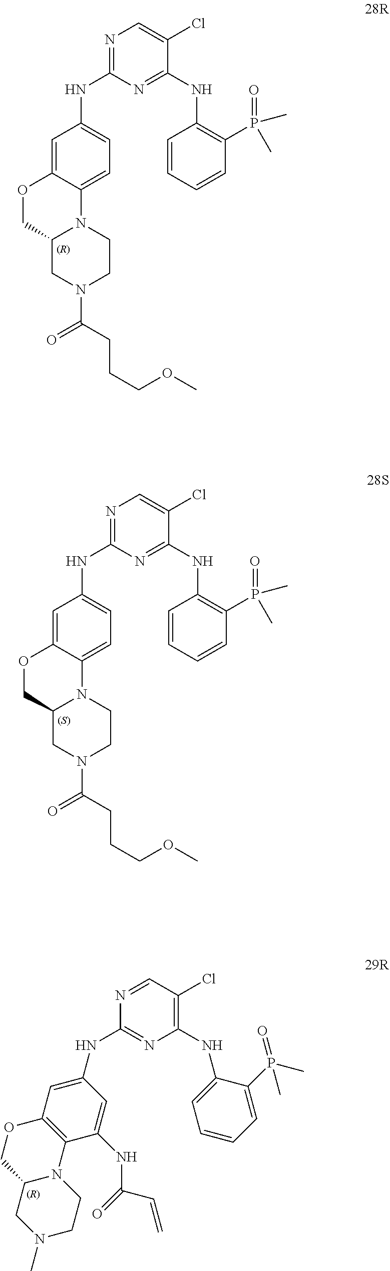

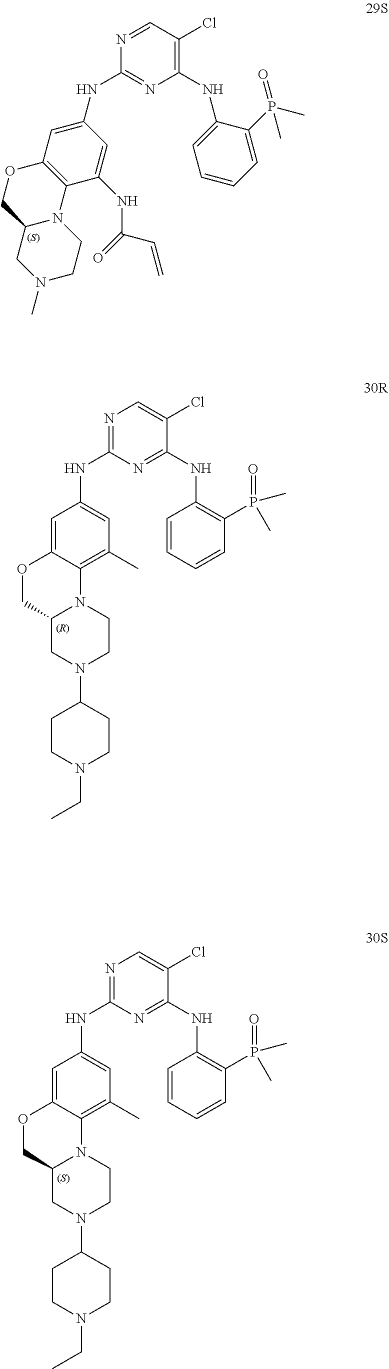

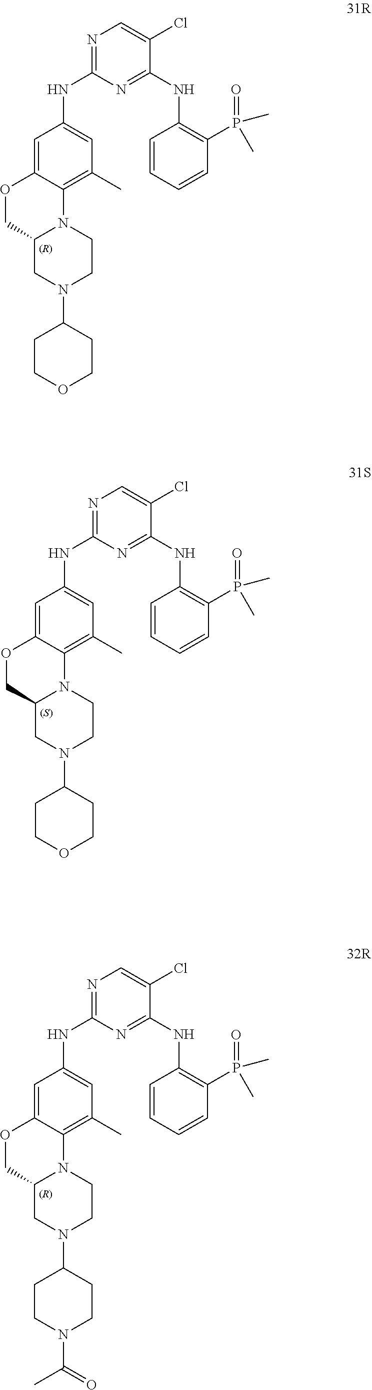

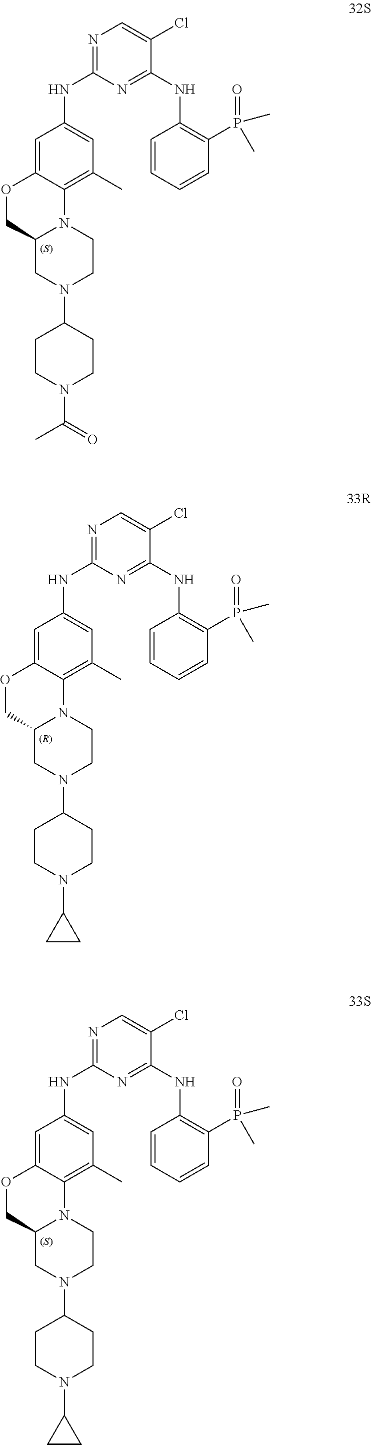

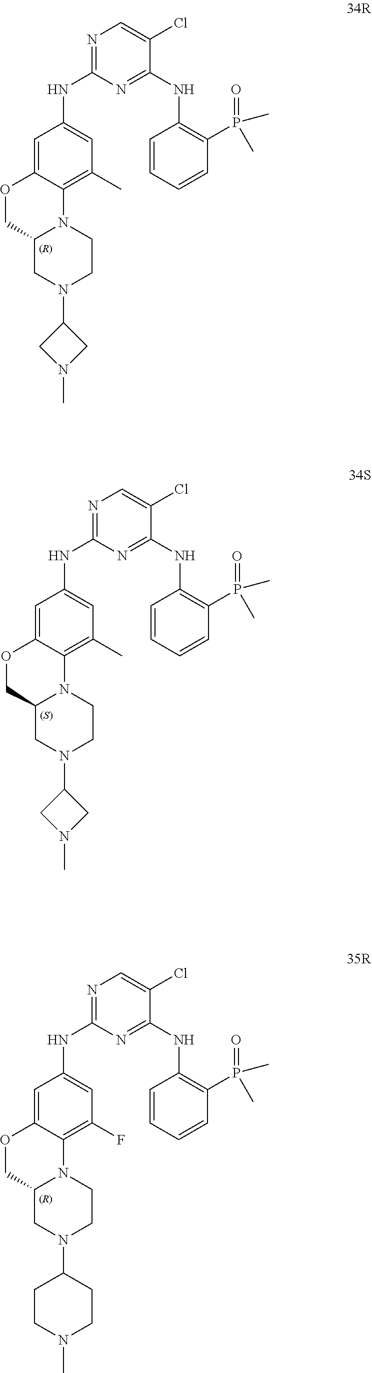

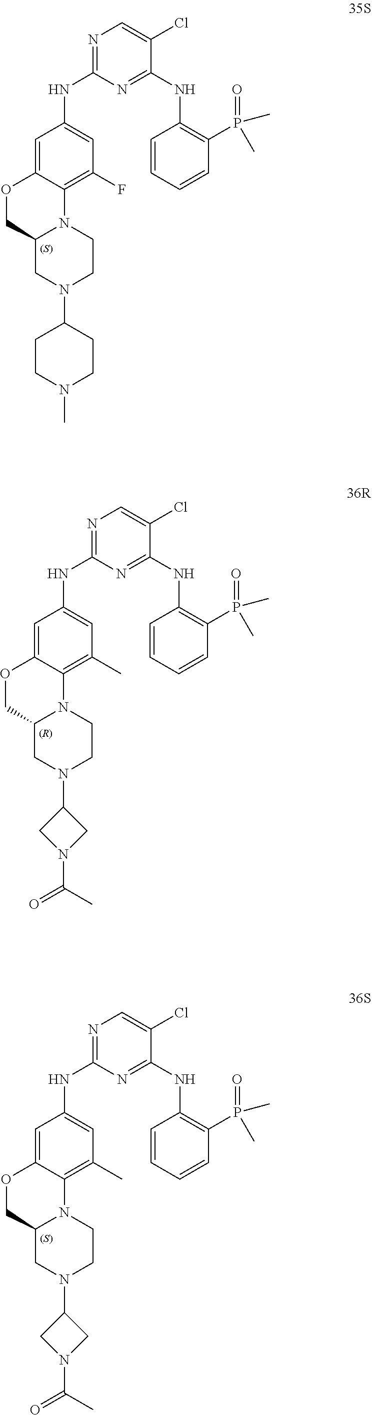

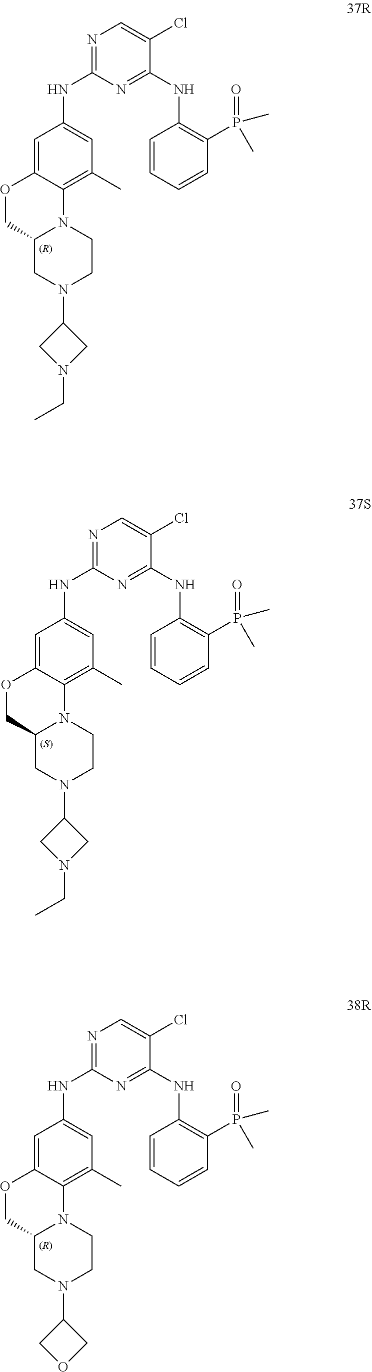

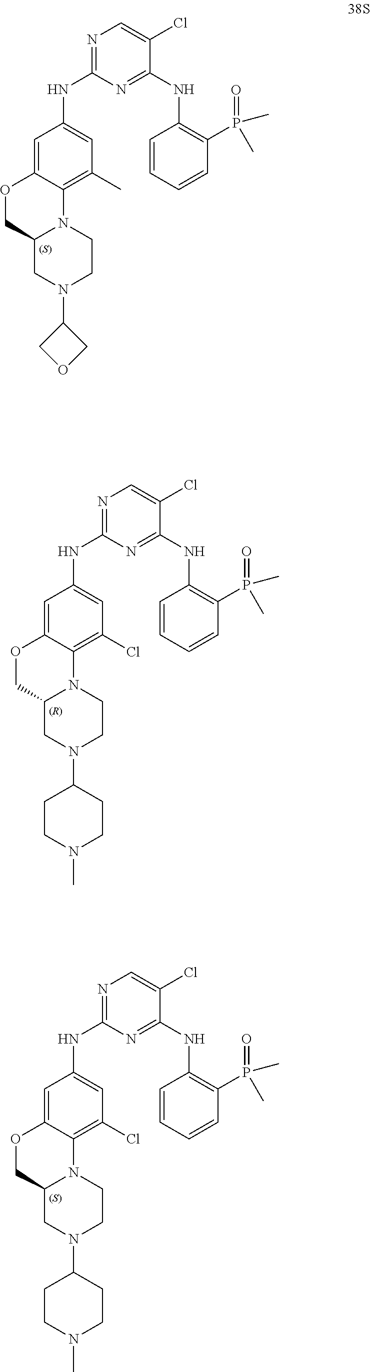

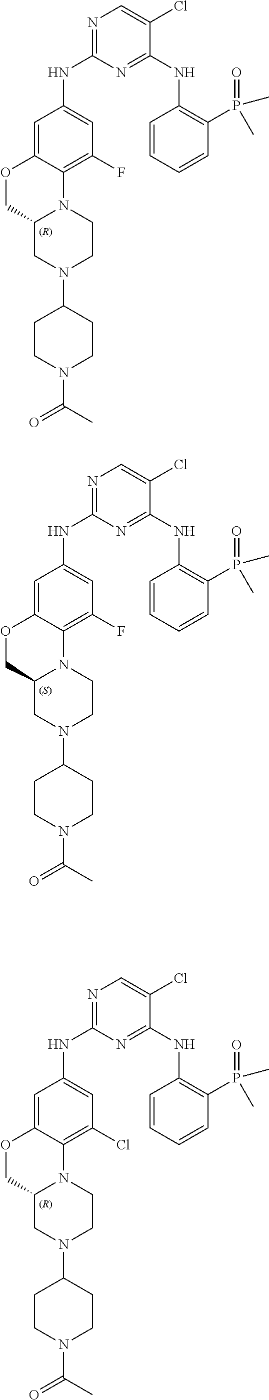

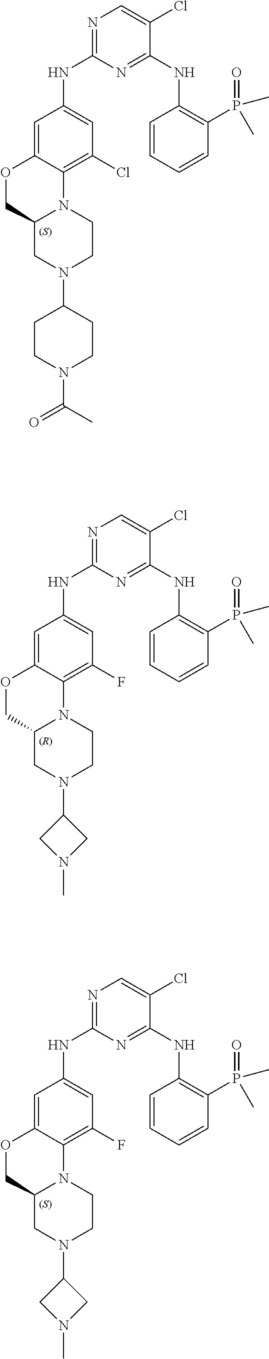

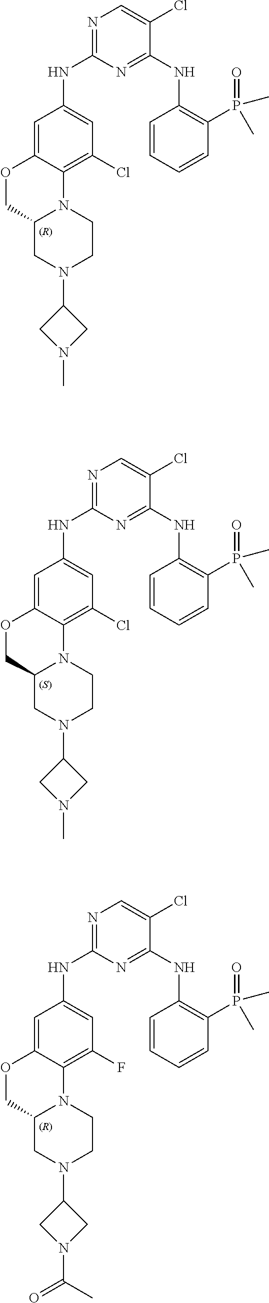

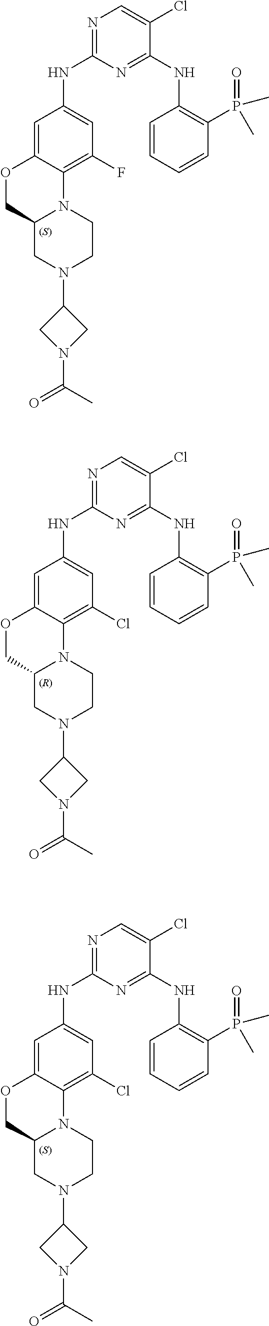

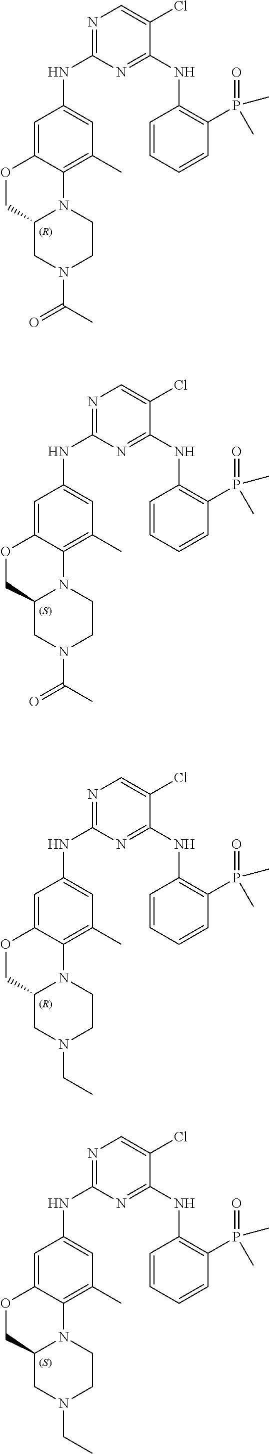

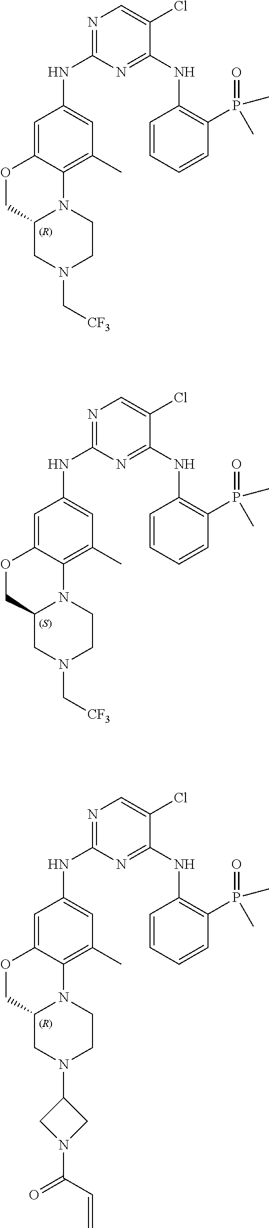

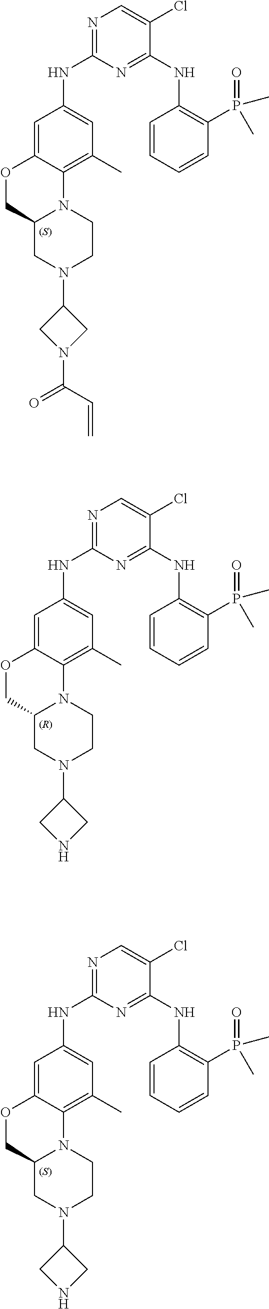

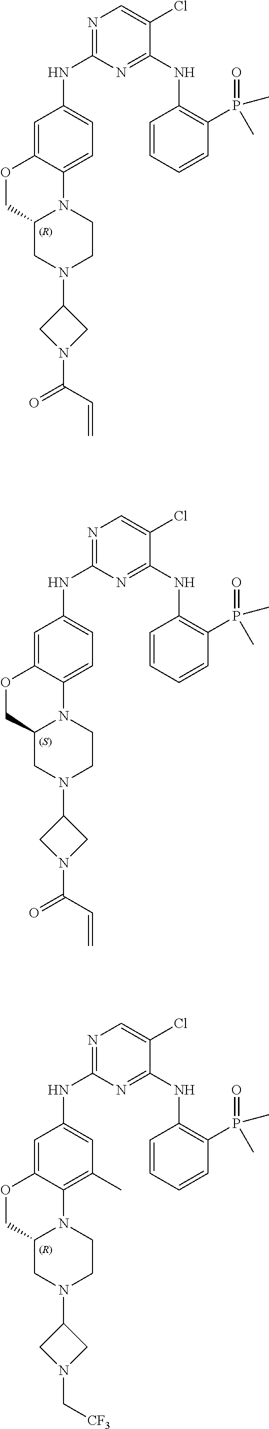

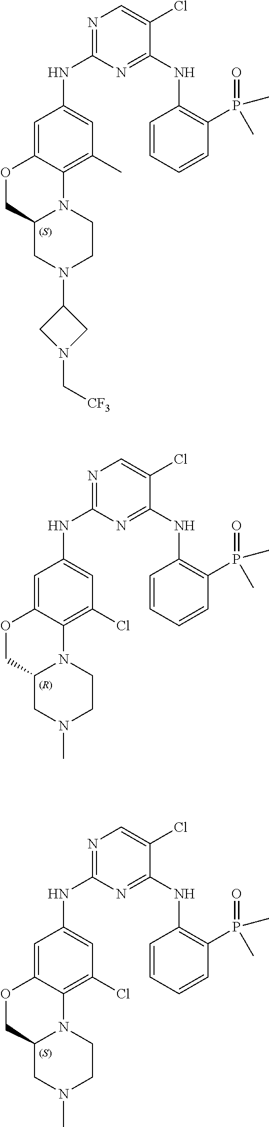

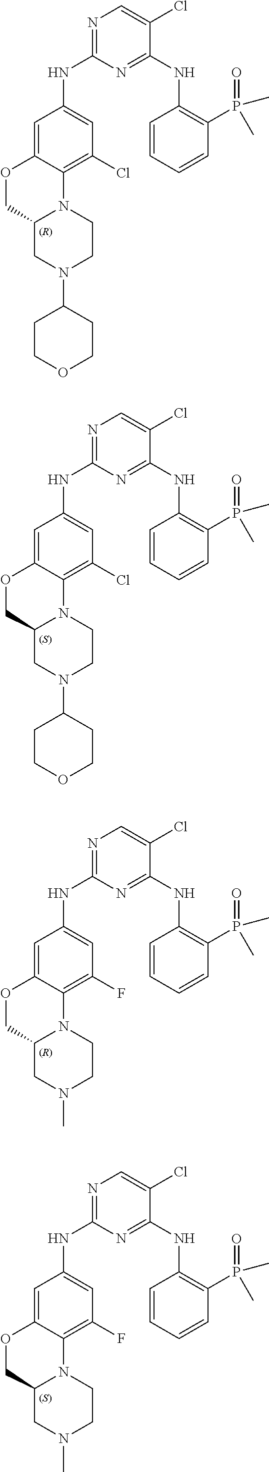

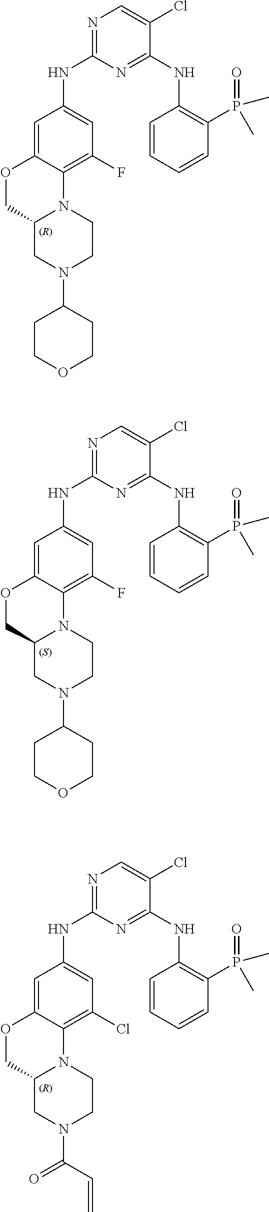

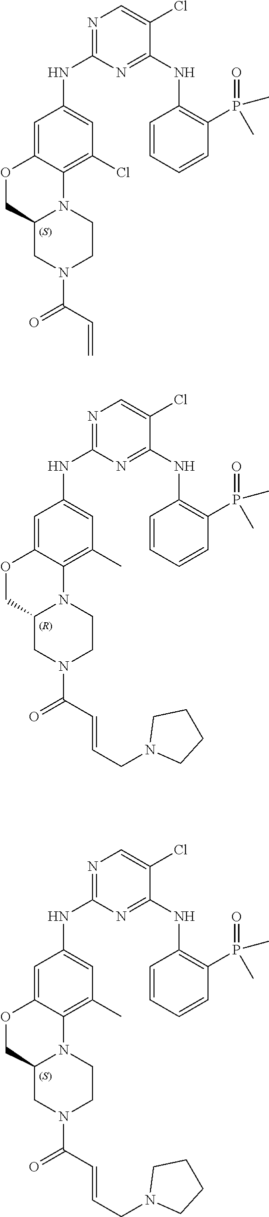

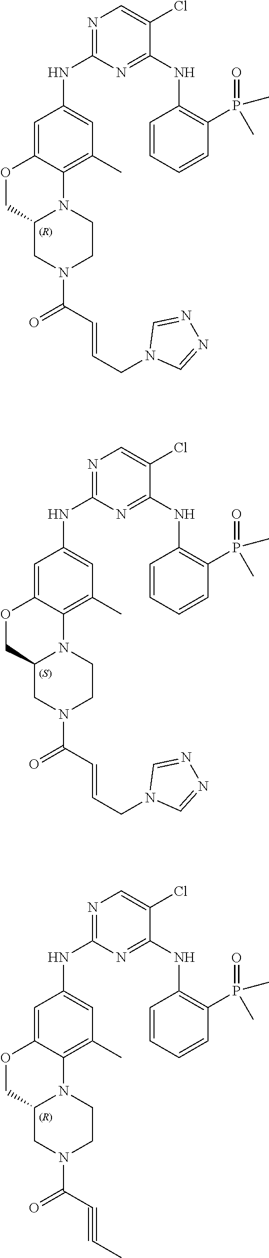

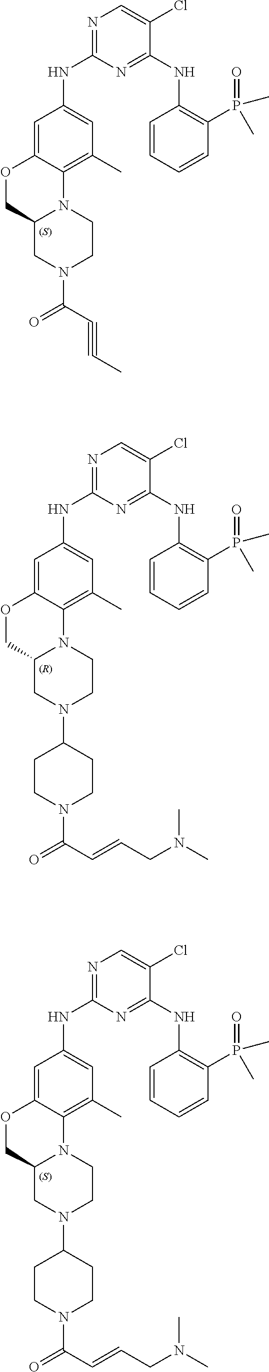

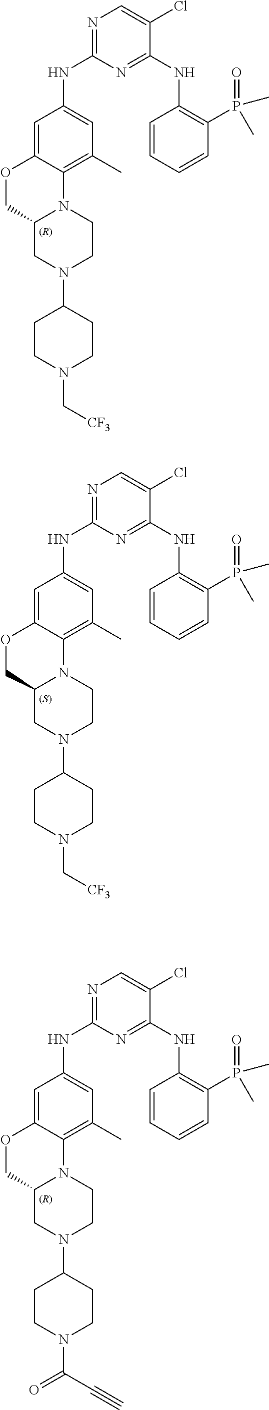

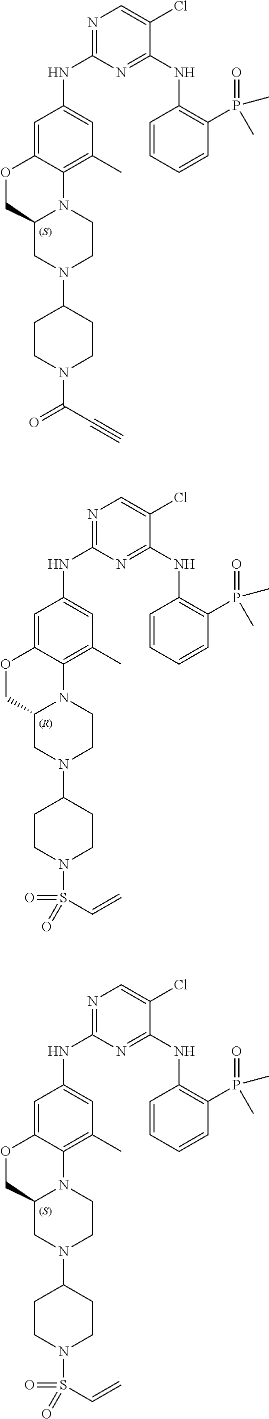

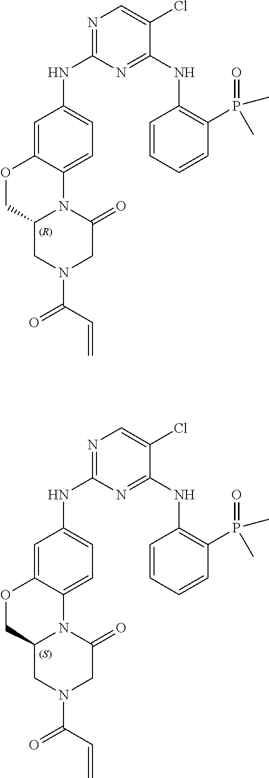

wherein, “*” indicates the chiral center, and when it have not been stated as R or S, the compound with “*” may be racemate, or may be R configuration or S configuration.

In another preferred embodiment, is used in:

(a) preparation of medicine for treating diseases associated with protein kinase activity or expression level;

(b) preparation of inhibitor targeting protein kinase; and/or

(c) in vitro non-therapeutic inhibition of protein kinase activity;

wherein the protein kinase is selected from the group consisting of EGFR, EGFR (C797S), ALK, HPK1, etc., or the combinations thereof.

In the second aspect of the present invention, a pharmaceutical composition is provided, which comprises: (i) therapeutically effective amount of compound of formula I in the first aspect of the invention, or its optical isomers, pharmaceutically acceptable salts, prodrugs, deuterated derivatives, hydrates, or solvates thereof, and (ii) pharmaceutically acceptable carriers.

In the third aspect of the present invention, a preparation method of compound of formula (I) is provided, wherein it comprises the following steps:

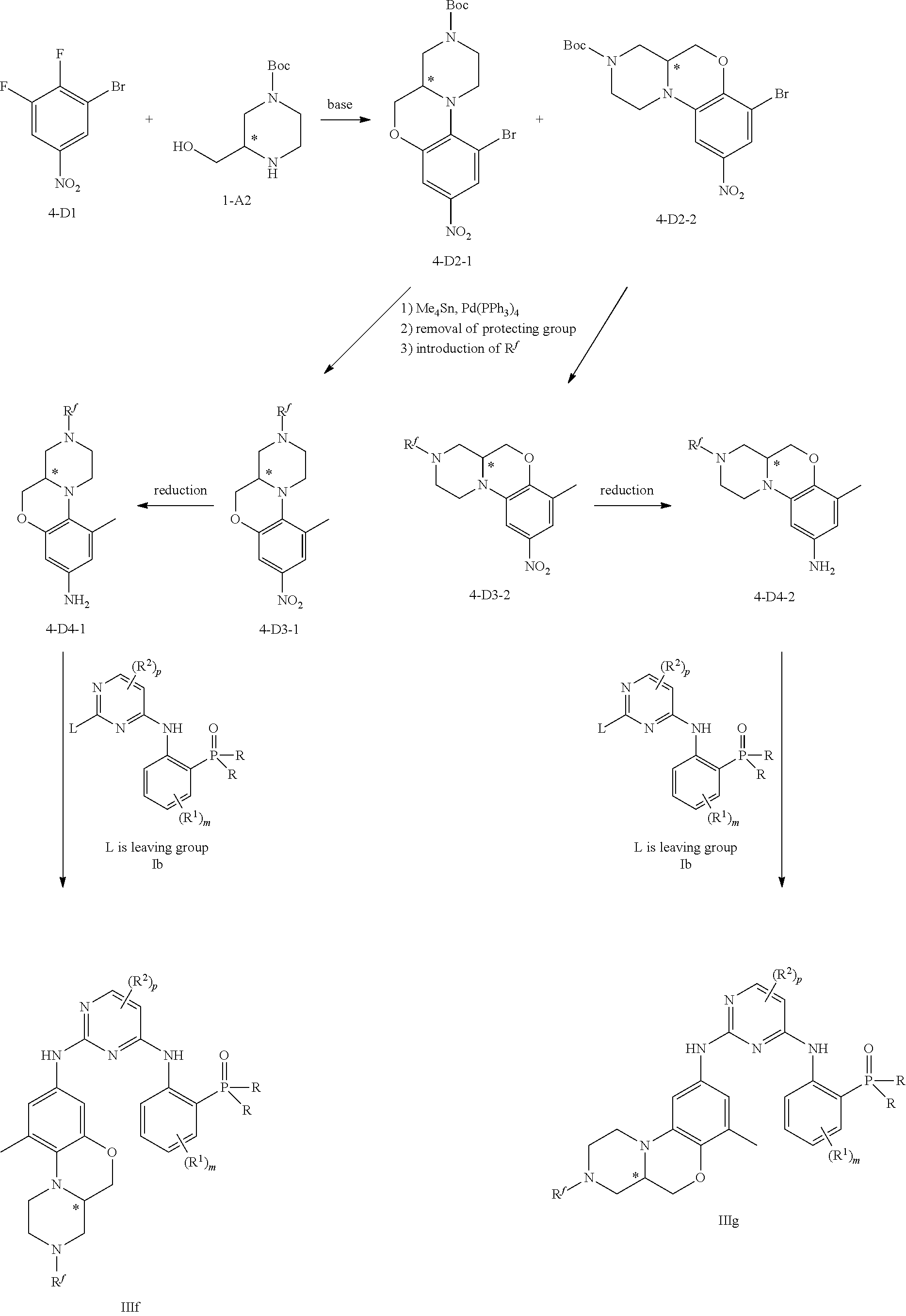

The reaction of the compound of formula 4-D1 with the compound of formula 1-A2 will produce a compound of formula 4-D2-1 or 4-D2-2;

In the presence of a palladium catalyst, reacting compound of formula 4-D2-1 or formula 4-D2-2 with Me₄Sn will produce the compound of formula 4-D3-1 or 4-D3-2;

The reduction of the compound of formula 4-D3-1 or formula 4-D3-2 will produce compound of formula 4-D4-1 or formula 4-D4-2;

Reacting compound of formula Ib with compound of formula 4-D4-1 or formula 4-D4-2 will produce compound of formula IIIf or IIIg. Compound IIIf or compound IIIg is part of the compounds in formula (I).

In the fourth aspect of the present invention, a preparation method of compound of formula (I) is provided, wherein it comprises the following steps:

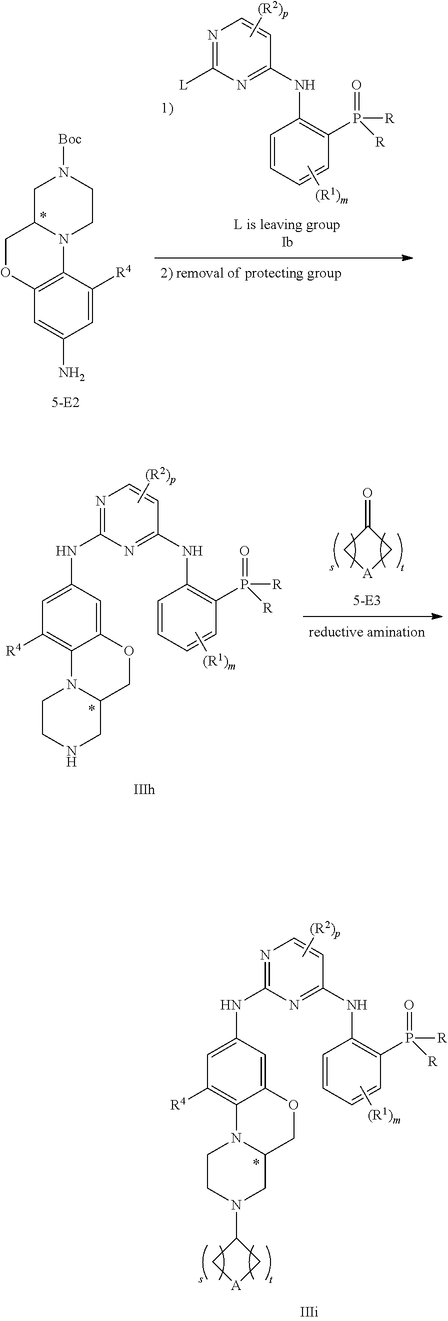

reaction of compound of formula 5-E2 with compound of formula Ib will produce a compound of formula IIIh;

The reductive amination using compound of formula IIIh and compound of formula 5-E3 will produce compound of formula IIIi. Compound IIIi is part of the compound of formula (I).

In the fifth aspect of the present invention, a method of inhibiting EGFR and/or EGFR (C797S) and/or ALK and/or HPK1 activity is provided, wherein comprising steps: administering an inhibitory effective amount of formula (I) compound, or optical isomers, pharmaceutically acceptable salts, prodrugs, deuterated derivatives, hydrates, or solvates thereof of the first aspect of the invention, or administering an inhibitory effective amount of pharmaceutical composition of the third aspect of the invention to an inhibition subject.

It should be understood that, in the present invention, the technical features (such as embodiments) specifically described in the context can be combined with each other to form a new or preferred technical solution, and no special instructions are required.

### DETAILED DESCRIPTION

After long-term and intensive research, the present inventors have unexpectedly discovered a class of protein kinase inhibitors containing tricyclic aryl compounds with novel structures, as well as their preparation methods and applications. These protein kinase inhibitors can inhibit protein kinases such as EGFR, EGFR (C797S), ALK, and HPK1, and the compounds of the present invention can be applied to the activities related to EGFR (including various mutations generated), ALK, and HPK1 Treatment of various diseases. Among them, inhibition of EGFR (C797S) is characterized by being able to overcome the drug resistance produced by the third generation of EGFR inhibitors. Specifically, the present invention includes a class of EGFR (especially EGFR (C797S)) inhibitors, which can effectively inhibit L858R/T790M double mutation and C797S mutation EGFR. In addition, the compounds of the present invention can inhibit the immune target HPK1, and can treat a variety of cancers and other diseases through single drug or in combination with other drugs. Based on the above findings, the inventor completed the present invention.

### Terminology

Unless otherwise stated, “or” as used herein has the same meaning as “and/or” (refers to “or” and “and”).

Unless otherwise specified, among all compounds of the present invention, each chiral carbon atom (chiral center) may optionally be in the R configuration or the S configuration, or a mixture of the R configuration and the S configuration.

As used herein, the term “alkyl”, alone or as part of another substituent, refers to a straight (ie, unbranched) or branched saturated hydrocarbon group containing only carbon atoms, or a combination of straight and branched chains. When the alkyl group has a carbon number limitation (e.g., C₁₋₁₀), it means that the alkyl group has 1 to 10 carbon atoms. For example, C₁₋₈ alkyl refers to an alkyl group containing from 1 to 8 carbon atoms, including methyl, ethyl, propyl, isopropyl, butyl, isobutyl, sec-butyl, tert-butyl, or the like.

As used herein, the term “alkenyl”, when used alone or as part of another substituent, refers to a straight or branched, carbon chain group having at least one carbon-carbon double bond. Alkenyl groups can be substituted or unsubstituted. When the alkenyl group has a carbon number limit (e.g., C₂₋₈), it means that the alkenyl group has 2-8 carbon atoms. For example, C₂₋₈ alkenyl refers to alkenyl groups having 2-8 carbon atoms, including ethenyl, propenyl, 1,2-butenyl, 2,3-butenyl, butadienyl, or the like.

As used herein, the term “alkynyl”, when used alone or as part of another substituent, refers to an aliphatic hydrocarbon group having at least one carbon-carbon triple bond. The alkynyl group can be straight or branched, or a combination thereof. When the alkynyl group has a carbon number limitation (e.g., C₂₋₈ alkynyl group), it means that the alkynyl group has 2 to 8 carbon atoms. For example, the term “C₂₋₈ alkynyl” refers to a straight or branched alkynyl group having 2-8 carbon atoms, including ethynyl, propynyl, isopropynyl, butynyl, isobutynyl, secondary butynyl, tert-butynyl, or the like.

As used herein, either used alone or as part of another substituent, the term “cycloalkyl” refers to a unit ring having a saturated or partially saturated ring, a bicyclic or polycyclic (fused ring, bridged or spiro) ring system. When a certain cycloalkyl group has a carbon number limitation (e.g., C₃₋₁₀), it means that the cycloalkyl group has 3 to 10 carbon atoms. In some preferred embodiments, the term “C₃₋₈ cycloalkyl” refers to a saturated or partially saturated monocyclic or bicyclic alkyl group having from 3 to 8 carbon atoms, including cyclopropyl, cyclobutyl, cyclopentyl, cycloheptyl, or the like. “Spirocycloalkyl” refers to a bicyclic or polycyclic group that shares a carbon atom (called a spiro atom) between the monocyclic rings. These may contain one or more double bonds, but none of the rings have fully conjugated π-electron system. “Fused cycloalkyl” refers to an all-carbon bi-cyclic or polycyclic group in which each ring share two neighboring carbon atoms with other ring(s), which may contain one or more double bonds, but none of the rings have a fully conjugated π-electron system “Bridge cycloalkyl” refers to an all-carbon polycyclic group in which two rings share two carbon atoms that are not directly bonded, which may contain one or more double bonds, but none of the rings have a fully conjugated π-electron system. The atoms contained in the cycloalkyl group are all carbon atoms. Some examples of cycloalkyl groups are as follows, and the present invention is not limited to the following cycloalkyl groups.

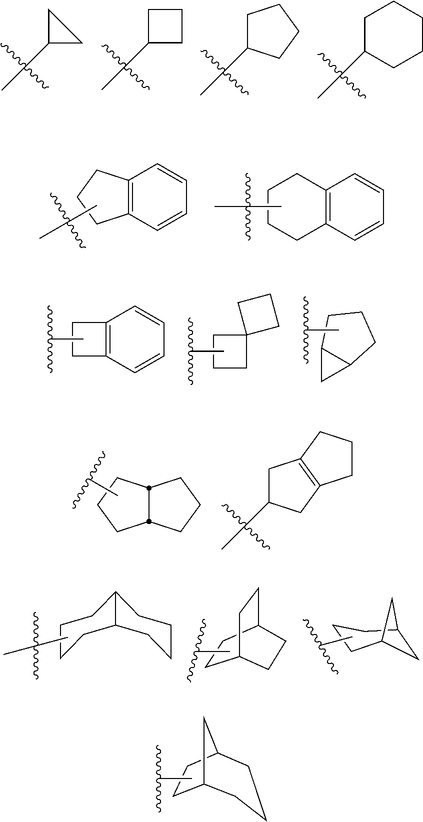

Unless otherwise stated, the following terms used in the instructions and claims have the following meanings. “Aryl” means an all-carbon monocyclic or fused polycyclic (ie, a ring that shares a pair of adjacent carbon atoms) groups having a conjugated π-electron system, such as phenyl and naphthyl. The aryl ring may be fused to other cyclic groups (including saturated and unsaturated rings), but may not contain heteroatoms such as nitrogen, oxygen, or sulfur, while the point of attachment to the parent must be on the carbon atoms of a ring in a conjugated π-electron system. The aryl group can be substituted or unsubstituted. The following are some examples of aryl groups, and the present invention is not limited to the aryl groups described below.

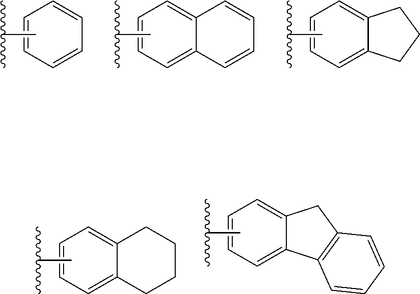

“Heteroaryl” refers to aromatic monocyclic or polycyclic groups containing one or more heteroatoms (optionally selected from nitrogen, oxygen, and sulfur), or refers to a polycyclic group consisting of a heterocyclic group (containing one or more heteroatoms optionally selected from nitrogen, oxygen, and sulfur) fused to an aryl group providing the attachment site is on the said aryl group. Heteroaryl groups can be optionally substituted or unsubstituted. The following are some examples of heteroaryl groups, and the present invention is not limited to the following heteroaryl groups described below.

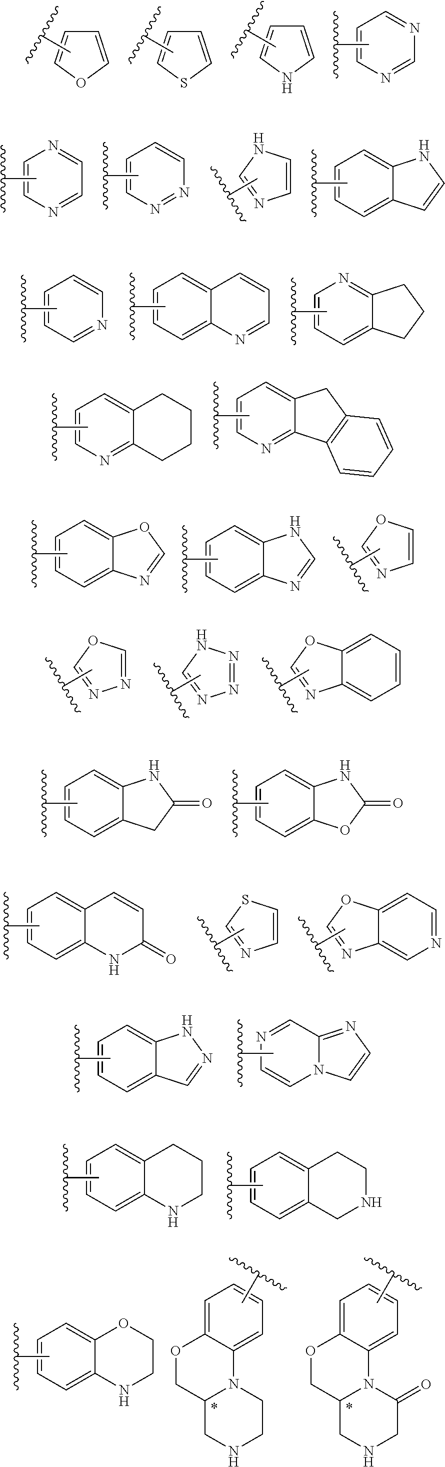

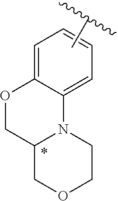

“Heterocyclyl” means a saturated or partially unsaturated monocyclic or polycyclic cyclic hydrocarbon substituent wherein one or more of the ring atoms are selected from nitrogen, oxygen or sulfur and the remaining ring atoms are carbon. Non-limiting examples of monocyclic heterocyclic groups include pyrrolidinyl, piperidinyl, piperazinyl, morpholinyl, thiomorpholinyl, homopiperazinyl. Polycyclic heterocyclic group refers to a heterocyclic group including a spiro ring, a fused ring, and a bridged ring. “Spirocyclic heterocyclyl” refers to a polycyclic heterocyclic group in which each ring of the system shares an atom (referred to as a spiro atom) with other rings in the system, wherein one or more of the ring atoms is selected from the group consisting of nitrogen, oxygen or sulfur, the remaining ring atoms are carbon. “Fused ring heterocyclyl” refers to a polycyclic heterocyclic group in which each ring of the system shares an adjacent pair of atoms with other rings in the system, and one or more rings may contain one or more double bonds, but none one ring has a fully conjugated π-electron system, and wherein one or more ring atoms are selected from nitrogen, oxygen or sulfur, and the remaining ring atoms are carbon. “Bridged heterocyclyl” refers to a polycyclic heterocyclic group in which any two rings share two atoms which are not directly bonded, these may contain one or more double bonds, but none of the rings have a fully conjugated π-electron system and wherein one or more of the ring atoms are selected from nitrogen, oxygen or sulfur, and the remaining ring atoms are carbon. If a heterocyclic group has both a saturated ring and an aromatic ring (for example, the saturated ring and the aromatic ring are fused together), the point attached to the parent must be on the saturated ring. Note: When the point attached to the parent is on the aromatic ring, it is called a heteroaryl group and is not called a heterocyclic group. Some examples of the heterocyclic group are as follows, and the present invention is not limited to the following heterocyclic group.

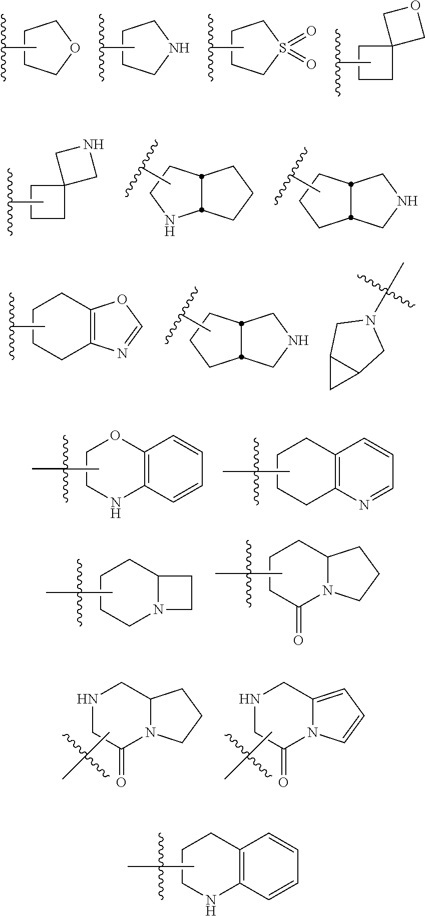

As used herein, the term “halogen”, when used alone or as part of another substituent, refers to F, Cl, Br, and I.

As used herein, the term “substituted” (when with or without “optionally”) means that one or more hydrogen atoms on a particular group are replaced by a particular substituent. Particular substituents are the substituents described above in the corresponding paragraphs, or the substituents which appear in the examples. Unless otherwise stated, an optionally substituted group may have a substituent selected from a particular group at any substitutable position of the group, and the substituents may be the same or different at each position. A cyclic substituent, such as a heterocyclic group, may be attached to another ring, such as a cycloalkyl group, to form a spirobicyclic ring system, i.e., the two rings have a common carbon atom. Those skilled in the art will appreciate that the combinations of substituents contemplated by the present invention are those that are stable or chemically achievable. The substituents are, for example but not limited to, C₁₋₈ alkyl, C₂₋₈ alkenyl, C₂₋₈ alkynyl, C₃₋₈ cycloalkyl, 3- to 12-membered heterocyclic, aryl, heteroaryl, halogen, hydroxy, carboxy (—COOH), C₁₋₈ aldehyde, C₂₋₁₀ acyl, C₂₋₁₀ ester group, amino.

For convenience and in accordance with conventional understanding, the term “optionally substituted” or “optionally substituted” applies only to sites which are capable of being substituted by a substituent, and does not include those which are not chemically achievable.

As used herein, unless otherwise specified, the term “pharmaceutically acceptable salt” refers to a salt that is suitable for contact with the tissue of a subject (eg, a human) without causing unpleasant side effects. In some embodiments, a pharmaceutically acceptable salt of a compound of the invention includes a salt (eg, a potassium salt, a sodium salt, a magnesium salt, a calcium salt) of a compound of the invention having an acidic group or is basic a salt of a compound of the invention (e.g., a sulfate, a hydrochloride, a phosphate, a nitrate, a carbonate).

Application:

The present invention provides a class of compounds of formula (I), or their deuterated derivatives, their salts, isomers (enantiomers or diastereomers, if they exist), prodrugs, hydrates, solvates, pharmaceutically acceptable carriers or excipients for inhibiting protein kinase. The protein kinases referred to here include EGFR, EGFR (C797S), ALK, and HPK1, but are not limited to the above kinases.

The compounds of the present invention can be used as one or more kinase inhibitors. For example, in some embodiments, certain types of compounds in the present invention can be used as EGFR and/or EGFR (C797S) and/or ALK and/or HPK1 kinase inhibitors Agent.

In cancer patients, the expression or activity of the various protein kinases mentioned above are significantly increased. These overexpression and/or abnormal protein kinase activity levels are directly related to the occurrence and development of tumors. The compounds of the invention are single and/or dual inhibitors of these protein kinases. By regulating the activity of these protein kinases, diseases can be prevented, alleviated or cured. The diseases referred to include liver cancer, rectal cancer, bladder cancer, throat cancer, non-small cell lung cancer, small cell lung cancer, lung adenocarcinoma, lung squamous cell carcinoma, breast cancer, prostate cancer, glioma, ovarian cancer, head and neck cancer Squamous cell carcinoma, cervical cancer, esophageal cancer, kidney cancer, pancreatic cancer, colon cancer, skin cancer, lymphoma, stomach cancer, multiple bone marrow cancer and solid tumors, etc.

From a certain perspective, dual protein kinase inhibitors interfere with two different kinases at the same time, and the anti-tumor effects produced are often additive, so they have the potential to treat various cancers more effectively.

The compounds of the present invention can be combined with biological agents such as PD-1 inhibitor Opdivo® and Keytruda® as a combination drug to treat various cancers and related diseases.

The compounds of the present invention and its deuterated derivatives, as well as pharmaceutically acceptable salts or isomers thereof (if present) or hydrates and/or compositions thereof can be combined with pharmaceutically acceptable excipients or The carrier is formulated together, and the resulting composition can be administered in vivo to mammals, such as men, women and animals, for the treatment of disorders, symptoms and diseases. The composition can be: tablets, pills, suspensions, solutions, emulsions, capsules, aerosols, sterile injections, sterile powder, etc. In some embodiments, pharmaceutically acceptable excipients include microcrystalline cellulose, lactose, sodium citrate, calcium carbonate, dibasic calcium phosphate, mannitol, hydroxypropyl-β-cyclodextrin, β-cyclodextrin (Increase), glycine, disintegrating agents (such as starch, croscarmellose sodium, composite silicate and macromolecular polyethylene glycol), granulation binders (such as polyvinylpyrrolidone, sucrose, gelatin and Gum arabic) and lubricants (such as magnesium stearate, glycerin and talc). In a preferred embodiment, the pharmaceutical composition is a dosage form suitable for oral administration, including but not limited to tablets, solutions, suspensions, capsules, granules, and powders. The amount of the compound or pharmaceutical composition administered to the patient is not fixed, and is usually administered in a pharmaceutically effective amount. At the same time, the amount of the compound actually administered can be determined by the physician according to the actual situation, including the disease to be treated, the route of administration selected, the actual compound administered, the individual condition of the patient, and so on. The dosage of the compound of the present invention depends on the specific use of the treatment, the mode of administration, the state of the patient, and the judgment of the physician. The ratio or concentration of the compound of the present invention in the pharmaceutical composition depends on a variety of factors, including dosage, physical and chemical properties, route of administration, and the like.

It should be understood that within the scope of the present invention, the above-mentioned technical features of the present invention and the technical features specifically described in the following (such as the embodiments) can be combined with each other to form a new or preferred technical solution.

General Synthetic Schemes for the Compounds in this Invention

The compound of formula I of the present invention can be prepared by the following method:

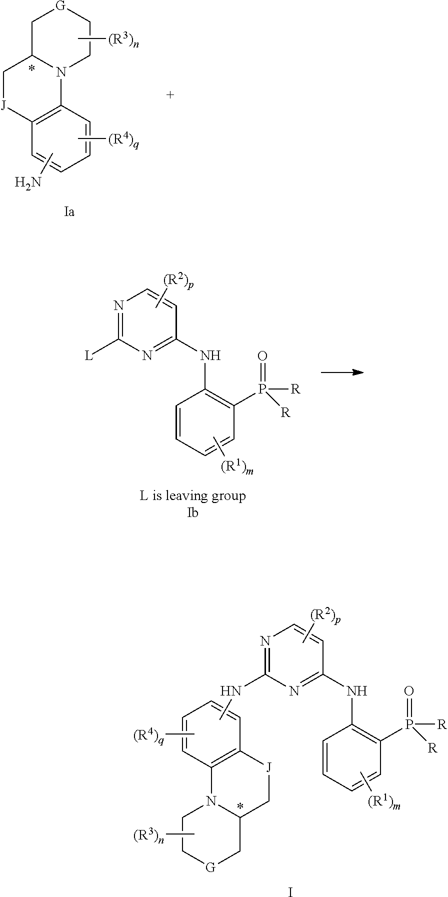

In an inert solvent, the compound (Ia) is reacted with the compound (Ib) to obtain the compound (I).

In the above formulas, the definition of each group is as described above. The reagents and conditions of each step can be selected from the conventional reagents or conditions of this type of preparation method in the art. After the structure of the compound of the present invention is disclosed, the above selection can be made by those skilled in the art according to the knowledge in the field.

More specifically, the compound represented by the general formula I of the present invention can be prepared by the following method, but the conditions of the method, such as reactants, solvent, base, amount of compound used, reaction temperature, reaction time required, etc. are not limited to the following explanation of. The compounds of the present invention can also be conveniently prepared by combining various synthetic methods described in this specification or known in the art, and such combinations can be easily performed by those skilled in the art to which the present invention belongs.

In the preparation method of the present invention, each reaction is usually carried out in an inert solvent, and the reaction temperature is usually −20 to 150° C. (preferably 0 to 120° C.). The reaction time of each step is usually 0.5 to 48 h, preferably 2-12 h.

Scheme 1 illustrates a general synthesis of intermediate 1-A5-1 and 1-A5-2:

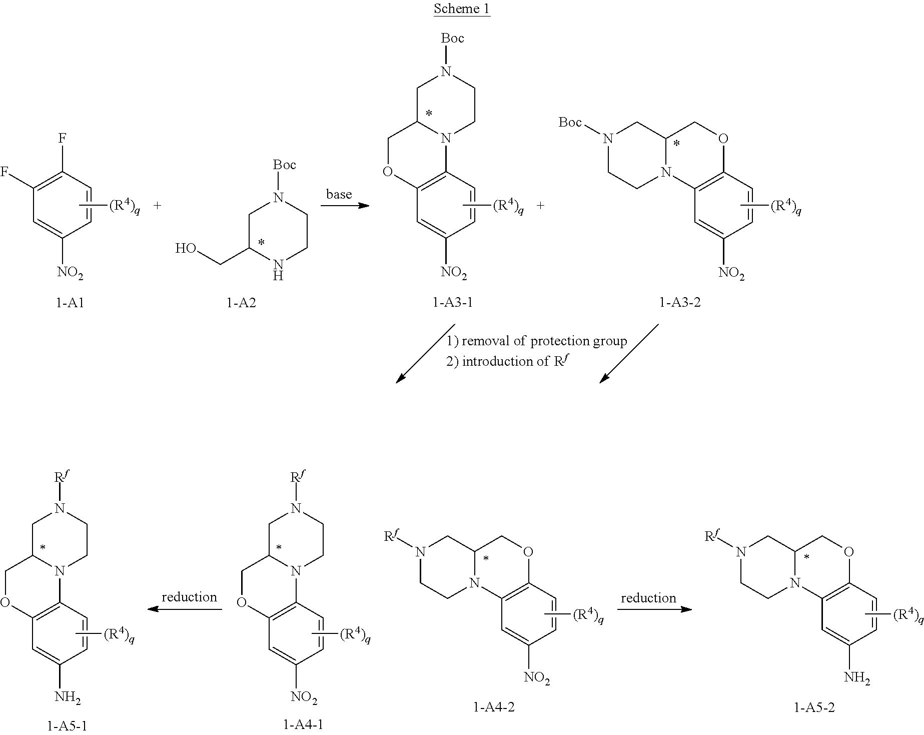

Scheme 2 illustrates a general synthesis of intermediates 2-B3-1 and 2-B3-2:

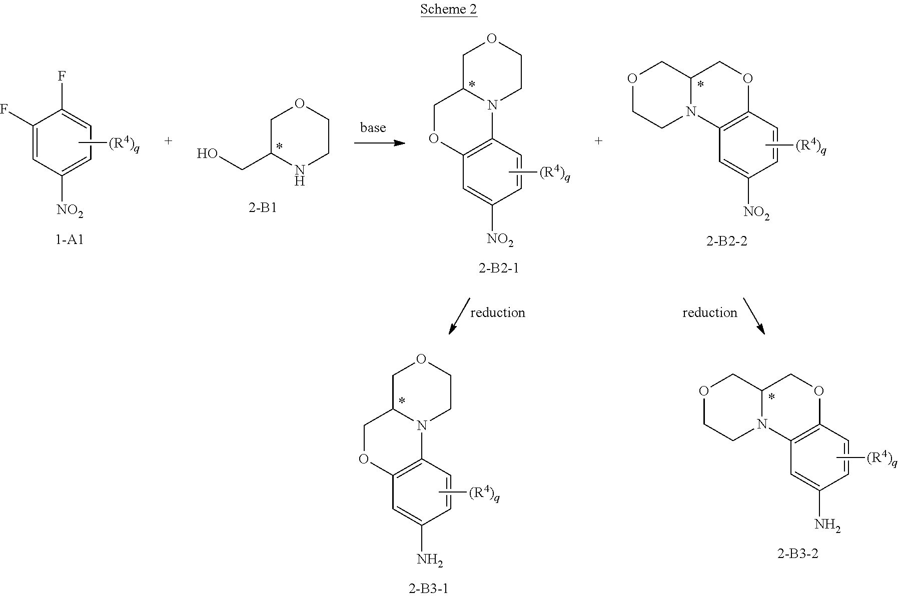

Scheme 3 illustrates a general synthesis of intermediate 3-C7:

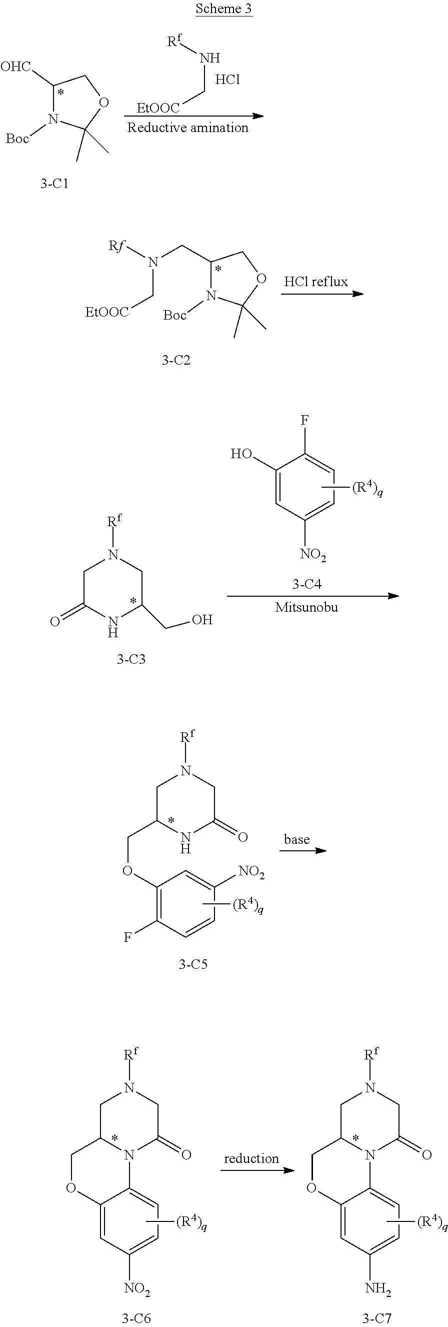

Compound IIIa is a part of compound I. Scheme 4 illustrates a general synthesis of compound IIIa:

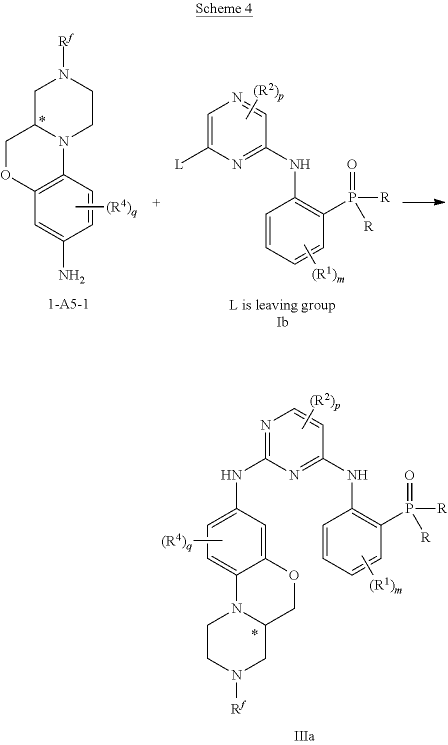

Compound IIIb is a part of compound I. Scheme 5 illustrates a general synthesis of compound IIIb:

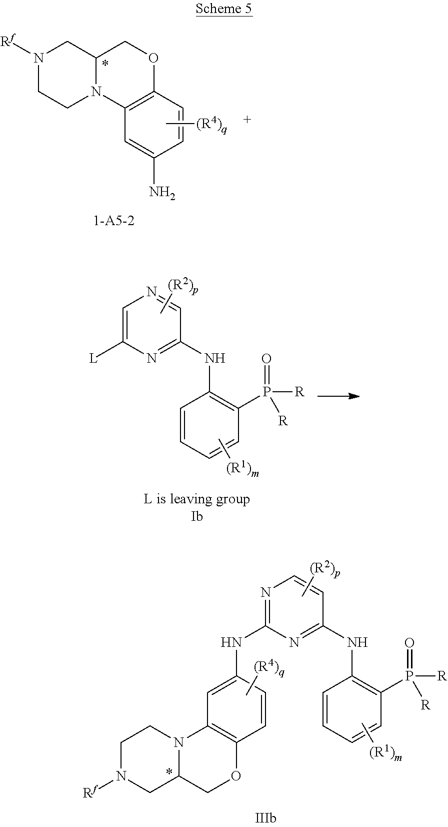

Compound IIIc is a part of compound I. Scheme 6 illustrates a general synthesis of compound IIIc:

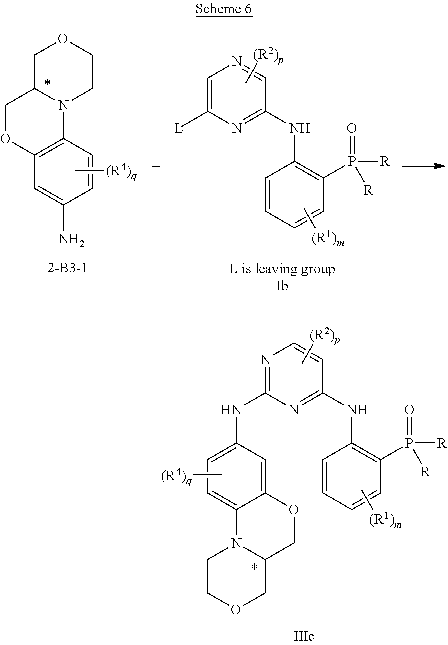

Compound IIId is a part of compound I. Scheme 7 illustrates a general synthesis of compound IIId:

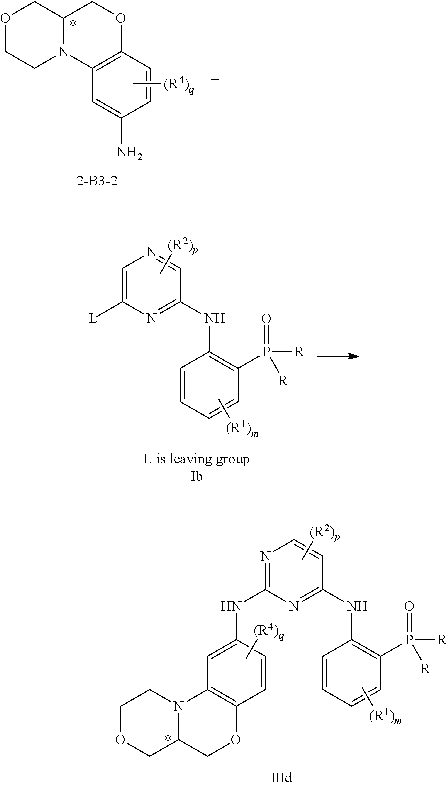

Compound IIIe is a part of compound I. Scheme 8 illustrates a general synthesis of compound IIIe:

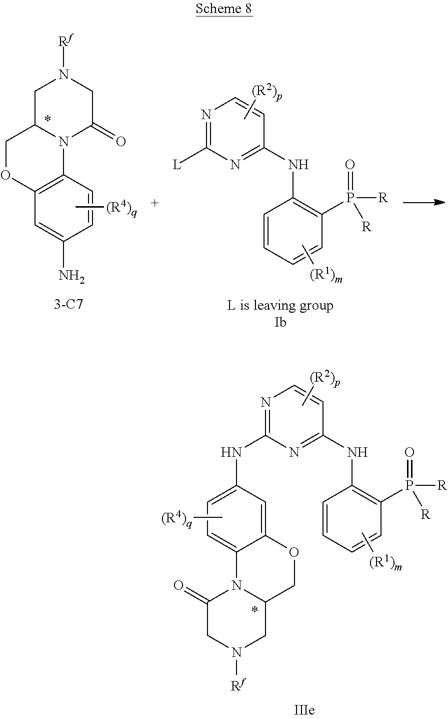

Compound IIIf and IIIg is a part of compound I. Scheme 9 illustrates a general synthesis of compound IIIf and IIIg:

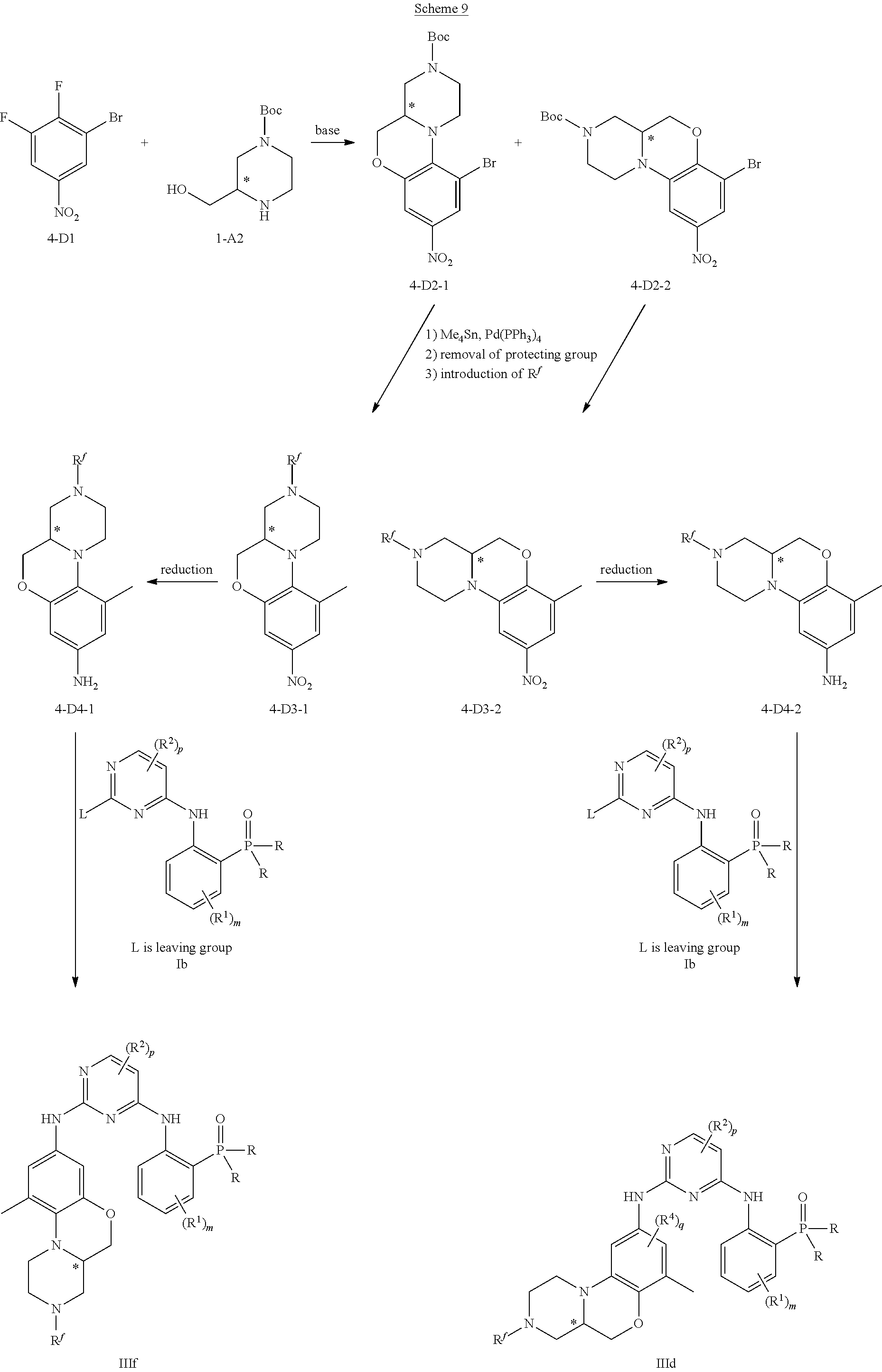

Compounds IIIh and IIIi are part of compound L. Scheme 10 illustrates a general synthesis of compounds IIIh and IIIi:

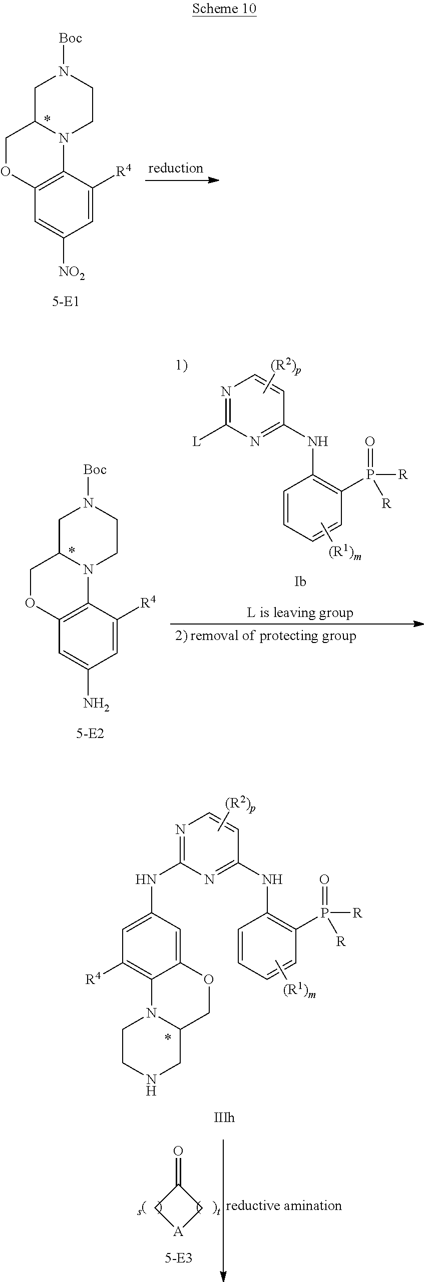

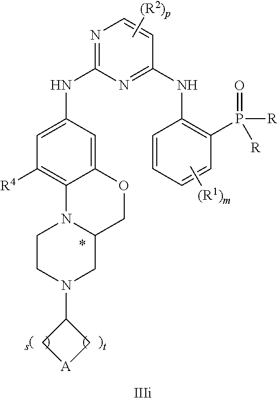

The definitions of R, R¹, R², R⁴, R^{f}, m, p, q, s, t, and A in the above schemes 1-10 are the same as those in Claim 1.

Pharmaceutical Composition and Method of Administration

Since the compounds of the present invention have excellent inhibitory activity against a series of protein kinases, the compounds of the present invention and their various crystal forms, pharmaceutically acceptable inorganic or organic salts, hydrates or solvates, and the pharmaceutical composition of the main active ingredients can be used to treat, prevent and alleviate diseases related to the activity or expression of protein kinases such as EGFR, EGFR (C797S), ALK, and HPK1.

The pharmaceutical compositions of the present invention comprise a safe or effective amount of a compound of the present invention, or a pharmaceutically acceptable salt thereof, and a pharmaceutically acceptable excipient or carrier. By “safe and effective amount” it means that the amount of the compound is sufficient to significantly improve the condition without causing serious side effects. In general, the pharmaceutical compositions contain from 1 to 2000 mg of the compound of the invention per agent, more preferably from 5 to 200 mg of the compound of the invention per agent. Preferably, the “one dose” is a capsule or tablet.

“Pharmaceutically acceptable carrier” means: one or more compatible solid or liquid fillers or gel materials which are suitable for human use and which must be of sufficient purity and of sufficiently low toxicity. By “compatibility” it is meant herein that the components of the composition are capable of intermingling with the compounds of the invention and with each other without significantly reducing the efficacy of the compound. Examples of pharmaceutically acceptable carriers are cellulose and its derivatives (such as sodium carboxymethylcellulose, sodium ethylcellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerin, mannitol, sorbitol, etc.), emulsifiers (such as Tween®), run Wet agents (such as sodium lauryl sulfate), colorants, flavoring agents, stabilizers, antioxidants, preservatives, pyrogen-free water, and the like.

The mode of administration of the compound or pharmaceutical composition of the present invention is not particularly limited, and representative modes of administration include, but are not limited to, oral, intratumoral, rectal, parenteral (intravenous, intramuscular or subcutaneous), and topical administration.

Solid dosage forms for oral administration include capsules, tablets, pills, powders, and granules. In these solid dosage forms, the active compound is mixed with at least one conventional inert excipient (or carrier), such as sodium citrate or dicalcium phosphate, or mixed with: (a) a filler or compatibilizer, for example, starch, lactose, sucrose, glucose, mannitol and silicic acid; (b) binders such as hydroxymethylcellulose, alginates, gelatin, polyvinylpyrrolidone, sucrose and acacia; (c) humectants, for example, glycerin; (d) a disintegrant such as agar, calcium carbonate, potato starch or tapioca starch, alginic acid, certain complex silicates, and sodium carbonate; (e) a slow solvent such as paraffin; (f) absorbing accelerators, for example, quaternary amine compounds; (g) wetting agents, such as cetyl alcohol and glyceryl monostearate; (h) adsorbents, for example, kaolin; and (i) lubricants, for example, talc, calcium stearate, magnesium stearate, solid polyethylene glycol, sodium lauryl sulfate, or a mixture thereof. In capsules, tablets and pills, the dosage form may also contain a buffer.

Solid dosage forms such as tablets, dragees, capsules, pills, and granules can be prepared with coatings and shells such as enteric coatings and other materials known in the art. They may contain opacifying agents and the release of the active compound or compound in such compositions may be released in a portion of the digestive tract in a delayed manner. Examples of embedding components that can be employed are polymeric and waxy materials. If necessary, the active compound may also be in microencapsulated form with one or more of the above-mentioned excipients.

Liquid dosage forms for oral administration include pharmaceutically acceptable emulsions, solutions, suspensions, syrups or elixirs. In addition to the active compound, the liquid dosage form may contain inert diluents conventionally employed in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil or a mixture of these substances.

In addition to these inert diluents, the compositions may contain adjuvants such as wetting agents, emulsifying and suspending agents, sweetening agents, flavoring agents and perfumes.

In addition to the active compound, the suspension may contain suspending agents, for example, ethoxylated isostearyl alcohol, polyoxyethylene sorbitol and sorbitan esters, microcrystalline cellulose, aluminum methoxide and agar or mixtures of these and the like.

Compositions for parenteral injection may comprise a physiologically acceptable sterile aqueous or nonaqueous solution, dispersion, suspension or emulsion, and a sterile powder for reconstitution into a sterile injectable solution or dispersion. Suitable aqueous and nonaqueous vehicles, diluents, solvents or vehicles include water, ethanol, polyols, and suitable mixtures thereof.

Dosage forms for the compounds of the invention for topical administration include ointments, powders, patches, propellants and inhalants. The active ingredient is admixed under sterile conditions with a physiologically acceptable carrier and any preservatives, buffers, or, if necessary, propellants.

The compounds of the invention may be administered alone or in combination with other pharmaceutically acceptable compounds.

When a pharmaceutical composition is used, a safe and effective amount of a compound of the invention is administered to a mammal (e.g., a human) in need of treatment wherein the dosage is a pharmaceutically effective effective dosage, for a 60 kg body weight, The daily dose is usually from 1 to 2000 mg, preferably from 5 to 500 mg. Of course, specific doses should also consider factors such as the route of administration, the health of the patient, etc., which are within the skill of the skilled physician.

The main advantages of the invention include:

1. Provided a compound of formula (I).

2. Provided a novel structure of EGFR, EGFR (C797S), ALK, and HPK1 inhibitors, and their preparation and applications. The said inhibitors can inhibit protein kinases in very low concentration.

3. A class of pharmaceutical compositions for treating diseases associated with EGFR, EGFR (C797S), ALK and HPK1 activity is provided.

The invention is further illustrated below in conjunction with specific embodiments. It is to be understood that the examples are not intended to limit the scope of the invention. The experimental methods in the following examples which do not specify the specific conditions are usually in accordance with conventional conditions or according to the conditions recommended by the manufacturer. Percentages and parts are by weight unless otherwise stated.

### Example 1: Preparation of Compound 1

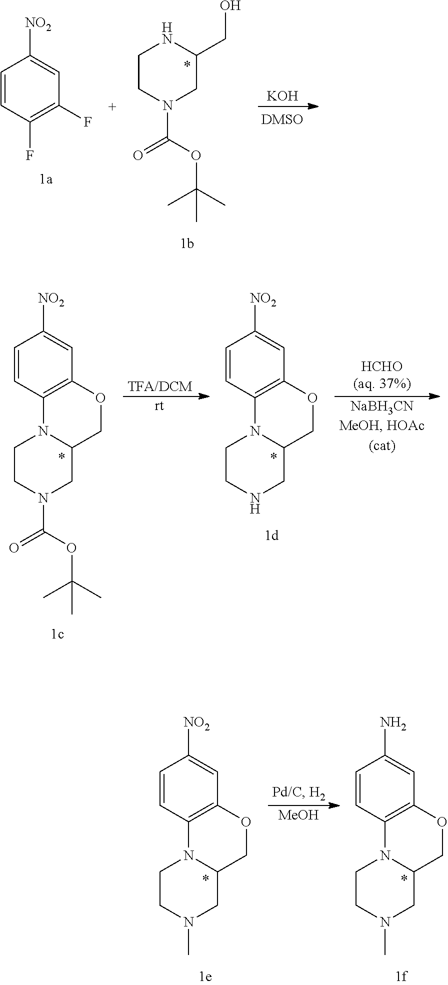

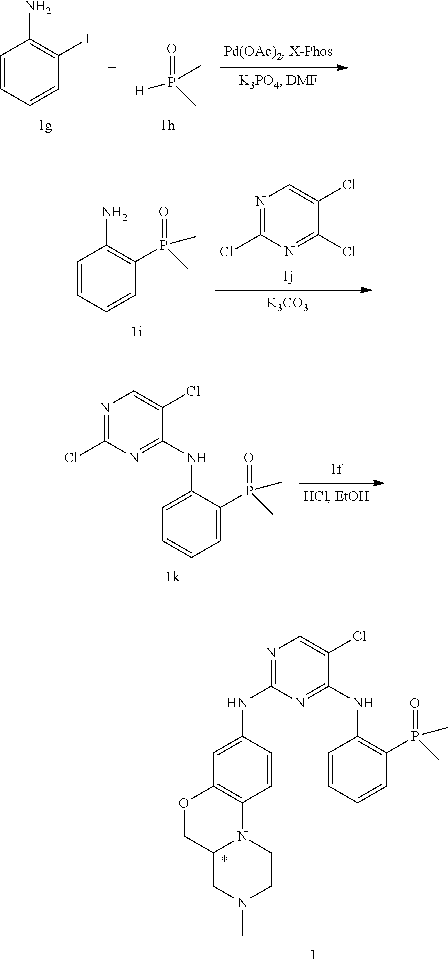

To a solution of 1,2-difluoro-4-nitrobenzene (1a, 1.11 g, 7.0 mmoL) and racemic tert-butyl 3-(hydroxymethyl)piperazine-1-carboxylate (1b, 1.30 g, 6.0 mmoL) in dry DMSO (15 mL) was added KOH (1.01 g, 18.0 mmoL). The reaction mixture was stirred at room temperature for 3 hours, and then it was heated to 60° C. for 8 hours. Ice water was added into the reaction mixture. The precipitate was collected by filtration, washed with water, and purified by column chromatography (PE:EtOAc=6:1) to afford compound 1c (1.41 g, yield 70.0%) as a yellow solid. ¹H NMR (600 MHz, CDCl₃): δ 7.79 (dd, J=9.1 Hz, 2.6 Hz, 1H), 7.66 (d, J=2.6 Hz, 1H), 6.76 (d, J=9.2 Hz, 1H), 4.30 (dd, J=11.0 Hz, 3.0 Hz, 1H), 4.18-4.09 (m, 2H), 3.99 (dd, J=11.1 Hz, 8.0 Hz, 1H), 3.79 (d, J=11.0 Hz, 1H), 3.35-3.31 (m, 1H), 3.04-2.93 (m, 2H), 2.67 (br, 1H), 1.49 (s, 9H); HRMS(+): m/z 358.1370 [M+Na]⁺. C₁₆H₂₁N₃O₅Na⁺ calcd: 358.1373.

To a solution of compound 1c (2.0 g, 6.0 mmol) in CH₂Cl₂ (20 mL) was added TFA (5 mL) at room temperature. The reaction mixture was stirred at room temperature for 1 hour. The reaction was monitored by TLC for completion. It was concentrated under reduced pressure to removed TFA. The residue was dissolved in CH₂Cl₂ (30 mL), and neutralized with 1 M NaHCO₃ aqueous solution to pH=9˜10. The aqueous phase was separated, and extracted with CH₂Cl₂ (2 times). The combined organic layers were washed with brine (30 mL), dried over anhydrous sodium sulfate, filtered, and concentrated under reduced pressure to afford compound 1d (1.2 g) as a yellow solid. MS m/z 236.1 [M+H]⁺.

To a solution of compound 1d (1.2 g) in MeOH (20 mL) was added 37% formaldehyde aqueous solution (6 mL) and acetic acid (2 drops). The reaction mixture was stirred at room temperature for 30 minutes. Sodium cyanoborohydride (0.8 g, 12.7 mmol) was added, and the reaction mixture was stirred at room temperature for 3 hours. The reaction was monitored by TLC for completion. It was concentrated under reduced pressure, and the residue was purified by silica gel column chromatography (CH₂Cl₂:MeOH=60:1) to afford compound 1e (1.0 g) as a yellow solid. MS m/z 250.2 [M+H]⁺.

Compound 1e (145 mg, 0.58 mmol) and 10% Pd—C (15 mg) were added to MeOH (3 mL) at room temperature. The reaction mixture was stirred under 1 atmospheric pressure of H₂ at room temperature for 1 hour. The reaction was monitored by TLC for completion. It was filtered through celite, and the filtrate was concentrated under reduce pressure to afford compound if (100 mg) as a brown solid which was used in next step. MS m/z 220.2 [M+H]⁺.

Compound 1g (3.0 g, 14 mmol) and compound 1h (1.2 g, 15 mmol) were dissolved in DMF (60 mL). Pd(OAc)₂ (0.2 g), X-Phos (0.3 g), and K₃PO₄ (5.9 g, 28 mmol) were added. The reaction was purged with nitrogen (3 times), and then heated at 120° C. under atmosphere of nitrogen until the reaction was completed. It was concentrated under reduce pressure. The residue was purified by silica gel column chromatography (CH₂Cl₂:MeOH=1:40) to afford compound 1i (0.75 g, yield 33%) as a white solid.

To a solution of compound 1i (750 mg, 4.4 mmol) and compound 1j (812 mg, 4.4 mmol) in IPA (20 mL) was added K₂CO₃ (1.2 g, 8.8 mmol). The resulted mixture was heated at 105° C. by microwave for 1 hour. It was concentrated under reduce pressure. The residue was purified by silica gel column chromatography (PE:EtOAc=1:1) to afford compound 1k (450 mg, yield 32%) as a white solid. Liquid Chromatography condition: XBrdige C18 column: 4.6 mm*30 mm*3.5 um, Mobile A: water (0.01 mol/L NH₄HCO₃), Mobile B: acetonitrile, flow rate 2.0 mL/min, from 10% of B to 95% of B within 0.5 minute, 95% of B for 1.5 minute. Purity: 95%, retention time: 0.95 min; MS m/z 316.0 [M+H]⁺.

A solution of compound 1f (22 mg, 0.1 mmol) and compound 1k (31.5 mg, 0.1 mmol) in 2 M HCl in EtOH (2 mL) was heated at 80° C. until the reaction was completed. The reaction mixture was concentrated under reduce pressure. The residue was purified by prep-HPLC to afford compound 1 (5.6 mg, yield 11%) as a light yellow solid. Liquid Chromatography condition: XBrdige C18 column: 4.6 mm*30 mm*3.5 um, Mobile A: water (0.01 mol/L NH₄HCO₃), Mobile B: acetonitrile, flow rate 2.0 mL/min, from 5% of B to 100% of B within 1.6 minute, 100% of B for 1.4 minute. Purity: 90%. Retention time: 1.56 min. ¹H NMR (400 MHz, CD₃OD) δ 8.48-8.52 (m, 1H), 8.03 (s, 1H), 7.53-7.61 (m, 2H), 7.24-7.26 (m, 1H), 7.03-7.04 (m, 1H), 6.89-6.91 (m, 1H), 6.75-6.77 (m, 1H), 4.21-4.24 (m, 1H), 3.94-3.99 (m, 1H), 3.72-3.75 (m, 1H), 3.08-3.09 (m, 1H), 2.97-3.00 (m, 1H), 2.87-2.90 (m, 1H), 2.72-2.73 (m, 1H), 2.38 (s, 3H), 2.29-2.36 (m, 1H), 1.87-1.92 (m, 1H), 1.86 (s, 3H), 1.84 (s, 3H). MS m/z 499.2 [M+H]⁺.

### Example 2: Preparation of Compound 1R

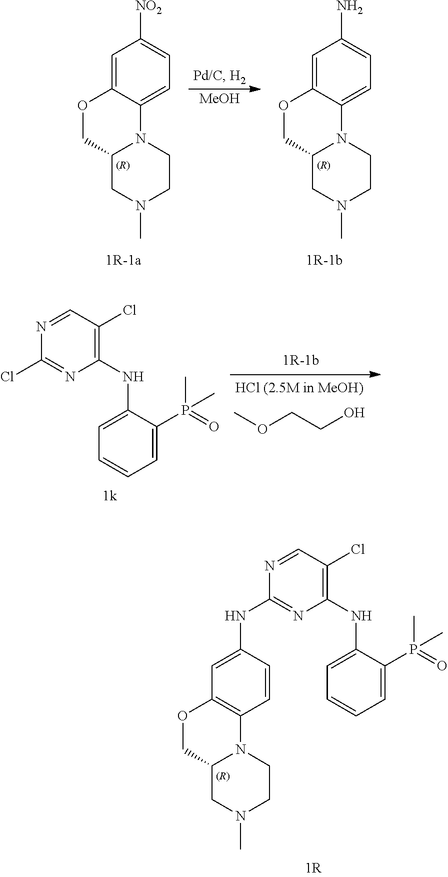

Compound 1R-1a (650 mg, 2.60 mmol) and 10% Pd—C (65 mg) were added to MeOH (15 mL) at room temperature. The reaction was purged with hydrogen (3 times). The reaction mixture was stirred at room temperature under 1 atmospheric pressure of H₂ overnight. The reaction was monitored by TLC for completion. It was filtered through celite. The filtrate was concentrated under reduce pressure to afford a brown crude which was purified by silica gel column chromatography (CH₂Cl₂:MeOH=40:1 to 20:1) to afford compound 1R-1b (180 mg, yield 32%) as a yellow oil. ¹H NMR (500 MHz, CDCl₃) δ 6.62 (d, J=8.4 Hz, 1H), 6.24 (dd, J=8.4, 2.6 Hz, 1H), 6.21 (d, J=2.5 Hz, 1H), 4.14 (dd, J=10.5, 2.6 Hz, 1H), 4.03-3.95 (m, 1H), 3.56 (dt, J=11.5, 2.7 Hz, 1H), 3.36 (s, br., 2H), 3.12-3.03 (m, 1H), 2.91 (dd, J=11.3, 2.2 Hz, 1H), 2.81-2.75 (m, 1H), 2.72 (td, J=11.7, 3.1 Hz, 1H), 2.33 (s, 3H), 2.25 (td, J=11.5, 3.2 Hz, 1H), 1.83 (t, J=10.6 Hz, 1H).

Compound 1R-1b (70 mg, 0.32 mmol) and compound 1k (72 mg, 0.23 mmol) were dissolved in 2-methoxyethanol (2 mL) followed by the addition of 2.5 M HCl in MeOH (0.24 mL). The reaction mixture in a sealed tube was heated to 120° C., and stirred overnight. The reaction was monitored by TLC for completion. It was cooled to room temperature, and concentrated under reduce pressure. The residue was purified by silica gel column chromatography (CH₂Cl₂:MeOH=40:1 to 20:1) to afford compound 1R (79 mg, yield 50%) as a white solid. ¹H NMR (500 MHz, CDCl₃) δ 10.88 (s, 1H), 8.65 (dd, J=8.4, 4.3 Hz, 1H), 8.06 (s, 1H), 7.48 (dd, J=8.4, 7.4 Hz, 1H), 7.29-7.22 (m, 1H), 7.11-7.06 (m, 2H), 6.92 (dd, J=8.6, 2.5 Hz, 1H), 6.74-6.70 (m, 2H), 4.20 (dd, J=10.5, 2.7 Hz, 1H), 4.03 (dd, J=10.4, 9.1 Hz, 1H), 3.67-3.61 (m, 1H), 3.22-3.15 (m, 1H), 2.94 (d, J=10.9 Hz, 1H), 2.84-2.77 (m, 2H), 2.36 (s, 3H), 2.26 (td, J=11.6, 3.2 Hz, 1H), 1.87 (d, J=10.7 Hz, 1H), 1.84 (s, 3H), 1.81 (s, 3H). MS m/z 499.4 [M+H]⁺.

### Example 3: Preparation of Compound 1S

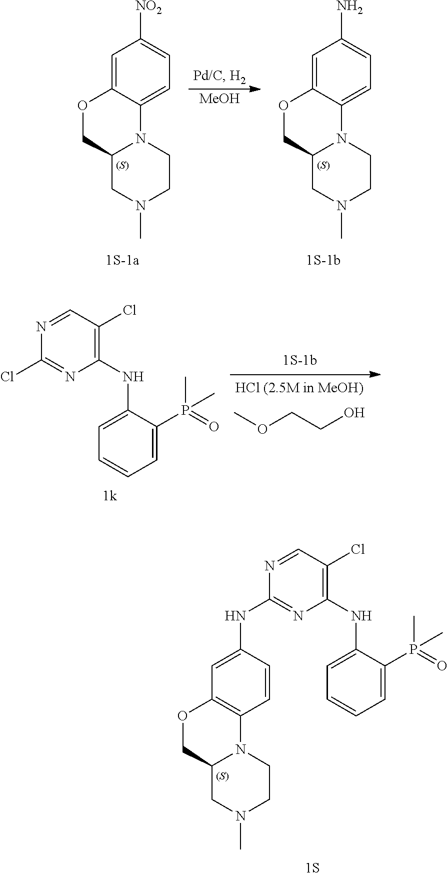

Compound 1S-1a (800 mg, 3.20 mmol) and 10% Pd—C (80 mg) were added to MeOH (10 mL) at room temperature. The reaction was purged with hydrogen (3 times). The reaction mixture was stirred at room temperature under 1 atmospheric pressure of H₂ overnight. The reaction was monitored by TLC for completion. It was filtered through celite, and the filtrate was concentrated under reduce pressure. The resulted brown residue was purified by silica gel column chromatography (CH₂Cl₂:MeOH=40:1 to 20:1) to afford compound 1S-1b (400 mg, yield 57%) as a yellow oil. ¹H NMR (500 MHz, DMSO-d₆) δ 6.54 (d, J=8.4 Hz, 1H), 6.06 (d, J=8.5 Hz, 1H), 6.01 (s, 1H), 4.47 (s, br., 2H), 4.10 (d, J=10.4 Hz, 1H), 3.80 (t, J=9.8 Hz, 1H), 3.49 (d, J=11.4 Hz, 1H), 2.82 (t, J=9.5 Hz, 2H), 2.72 (d, J=10.6 Hz, 1H), 2.48-2.40 (m, 1H), 2.19 (s, 3H), 2.07 (td, J=11.0, 1.9 Hz, 1H), 1.66 (t, J=10.5 Hz, 1H).

Compound 1S-1b (70 mg, 0.32 mmol) and compound 1k (72 mg, 0.23 mmol) were dissolved in 2-methoxyethanol (2 mL) followed by the addition of 2.5 M HCl in MeOH (0.24 mL). The reaction mixture in a sealed tube was heated to 120° C., and stirred overnight. The reaction was monitored by TLC for completion. It was cooled to room temperature, and concentrated under reduce pressure. The residue was purified by silica gel column chromatography (CH₂Cl₂:MeOH=40:1 to 20:1) to afford compound 1S (44 mg, yield 38%) as a white solid. ¹H NMR (500 MHz, CDCl₃) δ 10.88 (s, 1H), 8.65 (dd, J=8.3, 4.4 Hz, 1H), 8.05 (s, 1H), 7.48 (dd, J=8.4, 7.4 Hz, 1H), 7.28-7.22 (m, 1H), 7.11-7.06 (m, 2H), 6.92 (dd, J=8.7, 2.5 Hz, 1H), 6.79-6.72 (m, 2H), 4.20 (dd, J=10.5, 2.6 Hz, 1H), 4.03 (dd, J=10.4, 9.0 Hz, 1H), 3.67-3.61 (m, 1H), 3.22-3.15 (m, 1H), 2.95 (d, J=10.9 Hz, 1H), 2.85-2.78 (m, 2H), 2.36 (s, 3H), 2.27 (td, J=11.5, 3.2 Hz, 1H), 1.87 (d, J=10.7 Hz, 1H), 1.83 (s, 3H), 1.81 (s, 3H). MS m/z 499.4 [M+H]⁺.

### Example 4: Preparation of Compound 2R

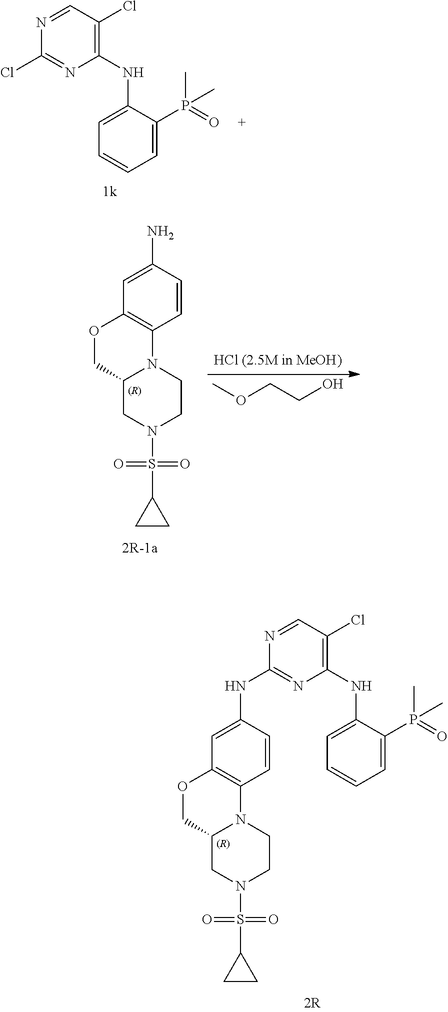

Compound 2R-1a (63 mg, 0.20 mmol) and compound 1k (46 mg, 0.14 mmol) were dissolved in 2-methoxyethanol (2 mL) followed by the addition of 2.5 M HCl in MeOH (0.15 mL). The reaction mixture in a sealed tube was heated to 120° C., and stirred overnight. The reaction was monitored by TLC for completion. It was cooled to room temperature, and concentrated under reduce pressure. The residue was purified by silica gel column chromatography (CH₂Cl₂:MeOH=40:1 to 20:1) to afford compound 2R (21 mg, yield 25%) as a white solid. ¹H NMR (500 MHz, CDCl₃) δ 10.91 (s, 1H), 8.64 (dd, J=8.3, 4.4 Hz, 1H), 8.06 (s, 1H), 7.48 (dd, J=8.4, 7.4 Hz, 1H), 7.30-7.23 (m, 1H), 7.16 (d, J=2.5 Hz, 1H), 7.13-7.09 (m, 1H), 6.95 (dd, J=8.7, 2.5 Hz, 1H), 6.80 (s, 1H), 6.75 (d, J=8.8 Hz, 1H), 4.26 (dd, J=10.7, 2.7 Hz, 1H), 4.04 (dd, J=10.7, 8.2 Hz, 1H), 3.86-3.77 (m, 2H), 3.75-3.70 (m, 1H), 3.28-3.22 (m, 1H), 3.10 (td, J=11.6, 2.8 Hz, 1H), 2.90 (td, J=11.9, 3.1 Hz, 1H), 2.75 (t, J=11.0 Hz, 1H), 2.30-2.23 (m, 1H), 1.84 (s, 3H), 1.82 (s, 3H), 1.23-1.68 (m, 2H), 1.05-1.00 (m, 2H). MS m/z 589.4 [M+H]⁺.

### Example 5: Preparation of Compound 3R

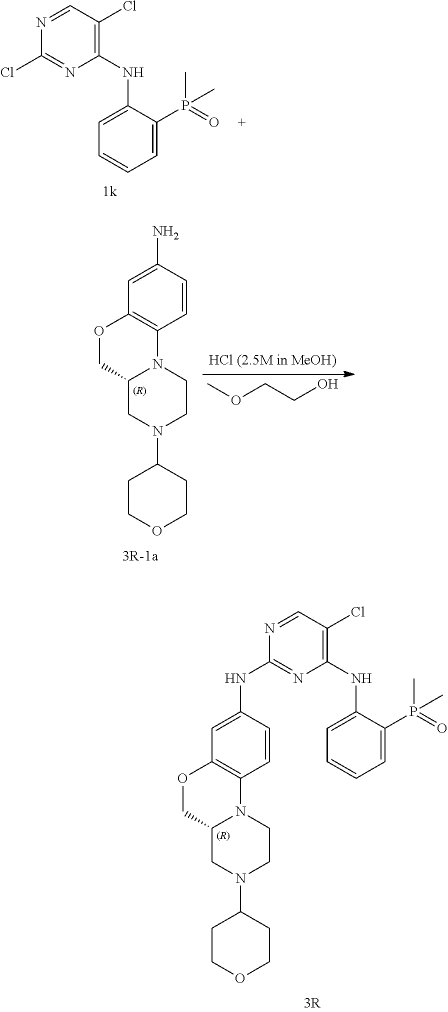

Compound 3R-1a (86 mg, 0.30 mmol) and compound 1k (67 mg, 0.20 mmol) were dissolved in 2-methoxyethanol (2 mL) followed by the addition of 2.5 M HCl in MeOH (0.22 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=20:1) to afford compound 3R (70 mg, yield 62%) as a white solid. ¹H NMR (500 MHz, CDCl₃) δ 10.89 (s, 1H), 8.66 (dd, J=8.2, 4.4 Hz, 1H), 8.05 (s, 1H), 7.48 (dd, J=8.4, 7.4 Hz, 1H), 7.28-7.22 (m, 1H), 7.11-7.06 (m, 2H), 6.92 (dd, J=8.7, 2.5 Hz, 1H), 6.75-6.70 (m, 2H), 4.20 (dd, J=10.4, 2.7 Hz, 1H), 4.09-3.98 (m, 3H), 3.67 (d, J=11.5 Hz, 1H), 3.39 (t, J=11.4 Hz, 2H), 3.17-3.10 (m, 1H), 3.06 (d, J=11.0, 1H), 2.91 (d, J=10.4 Hz, 1H), 2.77 (td, J=11.5, 2.9 Hz, 1H), 2.53-2.41 (m, 2H), 2.03 (t, J=10.5 Hz, 1H), 1.84 (s, 3H), 1.81 (s, 3H), 1.80-1.76 (m, 4H). MS m/z 569.6 [M+H]⁺.

### Example 6: Preparation of Compound 4R

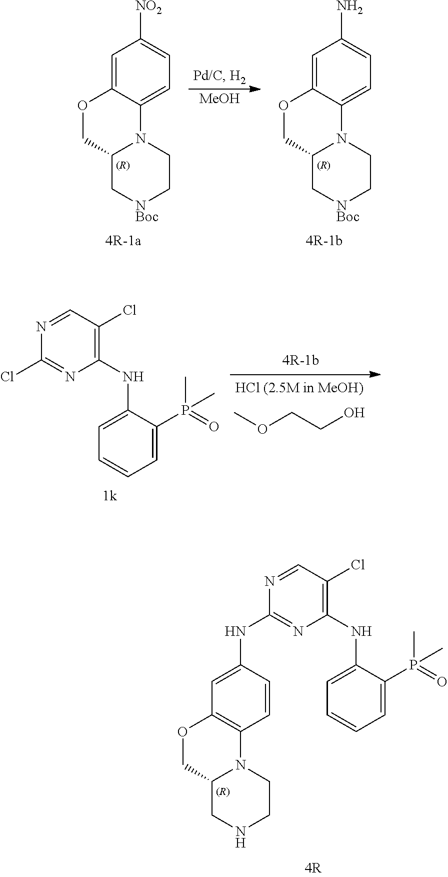

Compound 4R-1a (3.0 g, 8.94 mmol) and 10% Pd—C (250 mg) were added to MeOH (35 mL) at room temperature. The reaction was purged with hydrogen (3 times). The reaction mixture was stirred at room temperature under 1 atmospheric pressure of H₂ overnight. The reaction was monitored by TLC for completion. It was filtered through celite, and the filtrate was concentrated under reduce pressure. The brown residue was purified by silica gel column chromatography (PE:EtOAc=4:1 to 1:1) to afford compound 4R-1b (2.48 g, yield 91%) as a black purple solid. ¹H NMR (500 MHz, CDCl₃) δ 6.63 (d, J=8.5 Hz, 1H), 6.25 (dd, J=8.5, 2.5 Hz, 1H), 6.21 (d, J=2.5 Hz, 1H), 4.17 (dd, J=10.6, 2.6 Hz, 1H), 4.14-4.00 (m, 2H), 3.96 (dd, J=10.5, 9.1 Hz, 1H), 3.56 (d, J=11.0 Hz, 1H), 3.38 (s, 2H), 3.04 (s, 1H), 2.95 (ddd, J=11.7, 5.5, 2.7 Hz, 1H), 2.59 (td, J=11.8, 3.1 Hz, 2H), 1.47 (s, 9H).

Compound 4R-1b (100 mg, 0.33 mmol) and compound 1k (74 mg, 0.24 mmol) were dissolved in 2-methoxyethanol (2 mL) followed by the addition of 2.5 M HCl in MeOH (2.5 M, 0.25 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:acetone=1:1, 2% of Et₃N) to afford compound 4R (6 mg, yield 5%) as a white solid. ¹H NMR (500 MHz, CD₃OD) δ 8.49 (dd, J=8.2, 4.6 Hz, 1H), 8.03 (s, 1H), 7.63-7.52 (m, 2H), 7.26-7.22 (m, 1H), 7.08 (d, J=2.4 Hz, 1H), 6.93 (dd, J=8.7, 2.5 Hz, 1H), 6.80 (d, J=8.9 Hz, 1H), 4.26 (dd, J=10.7, 2.5 Hz, 1H), 4.00 (dd, J=10.8, 8.2 Hz, 1H), 3.90 (d, J=12.7 Hz, 1H), 3.27-3.16 (m, 3H), 3.11 (td, J=12.5, 3.5 Hz, 1H), 2.81 (td, J=12.6, 3.1 Hz, 1H), 2.75 (t, J=11.4 Hz, 1H), 1.86 (s, 3H), 1.83 (s, 3H). MS m/z 485.4 [M+H]⁺.

### Example 7: Preparation of Compound 4S

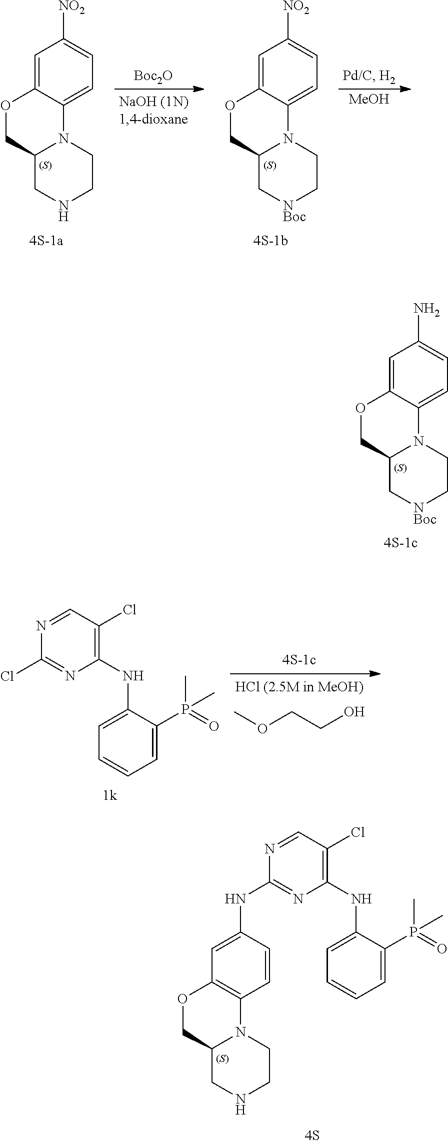

To a stirred solution of compound 4S-1a (500 mg, 2.10 mmol) in 1,4-dioxane (8 mL) in ice bath was added 1 N NaOH aqueous solution (2 mL) followed by Boc₂O (510 mg, 2.30 mmol). The reaction mixture was warmed to room temperature, and stirred for 30 minutes. The reaction was monitored by TLC for completion. It was poured into ice water, and extracted with CH₂Cl₂ (20 mL×3). The combined organic layers were washed with brine (30 mL), dried over anhydrous sodium sulfate, filtered, and concentrated under reduced pressure to afford compound 4S-1b as a yellow solid which was used in next step.

The crude 4S-1b and 10% Pd—C (80 mg) were added to MeOH (20 mL) at room temperature. The reaction was purged with hydrogen (3 times). The reaction mixture was stirred at room temperature under 1 atmospheric pressure of H₂ overnight. The reaction was monitored by TLC for completion. It was filtered through celite, and the filtrate was concentrated under reduce pressure. The brown residue was purified by silica gel column chromatography (CH₂Cl₂:MeOH=20:1) to afford compound 4S-1c (432 mg, yield of two steps 67%) as a white solid. MS m/z 306.3 [M+H]⁺.

Compound 4S-1c (48 mg, 0.16 mmol) and compound 1k (50 mg, 0.16 mmol) were dissolved in 2-methoxyethanol (2 mL) followed by the addition of 2.5 M HCl in MeOH (0.16 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=20:1, 2% of Et₃N) to afford compound 4S (45 mg, yield 57%) as a white solid. ¹H NMR (400 MHz, CDCl₃) δ 10.86 (s, 1H), 8.65 (dd, J=8.4, 4.5 Hz, 1H), 8.05 (s, 1H), 7.48 (dd, J=8.4, 7.4 Hz, 1H), 7.31-7.21 (m, 1H), 7.13-7.05 (m, 2H), 6.92 (dd, J=8.7, 2.5 Hz, 1H), 6.84 (s, 1H), 6.72 (d, J=8.8 Hz, 1H), 4.18 (dd, J=10.5, 2.5 Hz, 1H), 4.01 (dd, J=10.4, 9.1 Hz, 1H), 3.62 (d, J=11.4 Hz, 1H), 3.17 (d, J=12.3 Hz, 1H), 3.09-2.93 (m, 3H), 2.66 (td, J=11.7, 3.3 Hz, 1H), 2.54 (t, J=10.9 Hz, 1H), 1.84 (s, 3H), 1.81 (s, 3H). MS m/z 485.3 [M+H]⁺.

### Example 8: Preparation of Compound 5R

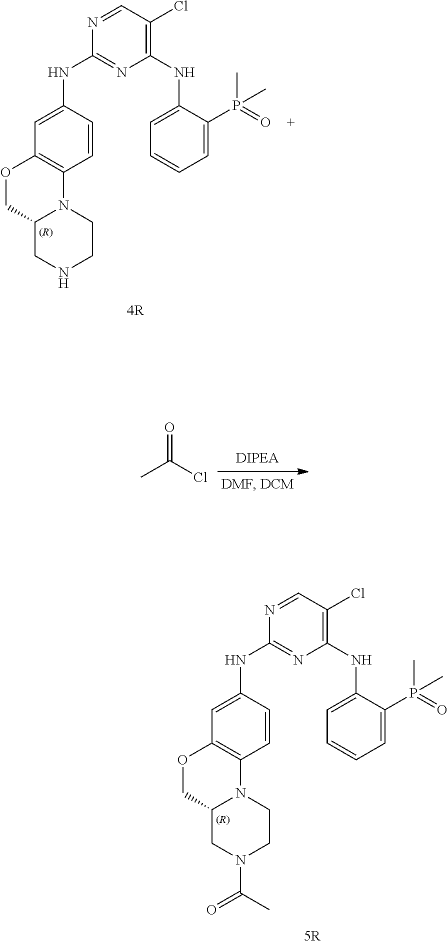

To a solution of compound 4R (60 mg, 0.12 mmol) in DMF (1.0 mL) at 0° C. was added DIPEA (32 mg, 0.24 mmol) followed by a solution of AcCl (12 mg, 0.15 mmol) in CH₂Cl₂ (1.0 mL). The reaction mixture was stirred at 0° C. for 1 hour. The reaction was monitored by TLC for completion. Water (10 mL) was added, and the mixture was extracted with CH₂Cl₂ (10 mL×3). The combined organic layers were washed with brine (20 mL), dried over anhydrous sodium sulfate, filtered, and concentrated under reduced pressure. The residue was purified by prep-TLC (CH₂Cl₂:acetone=2:1, 2% of Et₃N) to afford compound 5R (29 mg, yield 46%) as a white solid. ¹H NMR (500 MHz, CDCl₃) δ 10.90 (s, 1H), 8.65 (dd, J=7.9, 4.5 Hz, 1H), 8.06 (s, 1H), 7.48 (t, J=8.0 Hz, 1H), 7.30-7.23 (m, 1H), 7.19-7.08 (m, 2H), 6.94 (ddd, J=32.3, 8.7, 2.5 Hz, 1H), 6.82 (d, J=3.6 Hz, 1H), 6.74 (dd, J=14.9, 8.8 Hz, 1H), 4.75-4.57 (m, 1H), 4.26 (dd, J=10.7, 2.6 Hz, 1H), 4.04 (td, J=10.7, 8.6 Hz, 1H), 3.90 (d, J=13.2 Hz, 1H), 3.76-3.67 (m, 1H), 3.11-2.96 (m, 2H), 2.75-2.64 (m, 1H), 2.51-2.42 (m, 1H), 2.16 (S, 3H), 1.84 (s, 3H), 1.81 (s, 3H). MS m/z 527.5 [M+H]⁺.

### Example 9: Preparation of Compound 6R

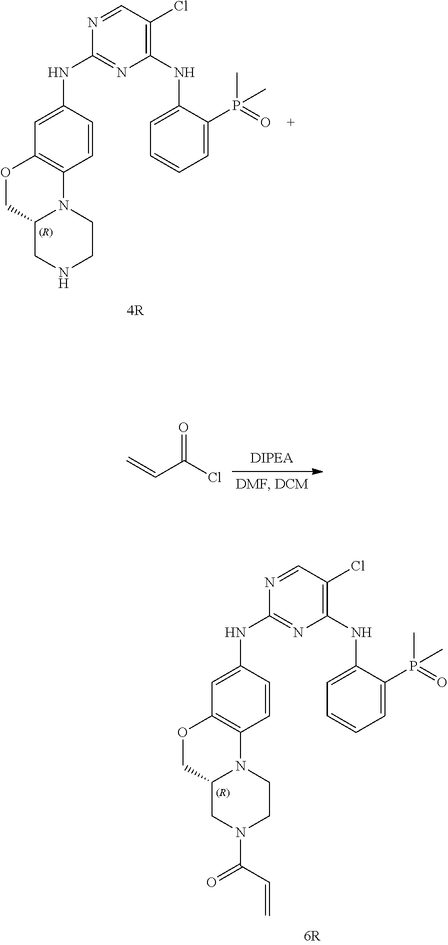

To a solution of compound 4R (60 mg, 0.12 mmol) in DMF (1.0 mL) at 0° C. was added DIPEA (32 mg, 0.24 mmol) followed by a solution of acrylic chloride (14 mg, 0.15 mmol) in CH₂Cl₂ (1.0 mL). The reaction mixture was stirred at 0° C. for 1 hour. The reaction was monitored by TLC for completion. Water (10 mL) was added, and the mixture was extracted with CH₂Cl₂ (10 mL×3). The combined organic layers were washed with brine (20 mL), dried over anhydrous sodium sulfate, filtered, and concentrated under reduced pressure. The residue was purified by prep-TLC (CH₂Cl₂:acetone=2:1, 2% of Et₃N) to afford compound 6R (21 mg, yield 32%) as a white solid. ¹H NMR (500 MHz, CDCl₃) δ 10.94 (s, 1H), 8.64 (dd, J=8.4, 4.4 Hz, 1H), 8.05 (s, 1H), 7.48 (t, J=8.1 Hz, 1H), 7.31-7.23 (m, 1H), 7.21-7.08 (m, 2H), 7.01-6.82 (m, 2H), 6.79-6.67 (m, 1H), 6.67-6.55 (m, 1H), 6.35 (d, J=16.8 Hz, 1H), 5.77 (dd, J=10.6, 1.7 Hz, 1H), 4.72 (dd, J=50.4, 11.5 Hz, 1H), 4.34-4.19 (m, 1H), 4.13-3.87 (m, 1H), 3.74 (d, J=11.4 Hz, 1H), 3.47-3.37 (m, 1H), 3.13-2.93 (m, 2H), 2.73 (m, 1H), 2.61-2.52 (m, 1H), 1.84 (s, 3H), 1.82 (s, 3H). MS m/z 539.5 [M+H]⁺.

### Example 10: Preparation of Compound 6S

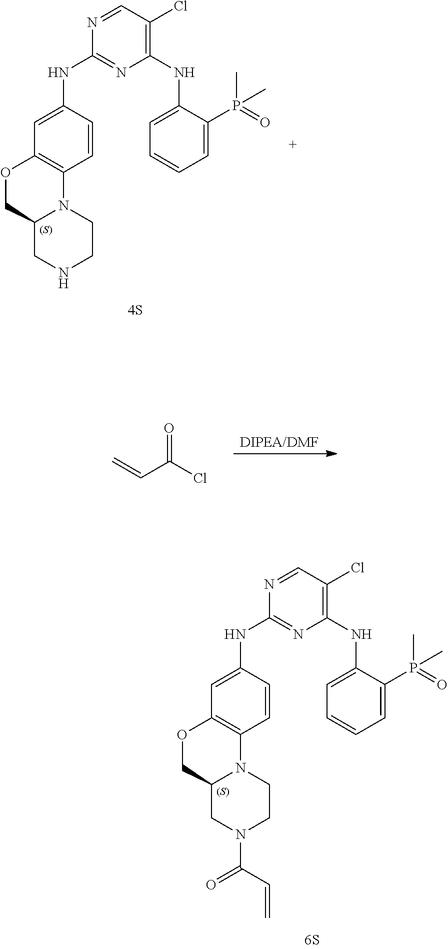

To a solution of compound 4S (60 mg, 0.12 mmol) in DMF (0.5 mL) at 0° C. was added DIPEA (32 mg, 0.24 mmol) followed by a solution of acrylic chloride (14 mg, 0.15 mmol) in CH₂Cl₂ (2.0 mL). The reaction mixture was stirred at 0° C. for 1 hour. The reaction was monitored by TLC for completion. Water (10 mL) was added, and the mixture was extracted with CH₂Cl₂ (10 mL×3). The combined organic layers were washed with brine (20 mL), dried over anhydrous sodium sulfate, filtered, and concentrated under reduced pressure. The residue was purified by prep-TLC (CH₂Cl₂:MeOH=20:1) to afford compound 6S (16 mg, yield 24%) as a yellow solid. ¹H NMR (400 MHz, CDCl₃) δ 10.89 (s, 1H), 8.64 (dd, J=8.4, 4.4 Hz, 1H), 8.06 (s, 1H), 7.48 (t, J=7.9 Hz, 1H), 7.31-7.22 (m, 1H), 7.20-7.07 (m, 2H), 7.00-6.80 (m, 2H), 6.79-6.69 (m, 1H), 6.68-6.54 (m, 1H), 6.34 (dd, J=16.8, 1.4 Hz, 1H), 5.77 (dd, J=10.5, 1.8 Hz, 1H), 4.71 (dd, J=40.3, 12.5 Hz, 1H), 4.36-4.20 (m, 1H), 4.13-3.88 (m, 1H), 3.73 (d, J=12.1 Hz, 1H), 3.48-3.34 (m, 1H), 3.14-2.96 (m, 2H), 2.79-2.66 (m, 1H), 2.64-2.50 (m, 1H), 1.84 (s, 3H), 1.81 (s, 3H). MS m/z 539.6 [M+H]⁺.

### Example 11: Preparation of Compound 7R

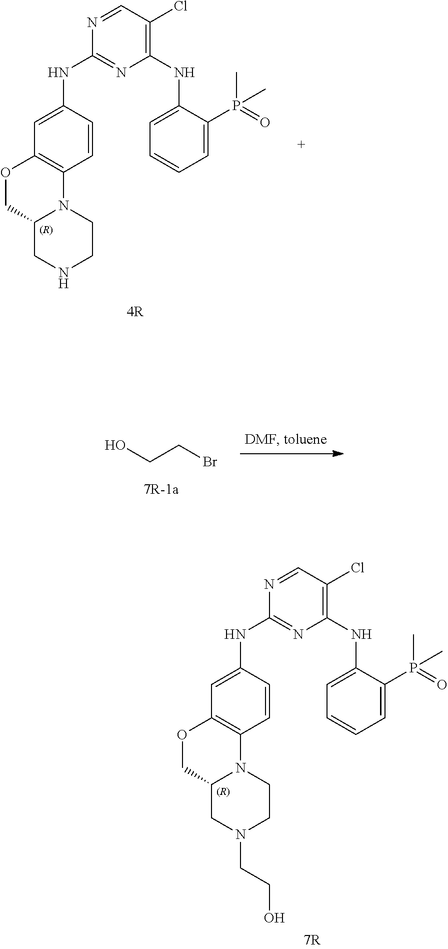

To a solution of compound 4R (48.5 mg, 0.10 mmol) in DMF (0.5 mL) was added a solution of 2-bromoethanol (7R-1a, 6.3 mg, 0.05 mmol) in toluene (1.5 mL). The reaction mixture in a sealed tube was heated to reflux, and stirred for 1 hour. The reaction was monitored by TLC for completion. It was cooled to room temperature, and concentrated under reduce pressure. The residue was purified by prep-TLC (CH₂Cl₂:MeOH=20:1) to afford compound 7R (13 mg, yield 50%) as a yellow solid. ¹H NMR (500 MHz, CDCl₃) δ 10.89 (s, 1H), 8.66 (dd, J=8.5, 4.4 Hz, 1H), 8.05 (s, 1H), 7.48 (dd, J=8.4, 7.6 Hz, 1H), 7.29-7.20 (m, 1H), 7.12-7.07 (m, 2H), 6.92 (dd, J=8.7, 2.5 Hz, 1H), 6.81 (s, 1H), 6.72 (d, J=8.8 Hz, 1H), 4.20 (dd, J=10.5, 2.6 Hz, 1H), 4.03 (dd, J=10.4, 9.1 Hz, 1H), 3.70-3.63 (m, 3H), 3.19-3.11 (m, 1H), 3.02 (d, J=10.9, 1H), 2.90-2.85 (m, 1H), 2.83-2.74 (m, 1H), 2.66-2.56 (m, 2H), 2.40 (td, J=11.5, 3.1 Hz, 1H), 2.04-1.95 (m, 1H), 1.84 (s, 3H), 1.81 (s, 3H). MS m/z 529.5 [M+H]⁺.

### Example 12: Preparation of Compound 8R

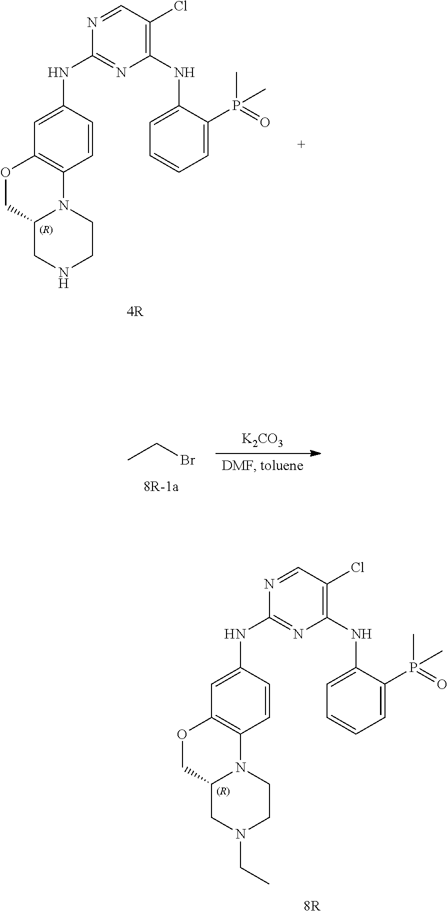

To a solution of compound 4R (48.5 mg, 0.10 mmol) in DMF (0.5 mL) was added K₂CO₃ (27.6 mg, 0.20 mmol) followed by a solution of 2-bromoethanol (compound 8R-1a, 10.9 mg, 0.10 mmol) in toluene (1.5 mL). The reaction mixture in a sealed tube was heated to reflux, and stirred for 3 hours. The reaction was monitored by TLC for completion. It was cooled to room temperature, and water (10 mL) was added. The mixture was extracted with CH₂Cl₂ (10 mL×3). The combined organic layers were washed with brine (20 mL), dried over anhydrous sodium sulfate, filtered, and concentrated under reduced pressure. The residue was purified by prep-TLC (CH₂Cl₂:acetone=2:1, 2% of Et₃N) to afford compound 8R (18 mg, yield 35%) as a white solid. ¹H NMR (500 MHz, CDCl₃) δ 10.88 (s, 1H), 8.65 (dd, J=8.5, 4.5 Hz, 1H), 8.05 (s, 1H), 7.48 (t, J=7.9 Hz, 1H), 7.29-7.21 (m, 1H), 7.12-7.04 (m, 2H), 6.92 (dd, J=8.7, 2.3 Hz, 1H), 6.78 (s, 1H), 6.72 (d, J=8.7 Hz, 1H), 4.21 (d, J=10.4, 1H), 4.03 (t, J=9.7 Hz, 1H), 3.65 (d, J=11.8 Hz, 1H), 3.18 (t, J=9.7 Hz, 1H), 3.05 (d, J=11.4 Hz, 1H), 2.94-2.87 (m, 1H), 2.85-2.75 (m, 1H), 2.53-2.45 (m, 2H), 2.24 (td, J=11.4, 2.6 Hz, 1H), 1.85-1.82 (s, 3H), 1.81 (s, 3H), 1.14 (t, J=7.2 Hz, 3H). MS m/z 513.3 [M+H]⁺.

### Example 13: Preparation of Compound 9R

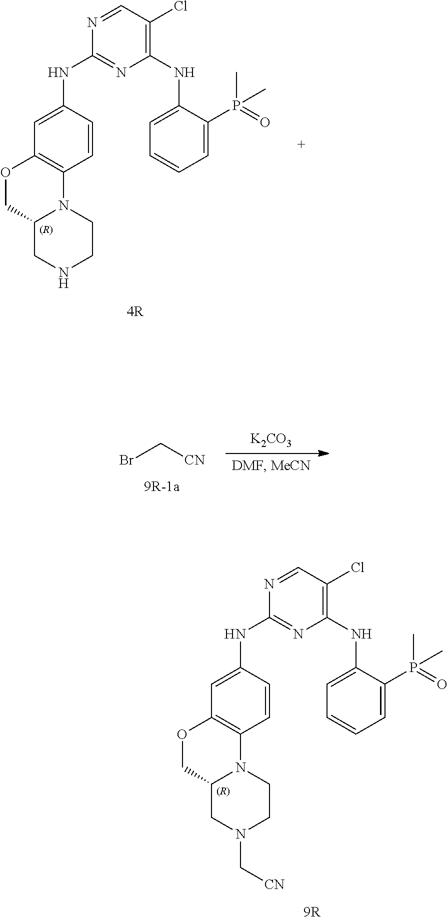

To a solution of compound 4R (48.5 mg, 0.10 mmol) in DMF (0.6 mL) was added K₂CO₃ (27.6 mg, 0.20 mmol) followed by a solution of bromoacetonitrile (9R-1a, 13.2 mg, 0.11 mmol) in CH₃CN (1.5 mL). The reaction mixture was stirred at room temperature overnight. The reaction was monitored by TLC for completion. It was concentrated under reduce pressure. The residue was purified by prep-TLC (CH₂Cl₂:MeOH=20:1, 2% of Et₃N) to afford compound 9R (26 mg, yield 50%) as a white solid. ¹H NMR (500 MHz, CDCl₃) δ 10.89 (s, 1H), 8.65 (dd, J=8.3, 4.4 Hz, 1H), 8.06 (s, 1H), 7.51-7.46 (m, 1H), 7.29-7.23 (m, 1H), 7.16-7.08 (m, 2H), 6.94 (dd, J=8.7, 2.5 Hz, 1H), 6.79 (s, 1H), 6.73 (d, J=8.8 Hz, 1H), 4.21 (dd, J=10.5, 2.7 Hz, 1H), 4.06 (dd, J=10.5, 9.0 Hz, 1H), 3.74-3.69 (m, 1H), 3.60 (s, 2H), 3.23-3.17 (m, 1H), 2.90 (dd, J=10.4, 2.0 Hz, 1H), 2.85-2.74 (m, 2H), 2.68 (td, J=11.1, 3.2 Hz, 1H), 2.30-2.23 (m, 1H), 1.84 (s, 3H), 1.81 (s, 3H). MS m/z 524.4 [M+H]⁺.

### Example 14: Preparation of Compound 10R

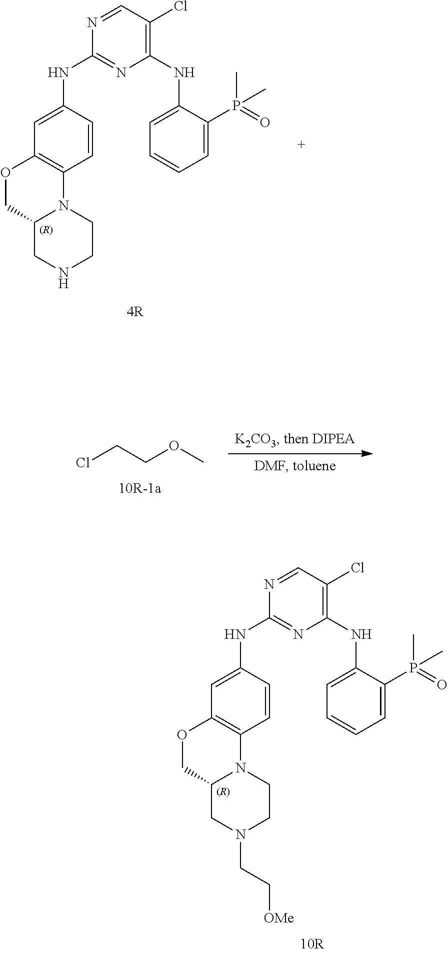

To a solution of compound 4R (48.5 mg, 0.10 mmol) in DMF (0.6 mL) was added K₂CO₃ (27.6 mg, 0.20 mmol) followed by a solution of 2-chloroethyl methyl ether (10R-1a, 9.5 mg, 0.10 mmol) in toluene (1.5 mL). The reaction mixture in a sealed tube was heated to 90° C., and stirred for 2 hours. Some of starting material was found by TLC. More DIPEA (25.9 mg, 0.20 mmol) was added, and the reaction was stirred for 2 hours. It was cooled to room temperature, and concentrated under reduce pressure. The residue was purified by prep-TLC (CH₂Cl₂: acetone=2:1, 2% of Et₃N) to afford compound 10R (12 mg, yield 22%) as a white solid. ¹H NMR (500 MHz, CDCl₃) δ 10.89 (s, 1H), 8.65 (dd, J=8.3, 4.3 Hz, 1H), 8.05 (s, 1H), 7.47 (dd, J=8.3, 7.4 Hz, 1H), 7.29-7.22 (m, 1H), 7.12-7.07 (m, 2H), 6.91 (dd, J=8.7, 2.5 Hz, 1H), 6.80 (s, 1H), 6.72 (d, J=8.8 Hz, 1H), 4.20 (dd, J=10.5, 2.6 Hz, 1H), 4.02 (dd, J=10.5, 8.8 Hz, 1H), 3.64 (d, J=11.7 Hz, 1H), 3.59 (t, J=5.3 Hz, 2H), 3.38 (s, 3H), 3.28 (dd, J=12.8, 7.3 Hz, 1H), 3.10 (d, J=10.8 Hz, 1H), 2.98 (d, J=10.7 Hz, 1H), 2.94-2.86 (m, 1H), 2.68 (d, J=3.3 Hz, 2H), 2.42-2.31 (m, 1H), 2.04-1.92 (m, 1H), 1.84 (s, 3H), 1.81 (s, 3H). MS m/z 543.4 [M+H]⁺.

### Example 15: Preparation of Compound 11R

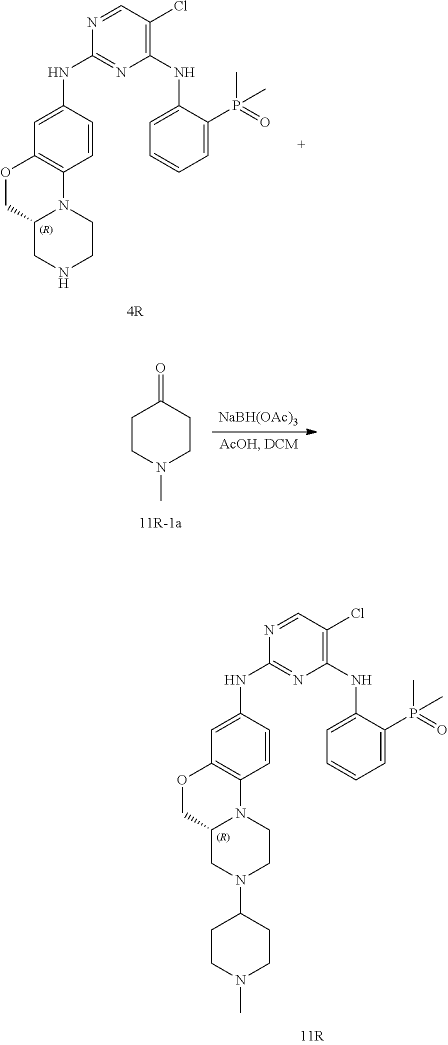

To a solution of compound 4R (60 mg, 0.12 mmol) in CH₂Cl₂ (1.5 mL) was added N-methyl-4-piperidone (11R-1a, 42 mg, 0.37 mmol) and AcOH (2 drops). The reaction mixture was stirred at room temperature for 1 hour. It was cooled to 0° C., and sodium triacetoxyborohydride (80 mg, 0.37 mmol) was added. The reaction mixture was stirred at room temperature overnight. The reaction was monitored by TLC for completion. The reaction was quenched with water. Saturated NaHCO₃ aqueous solution was added, and the mixture was extracted with EtOAc (10 mL×3). The combined organic layers were washed with brine (20 mL), dried over anhydrous sodium sulfate, filtered, and concentrated under reduced pressure. The residue was purified by prep-TLC (CH₂Cl₂:MeOH=20:1, 2% of Et₃N) to afford compound 11R (9 mg, yield 13%) as a white solid. ¹H NMR (500 MHz, CDCl₃) δ 10.86 (s, 1H), 8.65 (dd, J=8.5, 4.4 Hz, 1H), 8.04 (s, 1H), 7.47 (t, J=7.8 Hz, 1H), 7.28-7.21 (m, 1H), 7.11-7.05 (m, 2H), 6.91 (dd, J=8.6, 1.9 Hz, 1H), 6.81 (s, 1H), 6.71 (d, J=8.7 Hz, 1H), 4.18 (dd, J=10.5, 2.0 Hz, 1H), 4.01 (t, J=9.7 Hz, 1H), 3.65 (d, J=11.2 Hz, 1H), 3.12 (t, J=9.5 Hz, 1H), 3.01 (d, J=10.8 Hz, 1H), 2.95 (d, J=11.3 Hz, 2H), 2.86 (d, J=10.9 Hz, 1H), 2.79-2.66 (m, 1H), 2.35-2.30 (m, 3H), 2.06-1.95 (m, 2H), 1.83 (s, 3H), 1.81 (s, 3H), 1.69-1.50 (m, 2H). MS m/z 582.5 [M+H]⁺.

### Example 16: Preparation of Compound 12R

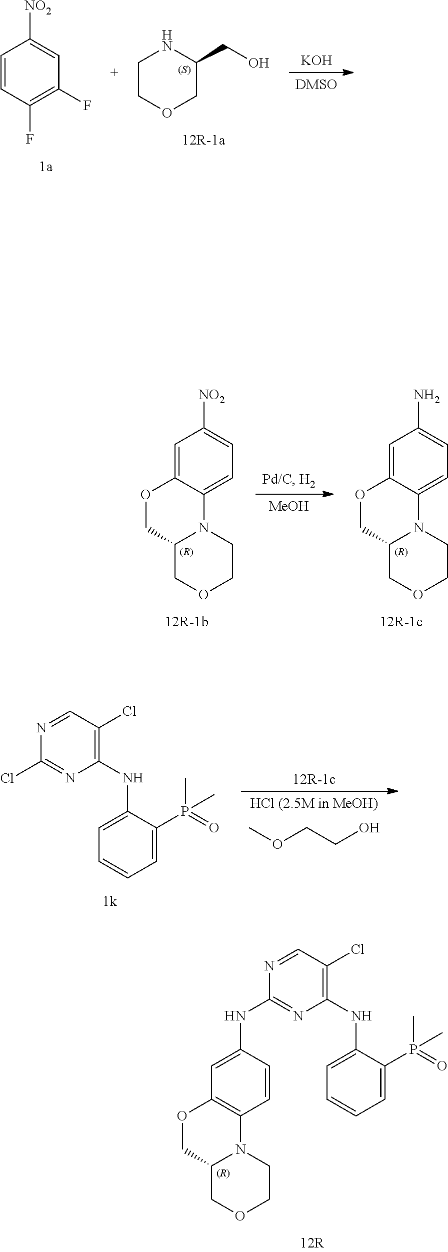

To a solution of 1,2-difluoro-4-nitrobenzene (1a, 326 mg, 2.05 mmol) and (S)-3-hydroxymethylmorpholine (12R-1a, 200 mg, 1.70 mmol) in dry DMSO (6 mL) was added KOH (286 mg, 5.10 mmol). The reaction mixture was stirred at room temperature for 3 hours. It was heated at 60° C. for 5 hours. The reaction was monitored by TLC for completion. It was cooled to room temperature, and ice water was added. The mixture was extracted with CH₂Cl₂ (20 mL×3). The combined organic layers were washed with brine (30 mL), dried over anhydrous sodium sulfate, filtered, and concentrated under reduced pressure. The residue was purified by silica gel column chromatography (PE:EtOAc=6:1) to afford compound 12R-1b (187 mg, yield 46%) as a yellow solid.

To a solution of compound 12R-1b in MeOH (10 mL) was added 10% Pd—C (50 mg) at room temperature. The reaction was purged with hydrogen (3 times). The reaction mixture was stirred at room temperature under 1 atmospheric pressure of H₂ overnight. The reaction was monitored by TLC for completion. It was filtered through celite, and the filtrate was concentrated to afford a brown crude which was purified by silica gel column chromatography (CH₂Cl₂:MeOH=50:1) to afford compound 12R-1c (38 mg, yield 24%) a colorless oil. MS m/z 207.8 [M+H]⁺.

To a solution of compound 12R-1c (38 mg, 0.18 mmol) and compound 1k (58 mg, 0.18 mmol) in 2-methoxyethanol (1.5 mL) was added 2.5 M HCl in MeOH (2.5 M, 0.22 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=20:1, 2% of Et₃N) to afford compound 12R (30 mg, yield 34%) as a yellow solid. ¹H NMR (500 MHz, CDCl₃) δ 10.87 (s, 1H), 8.65 (dd, J=8.5, 4.4 Hz, 1H), 8.06 (s, 1H), 7.48 (t, J=8.6, 1H), 7.33-7.20 (m, 3H), 7.13-7.06 (m, 2H), 6.93 (dd, J=8.6, 2.5 Hz, 1H), 6.77 (s, 1H), 6.69 (d, J=8.7 Hz, 1H), 4.16 (dd, J=10.5, 2.7 Hz, 1H), 4.05 (dd, J=11.5, 3.5 Hz, 1H), 3.98 (dd, J=10.5, 9.1 Hz, 1H), 3.88 (dd, J=10.9, 3.1 Hz, 1H), 3.76 (td, J=11.6, 2.8 Hz, 1H), 3.51-3.45 (m, 1H), 3.30 (t, J=10.7 Hz, 1H), 3.24-3.18 (m, 1H), 2.84 (td, J=11.8, 3.6 Hz, 1H), 1.84 (s, 3H), 1.81 (s, 3H). MS m/z 486.9 [M+H]⁺.

### Example 17: Preparation of Compound 12S

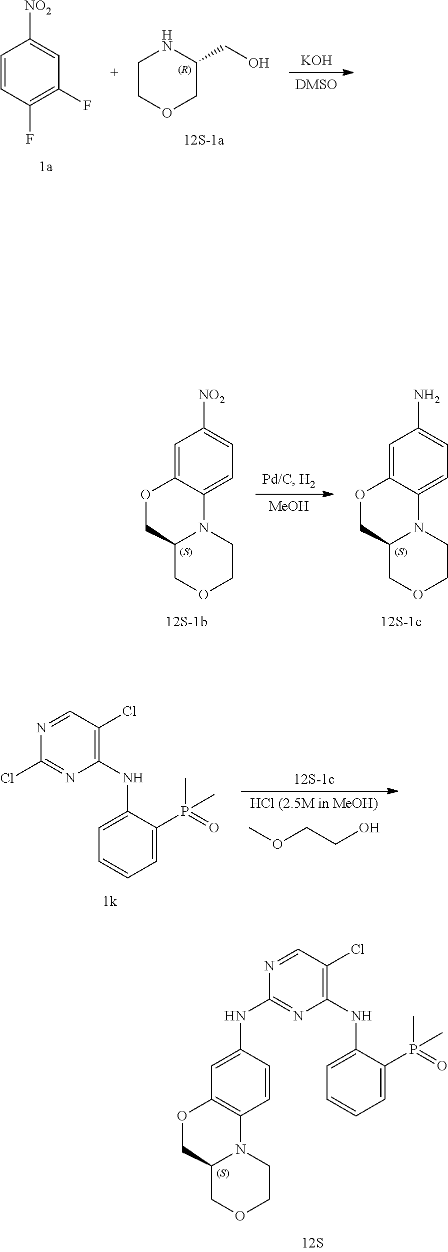

To a solution of 1,2-difluoro-4-nitrobenzene (1a, 317 mg, 2.05 mmol) and (R)-3-hydroxymethylmorpholine (12S-1a, 200 mg, 1.70 mmol) in dry DMSO (6 mL) was added KOH (286 mg, 5.10 mmol). The reaction mixture was stirred at room temperature for 3 hours. It was heated at 60° C. for 5 hours. The reaction was monitored by TLC for completion. It was cooled to room temperature, and ice water was added. The mixture was extracted with CH₂Cl₂ (25 mL×3). The combined organic layers were washed with brine (40 mL), dried over anhydrous sodium sulfate, filtered, and concentrated under reduced pressure. The residue was purified by silica gel column chromatography (PE:EtOAc=6:1) to afford compound 12S-1b as a yellow solid which was used in next step.

To a solution of compound 12S-1b in MeOH (15 mL) was added 10% Pd—C (100 mg) at room temperature. The reaction was purged with hydrogen (3 times). The reaction mixture was stirred at room temperature under 1 atmospheric pressure of H₂ overnight. The reaction was monitored by TLC for completion. It was filtered through celite, and the filtrate was concentrated to afford a brown crude which was purified by silica gel column chromatography (CH₂Cl₂:MeOH=50:1) to afford compound 12S-1c (44 mg, yield of two steps 13%) as a colorless oil. MS m/z 207.4 [M+H]⁺.

To a solution of compound 12S-1c (44 mg, 0.21 mmol) and compound 1k (68 mg, 0.21 mmol) in 2-methoxyethanol (2 mL) was added 2.5 M HCl in MeOH (0.22 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂: MeOH=20:1, 2% of Et₃N) to afford compound 12S (11 mg, yield 11%) as a yellow solid. ¹H NMR (400 MHz, CDCl₃) δ 10.87 (s, 1H), 8.65 (dd, J=8.4, 4.4 Hz, 1H), 8.05 (s, 1H), 7.48 (t, J=7.9 Hz, 1H), 7.30-7.21 (m, 1H), 7.14-7.06 (m, 2H), 6.93 (dd, J=8.7, 2.5 Hz, 1H), 6.83 (s, 1H), 6.69 (d, J=8.7 Hz, 1H), 4.16 (dd, J=10.5, 2.6 Hz, 1H), 4.05 (dd, J=11.5, 3.2 Hz, 1H), 3.98 (dd, J=10.3, 9.2 Hz, 1H), 3.88 (dd, J=10.8, 2.8 Hz, 1H), 3.76 (td, J=11.6, 2.8 Hz, 1H), 3.53-3.44 (m, 1H), 3.30 (t, J=10.6 Hz, 1H), 3.25-3.15 (m, 1H), 2.89-2.77 (m, 1H), 1.84 (s, 3H), 1.81 (s, 3H). MS m/z 486.5 [M+H]⁺.

### Example 18: Preparation of Compound 13R

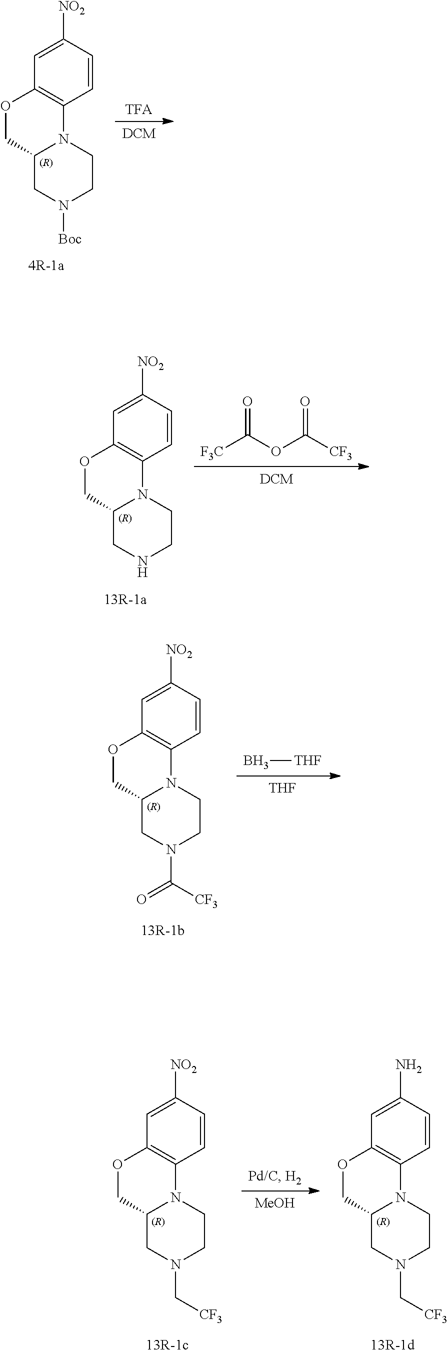

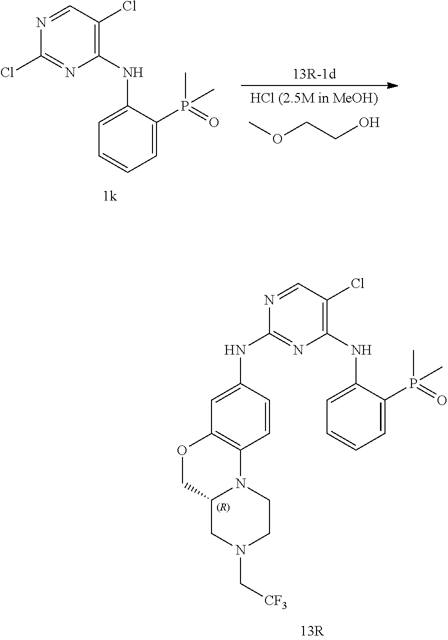

To a solution of compound 4R-1a (1.5 g, 4.5 mmol) in CH₂Cl₂ (10 mL) was added TFA (4.5 mL) at 0° C. The reaction mixture was stirred room temperature for 1 hour. The reaction was monitored by TLC for completion. It was concentrated under reduced pressure, neutralized with saturated NaHCO₃ aqueous solution, and extracted with CH₂Cl₂ (20 mL×3). The combined organic layers were washed with brine (40 mL), dried over anhydrous sodium sulfate, filtered, and concentrated under reduced pressure to afford compound 13R-1a (3.06 g) which was used in next step.

To a stirred solution of compound 13R-1a (3.06 g) in CH₂Cl₂ (20 mL) at 0° C. was added DIPEA (3.36 g, 26.0 mmol) followed by trifluoroacetic anhydride (3.0 g, 14.3 mmol). The reaction mixture was stirred at room temperature overnight. The reaction was monitored by TLC for completion. It was concentrated under reduce pressure, neutralized with saturated NaHCO₃ aqueous solution to pH=8, and extracted with CH₂Cl₂ (30 mL×3). The combined organic layers were washed with brine (40 mL), dried over anhydrous sodium sulfate, filtered, and concentrated under reduced pressure. The residue was purified by silica gel column chromatography (PE:EtOAc=2:1) to afford compound 13R-1b (1.2 g, yield of two steps 63%) as a yellow solid. MS m/z 332.5 [M+H]⁺.

To a solution of compound 13R-1b (950 mg, 2.87 mmol) in THF (10 mL) was added BH₃-THF (1.0 M, 7.17 mL). The reaction mixture was head to reflux, and stirred for 2 hours. The reaction was monitored by TLC for completion. It was cooled to room temperature, and concentrated under reduce pressure. The residue was purified by silica gel column chromatography (PE:EtOAc=8:1) to afford compound 13R-1c as a yellow solid which was used in next step.

To a solution of compound 13R-1c in MeOH (20 mL) was added 10% Pd—C (200 mg) at room temperature. The reaction was purged with hydrogen (3 times). The reaction mixture was stirred at room temperature under 1 atmospheric pressure of H₂ overnight. It was filtered through celite, and concentrated under reduce pressure to afford a brown residue. The residue was purified by silica gel column chromatography (CH₂Cl₂:MeOH=50:1 to 20:1) to afford compound 13R-1d (520 mg, yield of two steps 63%) as a yellow solid. MS m/z 288.4 [M+H]⁺.

To a solution of compound 13R-1d (91 mg, 0.32 mmol) and compound 1k (100 mg, 0.32 mmol) in 2-methoxyethanol (2 mL) was added 2.5 M HCl in MeOH (0.33 mL). The reaction in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=20:1, 2% of Et₃N) to afford compound 13R (71 mg, yield 40%) as a yellow solid. ¹H NMR (500 MHz, CDCl₃) δ 10.80 (s, 1H), 8.58 (dd, J=8.3, 4.4 Hz, 1H), 7.99 (s, 1H), 7.41 (dd, J=8.4, 7.4 Hz, 1H), 7.22-7.15 (m, 1H), 7.05-7.00 (m, 2H), 6.85 (dd, J=8.7, 2.5 Hz, 1H), 6.69 (s, 1H), 6.65 (d, J=8.8 Hz, 1H), 4.11 (dd, J=10.5, 2.7 Hz, 1H), 3.94 (dd, J=10.5, 8.9 Hz, 1H), 3.56 (dt, J=11.4, 2.4 Hz, 1H), 3.16-3.08 (m, 1H), 3.02-2.93 (m, 3H), 2.88-2.82 (m, 1H), 2.77 (td, J=11.6, 3.1 Hz, 1H), 2.62 (td, J=11.3, 2.9 Hz, 1H), 2.21 (t, J=10.6 Hz, 1H), 1.77 (s, 3H), 1.74 (s, 3H). MS m/z 567.6 [M+H]⁺.

### Example 19: Preparation of Compound 14R

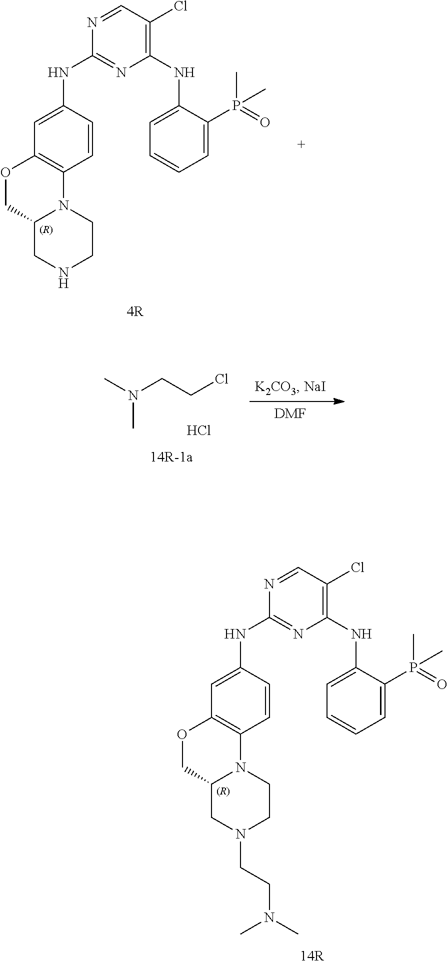

Compound 4R (50 mg, 0.10 mmol), dimethylaminochloroethane hydrochloride salt (14R-1a, 16 mg, 0.10 mmol), NaI (16 mg, 0.10 mmol), K₂CO₃ (42 mg, 0.30 mmol), and DMF (2 mL) in a sealed tube was heated to 80° C., and stirred overnight. Some of starting material was found by TLC. It was cooled to room temperature, and water (10 mL) was added. The mixture was extracted with CH₂Cl₂ (15 mL×3). The combined organic layers were washed with brine (25 mL), dried over anhydrous sodium sulfate, filtered, and concentrated under reduced pressure. The residue was purified by prep-TLC (CH₂Cl₂:MeOH=3:1) to afford compound 14R (2 mg, yield 4%) as a white solid. ¹H NMR (500 MHz, CDCl₃) δ 10.89 (s, 1H), 8.65 (dd, J=8.3, 4.5 Hz, 1H), 8.05 (s, 1H), 7.48 (t, J=7.5 Hz, 1H), 7.26 (d, J=20.4 Hz, 1H), 7.15-7.07 (m, 2H), 6.92 (dd, J=8.6, 2.0 Hz, 1H), 6.79 (s, 1H), 6.71 (d, J=8.8 Hz, 1H), 4.20 (dd, J=10.4, 2.2 Hz, 1H), 4.05-3.97 (m, 1H), 3.63 (d, J=11.6 Hz, 2H), 3.19 (dd, J=10.3, 9.0 Hz, 1H), 3.04 (d, J=10.3 Hz, 1H), 2.91 (d, J=10.1 Hz, 1H), 2.82 (td, J=11.5, 2.5 Hz, 1H), 2.73-2.63 (m, 2H), 2.56-2.39 (s, 6H), 2.37-2.29 (m, 2H), 2.24-2.18 (m, 1H), 1.84 (s, 3H), 1.81 (s, 3H). MS m/z 556.5 [M+H]⁺.

### Example 20: Preparation of Compound 15R

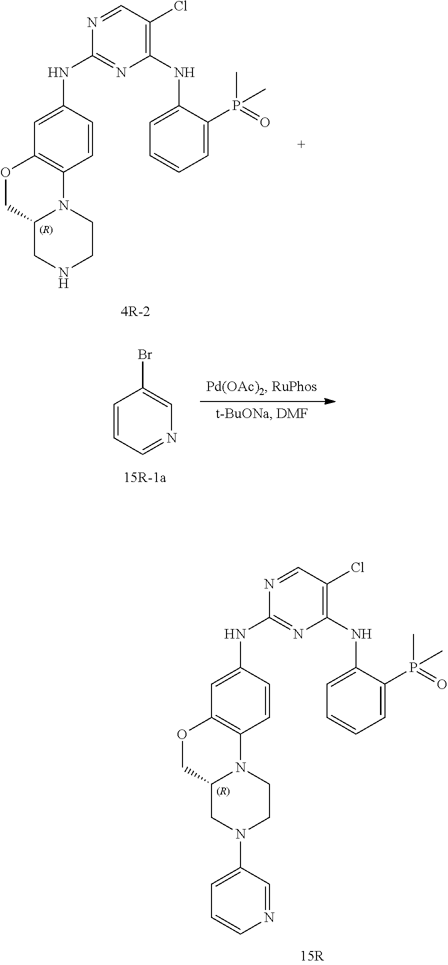

Compound 4R-2 (48 mg, 0.10 mmol), palladium acetate (0.2 mg, 0.001 mmol), RuPhos (0.9 mg, 0.002 mmol), sodium tert-butoxide (11.5 mg, 0.12 mmol), 3-bromopyridine (15R-1a, 17.4 mg, 0.11 mmol), and DMF (0.5 mL) in a sealed tube was heated to 110° C., and stirred overnight. Some of starting material was found by TLC. After the reaction mixture was cooled to room temperature, water (10 mL) was added, and extracted with CH₂Cl₂ (10 mL×3). The combined organic layers were washed with brine (25 mL), dried over anhydrous sodium sulfate, filtered, and concentrated under reduced pressure. The residue was purified by prep-TLC (CH₂Cl₂:MeOH=20:1, 2% of Et₃N) to afford compound 15R (6 mg, yield 11%) as a yellow solid. MS m/z 562.5 [M+H]⁺.

### Example 21: Preparation of Compound 16R

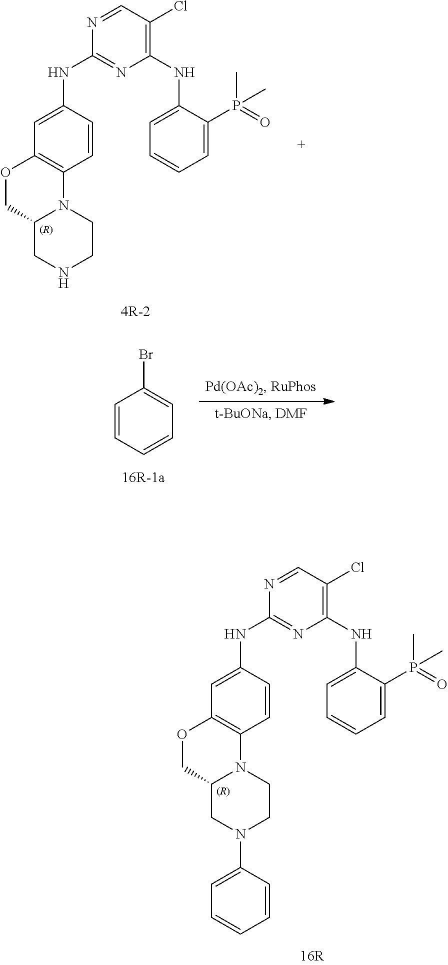

Compound 4R-2 (48 mg, 0.10 mmol), palladium acetate (0.2 mg, 0.001 mmol), RuPhos (0.9 mg, 0.002 mmol), sodium tert-butoxide (11.5 mg, 0.12 mmol) and bromobenzene (16R-1a, 17.3 mg, 0.11 mmol), and DMF (0.5 mL) in a sealed tube was heated to 110° C., and stirred overnight. Some of starting material was found by TLC. After the reaction mixture was cooled to room temperature, water (10 mL) was added, and the mixture was extracted with CH₂Cl₂ (10 mL×3). The combined organic layers were washed with brine (25 mL), dried over anhydrous sodium sulfate, filtered, and concentrated under reduced pressure. The residue was purified by prep-TLC (CH₂Cl₂:MeOH=20:1, 2% of Et₃N) to afford compound 16R (4 mg, yield 7%) as a yellow solid. ¹H NMR (400 MHz, CDCl₃) δ 10.82 (s, 1H), 8.59 (dd, J=8.5, 4.4 Hz, 1H), 7.99 (s, 1H), 7.43 (dd, J=8.7, 7.7 Hz, 1H), 7.27-7.16 (m, 3H), 7.07 (d, J=2.5 Hz, 1H), 7.03 (td, J=7.4, 1.4 Hz, 1H), 6.95-6.82 (m, 4H), 6.75-6.69 (m, 2H), 4.24 (dd, J=10.5, 2.6 Hz, 1H), 4.05 (dd, J=10.5, 9.0 Hz, 1H), 3.75-3.62 (m, 2H), 3.55-3.48 (m, 1H), 3.28-3.20 (m, 1H), 2.97-2.82 (m, 2H), 2.53 (t, J=11.1 Hz, 1H), 1.78 (s, 3H), 1.75 (s, 3H). MS m/z 561.6 [M+H]⁺.

### Example 22: Preparation of Compound 17R

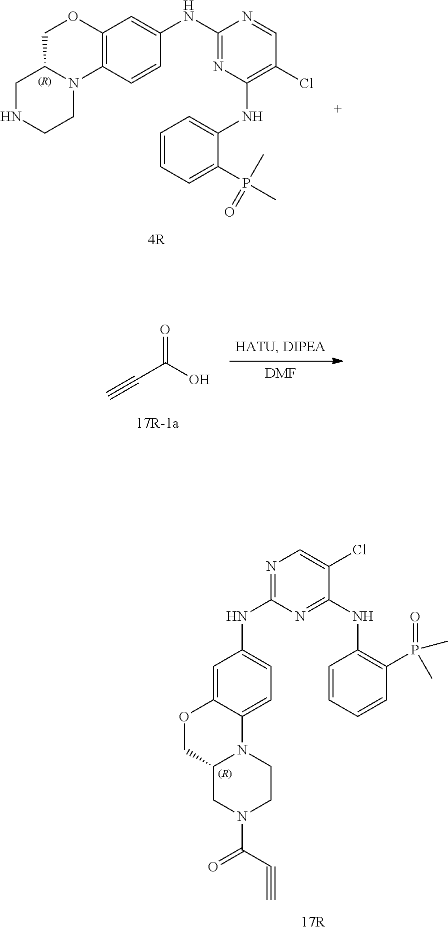

Compound 4R (48.5 mg, 0.1 mmol), compound 17R-1a (10.5 mg, 0.15 mmol), HATU (57 mg, 0.15 mmol), and DIPEA (20 mg, 0.15 mmol) were dissolved in DMF (1.5 mL) at room temperature. The reaction mixture was stirred for 0.5 hour. The reaction was monitored by TLC for completion. Water (10 mL) was added to the reaction mixture, and the mixture was extracted with CH₂Cl₂ (15 mL×3). The combined organic layers were washed with brine (20 mL), dried over anhydrous sodium sulfate, filtered, and concentrated under reduced pressure. The residue was purified by prep-TLC (CH₂Cl₂:MeOH=10:1) to afford compound 17R as a yellow solid. MS m/z 537.6 [M+H]⁺.

### Example 23: Preparation of Compound 18R

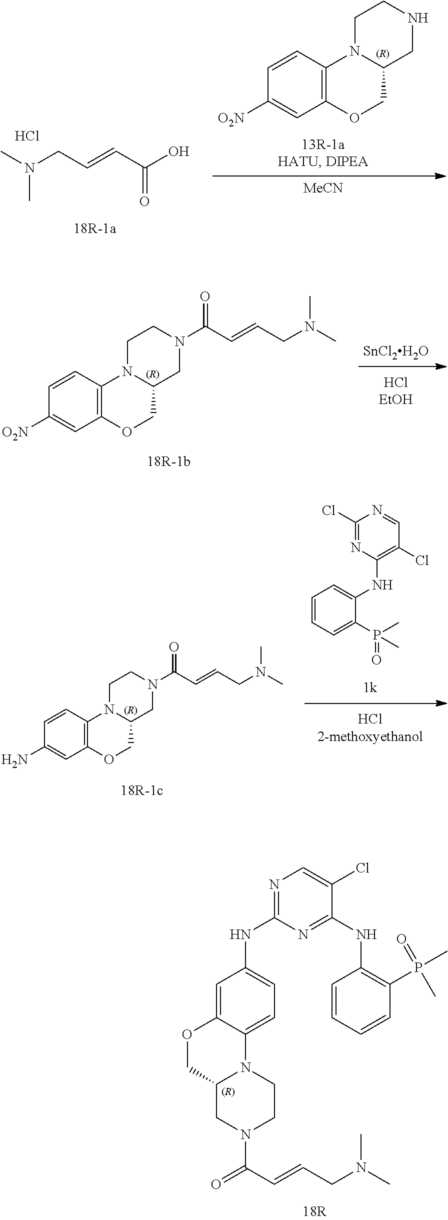

Compound 13R-1a (400 mg, 1.70 mmol), 18R-1a (422 mg, 2.55 mmol), HATU (970 mg, 2.55 mmol), and DIPEA (330 mg, 2.55 mmol) were dissolved in acetonitrile (20 mL). The reaction was stirred at room temperature for 30 minutes. The reaction was monitored by TLC for completion. It was concentrated under reduce pressure and the residue was purified by silica gel column chromatography (CH₂Cl₂: MeOH=20:1, 2% Et₃N) to afford compound 18R-1b (417 mg, yield 71%) as a yellow solid. MS m/z 347.5 [M+H]⁺.

Compound 18R-1b (326 mg, 0.94 mmol) and SnCl₂.2H₂O (977 mg, 4.71 mmol) were dissolved in ethanol (8 mL) followed by the addition of 2.0 M HCl (2.35 mL). The reaction mixture was stirred at 90° C. overnight. The reaction was monitored by TLC for completion. It was concentrated under reduce pressure. The residue was dissolved in a small amount of CH₂Cl₂, neutralized with saturated NaHCO₃ aqueous solution to pH=7-8, and extracted with CH₂Cl₂ (20×3 mL). The combined organic layers were washed with brine, dried over anhydrous sodium sulfate, filtered, and concentrated under reduced pressure to afford compound 18R-1c (203 mg, yield 68%) as a yellow solid. MS m/z 317.4 [M+H]⁺.

Compound 18R-1c (80 mg, 0.25 mmol) and compound 1k (80 mg, 0.25 mmol) were dissolved in 2-methoxyethanol (2 mL) followed by the addition of a solution of 2.5 M HCl in MeOH (0.26 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=9:1) to afford compound 18R (14 mg, yield 9%) as a light yellow solid. ¹H NMR (500 MHz, CDCl₃) δ 10.90 (s, 1H), 8.66-8.62 (m, 1H), 8.06 (s, 1H), 7.49 (t, J=7.9 Hz, 1H), 7.30-7.24 (m, 1H), 7.15 (d, J=2.4 Hz, 1H), 7.14-7.10 (m, 1H), 6.96-6.92 (m, 1H), 6.91-6.81 (m, 2H), 6.80 (s, 1H), 6.76-6.70 (m, 1H), 4.79-4.60 (m, 1H), 4.39-4.13 (m, 2H), 4.08-4.02 (m, 1H), 3.76 (d, J=11.6 Hz, 1H), 3.51-3.34 (m, 3H), 3.15-2.91 (m, 2H), 2.77-2.70 (m, 1H), 2.56 (s, 6H), 1.84 (s, 3H), 1.82 (s, 3H). MS m/z 596.5 [M+H]⁺.

### Example 24: Preparation of Compound 19R

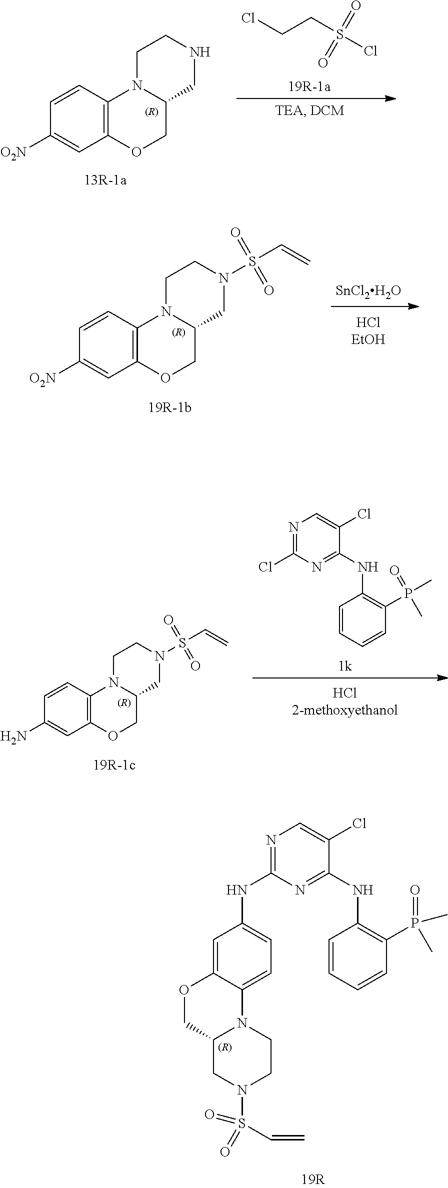

Compound 13R-1a (500 mg, 2.13 mmol) and TEA (258 mg, 2.55 mmol) were dissolved in CH₂Cl₂ (15 mL) at 0° C. followed by the addition of a solution of compound 19R-1a (416 mg, 2.55 mmol) in CH₂Cl₂ (5 mL). The reaction mixture was stirred at room temperature for 1 hour. The reaction was monitored by TLC for completion. It was concentrated under reduced pressure, and the residue was purified silica gel column chromatography (PE:EtOAc=2:1 to 1:2) to afford compound 19R-1b (153 mg, yield 22%) as a yellow solid. MS m/z 326.3 [M+H]⁺.

Compound 19R-1b (150 mg, 0.46 mmol) and tin(II) chloride dihydrate (479 mg, 2.31 mmol) were dissolved in ethanol (8 mL) followed by the addition of 2.0 M HCl aqueous solution (1.15 mL). The reaction mixture was stirred at 90° C. overnight. The reaction was monitored by TLC for completion. It was concentrated under reduced pressure. The residue was dissolved in a small amount of CH₂Cl₂, neutralized with saturated NaHCO₃ aqueous solution to pH=7-8, and extracted with CH₂Cl₂ (20×3 mL). The combined organic layers were washed with brine, dried over anhydrous sodium sulfate, filtered, and concentrated under reduced pressure. The residue was purified silica gel column chromatography (CH₂Cl₂:MeOH=20:1) to afford compound 19R-1c (107 mg, yield 78%) as a yellow solid. ¹H NMR (500 MHz, DMSO-d₆) δ 6.84 (dd, J=16.5, 10.0 Hz, 1H), 6.60 (d, J=8.6 Hz, 1H), 6.21 (d, J=10.0 Hz, 1H), 6.15 (d, J=16.5 Hz, 1H), 6.09 (dd, J=8.5, 2.4 Hz, 1H), 6.03 (d, J=2.4 Hz, 1H), 4.59 (bs, 2H), 4.22 (dd, J=10.6, 2.5 Hz, 1H), 3.82 (dd, J=10.5, 8.7 Hz, 1H), 3.72 (d, J=12.1 Hz, 1H), 3.51 (t, J=10.2 Hz, 2H), 2.96-2.90 (m, 1H), 2.81-2.74 (m, 1H), 2.57-2.51 (m, 1H), 2.42 (t, J=11.1 Hz, 1H).

Compound 19R-1c (50 mg, 0.17 mmol) and compound 1k (54 mg, 0.17 mmol) were dissolved in 2-methoxyethanol (2 mL) followed by the addition of 2.5 M HCl in MeOH (0.18 mL). The reaction mixture in a sealed tube was heated to 120° C., and stirred overnight. The reaction was monitored by TLC for completion. It was cooled to room temperature, and concentrated under reduce pressure. The residue was purified by silica gel column chromatography (CH₂Cl₂:MeOH=50:1, 2% of Et₃N) to afford compound 19R (32 mg, yield 32%) as a light yellow solid. ¹H NMR (500 MHz, CDCl₃) δ 10.93 (s, 1H), 8.65-8.61 (m, 1H), 8.05 (s, 1H), 7.50-7.45 (m, 1H), 7.30-7.24 (m, 1H), 7.16 (d, J=2.5 Hz, 1H), 7.14-7.09 (m, 1H), 7.03 (s, 1H), 6.96-6.92 (m, 1H), 6.73 (d, J=8.8 Hz, 1H), 6.44 (dd, J=16.6, 9.9 Hz, 1H), 6.30 (d, J=16.6 Hz, 1H), 6.10 (d, J=9.9 Hz, 1H), 4.26-4.22 (m, 1H), 4.04-3.99 (m, 1H), 3.81-3.74 (m, 2H), 3.68-3.64 (m, 1H), 3.31-3.23 (m, 1H), 2.94-2.84 (m, 2H), 2.54 (t, J=11.1 Hz, 1H), 1.84 (s, 3H), 1.82 (s, 3H). MS m/z 575.5 [M+H]⁺.

### Example 25: Preparation of Compound 20R

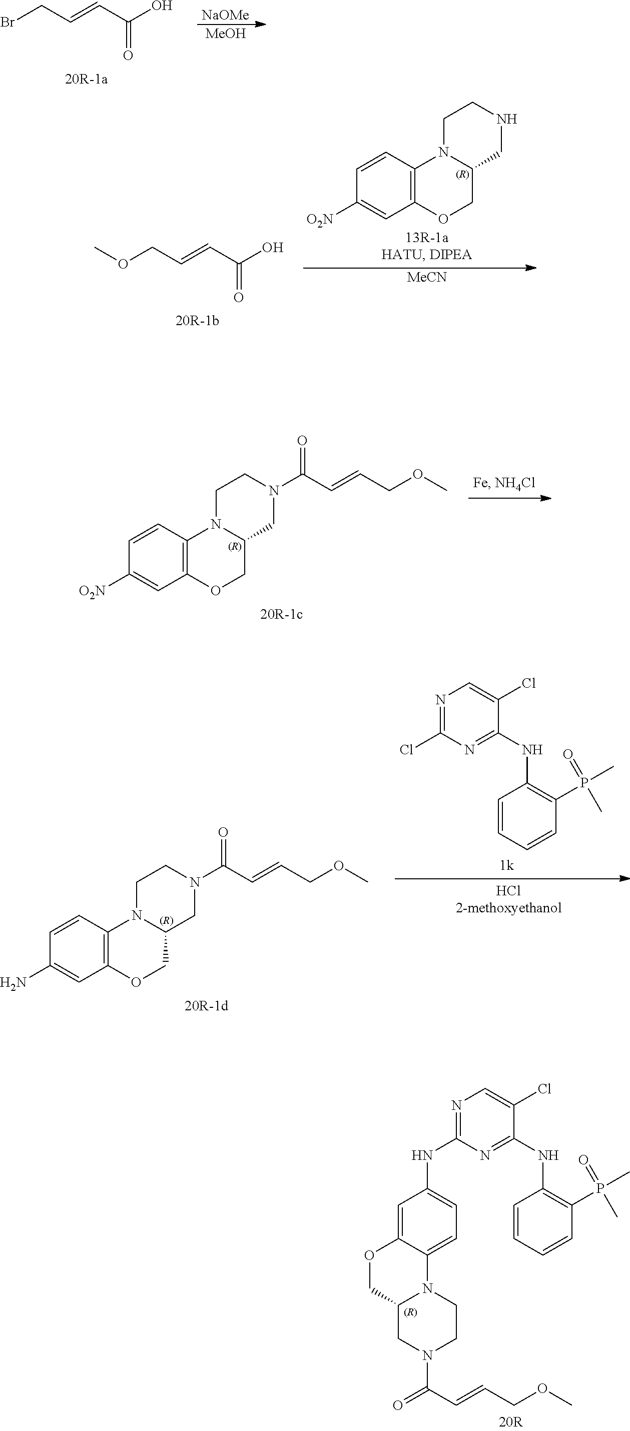

4-Bromocrotonic acid (20R-1a, 1.0 g, 6.06 mmol) was dissolved in MeOH (60 mL) at room temperature followed by the addition of a solution of sodium MeOHate in MeOH (1.64 g, 30.3 mmol, 10 mL). The reaction mixture was stirred at room temperature for 30 minutes, and then heated to reflux for 2 hours. The reaction was monitored by TLC for completion. It was cooled to room temperature, and concentrated under reduce pressure. The residue was dissolved in a mixture of water and EtOAc (v/v=50/50), acidified with 2.0 M HCl to pH=1, and extracted with EtOAc (3×20 mL). The combined organic layers were washed with brine, dried over anhydrous sodium sulfate, filtered, and concentrated under reduced pressure. The residue was purified by silica gel column chromatography (CH₂Cl₂:MeOH=2:1) to afford compound 20R-1b (622 mg, yield 88%) as a yellow solid. ¹H NMR (500 MHz, CDCl₃) δ 7.06 (dt, J=15.7, 4.1 Hz, 1H), 6.08 (dt, J=15.7, 2.1 Hz, 1H), 4.12 (dd, J=4.1, 2.1 Hz, 2H), 3.41 (s, 3H).

Compound 13R-1a (200 mg, 0.85 mmol), 4-methoxy-2-butenoic acid (20R-1b, 148 mg, 1.28 mmol), HATU (487 mg, 1.28 mmol), and DIPEA (165 mg, 1.28 mmol) were dissolved in acetonitrile (15 mL). The reaction mixture was stirred at room temperature for 30 minutes. The reaction was monitored by TLC for completion. It was concentrated under reduce pressure. The residue was purified by silica gel column chromatography (CH₂Cl₂:MeOH=20:1) to afford compound 20R-1c (700 mg) as a yellow solid which was used in next step. MS m/z 334.3 [M+H]⁺.

Compound 20R-1c (700 mg, 2.10 mmol) and iron powder (586 mg, 10.5 mmol) were added to a mixture of EtOAc/water (v/v=10 mL/6 mL) followed by the addition of NH₄Cl (702 mg, 13.12 mmol). The reaction mixture was stirred at room temperature for 4 hours. The reaction was monitored by TLC for completion. It was filtered, and filtrate was extracted with EtOAc (3×25 mL). The combined organic layers were washed with brine, dried over anhydrous sodium sulfate, filtered, and concentrated under reduced pressure. The residue was purified by silica gel column chromatography (CH₂Cl₂:MeOH=20:1) to afford compound 20R-1d (55 mg, yield of two steps 21%) as a yellow solid. MS m/z 304.4 [M+H]⁺.

Compound 20R-1d (55 mg, 0.18 mmol) and compound 1k (60 mg, 0.18 mmol) were dissolved in 2-methoxyethanol (2 mL) followed by the addition of a solution of 2.5 M HCl in MeOH (2.5 M, 0.20 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=20:1) to afford compound 20R (8.3 mg, yield 8%) as a yellow solid. ¹H NMR (500 MHz, CDCl₃) δ 10.95 (s, 1H), 8.66-8.63 (m, 1H), 8.04 (s, 1H), 7.48 (t, J=7.9 Hz, 1H), 7.30-7.24 (m, 1H), 7.20-7.09 (m, 2H), 7.04 (s, 1H), 6.98-6.89 (m, 2H), 6.77-6.70 (m, 1H), 6.61-6.51 (m, 1H), 4.82-4.60 (m, 1H), 4.32-4.25 (m, 1H), 4.17-3.90 (m, 4H), 3.73 (d, J=11.0 Hz, 1H), 3.43 (s, 3H), 3.11-2.92 (m, 2H), 2.77-2.66 (m, 1H), 2.61-2.50 (m, 1H), 1.84 (s, 3H), 1.82 (s, 3H). MS m/z 583.6 [M+H]⁺.

### Example 26: Preparation of Compound 21R

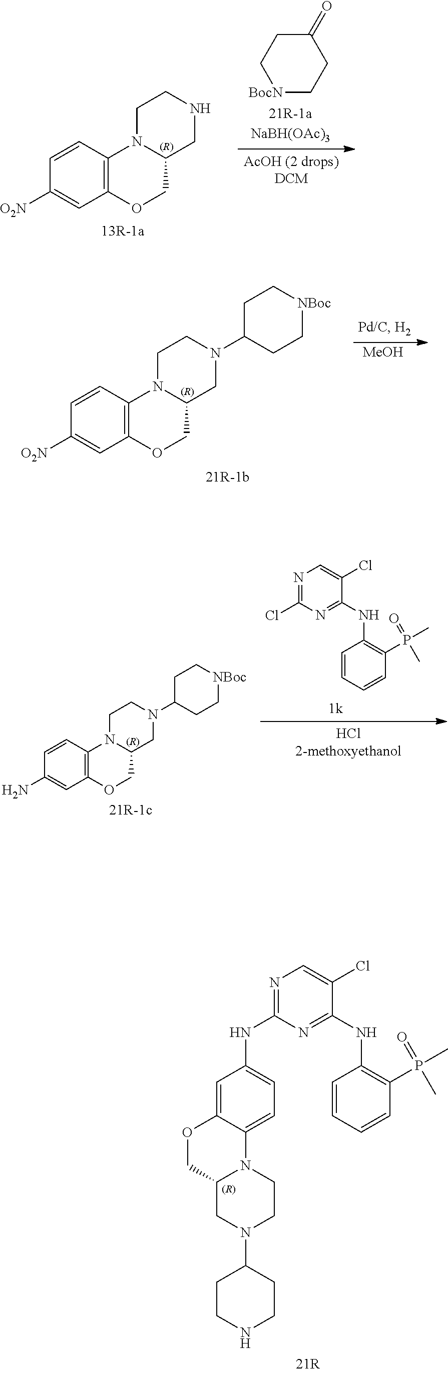

Compound 13R-1a (300 mg, 1.28 mmol) was dissolved in CH₂Cl₂ (10 mL) followed by the addition of N-Boc-piperidone (21R-1a, 381 mg, 1.91 mmol) and AcOH (2 drops). The reaction mixture was stirred at room temperature for 2 hours. Sodium triacetoxyborohydride was added (405 mg, 1.91 mmol) and the resulted mixture was stirred at room temperature overnight. After the reaction was completed, 1 N NaOH aqueous solution was added at 0° C. The mixture was poured into ice water, and extracted with CH₂Cl₂ (3×20 mL). The combined organic layers were washed with brine, dried over anhydrous sodium sulfate, filtered, and concentrated under reduced pressure. The residue was purified by silica gel column chromatography (CH₂Cl₂:MeOH=20:1) to afford compound 21R-1b (483 mg, yield 91%) as a yellow solid. MS m/z 419.5 [M+H]⁺.

Compound 21R-1b (200 mg, 0.48 mmol) and 10% Pd—C (50 mg) were added to MeOH (20 mL) at room temperature. The reaction mixture was stirred at room temperature under 1 atmospheric pressure of H₂ for 2 hours. The reaction was monitored by TLC for completion. It was filtered through celite, and the filtrate was concentrated under reduce pressure. The residue was purified by silica gel column chromatography (CH₂Cl₂:MeOH=10:1) to afford compound 21R-1c (77 mg, yield 41%) as a colorless oil. MS m/z 389.6 [M+H]⁺.

Compound 21R-1c (77 mg, 0.20 mmol) and compound 1k (63 mg, 0.20 mmol) were dissolved in 2-methoxyethanol (2 mL) followed by the addition of a solution of HCl in MeOH (2.5 M, 0.21 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=3:1, 2% of Et₃N) to afford compound 21R (26 mg, yield 23%) as a yellow solid. ¹H NMR (500 MHz, CDCl₃) δ 10.89 (s, 1H), 8.67-8.63 (m, 1H), 8.04 (s, 1H), 7.51-7.45 (m, 1H), 7.29-7.22 (m, 1H), 7.12-7.07 (m, 2H), 6.92 (dd, J=8.8, 2.5 Hz, 1H), 6.88 (s, 1H), 6.71 (d, J=8.8 Hz, 1H), 4.22-4.14 (m, 1H), 4.06-3.93 (m, 1H), 3.66 (d, J=11.4 Hz, 1H), 3.50-3.39 (m, 2H), 3.14-3.06 (m, 1H), 2.98 (d, J=10.5 Hz, 1H), 2.92-2.81 (m, 3H), 2.78-2.70 (m, 1H), 2.60-2.47 (m, 3H), 2.11-1.88 (m, 4H), 1.84 (s, 3H), 1.81 (s, 3H). MS m/z 568.5 [M+H]⁺.

### Example 27: Preparation of Compound 22R

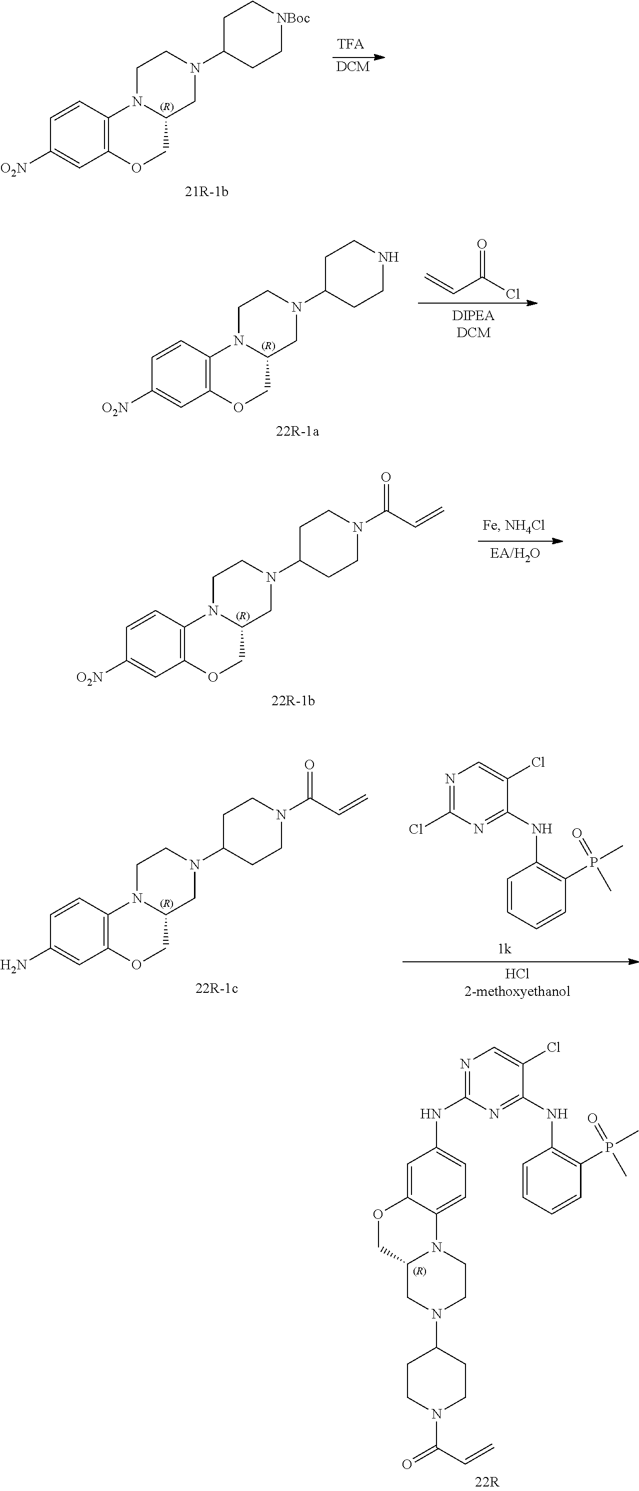

Compound 21R-1b (283 mg, 0.73 mmol) was dissolved in CH₂Cl₂ (10 mL) followed by the addition of TFA (1.5 mL) at 0° C. The reaction mixture was stirred at room temperature for 30 minutes. After the reaction was completed, it was concentrated under reduce pressure. The residue was purified by silica gel column chromatography (CH₂Cl₂:MeOH=10:1, 2% of Et₃N) to afford compound 22R-1a (150 mg, yield 65%) as a yellow solid. MS m/z 319.4 [M+H]⁺.

Compound 22R-1a (150 mg, 0.47 mmol) and DIPEA (67 mg, 0.52 mmol) were dissolved in CH₂Cl₂ (8 mL). A solution of acrylic chloride (47 mg, 0.52 mmol) in CH₂Cl₂ (2 mL) was slowly added at 0° C. The reaction mixture was stirred at room temperature for 30 minutes. After the reaction was completed, it was concentrated under reduce pressure. The residue was purified by silica gel column chromatography (CH₂Cl₂:MeOH=15:1) to afford compound 22R-1b (240 mg) as a yellow solid which was used in next step. MS m/z 373.5 [M+H]⁺.

Compound 22R-1b (240 mg, 0.64 mmol) and iron powder (180 mg, 3.22 mmol) were dispersed in a mixture of EtOAc/water (v/v=5 mL/3 mL) followed by the addition of NH₄Cl (215 mg, 4.03 mmol). The reaction mixture was stirred at room temperature overnight. After the reaction was completed, it was filtered through celite, and the filtrate was extracted with EtOAc (3×25 mL). The combined organic layers were washed with brine, dried over anhydrous sodium sulfate, filtered, and concentrated under reduced pressure. The residue was purified by silica gel column chromatography (CH₂Cl₂:MeOH=20:1) to afford compound 22R-1c (91 mg, yield of two steps 56%) as a yellow solid. MS m/z 343.5 [M+H]⁺.

Compound 22R-1c (91 mg, 0.27 mmol) and compound 1k (85 mg, 0.27 mmol) were dissolved 2-methoxyethanol (2 mL) followed by the addition of 2.5 M HCl in MeOH (0.28 mL). The reaction mixture in a sealed tube was heated to 120° C., and stirred overnight. The reaction was monitored by TLC for completion. It was cooled to room temperature, and concentrated under reduce pressure. The reaction was monitored by TLC for completion (CH₂Cl₂:MeOH=10:1) to afford compound 22R (12 mg, yield 7%) as a yellow solid. ¹H NMR (500 MHz, CDCl₃) δ 10.91 (s, 1H), 8.67-8.63 (m, 1H), 8.04 (s, 1H), 7.48 (t, J=7.9 Hz, 1H), 7.29-7.22 (m, 1H), 7.12-7.06 (m, 2H), 6.93 (dd, J=8.8, 2.5 Hz, 1H), 6.89 (s, 1H), 6.72 (d, J=8.8 Hz, 1H), 6.58 (dd, J=16.8, 10.6 Hz, 1H), 6.28 (dd, J=16.8, 1.8 Hz, 1H), 5.69 (dd, J=10.6, 1.8 Hz, 1H), 4.72 (d, J=10.5 Hz, 1H), 4.24-4.16 (m, 1H), 4.11-3.98 (m, 2H), 3.68 (d, J=11.4 Hz, 1H), 3.27-2.96 (m, 3H), 2.93-2.75 (m, 2H), 2.73-2.48 (m, 3H), 2.19-2.08 (m, 1H), 2.00-1.88 (m, 2H), 1.74-1.57 (m, 2H), 1.84 (s, 3H), 1.81 (s, 3H). MS m/z 622.6 [M+H]⁺.

### Example 28: Preparation of Compound 23R

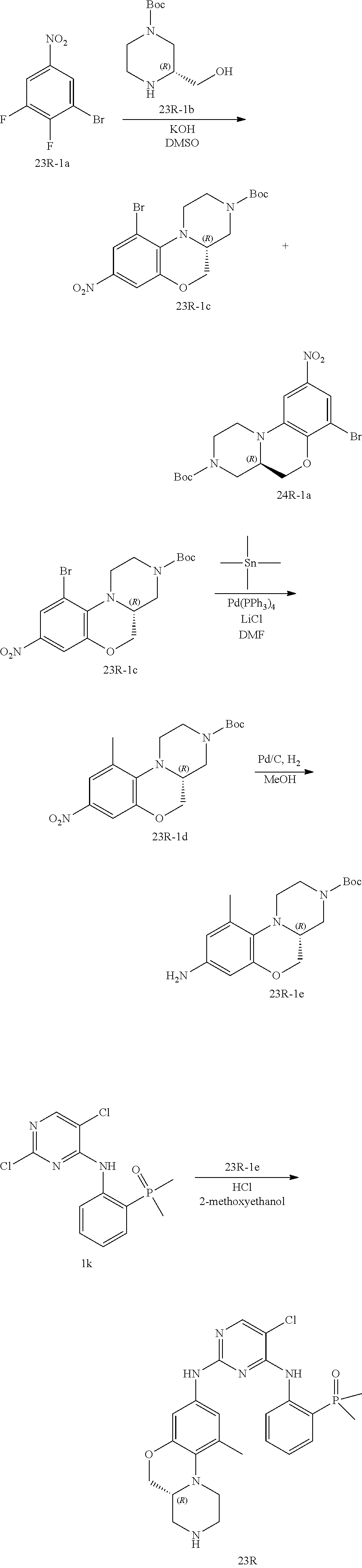

Compound 23R-1a (5.0 g, 21.01 mmol), 23R-1b (4.54 g, 21.01 mmol), and KOH (3.54 g, 63.03 mmol) were dissolved in DMSO (60 mL). The reaction mixture were stirred at room temperature for 3 hours, and then stirred at 60° C. for 3 hours. After the reaction was completed, it was poured into ice water, and stirred at room temperature for 1 hour. The mixture was extracted with CH₂Cl₂ (3×40 mL). The combined organic layers were washed with brine, dried over anhydrous sodium sulfate, filtered, and concentrated under reduced pressure. The residue was purified by silica gel column chromatography (PE:EtOAc=10:1) to afford compound 23R-1c (5.0 g, yield 57%) as a yellow solid and compound 24R-1a (1.4 g, yield 16%) as a yellow solid. 23R-1c ¹H NMR (500 MHz, CDCl₃) δ 8.04-8.02 (m, 1H), 7.72-7.70 (m, 1H), 4.25-4.14 (m, 2H), 3.87-3.76 (m, 2H), 3.64-3.55 (m, 1H), 3.51-3.32 (m, 2H), 3.29-3.16 (m, 2H), 1.49 (s, 9H); 24R-1a: ¹H NMR (500 MHz, CDCl₃) δ 7.94 (d, J=2.4 Hz, 1H), 7.63 (d, J=2.2 Hz, 1H), 4.50-4.43 (m, 1H), 4.28-4.05 (m, 3H), 3.75-3.70 (m, 1H), 3.22-3.15 (m, 1H), 3.12-2.99 (m, 1H), 2.84 (td, J=11.8, 2.9 Hz, 1H), 2.69-2.56 (m, 1H), 1.49 (s, 9H).

A mixture compound 23R-1c (2.0 g, 4.83 mmol), Sn(CH₃)₄ (1.73 g, 9.66 mmol), Pd(PPh₃)₄ (279 mg, 0.24 mmol), and LiCl (409 mg, 9.66 mmol) in DMF (35 mL) was heated to 95° C., and stirred overnight. After the reaction was completed, it was filtered, and the filtrate was concentrated under reduce pressure. The residue was purified by silica gel column chromatography (PE:EtOAc=15:1) to afford compound 23R-1d (1.37 g, yield 81%) as a yellow solid. ¹H NMR (500 MHz, CDCl₃) δ 7.64 (d, J=2.5 Hz, 1H), 7.58 (d, J=2.6 Hz, 1H), 4.25-4.13 (m, 2H), 3.87-3.80 (m, 2H), 3.60-3.52 (m, 1H), 3.35 (s, 1H), 3.23 (s, 1H), 3.10-2.97 (m, 2H), 2.36 (s, 3H), 1.48 (s, 9H).

Compound 23R-1d (200 mg, 0.57 mmol) and 10% Pd—C (80 mg) were added to MeOH (8 mL) at room temperature. The reaction mixture was stirred at room temperature under 1 atmospheric pressure of H₂ for 2 hours. The reaction was monitored by TLC for completion. It was filtered through celite, and the filtrate was concentrated under reduce pressure. The residue was purified by silica gel column chromatography (PE:EtOAc=2:1) to afford compound 23R-1e (162 mg, 89%) as an off-white solid. ¹H NMR (500 MHz, CDCl₃) δ 6.14 (d, J=2.4 Hz, 1H), 6.10 (d, J=2.4 Hz, 1H), 4.31 (t, J=10.9 Hz, 1H), 4.17-3.93 (m, 3H), 3.34 (s, 1H), 3.20-2.97 (m, 2H), 2.90 (d, J=12.2 Hz, 1H), 2.79-2.66 (m, 1H), 2.20 (s, 3H), 1.47 (s, 9H).

Compound 23R-1e (50 mg, 0.16 mmol) and compound 1k (50 mg, 0.16 mmol) were dissolved in 2-methoxyethanol (2 mL) followed the addition of a solution of 2.5 M HCl in MeOH (0.16 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=10:1, 2% of Et₃N) to afford compound 23R (59 mg, yield 75%) as a light yellow solid. ¹H NMR (500 MHz, CDCl₃) δ 10.86 (s, 1H), 8.67-8.63 (m, 1H), 8.07 (s, 1H), 7.53-7.47 (m, 1H), 7.30-7.23 (m, 1H), 7.13-7.09 (m, 1H), 7.08 (d, J=2.4 Hz, 1H), 6.92 (s, 1H), 6.81 (d, J=2.3 Hz, 1H), 4.67 (t, J=10.8 Hz, 1H), 4.05 (dd, J=10.8, 3.0 Hz, 1H), 3.35 (dd, J=12.8, 4.3 Hz, 1H), 3.23-3.18 (m, 1H), 3.17-3.00 (m, 4H), 2.94-2.87 (m, 1H), 2.25 (s, 3H), 1.84 (s, 3H), 1.82 (s, 3H). MS m/z 499.4 [M+H]⁺.

### Example 29: Preparation of Compound 23S

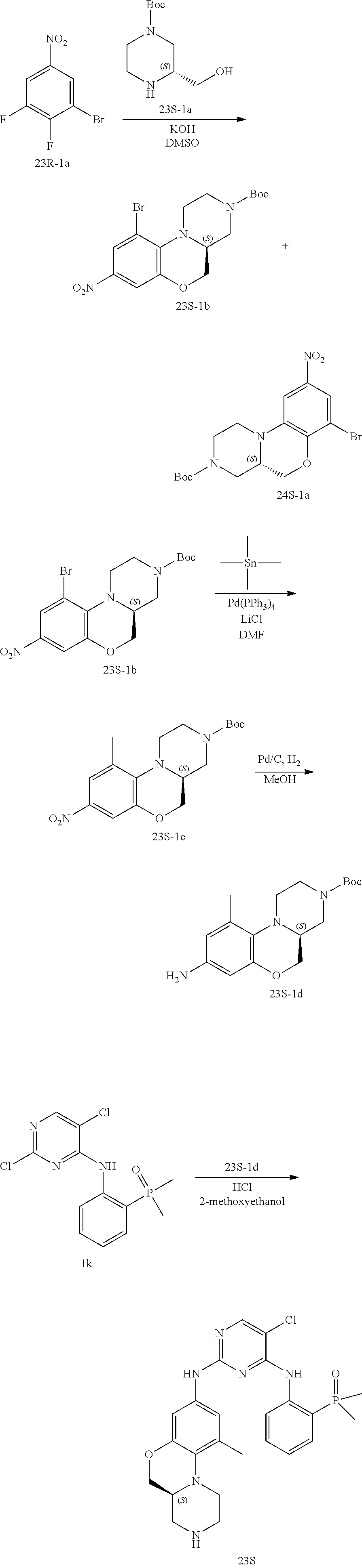

Compound 23R-1a (100 mg, 0.42 mmol), 23S-1a (91 mg, 0.42 mmol), and KOH (71 mg, 1.26 mmol) were dissolved in DMSO (5 mL). The reaction mixture was stirred at room temperature for 3 hours, and stirred at 60° C. for 3 hours. After the reaction was completed, it was poured into ice water and stirred at room temperature for 1 hour. The mixture was extracted with CH₂Cl₂ (3×25 mL). The combined organic layers were washed with brine, dried over anhydrous sodium sulfate, filtered, and concentrated under reduced pressure. The residue was purified by silica gel column chromatography (PE:EtOAc=10:1) to afford compound 23S-1b (64 mg, yield 37%) as a yellow solid, and compound 24S-1a (64 mg, yield 37%) as a yellow solid. 23S-1b: ¹H NMR (500 MHz, CDCl₃) δ 8.03 (d, J=2.5 Hz, 1H), 7.71 (d, J=2.6 Hz, 1H), 4.26-4.15 (m, 2H), 3.86-3.78 (m, 2H), 3.60 (dd, J=13.8, 4.1 Hz, 1H), 3.52-3.36 (m, 2H), 3.29-3.15 (m, 2H), 1.48 (s, 9H); 24S-1a: ¹H NMR (500 MHz, CDCl₃) δ 7.94 (d, J=2.5 Hz, 1H), 7.63 (d, J=2.5 Hz, 1H), 4.49-4.44 (m, 1H), 4.28-4.04 (m, 3H), 3.71 (d, J=11.3 Hz, 1H), 3.22-3.15 (m, 1H), 3.10-3.01 (m, 1H), 2.88-2.79 (m, 1H), 2.66-2.57 (m, 1H), 1.49 (s, 9H).

Compound 23S-1b (64 mg, 0.16 mmol), Sn(CH₃)₄ (56 mg, 0.31 mmol), Pd(PPh₃)₄ (9 mg, 0.01 mmol), and LiCl (13 mg, 0.31 mmol) in DMF (2 mL) was heated to 90° C., and stirred overnight. After the reaction was completed, it was filtered, and the filtrate was concentrated under reduce pressure. The residue was purified by silica gel column chromatography (PE:EtOAc=10:1) to afford compound 23S-1c (33 mg, yield 60%) as a yellow solid. ¹H NMR (500 MHz, CDCl₃) δ 7.65 (d, J=2.5 Hz, 1H), 7.59 (d, J=2.6 Hz, 1H), 4.24-4.14 (m, 2H), 3.89-3.78 (m, 2H), 3.60-3.49 (m, 1H), 3.39-3.30 (m, 1H), 3.28-3.18 (m, 1H), 3.10-2.98 (m, 2H), 2.37 (s, 3H), 1.48 (s, 9H).

Compound 23S-1c (33 mg, 0.09 mmol) and 10% Pd—C (40 mg) were added to MeOH (6 mL) at room temperature. The reaction mixture was stirred at room temperature under 1 atmospheric pressure of H₂ for 2 hours. The reaction was monitored by TLC for completion. It was filtered through celite, and the filtrate was concentrated under reduce pressure. The residue was purified by silica gel column chromatography (PE:EtOAc=2:1) to afford compound 23S-1d (23 mg, 76%) as a light yellow solid. MS m/z 320.4 [M+H]⁺.

Compound 23S-1d (23 mg, 0.07 mmol) and compound 1k (23 mg, 0.07 mmol) were dissolved in 2-methoxyethanol (1 mL) followed by the addition of 2.5 M HCl in MeOH (0.08 mL). The reaction mixture in a sealed tube was heated to 120° C., and stirred overnight. The reaction was monitored by TLC for completion. It was cooled to room temperature and concentrated under reduce pressure. The residue was purified by prep-TLC (CH₂Cl₂:MeOH=5:1) to afford compound 23S (14 mg, yield 38%) as a light yellow solid. ¹H NMR (500 MHz, CD₃OD) δ 8.50-8.45 (m, 1H), 8.04 (s, 1H), 7.62-7.53 (m, 2H), 7.27-7.22 (m, 1H), 6.98 (d, J=2.4 Hz, 1H), 6.82 (d, J=2.1 Hz, 1H), 4.53 (t, J=11.0 Hz, 1H), 4.02 (dd, J=10.7, 2.8 Hz, 1H), 3.30-3.26 (m, 1H), 3.18-3.09 (m, 2H), 3.07-2.99 (m, 3H), 2.87-2.80 (m, 1H), 2.20 (s, 3H), 1.86 (s, 3H), 1.83 (s, 3H). MS m/z 499.4 [M+H]⁺.

### Example 30: Preparation of Compound 24R

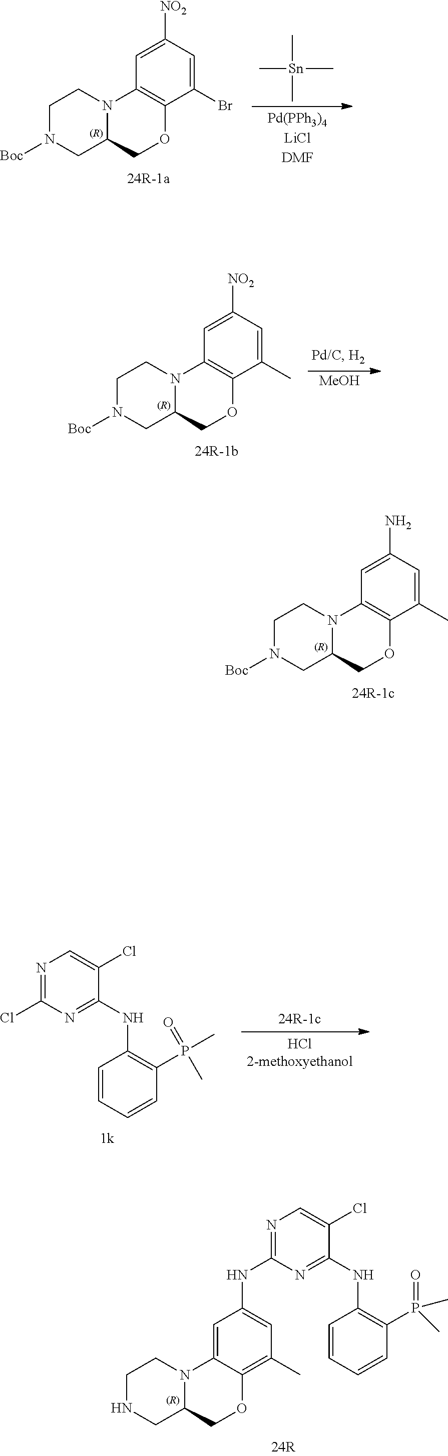

A mixture of compound 24R-1a (1.0 g, 2.41 mmol), Sn(CH₃)₄ (863 mg, 4.83 mmol), Pd(PPh₃)₄ (140 mg, 0.12 mmol), and LiCl (205 mg, 4.83 mmol) in DMF (15 mL) was heated to 95° C., and stirred overnight. After the reaction was completed, it was filtered, and the filtrate was concentrated under reduce pressure. The residue was purified by silica gel column chromatography (PE:EtOAc=15:1) to afford compound 24R-1b (861 mg, yield 100%) as a yellow solid. ¹H NMR (500 MHz, CDCl₃) δ 7.60-7.54 (m, 2H), 4.38 (dd, J=10.8, 2.8 Hz, 1H), 4.26-4.09 (m, 2H), 4.06 (dd, J=10.8, 8.8 Hz, 1H), 3.73-3.68 (m, 1H), 3.14-3.01 (m, 2H), 2.82-2.73 (m, 1H), 2.65-2.55 (m, 1H), 2.21 (s, 3H), 1.49 (s, 9H).

Compound 24R-1b (150 mg, 0.43 mmol) and 10% Pd—C (50 mg) were added to MeOH (6 mL) at room temperature. The reaction mixture was stirred at room temperature under 1 atmospheric pressure of H₂ for 2 hours. The reaction was monitored by TLC for completion. It was filtered through celite, and the filtrate was concentrated under reduce pressure. The residue was purified by silica gel column chromatography (PE:EtOAc=2:1) to afford compound 24R-1c (119 mg, 87%) as an off-white solid. ¹H NMR (500 MHz, CDCl₃) δ 6.11 (d, J=2.3 Hz, 1H), 6.05 (d, J=2.0 Hz, 1H), 4.19 (dd, J=10.7, 2.7 Hz, 1H), 4.15-3.98 (m, 2H), 3.91 (dd, J=10.6, 8.4 Hz, 1H), 3.62-3.57 (m, 1H), 3.10-3.03 (m, 1H), 3.02-2.97 (m, 1H), 2.74-2.67 (m, 1H), 2.64-2.59 (m, 1H), 2.09 (s, 3H), 1.48 (s, 9H).

Compound 24R-1c (50 mg, 0.16 mmol) and compound 1k (50 mg, 0.16 mmol) were dissolved in 2-methoxyethanol (2 mL) followed by the addition of 2.5 M HCl in MeOH (0.16 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=10:1, 2% of Et₃N) to afford compound 24R (52 mg, yield 66%) as a light yellow solid. ¹H NMR (500 MHz, DMSO-d₆) δ 11.14 (s, 1H), 9.04 (s, 1H), 8.67-8.52 (m, 1H), 8.14 (s, 1H), 7.61-7.55 (m, 1H), 7.46 (t, J=7.8 Hz, 1H), 7.16 (t, J=7.4 Hz, 1H), 6.98 (s, 1H), 6.91 (s, 1H), 4.32-4.28 (m, 1H), 3.96-3.91 (m, 1H), 3.59 (d, J=12.3 Hz, 1H), 3.30-3.24 (m, 3H), 2.92 (t, J=12.1 Hz, 1H), 2.83 (t, J=12.4 Hz, 1H), 2.69 (t, J=11.8 Hz, 1H), 2.05 (s, 3H), 1.80 (s, 3H), 1.77 (s, 3H). MS m/z 499.4 [M+H]⁺.

### Example 31: Preparation of Compound 24S

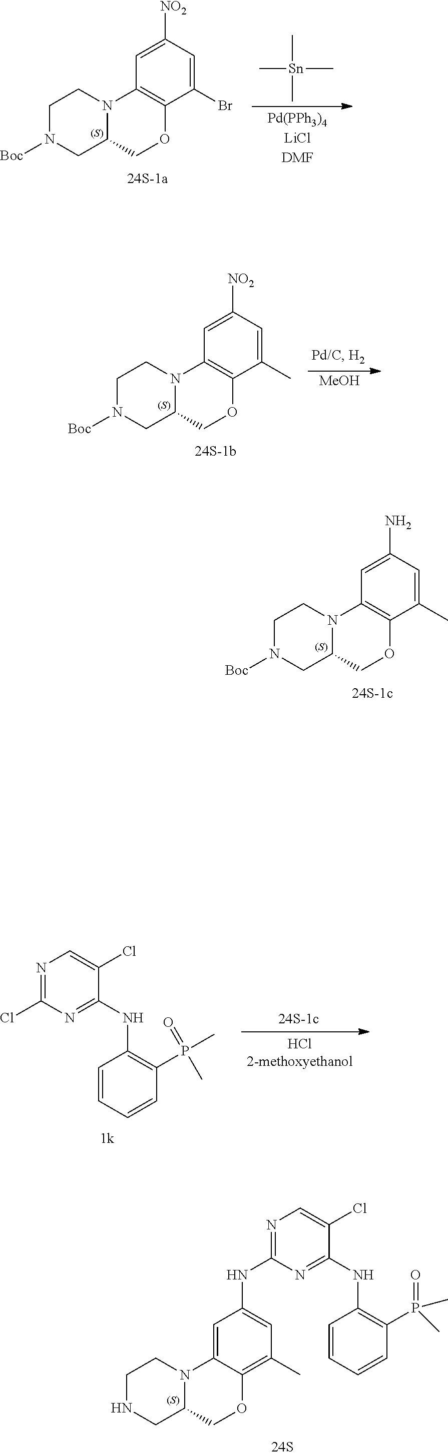

A mixture of compound 24S-1a (39 mg, 2.41 mmol), Sn(CH₃)₄ (34 mg, 0.19 mmol), Pd(PPh₃)₄ (6 mg, 0.004 mmol), and LiCl (8 mg, 0.19 mmol) in DMF (2 mL) was heated to 95° C., and stirred overnight. After the reaction was completed, it was filtered, and the filtrate was concentrated under reduce pressure. The residue was purified by silica gel column chromatography (PE:EtOAc=10:1) to afford compound 24S-1b (28 mg, yield 85%) as a yellow solid. ¹H NMR (500 MHz, CDCl₃) δ 7.60-7.55 (m, 2H), 4.40-4.35 (m, 1H), 4.25-4.02 (m, 3H), 3.71 (d, J=11.4 Hz, 1H), 3.16-3.00 (m, 2H), 2.83-2.73 (m, 1H), 2.66-2.57 (m, 1H), 2.21 (s, 3H), 1.49 (s, 9H).

Compound 24S-1b (28 mg, 0.08 mmol) and 10% Pd—C (40 mg) were added to MeOH (6 mL) at room temperature. The reaction mixture was stirred at room temperature under 1 atmospheric pressure of H₂ for 2 hours. The reaction was monitored by TLC for completion. It was filtered through celite, and the filtrate was concentrated under reduce pressure. The residue was purified by silica gel column chromatography (PE:EtOAc=2:1) to afford compound 24S-1c (12 mg, 47%) as an off-white solid. MS m/z 320.4 [M+H]⁺.

Compound 24S-1c (12 mg, 0.04 mmol) and compound 1k (12 mg, 0.04 mmol) were dissolved in 2-methoxyethanol (1 mL) followed by the addition of 2.5 M HCl in MeOH (2.5 M, 0.04 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=5:1) to afford compound 24S (8 mg, yield 42%) as a yellow solid. ¹H NMR (500 MHz, CD₃OD) δ 8.52-8.48 (m, 1H), 8.02 (s, 1H), 7.62-7.55 (m, 1H), 7.51 (t, J=7.9 Hz, 1H), 7.25-7.20 (m, 1H), 6.89 (d, J=2.0 Hz, 1H), 6.74 (d, J=1.9 Hz, 1H), 4.27-4.21 (m, 1H), 3.96-3.90 (m, 1H), 3.50 (d, J=11.9 Hz, 1H), 3.08-3.01 (m, 3H), 2.89-2.80 (m, 1H), 2.63-2.56 (m, 1H), 2.51 (t, J=11.7 Hz, 1H), 2.07 (s, 3H), 1.87 (d, J=2.9 Hz, 3H), 1.84 (d, J=2.9 Hz, 3H). MS m/z 499.4 [M+H]⁺.

### Example 32: Preparation of Compound 25R

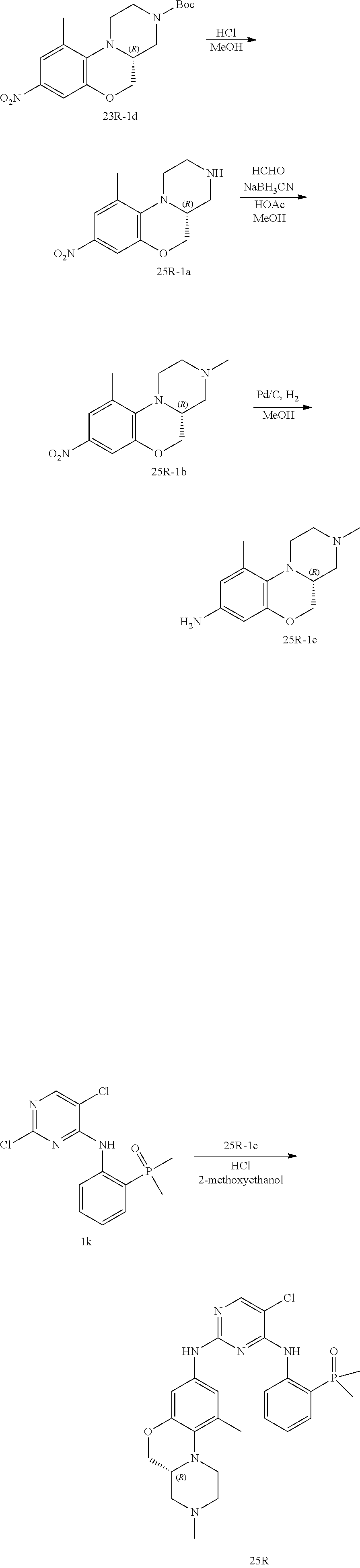

Compound 23R-1d (1.17 g, 3.35 mmol) was dissolved in MeOH (15 mL) at room temperature followed by the addition of 4.0 M HCl in MeOH (1.36 mL). The reaction mixture was stirred at 60° C. for 1 hour. After the reaction was completed, it was concentrated under reduce pressure. The residue was dissolved in CH₂Cl₂, and neutralized with saturated NaHCO₃ aqueous solution. The mixture was extracted with CH₂Cl₂ (3×20 mL). The combined organic layers were washed with brine, dried over anhydrous sodium sulfate, filtered, and concentrated under reduced pressure. The residue was purified by silica gel column chromatography (CH₂Cl₂:MeOH=20:1) to afford compound 25R-1a (465 mg, yield 56%) as a yellow solid. ¹H NMR (500 MHz, CDCl₃) δ 7.57 (d, J=2.5 Hz, 1H), 7.51 (d, J=2.6 Hz, 1H), 4.37-4.27 (m, 1H), 4.10-4.01 (m, 1H), 3.19-3.12 (m, 2H), 3.09-2.91 (m, 5H), 2.29 (s, 3H).

A mixture of compound 25R-1a (150 mg, 0.60 mmol), 37% formaldehyde aqueous solution (37%, 1.5 mL), and AcOH (2 drops) in MeOH (6 mL) was stirred at room temperature for 1 hour. Sodium cyanoborohydride (113 mg, 1.81 mmol) was added, and the reaction mixture was stirred at room temperature overnight. After the reaction was completed, it was concentrated under reduce pressure. Water (15 mL) was added, and the mixture was extracted with CH₂Cl₂ (3×15 mL). The combined organic layers were washed with brine, dried over anhydrous sodium sulfate, filtered, and concentrated under reduced pressure. The residue was purified by silica gel column chromatography (CH₂Cl₂:MeOH=30:1) to afford compound 25R-1b (155 mg, yield 98%) as a yellow solid. MS m/z 264.4 [M+H]J.

Compound 25R-1b (155 mg, 0.59 mmol) and 10% Pd—C (80 mg) were added to MeOH (8 mL) at room temperature. The reaction mixture was stirred at room temperature under 1 atmospheric pressure of H₂ for 2 hours. The reaction was monitored by TLC for completion. It was filtered through celite, and the filtrate was concentrated under reduce pressure. The residue was purified by silica gel column chromatography (PE:EtOAc=2:1) to afford compound 25R-1c (123 mg, 90%) as a light yellow solid. ¹H NMR (500 MHz, CDCl₃) δ 6.12 (d, J=2.6 Hz, 1H), 6.09 (d, J=2.6 Hz, 1H), 4.62 (t, J=10.7 Hz, 1H), 4.01-3.96 (m, 1H), 3.42 (bs, 2H), 3.18 (d, J=10.6 Hz, 1H), 3.02-2.76 (m, 4H), 2.59 (d, J=9.4 Hz, 1H), 2.37-2.33 (m, 1H), 2.31 (s, 3H), 2.19 (s, 3H).

Compound 25R-1c (50 mg, 0.22 mmol) and compound 1k (68 mg, 0.22 mmol) were dissolved in 2-methoxyethanol (1.5 mL) followed by the addition of 2.5 M HCl in MeOH (0.22 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=20:1) to afford compound 25R (73 mg, yield 66%) as a light yellow solid. ¹H NMR (500 MHz, CDCl₃) δ 10.89 (s, 1H), 8.68-8.64 (m, 1H), 8.07 (s, 1H), 7.49 (t, J=7.9 Hz, 1H), 7.30-7.22 (m, 1H), 7.12-7.05 (m, 2H), 6.96 (s, 1H), 6.80 (d, J=2.3 Hz, 1H), 4.62 (t, J=10.7 Hz, 1H), 4.05 (dd, J=10.8, 2.8 Hz, 1H), 3.21 (d, J=10.8 Hz, 1H), 3.07-3.02 (m, 1H), 2.98-2.91 (m, 1H), 2.87 (d, J=11.8 Hz, 1H), 2.80 (d, J=10.0 Hz, 1H), 2.63-2.56 (m, 1H), 2.39-2.32 (m, 1H), 2.31 (s, 3H), 2.25 (s, 3H), 1.84 (s, 3H), 1.81 (s, 3H). MS m/z 513.4 [M+H]⁺.

### Example 33: Preparation of Compound 26R

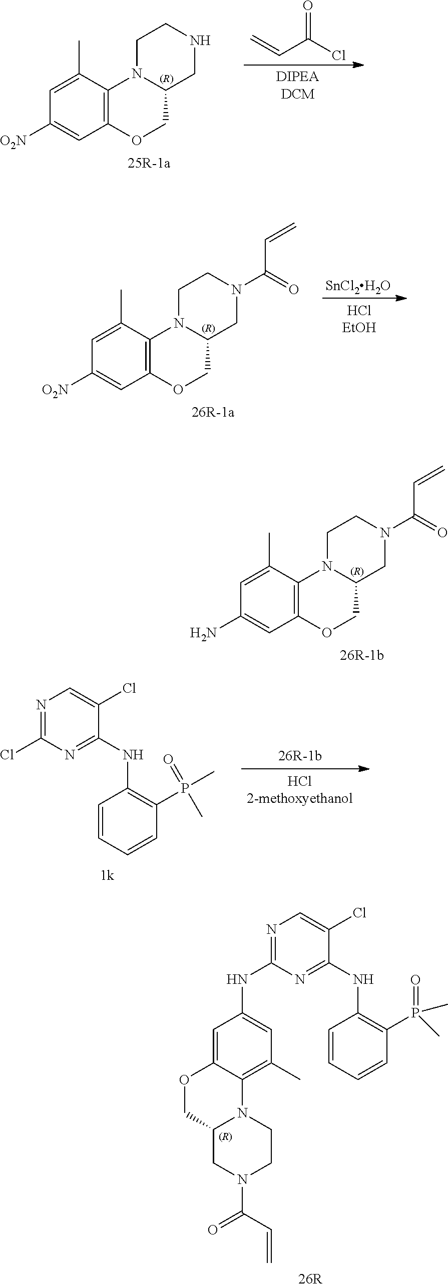

Compound 25R-1a (150 mg, 0.60 mmol) and DIPEA (156 mg, 1.20 mmol) were dissolved in CH₂Cl₂ (6 mL) followed by the addition of a solution of acrylic chloride (60 mg, 0.66 mmol) in CH₂Cl₂ (4 mL) at 0° C. The reaction mixture was stirred at room temperature for 1 hour. After the reaction was completed, it was concentrated under reduce pressure. The residue was purified by silica gel column chromatography (PE:EtOAc=2:1) to afford compound 26R-1a (190 mg, yield 100%) as a yellow solid. MS m/z 304.3 [M+H]⁺.

To a mixture of compound 26R-1a (190 mg, 0.63 mmol) and tin(II) chloride dihydrate (650 mg, 3.13 mmol) in ethanol (10 mL) was added 2 M HCl (1.57 mL). The reaction mixture was stirred at 90° C. for 6 hours. The reaction was monitored by TLC for completion. It was concentrated under reduce pressure, dissolved in CH₂Cl₂, neutralized with saturated NaHCO₃ aqueous solution, and extracted with CH₂Cl₂ (3×20 mL). The combined organic layers were washed with brine, dried over anhydrous sodium sulfate, filtered, and concentrated under reduced pressure. The residue was purified by silica gel column chromatography (CH₂Cl₂:MeOH=20:1) to afford compound 26R-1b (103 mg, yield 60%) as a light yellow solid. MS m/z 274.3 [M+H]⁺.

Compound 26R-1b (50 mg, 0.18 mmol) and compound 1k (58 mg, 0.18 mmol) were dissolved in 2-methoxyethanol (1.5 mL) followed by the addition of 2.5 M HCl in MeOH (2.5 M, 0.19 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=20:1, 2% of Et₃N) to afford compound 26R (16 mg, yield 16%) as a white solid. ¹H NMR (500 MHz, CDCl₃) δ 10.97 (s, 1H), 8.65-8.61 (m, 1H), 8.06 (s, 1H), 7.53-7.47 (m, 1H), 7.31-7.24 (m, 1H), 7.17-7.08 (m, 2H), 7.00 (s, 1H), 6.87-6.75 (m, 1H), 6.63-6.51 (m, 1H), 6.36 (d, J=16.7 Hz, 1H), 5.76 (d, J=10.0 Hz, 1H), 4.70-4.55 (m, 1H), 4.38-4.20 (m, 1H), 4.16-3.89 (m, 2H), 3.55-3.45 (m, 1H), 3.37-3.19 (m, 2H), 3.07 (d, J=12.1 Hz, 1H), 2.80 (t, J=12.3 Hz, 1H), 2.27 (s, 3H), 1.85 (s, 3H), 1.82 (s, 3H). MS m/z 553.5 [M+H]⁺.

### Example 34: Preparation of Compound 27R

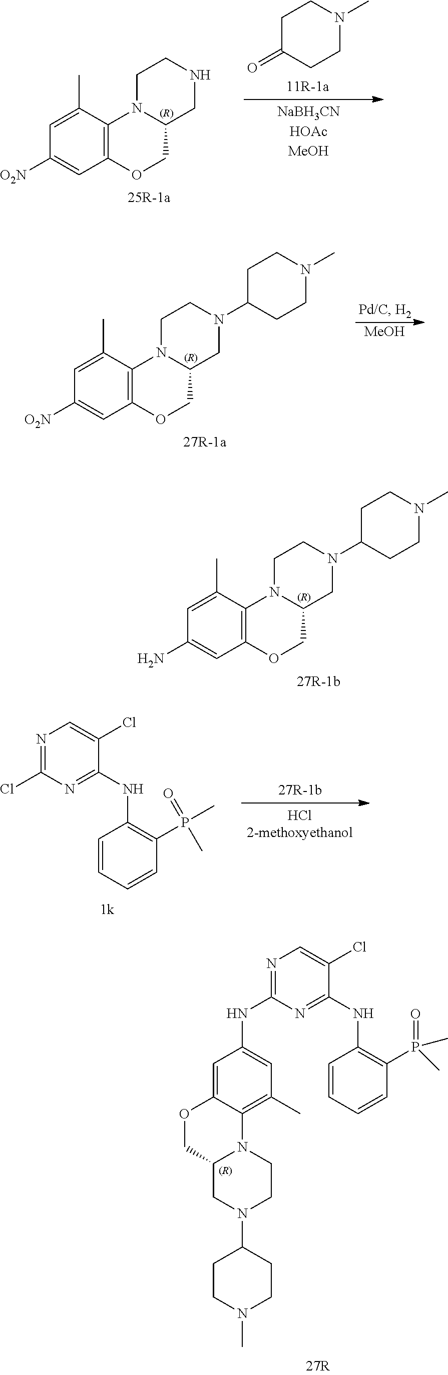

A mixture of compound 25R-1a (150 mg, 0.60 mmol), N-methylpiperidone (1R-1a, 340 mg, 3.01 mmol), and AcOH (2 drops) in MeOH (8 mL) was stirred at room temperature for 1 hour. Sodium cyanoborohydride (113 mg, 1.81 mmol) was added and the reaction mixture was stirred at room temperature overnight. After the reaction was completed, it was concentrated under reduce pressure. The residue was extracted with CH₂Cl₂ (3×15 mL). The combined organic layers were washed with brine, dried over anhydrous sodium sulfate, filtered, and concentrated under reduced pressure. The residue was purified by silica gel column chromatography (CH₂Cl₂:MeOH=30:1) to afford compound 27R-1a (219 mg) as a yellow solid which was used in next step. MS m/z 347.4 [M+H]⁺.

Compound 27R-1a (219 mg, 0.63 mmol) and 100 Pd—C (80 mg) were added to MeOH (10 mL) at room temperature. The reaction mixture was stirred at room temperature under 1 atmospheric pressure of H₂ for 2 hours. The reaction was monitored by TLC for completion. It was filtered through celite, and the filtrate was concentrated under reduce pressure. The residue was purified by silica gel column chromatography (CH₂Cl₂:MeOH=20:1, 2% of Et₃N) to afford compound 27R-1b (182 mg, yield of two steps 96%) as an off-white solid. MS m/z 317.4 [M+H]n.

Compound 27R-1b (50 mg, 0.16 mmol) and compound 1k (50 mg, 0.16 mmol) were dissolved in 2-methoxyethanol (1.5 mL) followed by the addition of 2.5 M HCl in MeOH (2.5 M, 0.16 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=50:1, 2% of Et₃N) to afford compound 27R (43 mg, yield 46%) as a light yellow solid. MH NMR (500 MHz, CD₃OD) 8.51-8.45 (m, 1H), 8.02 (s, 1H), 7.60-7.50 (m, 2H), 7.22 (t, J=6.5 Hz, 1H), 6.96 (d, J=2.3 Hz, 1H), 6.79 (d, J=2.3 Hz, 1H), 4.53 (t, J=10.6 Hz, 1H), 3.94 (dd, J=10.6, 2.7 Hz, 1H), 3.11-3.01 (m, 2H), 3.00-2.77 (m, 5H), 2.69 (dd, J=11.7, 4.1 Hz, 1H), 2.51-2.42 (m, 1H), 2.26 (s, 3H), 2.23-2.15 (m, 1H), 2.17 (s, 3H), 2.05 (t, J=11.0 Hz, 2H), 1.88-1.80 (m, 2H), 1.84 (s, 3H), 1.82 (s, 3H), 1.63-1.49 (m, 2H). MS m/z 596.6 [M+H]⁺.

### Example 35: Preparation of Compound 27S

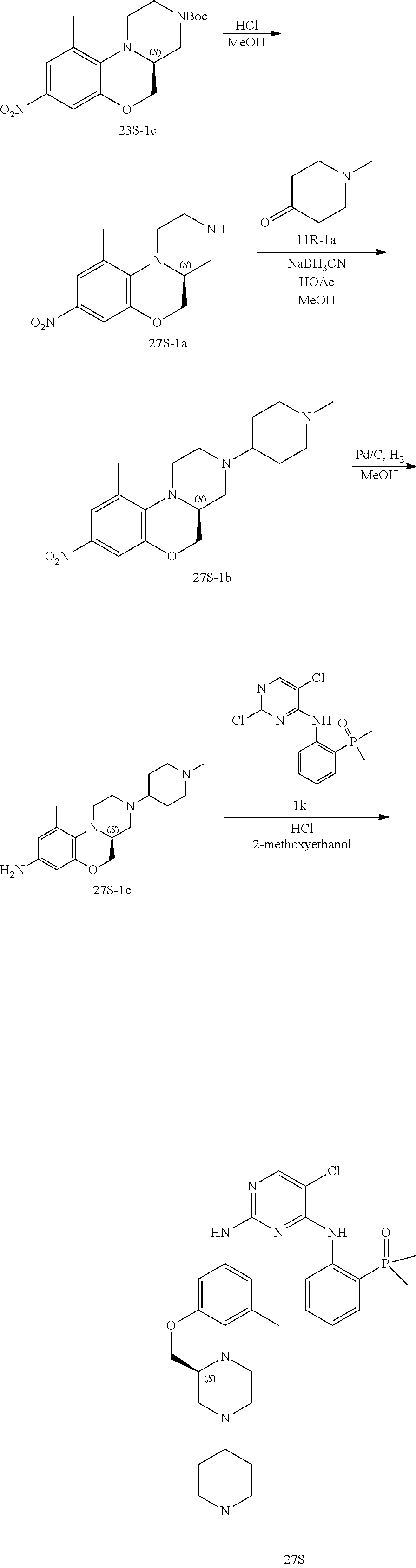

Compound 23S-1c (200 mg, 0.57 mmol) was dissolved MeOH (2 mL). 4 M HCl in MeOH (1 mL) was added under ice bath. The reaction mixture was stirred at room temperature 1 hour. The reaction was monitored by TLC for completion. It was concentrated under reduce pressure, neutralized with saturated NaHCO₃ aqueous solution (10 mL), and extracted with EtOAc (3×10 mL). The combined organic layers were washed with brine, dried over anhydrous sodium sulfate, filtered, and concentrated under reduced pressure compound 27S-1a (100 mg, yield 70%) as a yellow solid. MS m/z 250.3 [M+H]⁺.

A mixture of compound 27S-1a (100 mg, 0.40 mmol), N-methylpiperidone (11R-1a, 227 mg, 2.00 mmol), and AcOH (2 drops) in MeOH (5 mL) was stirred at room temperature for 1 hour. Sodium cyanoborohydride (75 mg, 1.21 mmol) was added, and the reaction mixture was stirred at room temperature overnight. After the reaction was completed, it was concentrated under reduce pressure, and extracted with CH₂Cl₂ (3×15 mL). The combined organic layers were washed with brine, dried over anhydrous sodium sulfate, filtered, and concentrated under reduced pressure. The residue was purified by silica gel column chromatography (CH₂Cl₂:MeOH=30:1) to afford compound 27S-1b (140 mg) as a yellow solid which was used in next step. MS m/z 347.4 [M+H]⁺.

Compound 27S-1b (55 mg, 0.16 mmol) and 10% Pd—C (10 mg) were added to MeOH (10 mL) at room temperature. The reaction mixture was stirred at room temperature under 1 atmospheric pressure of H₂ for 2 hours. The reaction was monitored by TLC for completion. It was filtered through celite, and the filtrate was concentrated under reduce pressure to afford compound 27S-1c (50 mg, yield 99%) as an off-white solid. MS m/z 317.4 [M+H]⁺.

Compound 27S-1c (50 mg, 0.16 mmol) and compound 1k (50 mg, 0.16 mmol) were dissolved in 2-methoxyethanol (1.5 mL) followed by the addition of 2.5 M HCl in MeOH (2.5 M, 0.16 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=50:1, 2% of Et₃N) to afford compound 27S (33 mg, yield 35%) as a light yellow solid. ¹H NMR (500 MHz, CD₃OD) δ 8.51-8.45 (m, 1H), 8.03 (s, 1H), 7.60-7.51 (m, 2H), 7.23 (t, J=6.5 Hz, 1H), 6.96 (s, 1H), 6.80 (s, 1H), 4.55 (t, J=10.8 Hz, 1H), 3.97 (dd, J=10.5, 2.0 Hz, 1H), 3.13-3.05 (m, 2H), 3.00-2.94 (m, 3H), 2.90-2.82 (m, 2H), 2.72 (dd, J=12.0, 4.0 Hz, 1H), 2.51-2.42 (m, 1H), 2.32 (s, 3H), 2.26-2.12 (m, 3H), 2.18 (s, 3H), 1.93-1.80 (m, 2H), 1.85 (s, 3H), 1.82 (s, 3H), 1.65-1.52 (m, 2H). MS m/z 596.6 [M+H]⁺.

### Example 36: Preparation of Compound 28R

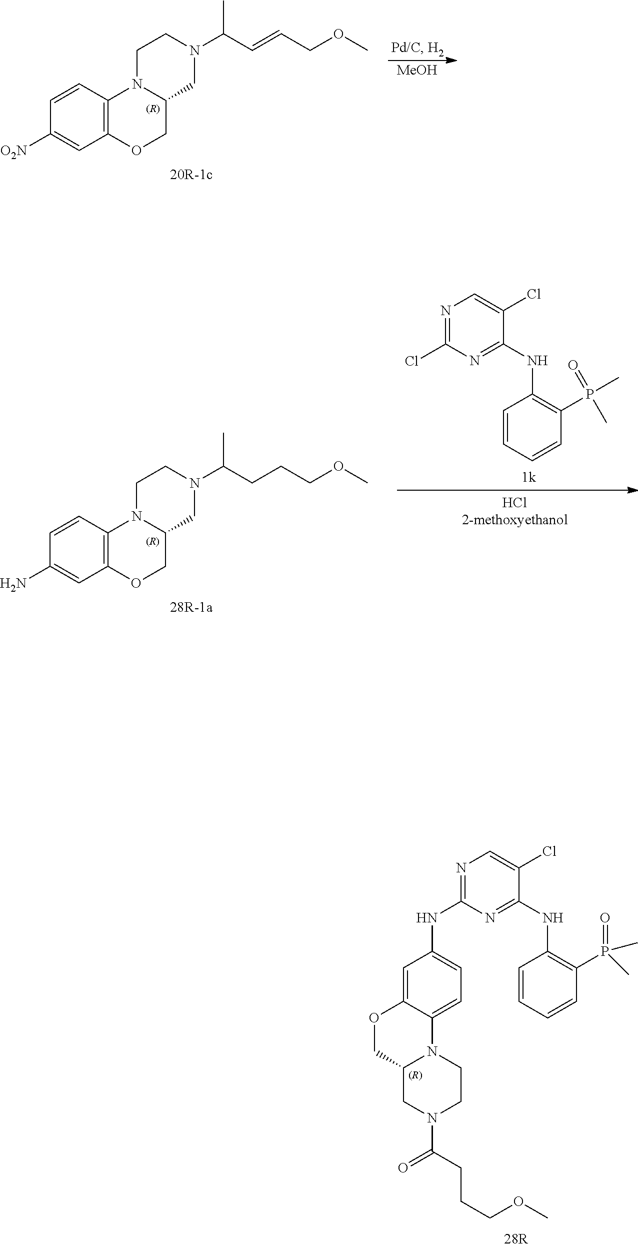

Compound 20R-1c (123 mg, 0.37 mmol) and 10% Pd—C (30 mg) were dissolved in MeOH (6 mL) at room temperature. The reaction mixture was stirred at room temperature under 1 atmospheric pressure of H₂ for 2 hours. The reaction was monitored by TLC for completion. It was filtered through celite, and the filtrate was concentrated under reduce pressure. The residue was purified by silica gel column chromatography (CH₂Cl₂:MeOH=15:1) to afford compound 28R-1a (73 mg, 65%) as a yellow solid. MS m/z 306.5 [M+H]⁺.

Compound 28R-1a (73 mg, 0.24 mmol) and compound 1k (76 mg, 0.24 mmol) were dissolved in 2-methoxyethanol (2 mL) followed by the addition of 2.5 M HCl in MeOH (2.5 M, 0.25 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=10:1) to afford compound 28R (28 mg, yield 21%) as a yellow solid. ¹H NMR (500 MHz, CDCl₃) δ 10.95 (s, 1H), 8.67-8.58 (m, 1H), 8.05 (s, 1H), 7.48 (t, J=7.7 Hz, 1H), 7.30-7.24 (m, 1H), 7.19-7.09 (m, 2H), 6.99-6.88 (m, 2H), 6.77-6.70 (m, 1H), 4.68 (dd, J=53.0, 13.0 Hz, 1H), 4.30-4.22 (m, 1H), 4.08-3.81 (m, 2H), 3.71 (d, J=11.8 Hz, 1H), 3.44 (t, J=5.9 Hz, 2H), 3.34 (s, 3H), 3.10-2.84 (m, 2H), 2.74-2.63 (m, 1H), 2.57-2.41 (m, 3H), 1.98-1.90 (m, 2H), 1.84 (s, 3H), 1.82 (s, 3H). MS m/z 585.6 [M+H]⁺.

### Example 37: Preparation of Compound 29R

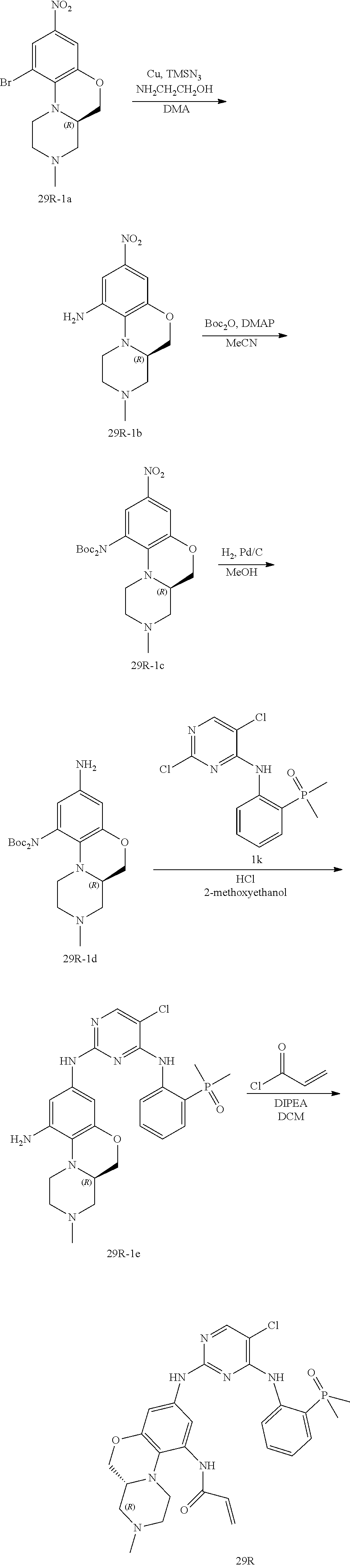

A mixture of compound 29R-1a (2.0 g, 6.09 mmol), TMSN₃ (1.4 g, 12.19 mmol), 2-aminoethanol (820 mg, 13.14 mmol), and copper powder (774 mg, 12.19 mmol) in DMA (15 mL) was heated to 95° C., and stirred under the atmosphere of nitrogen until the reaction was completed. Water (15 mL) was added, and extracted with EtOAc (3×15 mL). The combined organic layers were dried over anhydrous sodium sulfate, filtered, and concentrated under reduced pressure to remove the solvent. The residue was purified by silica gel column chromatography to afford compound 29R-1b (600 mg, 37%) as a yellow oil. MS m/z 265.3 [M+H]⁺.

Compound 29R-1b (600 mg, 2.27 mmol) and DMAP (55 mg, 0.45 mmol) were dissolved in acetonitrile (10 mL) in ice bath followed by the addition of Boc₂O (1.49 g, 6.81 mmol). The reaction mixture was heated 50° C., and stirred until the reaction was completed. Water (15 mL) was added, and extracted with EtOAc (3×15 mL). The combined organic layers were dried over anhydrous sodium sulfate, filtered, and concentrated under reduced pressure. The residue was purified by silica gel column chromatography to afford compound 29R-1c (600 mg, 57%) as a yellow oil. ¹H NMR (500 MHz, CDCl₃) δ 7.69 (d, J=2.6 Hz, 1H), 7.59 (d, J=2.6 Hz, 1H), 4.19-4.12 (m, 2H), 3.67-3.24 (m, 3H), 2.75-2.66 (m, 2H), 2.41-2.34 (m, 5H), 1.47 (s, 9H), 1.44 (s, 9H).

Compound 29R-1c (140 mg, 0.30 mmol) and 10% Pd—C (32 mg, 0.03 mmol) were dissolved in MeOH (3 mL). The reaction mixture was stirred at room temperature under 1 atmospheric pressure of H₂ for 2 hours. The residue was purified by prep-TLC. It was filtered through celite, and the filtrate was concentrated under reduce pressure. The residue was purified by prep-TLC (CH₂Cl₂:MeOH=20:1) to afford compound 29R-1d (60 mg, 46%) as alight yellow solid. MS m/z 435.6 [M+H]⁺.

Compound 29R-1d (60 mg, 0.14 mmol) and compound 1k (43 mg, 0.14 mmol) were dissolved in compound 2-methoxyethanol (1.5 mL) followed by the addition of 2.5 M HCl in MeOH (2.5 M, 0.35 mL). The reaction mixture in a sealed tube was heated to 120° C., and stirred overnight. The reaction was monitored by TLC for completion. It was cooled to room temperature, and concentrated under reduce pressure. The residue was purified by prep-TLC (CH₂Cl₂:MeOH=20:1) to afford compound 29R-1e (12 mg, yield 13%) as a light yellow solid. ¹H NMR (500 MHz, CD₃OD) δ 8.53-8.50 (m, 1H), 8.01 (s, 1H), 7.58-7.55 (m, 2H), 7.23-7.20 (m, 1H), 6.52-6.50 (m, 1H), 6.47-6.45 (m, 1H), 4.53 (t, J=11.0 Hz, 1H), 4.01 (dd, J=10.8, 1.8 Hz, 1H), 3.30-3.29 (m, 1H), 3.17 (d, J=10.5 Hz, 1H), 2.99 (d, J=12.0 Hz, 1H), 2.87 (d, J=10.5 Hz, 1H), 2.77 (t, J=11.8 Hz, 1H), 2.67-2.63 (m, 1H), 2.44 (t, J=10.0 Hz, 1H), 2.34 (s, 3H), 1.85 (s, 3H), 1.82 (s, 3H).

Compound 29R-1e (12 mg, 0.023 mmol) was dissolved in CH₂Cl₂ (1 mL) in ice bath. DIPEA (30 mg, 0.23 mmol) and a solution of acrylic chloride (2.1 mg, 0.023 mmol) in CH₂Cl₂ (1 mL) were added. The reaction mixture was stirred at room temperature for 1 hour. The reaction was monitored by TLC for completion. The reaction was quenched with water, and extracted with CH₂Cl₂. The combined organic layers were washed with brine, dried over anhydrous sodium sulfate, filtered, and concentrated under reduced pressure. The residue was purified by prep-TLC to afford compound 29R (6 mg, 45%). MS m/z 568.6 [M+H]⁺.

### Example 38: Preparation of Compound 30R

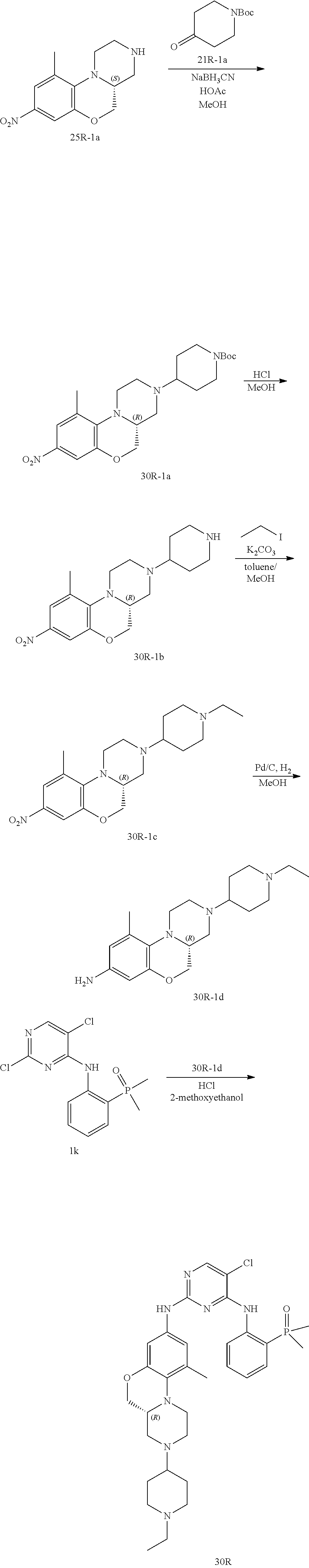

A mixture of Compound 25R-1a (400 mg, 1.60 mmol), N-tert-Butoxycarbonyl-4-piperidone (1.6 g, 8.02 mmol) and AcOH (3 drops) in MeOH (15 mL) was stirred at room temperature for 1 hour. Sodium cyanoborohydride (303 mg, 4.81 mmol) was added, and the reaction mixture was stirred at room temperature for 3 hours. After the reaction was completed, it was concentrated under reduce pressure. The residue was purified by silica gel column chromatography (CH₂Cl₂:MeOH=30:1) to afford compound 30R-1a (694 mg) as a yellow oil which was used in next step. MS m/z 433.5 [M+H]⁺.

Compound 30R-1a (694 mg, 1.60 mmol) was dissolved in MeOH (15 mL) at room temperature. 4.0 M HCl in MeOH (0.80 mL) was added. The reaction mixture was stirred at 60° C. 2 hours. After the reaction was completed, it was concentrated under reduce pressure. The residue was dissolved in CH₂Cl₂, and neutralized with saturated NaHCO₃ aqueous solution. The mixture was extracted with CH₂Cl₂ (3×20 mL). The combined organic layers were washed with brine, dried over anhydrous sodium sulfate, filtered, and concentrated under reduced pressure. The residue was purified by silica gel column chromatography (CH₂Cl₂:MeOH=20:1) to afford compound 30R-1b (400 mg, yield of two steps 75%) as a yellow solid.

A mixture of compound 30R-1b (150 mg, 0.45 mmol), CH₃I (106 mg, 0.68 mmol), and K₂CO₃ (125 mg, 0.90 mmol) in toluene/MeOH (2 mL/1 mL) was stirred at 50° C. for 5 hours. The reaction was monitored by TLC for completion. It was concentrated under reduce pressure. The residue was purified by silica gel column chromatography (CH₂Cl₂:MeOH=20:1) to afford compound 30R-1c (115 mg, yield 71%) as a yellow solid. MS m/z 361.4 [M+H]⁺.

Compound 30R-1c (115 mg, 0.31 mmol) and 10% Pd—C (50 mg) were added to MeOH (6 mL) at room temperature. The reaction mixture was stirred at room temperature under 1 atmospheric pressure of H₂ for 1 hour. The reaction was monitored by TLC for completion. It was filtered through celite, and the filtrate was concentrated under reduce pressure. The residue was purified by silica gel column chromatography (CH₂Cl₂:MeOH=20:1, 2% aqueous ammonium hydroxide solution) to afford compound 30R-1d (91 mg, yield 86%) as a brown solid. MS m/z 331.5 [M+H]⁺.

Compound 30R-1d (91 mg, 0.28 mmol) and compound 1k (104 mg, 0.33 mmol) were dissolved in 2-methoxyethanol (2 mL) followed by the addition of 2.5 M HCl in MeOH (2.5 M, 0.29 mL). The reaction mixture in a sealed tube was heated to 120° C., and stirred overnight. The reaction was monitored by TLC for completion. It was cooled to room temperature, and concentrated under reduce pressure. The residue was purified by silica gel column chromatography (CH₂Cl₂:MeOH=20:1, 2% aqueous ammonium hydroxide solution) to afford compound 30R (142 mg, yield 85%) as a light yellow solid. ¹H NMR (500 MHz, CD₃OD) δ 8.51-8.45 (m, 1H), 8.04 (s, 1H), 7.62-7.52 (m, 2H), 7.24 (t, J=7.5 Hz, 1H), 6.96 (s, 1H), 6.80 (s, 1H), 4.56 (t, J=10.6 Hz, 1H), 3.98 (dd, J=10.6, 2.0 Hz, 1H), 3.16-2.99 (m, 5H), 2.94-2.82 (m, 2H), 2.73 (dd, J=11.7, 4.0 Hz, 1H), 2.54-2.46 (m, 3H), 2.30-2.22 (m, 1H), 2.19 (s, 3H), 2.11 (t, J=11.8 Hz, 2H), 1.95-1.88 (m, 2H), 1.86 (s, 3H), 1.83 (s, 3H), 1.64-1.52 (m, 2H), 1.13 (t, J=7.1 Hz, 3H). MS m/z 610.7 [M+H]⁺.

### Example 39: Preparation of Compound 30S

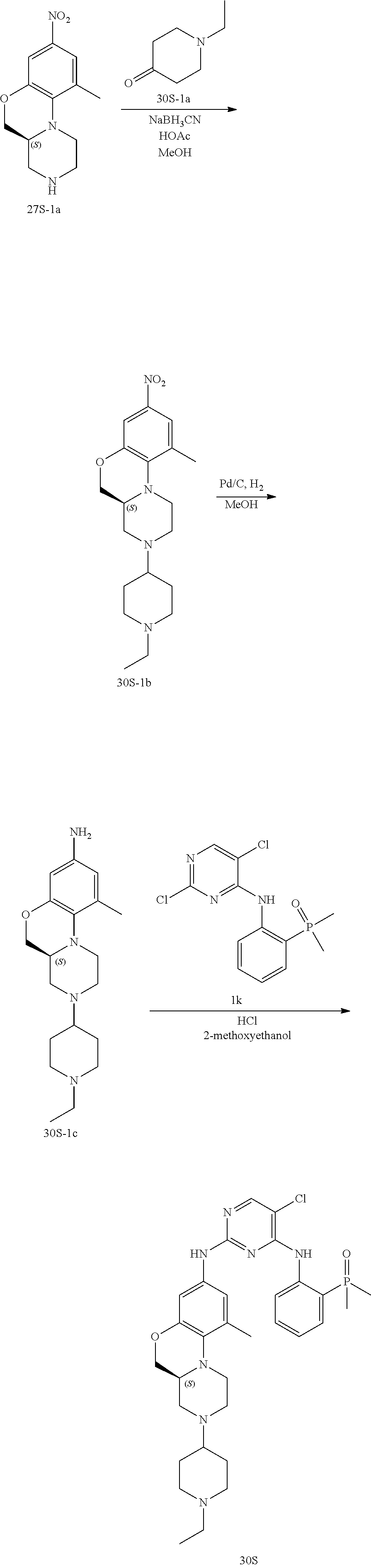

A mixture of compound 27S-1a (157 mg, 0.63 mmol), N-ethyl-4-piperidone (30S-1a, 160 mg, 1.26 mmol), and AcOH (2 drops) in MeOH (5 mL) was stirred at room temperature for 1 hour. Sodium cyanoborohydride (118 mg, 1.89 mmol) was added, and the reaction mixture was stirred at room temperature for 2 hours. After the reaction was completed, it was concentrated under reduce pressure. The residue was purified by silica gel column chromatography (CH₂Cl₂:MeOH=25:1, 2% aqueous ammonium hydroxide solution) to afford compound 30S-1b (230 mg) as a light yellow solid which was used in next step. MS m/z 361.5 [M+H]⁺.

Compound 30S-1b (230 mg, 0.53 mmol) and 10% Pd—C (80 mg) were added to MeOH (10 mL) at room temperature. The reaction was purged with hydrogen (3 times). The reaction mixture was stirred at room temperature under 1 atmospheric pressure of H₂ for 1 hour. The reaction was monitored by TLC for completion. It was filtered through celite, and the filtrate was concentrated under reduce pressure. The residue was purified by silica gel column chromatography (CH₂Cl₂:MeOH=25:1, 2% aqueous ammonium hydroxide solution) to afford compound 33S-1c (136 mg, yield of two steps 64%) as a white solid. MS m/z 331.4 [M+H]⁺.

Compound 30S-1c (70 mg, 0.2 mmol) and compound 1k (67 mg, 0.2 mmol) were dissolved in 2-methoxyethanol (2 mL) followed by the addition of 2.5 M HCl in MeOH (2.5 M, 0.2 mL). The reaction mixture in a sealed tube was heated to 120° C., and stirred overnight. The reaction was monitored by TLC for completion. It was cooled to room temperature, and concentrated under reduce pressure. The residue was dissolved in a small amount of CH₂Cl₂, neutralized with saturated NaHCO₃ aqueous solution, and extracted with CH₂Cl₂ (3×10 mL). The combined organic layers were washed with brine, dried over anhydrous sodium sulfate, filtered, and concentrated under reduced pressure. The residue was purified by silica gel column chromatography (CH₂Cl₂:MeOH=20:1, 2% aqueous ammonium hydroxide solution) to afford compound 30S (50 mg, yield 39%) as a white solid. ¹H NMR (500 MHz, CD₃OD) δ 8.52-8.42 (m, 1H), 8.04 (s, 1H), 7.63-7.49 (m, 2H), 7.27-7.20 (m, 1H), 6.96 (d, J=2.4 Hz, 1H), 6.80 (d, J=2.0 Hz, 1H), 4.56 (t, J=10.6 Hz, 1H), 3.98 (dd, J=10.6, 2.8 Hz, 1H), 3.18-2.98 (m, 5H), 2.95-2.80 (m, 2H), 2.74 (dd, J=11.7, 4.2 Hz, 1H), 2.55-2.47 (m, 3H), 2.27 (t, J=11.0 Hz, 1H), 2.20 (s, 3H), 2.15-2.06 (m, 2H), 1.95-1.88 (m, 2H), 1.86 (s, 3H), 1.83 (s, 3H), 1.65-1.55 (m, 2H), 1.13 (t, J=7.2 Hz, 3H). MS m/z 610.6 [M+H]⁺.

### Example 40: Preparation of Compound 31R

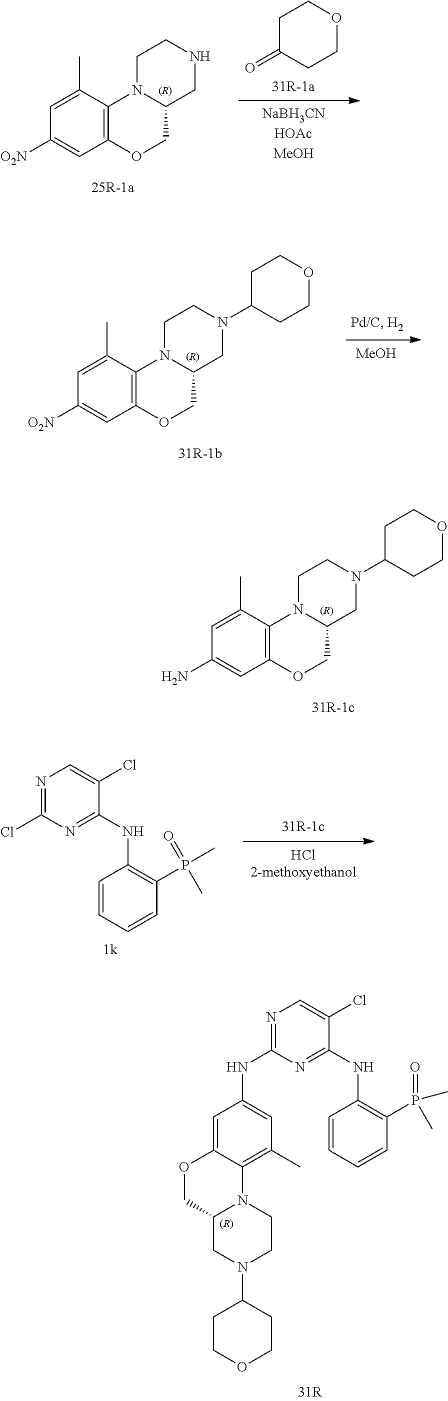

A mixture of compound 25R-1a (150 mg, 0.60 mmol), tetrahydro-4H-pyran-4-one (31R-1a, 301 mg, 3.01 mmol), and AcOH (2 drops) in MeOH (5 mL) was stirred at room temperature for 1 hour. Sodium cyanoborohydride (113 mg, 1.81 mmol) was added, and the reaction mixture was stirred at room temperature for 3 hours. After the reaction was completed, it was concentrated under reduce pressure. The residue was purified by silica gel column chromatography (CH₂Cl₂:MeOH=20:1) to afford compound 31R-1b (201 mg) as a yellow solid which was used in next step. MS m/z 334.5 [M+H]⁺.

Compound 31R-1b (201 mg, 0.60 mmol) and 10% Pd—C (80 mg) were added to MeOH (6 mL). The reaction mixture was stirred at room temperature under 1 atmospheric pressure of H₂ for 2 hours. The reaction was monitored by TLC for completion. It was filtered through the celite, and the filtrate was concentrated under reduce pressure. The residue was purified by silica gel column chromatography (CH₂Cl₂:MeOH=20:1) to afford compound 31R-1c (209 mg, yield 86%) as a brown solid.

Compound 31R-1c (209 mg, 0.69 mmol) and compound 1k (218 mg, 0.69 mmol) were dissolved in 2-methoxyethanol (4 mL) followed by the addition of 2.5 M HCl in MeOH (0.72 mL). The reaction mixture in a sealed tube was heated to 120° C., and stirred overnight. The reaction was monitored by TLC for completion. It was cooled to room temperature, and concentrated under reduce pressure. The residue was purified by silica gel column chromatography (CH₂Cl₂:MeOH=20:1, 2% aqueous ammonium hydroxide solution) to afford compound 31R (104 mg, yield 26%) as a yellow solid. ¹H NMR (500 MHz, CDCl₃) δ 10.91 (s, 1H), 8.69-8.64 (m, 1H), 8.07 (s, 1H), 7.51 (t, J=7.9 Hz, 1H), 7.30-7.23 (m, 1H), 7.14-7.06 (m, 2H), 6.84 (s, 1H), 6.80 (s, 1H), 4.79-4.48 (m, 1H), 4.10-3.97 (m, 3H), 3.40 (t, J=11.6 Hz, 2H), 3.30-2.72 (m, 6H), 2.66-2.35 (m, 2H), 2.25 (s, 3H), 1.84 (s, 3H), 1.81 (s, 3H), 1.79-1.73 (m, 2H), 1.66-1.56 (m, 2H). MS m/z 583.6 [M+H]⁺.

### Example 41: Preparation of Compound 31S

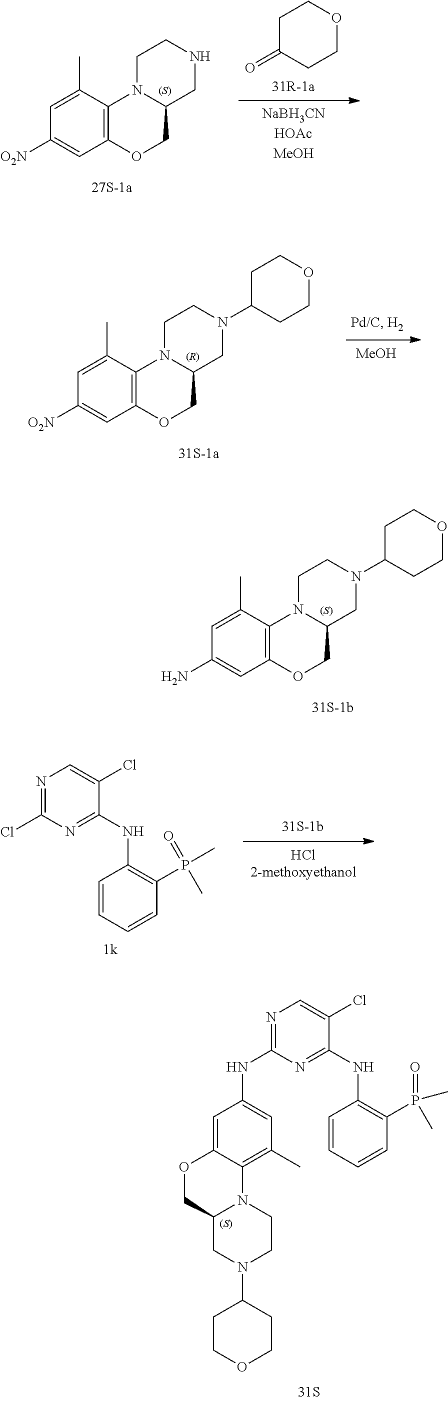

A mixture of compound 27S-1a (100 mg, 0.40 mmol), tetrahydro-4H-pyran-4-one (31R-1a, 120 mg, 1.20 mmol), and AcOH (2 drops) in MeOH (4 mL) was stirred at room temperature for 1 hour. Sodium cyanoborohydride (76 mg, 1.20 mmol) was added, and the reaction mixture was stirred at room temperature for 2 hours. After the reaction was completed, it was concentrated under reduce pressure. The residue was purified by silica gel column chromatography (CH₂Cl₂:MeOH=20:1, 2% aqueous ammonium hydroxide solution) to afford compound 31S-1a (157 mg) as a yellow solid which was used in next step. MS m/z 334.5 [M+H]⁺.

Compound 31S-1a (157 mg, 0.42 mmol) and 10% Pd—C (60 mg) were added to MeOH (4 mL) at room temperature. The reaction was purged with hydrogen (3 times). The reaction mixture was stirred at room temperature under 1 atmospheric pressure of H₂ for 1 hour. The reaction was monitored by TLC for completion. It was filtered through the celite, and the filtrate was concentrated under reduce pressure. The residue was purified by silica gel column chromatography (CH₂Cl₂:MeOH=20:1, 2% aqueous ammonium hydroxide solution) to afford compound 31S-1b (126 mg, yield of two steps 100%) as a yellow solid. MS m/z 304.5 [M+H]⁺.

Compound 31S-1b (50 mg, 0.16 mmol) and compound 1k (52 mg, 0.16 mmol) were dissolved 2-methoxyethanol (2 mL) followed by the addition of 2.5 M HCl in MeOH solution (0.17 mL). The reaction mixture in a sealed tube was heated to 120° C., and stirred overnight. The reaction was monitored by TLC for completion. It was cooled to room temperature, and concentrated under reduce pressure. The residue was dissolved in a small amount of CH₂Cl₂, neutralized with saturated aqueous NaHCO₃, and extracted with CH₂Cl₂ (3×10 mL). The combined organic layers were washed with brine, dried over anhydrous sodium sulfate, filtered, and concentrated under reduced pressure. The residue was purified by silica gel column chromatography (CH₂Cl₂:MeOH=20:1, 2% aqueous ammonium hydroxide solution) to afford compound 31S (57 mg, yield 59%) as a white solid. ¹H NMR (500 MHz, CDCl₃) δ 10.91 (s, 1H), 8.73-8.54 (m, 1H), 8.07 (s, 1H), 7.51 (t, J=7.9 Hz, 1H), 7.30-7.27 (m, 1H), 7.13-7.08 (m, 2H), 6.83 (s, 1H), 6.80 (s, 1H), 4.78-4.50 (m, 1H), 4.08-3.97 (m, 3H), 3.40 (t, J=11.0 Hz, 2H), 3.32-2.77 (m, 6H), 2.66-2.41 (m, 2H), 2.25 (s, 3H), 1.84 (s, 3H), 1.81 (s, 3H), 1.81-1.76 (m, 2H), 1.68-1.59 (m, 2H). MS m/z 583.7 [M+H]⁺.

### Example 42: Preparation of Compound 32R

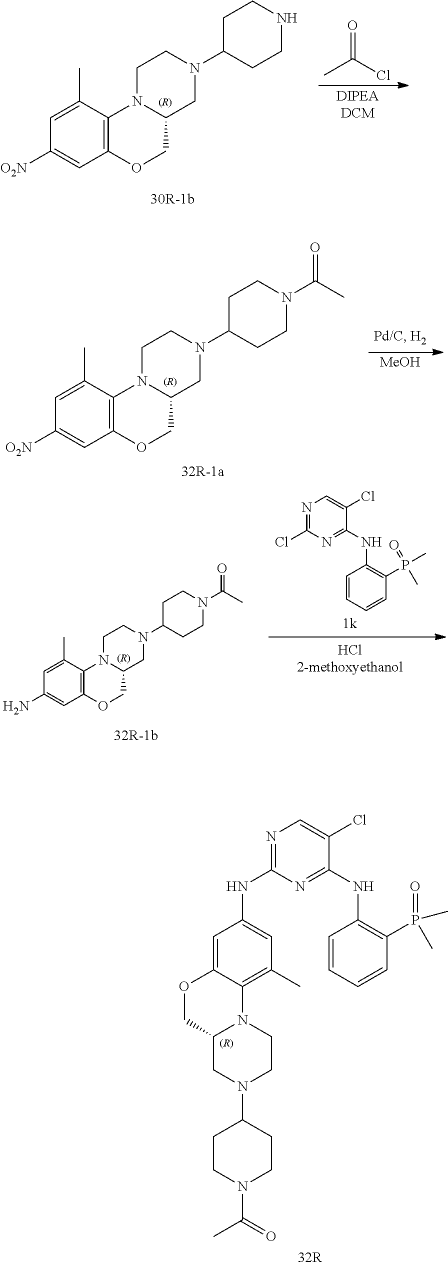

Compound 30R-1b (150 mg, 0.45 mmol) and N,N-diisopropylethylamine (117 mg, 0.90 mmol) were dissolved in CH₂Cl₂ (3 mL) at 0° C. A solution of acetylchloride (53 mg, 0.68 mmol) in CH₂Cl₂ (1 mL) was slowly added. The reaction mixture was stirred at room temperature for 1 hour. The reaction was monitored by TLC for completion. It was concentrated under reduced pressure. The residue was purified by silica gel column chromatography (CH₂Cl₂:MeOH=20:1) to afford compound 32R-1a (142 mg, yield 84%) as a yellow solid. MS m/z 375.4 [M+H]⁺.

Compound 32R-1a (142 mg, 0.38 mmol) and 10% Pd—C (50 mg) were added to MeOH (5 mL) at room temperature. The reaction mixture was stirred at room temperature under 1 atmospheric pressure of H₂ for 1 hour. The reaction was monitored by TLC for completion. It was filtered through the celite, and the filtrate was concentrated under reduce pressure. The residue was purified by silica gel column chromatography (CH₂Cl₂:MeOH=15:1) to afford compound 32R-1b (118 mg, yield 90%) as a light yellow solid. MS m/z 345.5 [M+H]⁺.

Compound 32R-1b (60 mg, 0.17 mmol) and compound 1k (66 mg, 0.21 mmol) were dissolved in 2-methoxyethanol (2 mL) followed by the addition of 2.5 M HCl in MeOH (0.18 mL). The reaction mixture in a sealed tube was heated to 120° C., and stirred overnight. The reaction was monitored by TLC for completion. It was cooled to room temperature, and concentrated under reduce pressure. The residue was purified by silica gel column chromatography (CH₂Cl₂:MeOH=20:1, 2% aqueous ammonium hydroxide solution) to afford compound 32R (45 mg, yield 41%) as a light yellow solid. ¹H NMR (500 MHz, CD₃OD) δ 8.50-8.45 (m, 1H), 8.03 (s, 1H), 7.62-7.51 (m, 2H), 7.23 (td, J=7.5, 1.3 Hz, 1H), 6.96 (d, J=2.4 Hz, 1H), 6.79 (d, J=2.2 Hz, 1H), 4.54 (t, J=10.7 Hz, 1H), 4.50-4.44 (m, 1H), 4.00-3.89 (m, 2H), 3.15-3.04 (m, 3H), 3.03-2.97 (m, 1H), 2.92-2.81 (m, 2H), 2.79-2.73 (m, 1H), 2.72-2.64 (m, 1H), 2.59-2.44 (m, 2H), 2.18 (s, 3H), 2.09 (s, 3H), 1.94-1.86 (m, 2H), 1.85 (s, 3H), 1.82 (s, 3H), 1.55-1.32 (m, 2H). MS m/z 624.7 [M+H]⁺.

### Example 43: Preparation of Compound 32S

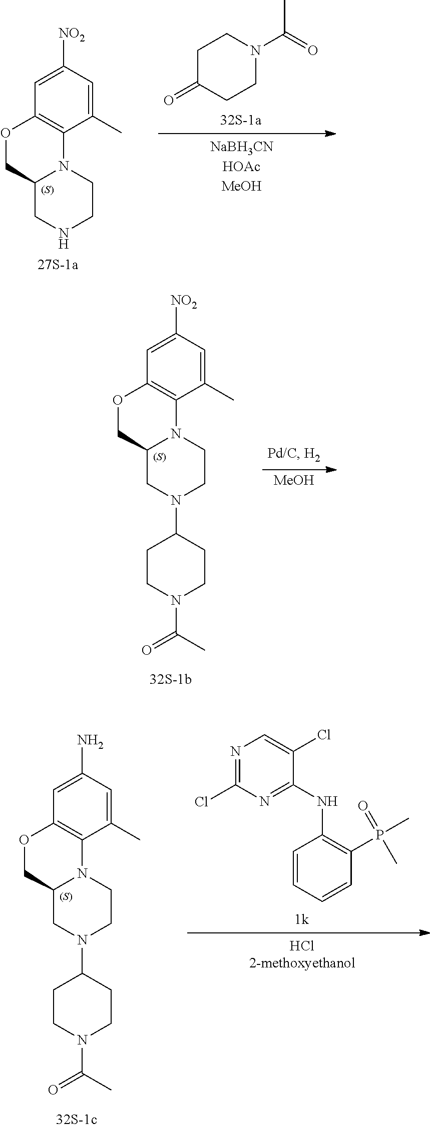

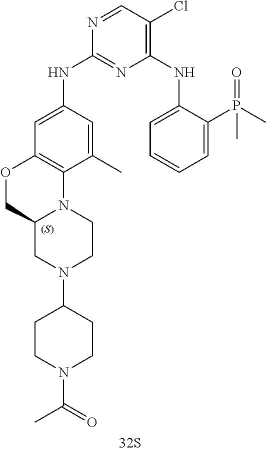

A mixture of compound 27S-1a (100 mg, 0.40 mmol), N-acetyl-4-piperidone (32S-1a, 170 mg, 1.20 mmol), and AcOH (2 drops) in MeOH (4 mL) was stirred at room temperature for 1 hour. Sodium cyanoborohydride (76 mg, 1.20 mmol) was added, and the reaction mixture was stirred at room temperature for 2 hours. After the reaction was completed, it was concentrated under reduce pressure. The residue was purified by silica gel column chromatography (CH₂Cl₂:MeOH=20:1, 2% aqueous ammonium hydroxide solution) to afford compound 32S-1b (200 mg) as a yellow solid which was used in next step. MS m/z 375.5 [M+H]⁺.

Compound 32S-1b (200 mg, 0.53 mmol) and 10% Pd—C (80 mg) were added to MeOH (5 mL) at room temperature. The reaction was purged with hydrogen (3 times). The reaction mixture was stirred at room temperature under 1 atmospheric pressure of H₂ for 1 hour. The reaction was monitored by TLC for completion. It was filtered through the celite, and the filtrate was concentrated under reduce pressure. The residue was purified by silica gel column chromatography (CH₂Cl₂:MeOH=20:1, 2% aqueous ammonium hydroxide solution) to afford compound 32S-1c (190 mg, yield of two steps 100%) as yellow solid. MS m/z 345.5 [M+H]⁺.

Compound 32S-1c (50 mg, 0.15 mmol) and compound 1k (46 mg, 0.15 mmol) were dissolved 2-methoxyethanol (2 mL) followed by the addition of 2.5 M HCl in MeOH solution (0.15 mL). The reaction mixture in a sealed tube was heated to 120° C., and stirred overnight. The reaction was monitored by TLC for completion. It was cooled to room temperature, and concentrated under reduce pressure. The residue was dissolved in a small amount of CH₂Cl₂, neutralized with saturated aqueous NaHCO₃, and extracted with CH₂Cl₂ (3×10 mL). The combined organic layers were washed with brine, dried over anhydrous sodium sulfate, filtered, and concentrated under reduced pressure. The residue was purified by silica gel column chromatography (CH₂Cl₂:MeOH=20:1, 2% aqueous ammonium hydroxide solution) to afford compound 32S (36 mg, yield 40%) as a yellow solid. ¹H NMR (500 MHz, CDCl₃) δ 10.92 (s, 1H), 8.68-8.61 (m, 1H), 8.06 (s, 1H), 7.50 (t, J=7.9 Hz, 1H), 7.26-7.21 (m, 1H), 7.14-7.07 (m, 2H), 6.87 (s, 1H), 6.80 (s, 1H), 4.69-4.56 (m, 2H), 3.99 (dd, J=10.7, 2.5 Hz, 1H), 3.87 (d, J=14.6 Hz, 1H), 3.27-3.20 (m, 1H), 3.08 (t, J=12.6 Hz, 2H), 2.97-2.81 (m, 4H), 2.66-2.46 (m, 3H), 2.25 (s, 3H), 2.10 (s, 3H), 1.84 (s, 3H), 1.82 (s, 3H), 1.73-1.61 (m, 2H), 1.54-1.42 (m, 2H). MS m/z 624.7 [M+H]⁺.

### Example 44: Preparation of Compound 33R

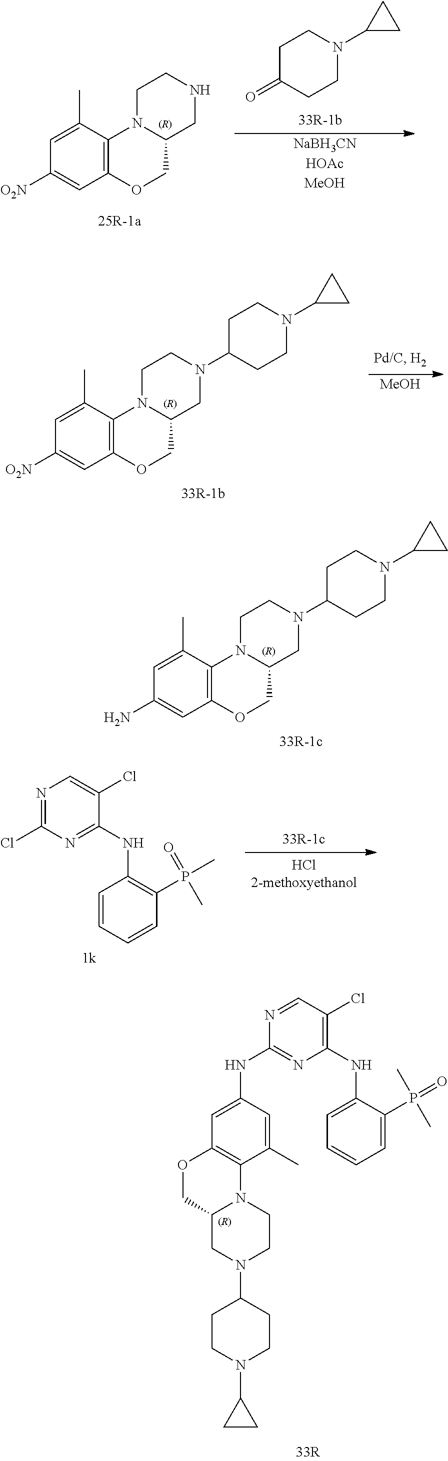

A mixture of compound 25R-1a (100 mg, 0.40 mmol), 1-cyclopropyl-4-piperidone (33R-1a, 168 mg, 1.20 mmol), and AcOH (2 drops) in MeOH (6 mL) was stirred at room temperature for 1 hour. Sodium cyanoborohydride (76 mg, 1.20 mmol) was added, and the reaction mixture was stirred at room temperature for 3 hours. After the reaction was completed, it was concentrated under reduce pressure. The residue was purified by silica gel column chromatography (CH₂Cl₂:MeOH=20:1, 2% aqueous ammonium hydroxide solution) to afford compound 33R-1b (150 mg, yield 100%) as a yellow solid. MS m/z 373.4 [M+H]⁺.

Compound 33R-1b (150 mg, 0.40 mmol) and 10% Pd—C (50 mg) were added to MeOH (5 mL) at room temperature. The reaction mixture was stirred at room temperature under 1 atmospheric pressure of H₂ for 2 hours. The reaction was monitored by TLC for completion. The reaction mixture was filtered through the celite, and the filtrate was concentrated under reduce pressure. The residue was purified by silica gel column chromatography (CH₂Cl₂:MeOH=20:1, 2% aqueous ammonium hydroxide solution) to afford compound 33R-1c (116 mg, yield 84%) as a brown solid. MS m/z 343.4 [M+H]⁺.

Compound 33R-1c (50 mg, 0.15 mmol) and compound 1k (55 mg, 0.18 mmol) were dissolved 2-methoxyethanol (1.5 mL) followed by the addition of 2.5 M HCl in MeOH (0.15 mL). The reaction mixture in a sealed tube was heated to 120° C., and stirred overnight. The reaction was monitored by TLC for completion. It was cooled to room temperature, and concentrated under reduce pressure. The residue was purified by silica gel column chromatography (CH₂Cl₂:MeOH=20:1, 2% aqueous ammonium hydroxide solution) to afford compound 33R (70 mg, yield 77%) as a light yellow solid. ¹H NMR (500 MHz, CD₃OD) δ 8.52-8.45 (m, 1H), 8.04 (s, 1H), 7.62-7.51 (m, 2H), 7.24 (t, J=7.0 Hz, 1H), 6.96 (d, J=2.1 Hz, 1H), 6.80 (d, J=2.1 Hz, 1H), 4.55 (t, J=10.7 Hz, 1H), 3.97 (dd, J=10.6, 2.6 Hz, 1H), 3.15-3.04 (m, 4H), 3.00 (d, J=11.7 Hz, 1H), 2.91-2.80 (m, 2H), 2.73 (dd, J=11.7, 4.2 Hz, 1H), 2.54-2.45 (m, 1H), 2.32-2.21 (m, 3H), 2.19 (s, 3H), 1.91-1.78 (m, 2H), 1.85 (s, 3H), 1.82 (s, 3H), 1.69-1.61 (m, 1H), 1.57-1.43 (m, 2H), 0.55-0.39 (m, 4H). MS m/z 622.7 [M+H]⁺.

### Example 45: Preparation of Compound 34R

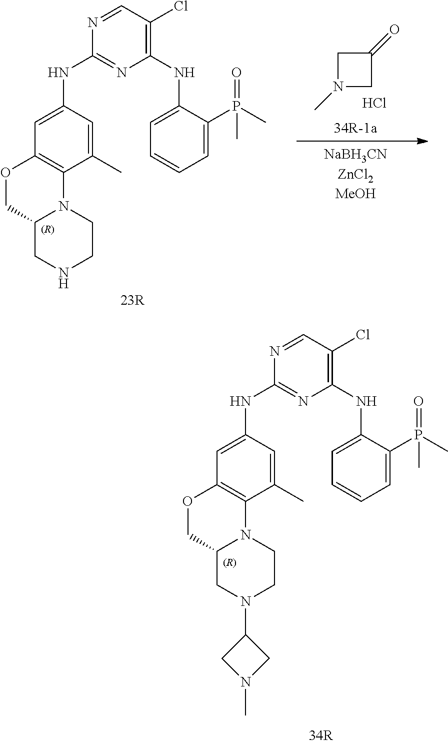

Compound 23R (50 mg, 0.10 mmol), 1-methyl-3-azetidinone hydrochloride (34R-1a, 18 mg, 0.15 mmol), and anhydrous zinc chloride (41 mg, 0.30 mmol) were dissolved in MeOH (3 mL) followed by the addition of sodium cyanoborohydride (19 mg, 0.30 mmol). The reaction mixture in a sealed tube was heated to 100° C., and stirred for 2 hours. The reaction was monitored by TLC for completion. It was cooled to room temperature, and concentrated under reduce pressure. The residue was purified by silica gel column chromatography (CH₂Cl₂:MeOH=20:1, 2% aqueous ammonium hydroxide solution) to afford compound 34R (21 mg, yield 37%) as white solid. ¹H NMR (500 MHz, CDCl₃) δ 10.90 (s, 1H), 8.69-8.63 (m, 1H), 8.07 (s, 1H), 7.54-7.47 (m, 1H), 7.30-7.23 (m, 1H), 7.13-7.06 (m, 2H), 6.81 (d, J=2.3 Hz, 1H), 6.77 (s, 1H), 4.61 (t, J=10.7 Hz, 1H), 4.01 (dd, J=10.6, 2.8 Hz, 1H), 3.74-3.65 (m, 2H), 3.22-3.17 (m, 1H), 3.13-3.00 (m, 4H), 2.91-2.84 (m, 1H), 2.73-2.63 (m, 2H), 2.48 (s, 3H), 2.47-2.45 (m, 1H), 2.24 (s, 3H), 2.23-2.19 (m, 1H), 1.84 (s, 3H), 1.81 (s, 3H). MS m/z 568.6 [M+H]⁺.

### Example 46: Preparation of Compound 34S

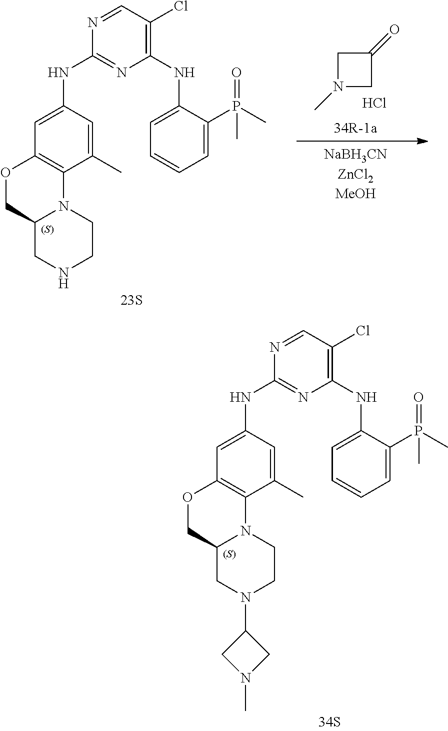

Compound 23S (38 mg, 0.08 mmol), 1-methyl-3-azetidinone hydrochloride (34R-1a, 14 mg, 0.11 mmol), and anhydrous zinc chloride (31 mg, 0.23 mmol) were dissolved in MeOH (3 mL) followed by the addition of sodium cyanoborohydride (14 mg, 0.23 mmol). The reaction mixture in a sealed tube was heated 100° C., and stirred for 2 hours. The reaction was monitored by TLC for completion. It was cooled to room temperature, and concentrated under reduce pressure. The residue was purified by silica gel column chromatography (CH₂Cl₂:MeOH=20:1, 2% aqueous ammonium hydroxide solution) to afford compound 34S (16 mg, yield 37%) as a white solid. ¹H NMR (500 MHz, CDCl₃) δ 10.90 (s, 1H), 8.70-8.62 (m, 1H), 8.07 (s, 1H), 7.50 (t, J=7.9 Hz, 1H), 7.31-7.27 (m, 1H), 7.14-7.07 (m, 2H), 6.82 (s, 1H), 6.77 (s, 1H), 4.60 (t, J=10.7 Hz, 1H), 4.01 (dd, J=10.6, 2.8 Hz, 1H), 3.94-3.84 (m, 2H), 3.33-3.13 (m, 4H), 3.08-3.03 (m, 1H), 2.88 (td, J=11.6, 2.5 Hz, 1H), 2.73-2.64 (m, 2H), 2.61 (s, 3H), 2.50 (dd, J=11.5, 4.2 Hz, 1H), 2.28-2.24 (m, 1H), 2.24 (s, 3H), 1.84 (s, 3H), 1.82 (s, 3H). MS m/z 568.7 [M+H]⁺.

### Example 47: Preparation of Compound 35S

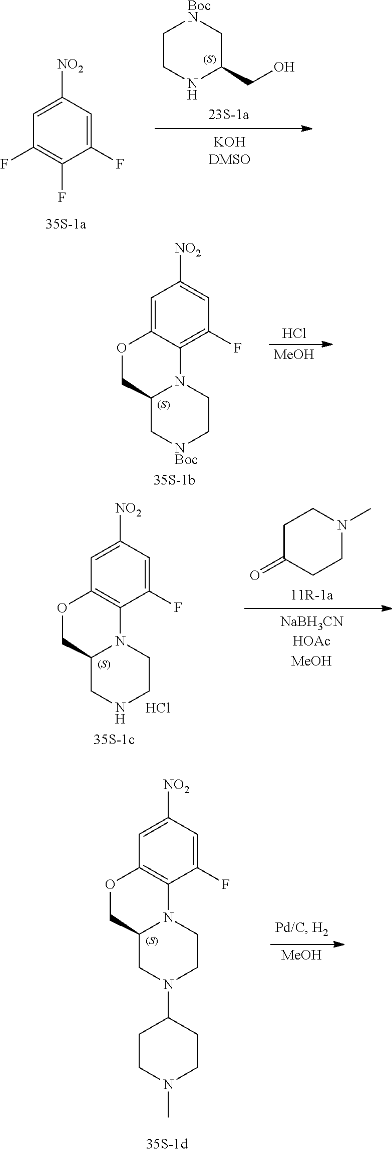

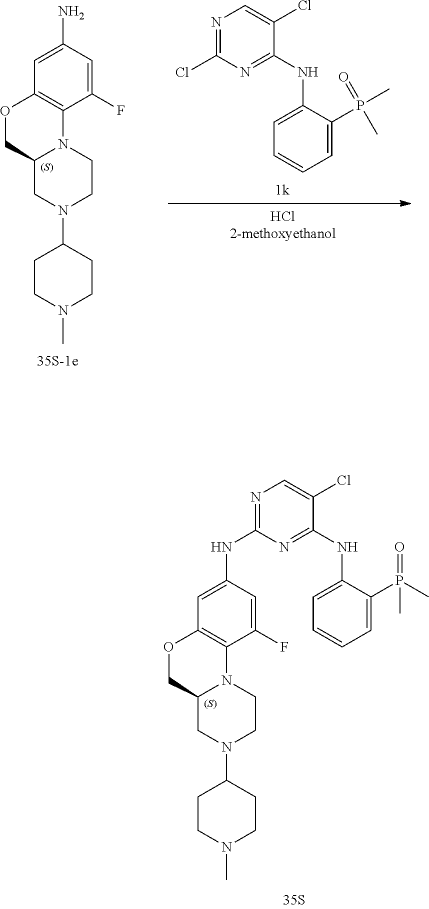

Compound 35S-1a (1.0 g, 5.65 mmol), 23S-1a (1.22 g, 5.65 mmol), and KOH (950 mg, 16.94 mmol) were dissolved in DMSO (15 mL). The reaction mixture was stirred at room temperature for 3 hours, and then stirred at 60° C. for 3 hours. After the reaction was completed, it was cooled to room temperature, poured into ice water, and stirred at room temperature for 1 hour. The mixture was extracted with CH₂Cl₂ (3×30 mL). The combined organic layers were washed with brine, dried over anhydrous sodium sulfate, filtered, and concentrated under reduced pressure. The residue was purified by silica gel column chromatography (PE:EtOAC:CH₂Cl_{2=5:1:1}) to afford compound 35S-1b (1.45 g, yield 73%) as a yellow solid. MS m/z 354.4 [M+H]⁺.

Compound 35S-1b (1.45 g, 4.10 mmol) was dissolved in MeOH (15 mL) at room temperature followed by the addition of 4.0 M HCl in MeOH (4.0 M, 4 mL). The reaction mixture was stirred at 60° C. for 1 hour. The reaction was monitored by TLC for completion. It was concentrated under reduced pressure to remove most of MeOH. The precipitate was collected by filtration, and dried to afford compound 35S-1c (1.08 g, yield 91%) as a yellow solid. MS m/z 254.3 [M+H]⁺.

A mixture of compound 35S-1c (500 mg, 1.73 mmol), N-methyl-4-piperidone (11R-1a, 587 mg, 5.18 mmol), and AcOH (4 drops) in MeOH (8 mL) was stirred at room temperature for 1 hour. Sodium cyanoborohydride (326 mg, 5.18 mmol) was added, and the reaction mixture was stirred at room temperature overnight. After the reaction was completed, it was concentrated under reduce pressure. The residue was purified by silica gel column chromatography (CH₂Cl₂:MeOH=20:1, 2% aqueous ammonium hydroxide solution) to afford compound 35S-1d (704 mg) as a yellow solid which was used in next step. MS m/z 351.4 [M+H]⁺.

Compound 35S-1d (100 mg, 0.29 mmol) and 10% Pd—C (80 mg) were added to MeOH (3 mL) at room temperature. The reaction was purged with hydrogen (3 times). The reaction mixture was stirred at room temperature under 1 atmospheric pressure of H₂ for 1 hour. The reaction was monitored by TLC for completion. It was filtered through the celite, and the filtrate was concentrated under reduce pressure. The residue was purified by silica gel column chromatography (CH₂Cl₂:MeOH=20:1, 2% aqueous ammonium hydroxide solution) to afford compound 35S-1e (38 mg, yield 41%) as a yellow solid. MS m/z 321.5 [M+H]⁺.

Compound 35S-1e (38 mg, 0.12 mmol) and compound 1k (41 mg, 0.13 mmol) were dissolved in 2-methoxyethanol (1.5 mL) followed by the addition of 2.5 M HCl in MeOH solution (0.12 mL). The reaction mixture in a sealed tube was heated to 120° C., and stirred overnight. The reaction was monitored by TLC for completion. It was cooled to room temperature, and concentrated under reduce pressure. The residue was dissolved in a small amount of CH₂Cl₂, neutralized with saturated aqueous NaHCO₃, and extracted with CH₂Cl₂ (3×10 mL). The combined organic layers were washed with brine, dried over anhydrous sodium sulfate, filtered, and concentrated under reduced pressure. The residue was purified by silica gel column chromatography (CH₂Cl₂:MeOH=20:1, 2% aqueous ammonium hydroxide solution) to afford compound 35S (60 mg, yield 84%) as a white solid. ¹H NMR (500 MHz, CDCl₃) δ 10.89 (s, 1H), 8.61-8.56 (m, 1H), 8.07 (s, 1H), 7.54 (t, J=7.9 Hz, 1H), 7.31-7.27 (m, 1H), 7.13 (t, J=6.8 Hz, 1H), 7.07 (dd, J=14.8, 2.4 Hz, 1H), 6.81 (s, 1H), 6.75 (s, 1H), 4.18-4.05 (m, 2H), 3.72-3.64 (m, 1H), 3.25-3.18 (m, 1H), 3.15-3.04 (m, 3H), 2.85 (dd, J=10.9, 2.8 Hz, 1H), 2.81-2.74 (m, 1H), 2.67-2.59 (m, 1H), 2.50-2.42 (m, 1H), 2.47 (s, 3H), 2.40-2.22 (m, 3H), 1.95-1.86 (m, 4H), 1.84 (s, 3H), 1.82 (s, 3H). MS m/z 600.7 [M+H]⁺.

### Example 48: Preparation of Compound 36R

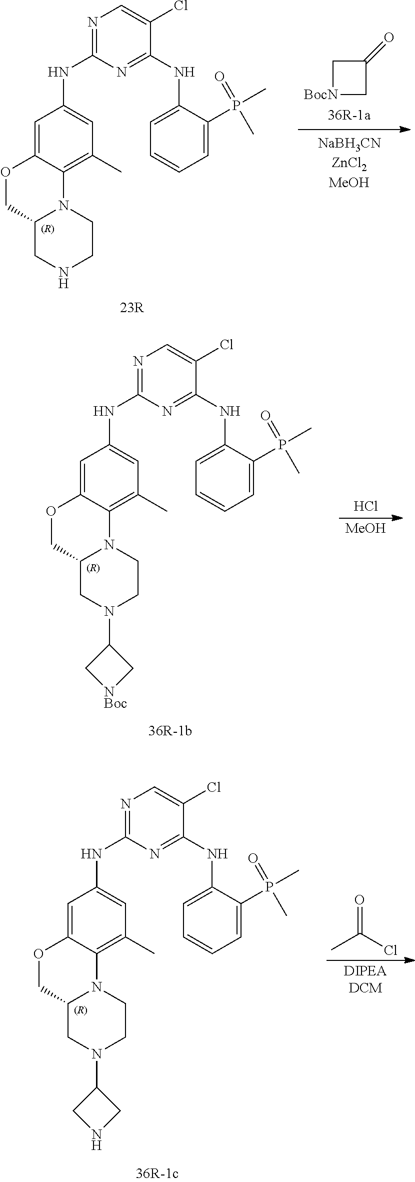

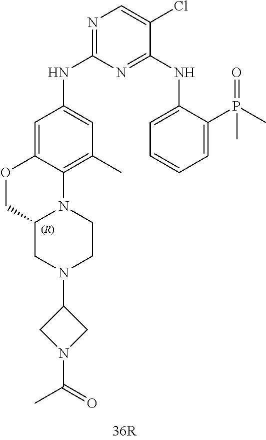

Compound 23R (200 mg, 0.40 mmol), tert-butyl 3-oxoazetidine-1-carboxylate (36R-1a, 103 mg, 0.60 mmol), and anhydrous zinc chloride (164 mg, 1.20 mmol) were dissolved in MeOH (10 mL) followed the addition of sodium cyanoborohydride (76 mg, 1.20 mmol). The reaction mixture in a seal tube was heated to 100° C., and stirred for 1 hour. The reaction was monitored by TLC for completion. It was cooled to room temperature, and concentrated under reduce pressure. The residue was purified by silica gel column chromatography (CH₂Cl₂:MeOH=20:1, 2% aqueous ammonium hydroxide solution) to afford compound 36R-1b (258 mg) as a light yellow solid which was used in next step. MS m/z 654.7 [M+H]⁺.

Compound 36R-1b (258 mg, 0.52 mmol) was dissolved in MeOH (10 mL) followed by the addition of 4.0 M HCl in 1,4-dioxane (3 mL). The reaction mixture was stirred at 40° C. for 2 hours. The reaction was monitored by TLC for completion. It was cooled to room temperature, and concentrated under reduce pressure. The residue was dissolved in a small amount of MeOH, neutralized with aqueous ammonium hydroxide solution, and concentrated under reduce pressure. The residue was purified by silica gel column chromatography (CH₂Cl₂:MeOH=12:1, 2% aqueous ammonium hydroxide solution) to afford compound 36R-1c (185 mg, yield of two steps 83%) as a light yellow solid. MS m/z 554.6 [M+H]⁺.

Compound 36R-1c (70 mg, 0.13 mmol) and N,N-diisopropylethylamine (34 mg, 0.26 mmol) were dissolved in CH₂Cl₂ (10 mL). A solution of acetylchloride (10 mg, 0.13 mmol) in CH₂Cl₂ (1 mL) was slowly added at 0° C. The reaction mixture was stirred at room temperature for 15 minutes. After the reaction was completed, it was concentrated under reduce pressure. The residue was purified by silica gel column chromatography (CH₂Cl₂:MeOH=15:1, 2% aqueous ammonium hydroxide solution) to afford compound 36R (31 mg, yield 41%) as a white solid. ¹H NMR (500 MHz, CDCl₃) δ 11.21 (s, 1H), 8.67-8.62 (m, 1H), 8.01 (d, J=1.2 Hz, 1H), 7.60-7.47 (m, 2H), 7.32-7.27 (m, 1H), 7.18-7.08 (m, 2H), 6.80 (dd, J=10.7, 2.2 Hz, 1H), 4.65-4.56 (m, 1H), 4.17-4.11 (m, 1H), 4.07-3.91 (m, 3H), 3.89-3.78 (m, 1H), 3.23 (d, J=10.3 Hz, 1H), 3.17-3.06 (m, 2H), 2.94-2.86 (m, 1H), 2.83-2.70 (m, 2H), 2.57-2.49 (m, 1H), 2.32-2.25 (m, 1H), 2.25 (s, 3H), 1.89 (d, J=1.9 Hz, 3H), 1.85 (s, 3H), 1.82 (s, 3H). MS m/z 596.5 [M+H]⁺.

### Example 49: Preparation of Compound 36S

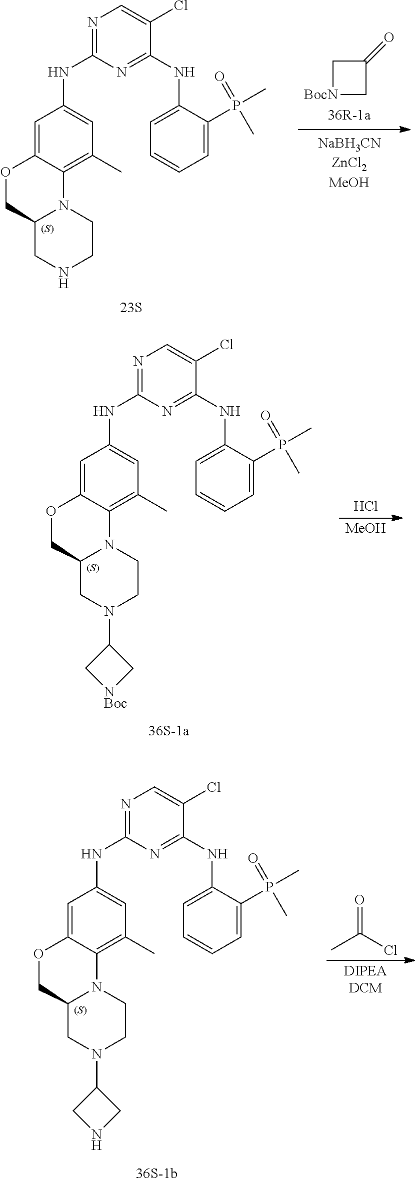

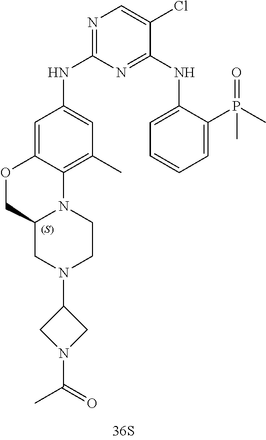

Compound 23S (237 mg, 0.47 mmol), tert-butyl 3-oxoazetidine-1-carboxylate (36R-1a, 122 mg, 0.71 mmol), and anhydrous zine chloride (194 mg, 1.42 mmol) were dissolved in MeOH (15 mL) followed by the addition of sodium cyanoborohydride (90 mg, 1.42 mmol). The reaction mixture in a sealed tube was heated to 100° C., and stirred for 1 hour. The reaction was monitored by TLC for completion. It was cooled to room temperature, and concentrated under reduced pressure. The residue was purified by silica gel column chromatography (CH₂Cl₂:MeOH=20:1, 2% aqueous ammonium hydroxide solution) to afford compound 36S-1a (340 mg) as a yellow solid which was used in next step. MS m/z 654.7 [M+H]J.

Compound 36S-1a (340 mg, 0.47 mmol) was dissolved in MeOH (15 mL) followed by the addition of 4.0 M HCl in 1,4-dioxane (4.0 M, 3 mL). The reaction mixture was stirred at 40° C. for 2 hours. The reaction was monitored by TLC for completion. It was cooled to room temperature, and concentrated under reduced pressure. The residue was dissolved in a small amount of MeOH, neutralized with aqueous ammonium hydroxide solution, and concentrated under reduced pressure. The residue was purified by silica gel column chromatography (CH₂Cl₂:MeOH=12:1, 2% aqueous ammonium hydroxide solution) to afford compound 36S-1b (206 mg, yield of 2 steps 78%) as a light yellow solid. MS m/z 554.6 [M+H]⁺.

Compound 36S-1b (40 mg, 0.07 mmol) and N,N-diisopropylethylamine (19 mg, 0.14 mmol) were dissolved in CH₂Cl₂ (6 mL). A solution of acetylchloride (6 mg, 0.07 mmol) in CH₂Cl₂ (1 mL) was slowly added at 0° C. The reaction mixture was stirred at room temperature for 15 minutes. It was concentrated under reduced pressure. The residue was purified by silica gel column chromatography (CH₂Cl₂:MeOH=15:1, 2% aqueous ammonium hydroxide solution) to afford compound 36S (24 mg, yield 56%) as a light yellow solid. ¹H NMR (500 MHz, CDCl₃) δ 11.10 (s, 1H), 8.69-8.59 (m, 1H), 8.03 (s, 1H), 7.54-7.46 (m, 1H), 7.35-7.27 (m, 2H), 7.17-7.07 (m, 2H), 6.80 (dd, J=10.5, 2.3 Hz, 1H), 4.65-4.56 (m, 1H), 4.19-4.10 (m, 1H), 4.07-3.91 (m, 3H), 3.89-3.77 (m, 1H), 3.24 (d, J=11.1 Hz, 1H), 3.19-3.04 (m, 2H), 2.95-2.85 (m, 1H), 2.83-2.68 (m, 2H), 2.59-2.47 (m, 1H), 2.31-2.27 (m, 1H), 2.25 (s, 3H), 1.89 (d, J=1.9 Hz, 3H), 1.85 (s, 3H), 1.82 (s, 3H). MS m/z 596.7 [M+H]⁺.

### Example 50: Preparation of Compound 37R

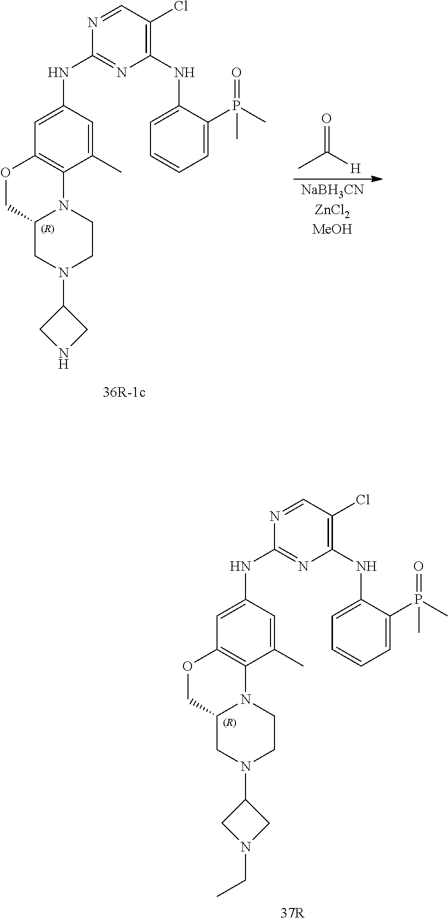

Compound 36R-1c (50 mg, 0.09 mmol), acetaldehyde (6 mg, 0.14 mmol), and anhydrous zinc chloride (37 mg, 0.27 mmol) were dissolved in MeOH (5 mL) followed by the addition of sodium cyanoborohydride (17 mg, 0.27 mmol). The reaction mixture in a sealed tube was heated to 70° C., and stirred for 2 hours. The reaction was monitored by TLC for completion. It was cooled to room temperature, and concentrated under reduced pressure. The residue was purified by silica gel column chromatography (CH₂Cl₂:MeOH=20:1, 2% aqueous ammonium hydroxide solution) to afford compound 37R (24 mg, yield 46%) as a white solid. ¹H NMR (500 MHz, CDCl₃) δ 10.90 (s, 1H), 8.68-8.63 (m, 1H), 8.07 (s, 1H), 7.53-7.47 (m, 1H), 7.31-7.22 (m, 1H), 7.13-7.05 (m, 2H), 6.81 (d, J=2.2 Hz, 1H), 6.76 (s, 1H), 4.60 (t, J=10.7 Hz, 1H), 4.00 (dd, J=10.6, 2.8 Hz, 1H), 3.80-3.65 (m, 2H), 3.23-3.17 (m, 1H), 3.16-2.96 (m, 4H), 2.91-2.83 (m, 1H), 2.76-2.61 (m, 4H), 2.49 (dd, J=11.4, 4.1 Hz, 1H), 2.27-2.21 (m, 1H), 2.24 (s, 3H), 1.84 (s, 3H), 1.82 (s, 3H), 1.16-1.01 (m, 3H). MS m/z 582.5 [M+H]⁺.

### Example 51: Preparation of Compound 37S

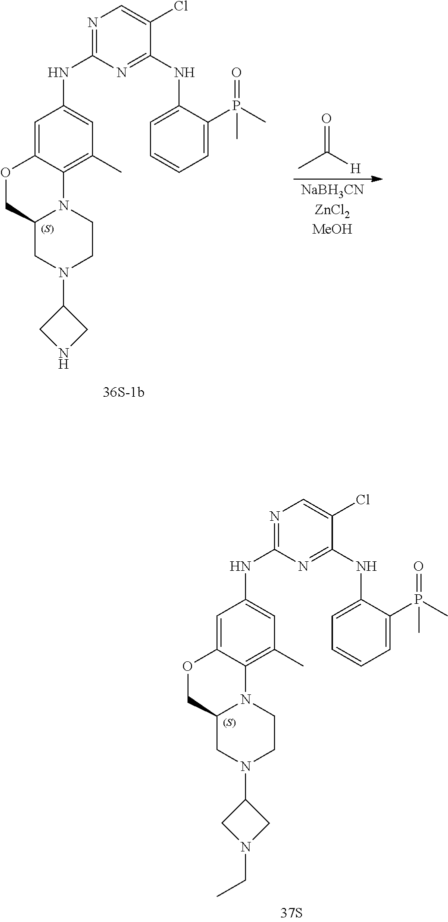

Compound 36S-1b (40 mg, 0.07 mmol), acetaldehyde (5 mg, 0.11 mmol), and anhydrous zinc chloride (30 mg, 0.22 mmol) were dissolved in MeOH (8 mL) followed by the addition of sodium cyanoborohydride (14 mg, 0.22 mmol). The reaction mixture in a sealed tube was heated to 70° C., and stirred for 1 hour. The reaction was monitored by TLC for completion. It was cooled to room temperature, and concentrated under reduced pressure. The residue was purified by silica gel column chromatography (CH₂Cl₂:MeOH=15:1, 2% aqueous ammonium hydroxide solution) to afford compound 37S (30 mg, yield 71%) as a white solid. ¹H NMR (500 MHz, CDCl₃) δ 10.90 (s, 1H), 8.72-8.59 (m, 1H), 8.07 (s, 1H), 7.50 (t, J=7.9 Hz, 1H), 7.30-7.26 (m, 1H), 7.13-7.05 (m, 2H), 6.81 (d, J=2.3 Hz, 1H), 6.76 (s, 1H), 4.60 (t, J=10.7 Hz, 1H), 4.00 (dd, J=10.6, 2.8 Hz, 1H), 3.82-3.61 (m, 2H), 3.20 (d, J=10.7 Hz, 1H), 3.15-2.93 (m, 4H), 2.92-2.82 (m, 1H), 2.75-2.61 (m, 4H), 2.49 (dd, J=11.5, 4.3 Hz, 1H), 2.24 (s, 3H), 2.22-2.17 (m, 1H), 1.84 (s, 3H), 1.82 (s, 3H), 1.15-1.01 (m, 3H). MS m/z 582.8 [M+H]*.

### Example 52: Preparation of Compound 38 R

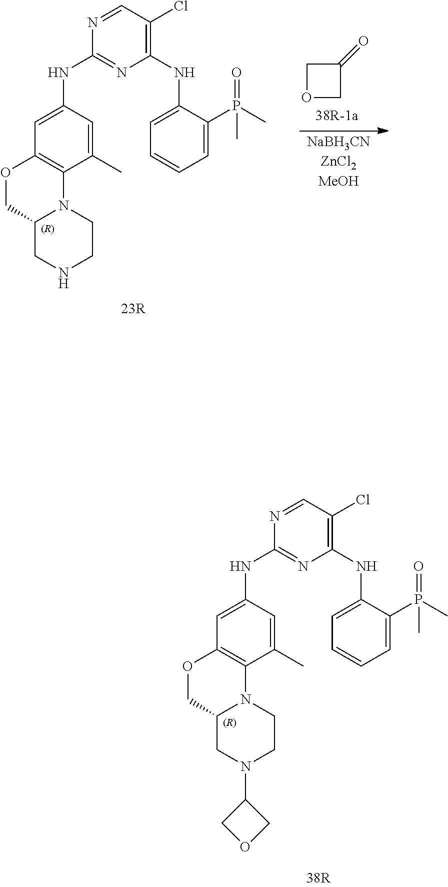

To a mixture of compound 23R (40 mg, 0.08 mmol), 3-oxacyclobutanone (38R-1a, 9 mg, 0.12 mmol), and anhydrous zinc chloride (33 mg, 0.24 mmol) in MeOH (3 mL) was added NaBH₃CN (15 mg, 0.24 mmol). The reaction in a sealed tube was stirred to 100° C., and stirred for 2 hours. The reaction was monitored by TLC for completion. It was cooled to room temperature, and concentrated under reduced pressure. The residue was purified by silica gel column chromatography (CH₂Cl₂:MeOH=20:1, 2% aqueous ammonium hydroxide solution) to afford compound 38R (25 mg, yield 56%) as a white solid. ¹H NMR (500 MHz, CDCl₃) δ 11.29 (s, 1H), 8.68-8.62 (m, 1H), 7.99 (s, 1H), 7.74 (bs, 1H), 7.54-7.48 (m, 1H), 7.32-7.27 (m, 1H), 7.19-7.13 (m, 1H), 7.11 (d, J=2.4 Hz, 1H), 6.80 (d, J=2.2 Hz, 1H), 4.74-4.53 (m, 5H), 4.05 (dd, J=10.7, 2.8 Hz, 1H), 3.53-3.44 (m, 1H), 3.26-3.20 (m, 1H), 3.11-3.05 (m, 1H), 2.98-2.91 (m, 1H), 2.76-2.67 (m, 2H), 2.54-2.49 (m, 1H), 2.31-2.21 (m, 1H), 2.25 (s, 3H), 1.85 (s, 3H), 1.83 (s, 3H). MS m/z 555.4 [M+H]⁺.

### Example 53: Kinase Activity Inhibition Experiment

1. EGFR (C797S) Kinase Activity Inhibition Experiment:

The Lantha screen Assay method was used to test the inhibitory activity of the compounds at a kinase EGFR (C797S) concentration of 10 nM and an ATP concentration of 0.1 μM. The control samples were Staurosporine.

Experimental Steps:

1) Buffer preparation: 50 mM HEPES, pH 7.5, 0.0015% Brij-35.

2) Control sample Staurosporine and test samples preparation: Staurosporine and the compounds of the embodiment of the present invention are formulated into a gradient concentration solution in 100% DMSO, and diluted to 10% DMSO with the above-mentioned buffer, and added to a 384-well plate. For example, if the initial concentration of the compound is 10 μM, 100% DMSO is used to prepare a 1000 μM solution, and 10 concentration gradients are diluted, and 100 nL solution is transferred to a 384-well plate with Echo*LIQUID HANDLE RS (LABCYTE, USA).

3) The kinase EGFR (C797S) was diluted with the following buffer to the required concentration: 50 mM HEPES, pH 7.5, 0.0015% Brij-35, 2 mM DTT. Transfer 5 μL to the 384-well plate and incubate with the compound for 10-15 minutes.

4) Dilute the substrate with the following buffer to the desired concentration: 50 mM HEPES, pH 7.5, 0.0015% Brij-35, 10 mM MgCl₂. Transfer 5 μL to the 384-well plate to initiate the reaction, and incubate at room temperature for 30 minutes.

The concentration of each reagent in the test is shown in Table 1 below.

TABLE 1

| | Substrates | | Kinase | ATP |
|---|---|---|---|---|
| | | Final | Final | Final |
| Kinase | Peptide | Concentration | Concentration | Concentration |
| EGFR | Fluorescein- | 0.2 μM | 10 nM | 0.1 μM |
| (C7975) | Poly GT | | | |

5) Use TR-Fret dilution buffer to prepare 2-fold detection solution. The final concentration of Antibody is 2 nM and the final concentration of EDTA is 10 mM. Transfer 10 μL of detection solution to the reaction wells of the 384-well plate, shake and mix, and incubate at room temperature for 60 min.

6) Read the ratio of excitation at 340 nm and emission at 520 nm to 495 nm on Envision, and calculate the inhibition rate.

7) Fit IC₅₀ with XL-fit software.

The activities of representative compounds are shown in Table 2. IC₅₀ values are shown in the following way:

A: 1 nM<IC₅₀ value ≤50 nM; B: 50 nM<IC₅₀ value ≤250 nM; C: 250 nM<IC₅₀ value ≤1000 nM; D: IC₅₀ value >1000 nM.

TABLE 2
EGFR (C797S) kinase activity inhibition (IC₅₀ value)

| Compounds | EGFR(C797S) |
|---|---|
| 1 | A |
| 1R | A |
| 1S | A |
| 2R | B |
| 3R | A |
| 4R | A |
| 4S | A |
| 5R | B |
| 6R | B |
| 6S | A |
| 7R | B |
| 8R | A |
| 9R | C |
| 10R | A |
| 11R | A |
| 12R | B |
| 12S | B |
| 13R | B |
| 14R | B |
| 15R | B |
| 16R | B |

2. ALK Kinase Activity Inhibition Experiment:

Method 1: In vitro enzymatic activity of ALK was measured using Caliper mobility shift assay. Compounds were dissolved in DMSO and diluted with kinase buffer: 50 mM HEPES, pH 7.5, 0.0015% Brij-35, 10 mM MgCl₂, 2 mM DTT; 5 μL of the 5-fold final concentration of compound was added to a 384-well plate (10% DMSO). Add 10 μL of 2.5-fold enzyme solution and incubate at room temperature for 10 minutes, then add 10 μL of 2.5-fold substrates (Peptide FAM-P22 and ATP) solution. After incubating at 28° C. for 60 minutes, add 25 μL of stop solution (100 mM HEPES, pH 7.5, 0.015% Brij-35, 0.2% Coating Reagent #3, 50 mM EDTA) to stop the reaction. Read conversion rate data on Caliper EZ Reader II (Caliper Life Sciences). Convert the conversion rate into inhibition rate (% inhibition=(max−conversion rate)/(max−min)*100). Max refers to the conversion rate of the DMSO control, and min refers to the conversion rate without enzyme activity. With the compound concentration and inhibition rate on the abscissa and ordinate, draw the curve, use XLFit excel add-in version 5.4.0.8 software to fit the curve and calculate the IC₅₀.

Method 2: Caliper mobility shift assay was used to measure ALK protein kinase activity. The method is basically the same as method 1, with individual parameters adjusted. The initial concentration of the compound is 1 mM, and sequentially diluted 4-fold to make 6 points (7 points for some individual compounds). Use a dispenser Echo 550 to transfer 250 nL of 100-fold final concentration of the compound to the target plate 3573, add 10 μL ALK with final concentration of 1.25 nM, incubate at room temperature for 10 minutes (the negative control well contains 10 μL kinase buffer and 250 nL 100% DMSO; the positive control well contains 10 μL kinase solution and 250 nL 100% DMSO). On the kinase ALK, add 15 μL of ATP with a final concentration of 30 μM and 3 μM of the substrate peptide No. 22 mixed solution to react for 25 minutes; add 30 μL of stop detection solution containing EDTA to stop the kinase reaction. Use Caliper EZ Reader to read the conversion rate. Inhibition rate %=(Average positive control conversion rate %−Sample conversion rate %/(Average positive control conversion rate %−Average negative control conversion rate %). Among them: negative control wells represent the conversion rate readings without enzyme activity; positive control wells represent the conversion rate readings of wells without compound inhibition. Take the log value of the concentration as the X-axis, and the percentage inhibition rate on the Y-axis. Log (inhibitor) vs. response—Variable slope of analysis software GraphPad Prism 5 was used to fit the curve to obtain the IC₅₀ value of each compound on the enzyme activity. Calculation formula: Y=Bottom+(Top−Bottom)/(1+10\hat{ }((Log IC₅₀−X)*HillSlope)).

The activities of representative compounds are shown in Table 3. IC₅₀ values are shown in the following way:

A: 1 nM<IC₅₀ value ≤50 nM; B: 50 nM<IC₅₀ value ≤250 nM; C: 250 nM<IC₅₀ value ≤1000 nM; D: IC₅₀ value >1000 nM.

TABLE 3
ALK kinase activity inhibition (IC₅₀ value)

| Compounds | ALK |
|---|---|
| 1 | A |
| 1R | A |
| 1S | A |
| 2R | A |
| 3R | A |
| 4R | A |
| 4S | A |
| 5R | A |
| 6R | A |
| 6S | A |
| 7R | A |
| 8R | A |
| 9R | A |
| 10R | A |
| 11R | A |
| 12R | B |
| 12S | A |
| 13R | B |
| 14R | B |
| 15R | B |
| 16R | B |
| 25R | A |
| 26R | A |
| 27R | A |
| 27S | A |
| 30R | A |
| 31R | A |
| 32R | A |

Note:
Compounds1-16R were tested using method 1, compounds 25R-32R were tested using method 2.

3. EGFR (T790M/L858R/C797S) Kinase Activity Inhibition Experiment:

Caliper mobility shift assay was used to detect the inhibitory effect of the compound on EGFR (T790M/L858R/C797S) kinase activity. The basic method is the same as ALK activity test method 2. The test concentration of the compound starts at 2 μM, and is diluted 5 folds into 6 (7 for individual compounds) concentration points. Use a dispenser Echo 550 to transfer 250 nL 100× final concentration of the compound to the target plate 3573, add 10 μL of EGFR (T790M/L858R/C797S) kinase solution with a final concentration of 2.5 nM, and incubate for 10 minutes at room temperature (the negative control well contains 10 μL kinase buffer and 250 nL 100% DMSO; the positive control well contains 10 μL kinase solution and 250 nL 100% DMSO). Add 15 μL of ATP with a final concentration of 40 μM and 3 μM substrate peptide No. 22 mixed solution to react for 60 minutes. Add 30 μL of stop solution containing EDTA to stop the kinase reaction. Use Caliper EZ Reader to read the conversion rate. Inhibition rate %=(Average positive control conversion rate %−Sample conversion rate %/(Average positive control conversion rate %−Average negative control conversion rate %). Among them: negative control wells represent the conversion rate readings without enzyme activity; positive control wells represent the conversion rate readings of wells without compound inhibition. Take the log value of the concentration as the X-axis, and the percentage inhibition rate on the Y-axis. Log (inhibitor) vs. response—Variable slope of analysis software GraphPad Prism 5 was used to fit the curve to obtain the IC₅₀ value of each compound on the enzyme activity. Calculation formula: Y=Bottom+(Top−Bottom)/(1+10\hat{ }((Log IC₅₀−X)*HillSlope)).

The activities of representative compounds are shown in Table 4. IC₅₀ values are shown in the following way:

A: IC₅₀ value ≤3 nM; B: 3 nM<IC₅₀ value ≤15 nM; C: 15 nM<IC₅₀ value ≤100 nM; D: IC₅₀ value >100 nM.

TABLE 4
EGFR (T790M/L858R/C797S) kinase activity inhibition
(IC₅₀ value)

| Compounds | EGFR (T790M/L858R/C797S) |
|---|---|
| Brigatinib | B |
| 16R | C |
| 18R | A |
| 21R | A |
| 22R | B |
| 23S | A |
| 24R | B |
| 25R | A |
| 26R | B |
| 27R | A |
| 27S | A |
| 29R | B |
| 30R | A |
| 31R | A |
| 31S | A |
| 32R | A |
| 32S | A |
| 33R | A |
| 34S | A |
| 35S | A |

4. MAP4K1 (HPK1) Kinase Activity Inhibition Experiment:

Caliper mobility shift assay was used to detect the inhibitory effect of the compound on MAP4K1 (HPK1) kinase activity. The test concentration of the compound starts at 500 nM, and is diluted 5 folds into 5 concentration points. Use a dispenser Echo 550 to transfer 250 nL 100-fold final concentration of the compound to the target plate 3573, add 10 μL of MAP4K1 (HPK1) kinase solution with a final concentration of 1.25 nM, and incubate for 10 minutes at room temperature (the negative control well contains 10 μL kinase buffer and 250 nL 100% DMSO; the positive control well contains 10 μL kinase solution and 250 nL 100% DMSO). On the kinase MAP4K1 (HPK1), add 15 μL of ATP with a final concentration of 10.1 μM and 3 μM substrate peptide No. 25 mixed solution to react for 120 minutes. Add 30 μL of stop solution containing EDTA to stop the kinase reaction. Use Caliper EZ Reader to read the conversion rate. Inhibition rate %=(Average positive control conversion rate %−Sample conversion rate %/(Average positive control conversion rate %−Average negative control conversion rate %). Among them: negative control wells represent the conversion rate readings without enzyme activity; positive control wells represent the conversion rate readings of wells without compound inhibition. Take the log value of the concentration as the X-axis, and the percentage inhibition rate on the Y-axis. Log (inhibitor) vs. response—Variable slope of analysis software GraphPad Prism 5 was used to fit the curve to obtain the IC₅₀ value of each compound on the enzyme activity. Calculation formula: Y=Bottom+(Top−Bottom)/(1+10 \hat{ }((Log IC₅₀−X)*HillSlope)).

The activities of representative compounds are shown in Table 5. IC₅₀ values are shown in the following way:

A: IC₅₀ value ≤1 nM; B: 1 nM<IC₅₀ value ≤10 nM; C: 10 nM<IC₅₀ value ≤100 nM; D: IC₅₀ value >100 nM.

TABLE 5
MAP4K1 (HPK1) kinase activity inhibition (IC₅₀ value)

| Compounds | MAP4K1(HPK1) |
|---|---|
| Brigatinib | C |
| 12R | A |
| 14R | B |
| 15R | B |
| 16R | B |
| 18R | A |
| 19R | A |
| 21R | A |
| 22R | A |
| 23S | A |
| 24R | B |
| 25R | A |
| 26R | A |
| 27R | A |
| 27S | A |
| 29R | A |
| 30R | A |
| 31R | A |
| 31S | A |
| 32R | A |
| 32S | A |
| 33R | A |
| 34S | A |
| 35S | A |

### Example 54: Ba/F3_EGFR (del19-T790M-C797S) Cell Proliferation Inhibition Experiment

Method 1: Cell culture: Ba/F3_EGFR del19/T790M/C797S cell culture medium is RPMI-1640+10% FBS+1% dual antibiotic solution. The cells were cultured in a 37° C., 5% CO₂ incubator.

Cell plating and compound treatment: 1) Cells are routinely cultured until the cell saturation is 80%-90%, and when the number reaches the requirement, the cells are collected. 2) Resuspend in the corresponding fresh medium, take a small amount of cells to count, and prepare a cell suspension with a suitable density. 3) Inoculate the cell suspension into a 384-well plate at 700 cells/well, 30 μL per well. 4) Use Echo to add the compound to the corresponding cell well. The maximum starting concentration of the compound is 1-10 μM, 3 folds dilution, 8 concentrations. The blank control wells contain cells plus 0.1% DMSO, and the positive control wells contain cells plus 10 μM Brigatinib. Cells were cultured in a 37° C., 5% CO₂ incubator for 72 h.

CTG method detection: 1) Add 30 μL of CTG reagent (CelltiterGlo kit) to each well, and let it stand for 30 minutes at 37° C. and 5% CO₂ in the dark. 2) Use Envision instrument to read the chemiluminescence signal value.

Data analysis: IC₅₀ results are analyzed by GraphPad Prism 6 software. Following nonlinear fitting formula is used to obtain the IC₅₀ (half inhibitory concentration) of the compounds: Y=Bottom+(Top−Bottom)/(1+10 \hat{ }((Log IC₅₀−X)×HillSlope)). X: log value of compound concentration; Y: inhibition rate (% inhibition); inhibition rate (% inhibition)=(blank control well reading−compound well reading)/(blank control well reading−positive control reading)×100.

Method 2: Cell plating and compound treatment: 1) Take Ba/F3_EGFR Del19/T790M/C797S cells, centrifuge at 800 rpm for 5 minutes, discard the supernatant, and resuspend in fresh medium (RPMI-1640+10% FBS). Take a small amount of cells and count them with ViCell. 2) Adjust the cell density, inoculate the cells in a 384-well plate at 2000/well, and incubate in a 37° C., 5% CO₂ incubator for 4 hours. 3) Set up the program of Tecan HP D300, add the compound to the well plate. The maximum initial concentration of the compound is 1-4 μM, 3 folds dilution, 9 concentrations, and the DMSO content in each well is unified to 0.2%. The cells were cultured in a 37° C., 5% CO₂ incubator for 72 h.

CTG method detection: 1) Take out the CTG reagent and cell plate and equilibrate at room temperature for 30 min, add 25 μL CTG to each well, shake at medium speed for 2 min, centrifuge at 1000 rpm for 1 minutes, and stand still at room temperature for 10 minutes. 2) Envision instrument reads the chemiluminescence signal value.

Data analysis: IC50 results are analyzed by IDBS's XL-fit 5.0 software.

The activities of representative compounds are shown in Table 6. IC₅₀ values are shown in the following way:

A: IC₅₀ value ≤20 nM; B: 20 nM<IC₅₀ value ≤100 nM; C: 100 nM<IC₅₀ value ≤200 nM; D: IC₅₀ value >200 nM.

TABLE 6
Ba/F3_EGFR (del19-T790M-C797S) cell proliferation inhibition
(IC₅₀ value)

| | BaF3_EGFR(del19-T790M-C797S) | |
|---|---|---|
| Compounds | Method 1 | Method 2 |
| Brigatinib | C | D |
| 1R | A | |
| 1S | A | |
| 2R | B | |
| 3R | B | |
| 4R | B | |
| 4S | B | |
| 5R | C | |
| 6R | B | |
| 6S | B | |
| 8R | B | |
| 9R | B | |
| 10R | B | |
| 11R | A | |
| 12R | B | |
| 13R | B | |
| 14R | C | |
| 17R | A | |
| 18R | C | |
| 19R | A | |
| 20R | B | |
| 21R | D | |
| 22R | A | |
| 23R | B | |
| 23S | A | |
| 24R | B | |
| 24S | B | |
| 25R | A | |
| 26R | A | |
| 27R | A | A |
| 27S | | A |
| 28R | B | |
| 29R | B | |
| 30R | | B |
| 31R | | B |
| 31S | | B |
| 32R | | B |
| 32S | B | |
| 33R | | B |
| 34S | | A |
| 35S | | B |

### Example 55: Pharmacokinetic Study in Rats

Instruments: XEVO TQ-S LC/MS instrument produced by Waters. All measurement data is collected and processed by Masslynx V4.1 software, and the data is calculated and processed by Microsoft Excel. Using WinNonLin 8.0 software, the statistical moment method was used to calculate the pharmacokinetic parameters. Mainly include kinetic parameters Tₘₐₓ, T_{1/2}, Cₘₐₓ, AUCₗₐₛₜ etc. Column: ACQUITY UPLC BEH C18 (2.1 mm×50 mm, 1.7 μm); column temperature 40° C.; mobile phase A is water (0.1% formic acid), mobile phase B is acetonitrile, flow rate is 0.350 ml/min, gradient elution is adopted, the elution gradient is 0.50 min: 10% B; 1.50 min: 10% B; 2.30 min: 95% B; 2.31 min: 10% B; 3.00 min: stop. Injection volume: 5 μL.

Animals: 3 male SD rats with a weight range of 200-220 g. After purchase, they will be kept in the laboratory of the Experimental Animal Center for 2 days and then used. They will be fasted for 12 hours predose and 4 hours after dosing. Drinking water is free during the test. After the rats were gavage, blood samples were taken according to the established time point.

Solvent: 0.5% Methylcellulose (aqueous solution containing 0.4% Tween 80 and 1% ethanol). Preparation of the solution for intragastrical administration: accurately weigh the compound, add it to the solvent, and ultrasonically at room temperature for 5 minutes to completely dissolve the drug, and prepare a 0.3 mg/ml medicinal solution.

Pharmaceutical samples: representative compounds of the structure shown in the patented formula (I) of the present invention, generally, multiple samples with similar structures (with a molecular weight difference of more than 2 units) are taken, accurately weighed, and administered together (cassette PK). In this way, multiple compounds can be screened at the same time and their oral absorption rates can be compared. A single administration was also used to study the pharmacokinetics of the drug sample in rats.

After intragastrical administration, blood was taken from the orbit at 0.25, 0.5, 1, 2, 4, 9, 12 and 24 hours, and placed in a plastic centrifuge tube pretreated with sodium heparin. After centrifugation, the supernatant plasma was used for LC-MS/MS analysis.

Accurately weigh the compounds to prepare different concentrations, perform quantitative analysis on mass spectrometry to establish a standard curve, and then test the concentration of the above-mentioned compound in the plasma to obtain the compound concentration at different time points. All measurement data are collected and processed by relevant software, and the statistical moment method is used to calculate the pharmacokinetic parameters (mainly including kinetic parameters Tₘₐₓ, T_{1/2}, Cₘₐₓ, AUCₗₐₛₜ etc). The results are as follows:

Table 7: Pharmacokinetic Parameters of the Compounds after Intragastrical Administration in SD Rats

| | | T_{1/2} | Tₘₐₓ | Cₘₐₓ | AUC_{0-24 h} |
|---|---|---|---|---|---|
| Compounds | Dosage | (h) | (h) | (ng/mL) | (h * ng/mL) |
| 1R | 4 mg/kg | 12.67 | 1.08 | 23.10 | 101.89 |
| 25R | 3 mg/kg | 6.75 | 6.33 | 61.44 | 585.35 |
| 27S | 3 mg/kg | 5.20 | 10.00 | 140.33 | 2102.82 |
| 27R | 3 mg/kg | 5.17 | 5.33 | 195.84 | 2751.90 |
| 31R | 3 mg/kg | 5.18 | 6.33 | 78.37 | 686.57 |
| 31S | 3 mg/kg | 6.27 | 1.00 | 160.17 | 1345.22 |
| 32R | 3 mg/kg | 1.95 | 2.00 | 306.38 | 3419.6 |
| 32S | 3 mg/kg | 2.22 | 3.33 | 351.99 | 3263.69 |
| 33R | 3 mg/kg | 3.88 | 7.33 | 120.36 | 1775.84 |
| 35S | 3 mg/kg | 3.97 | 4.33 | 15.03 | 179.27 |

All literatures mentioned in the present application are incorporated herein by reference, as though each one is individually incorporated by reference. Additionally, it should be understood that after reading the above contents, those skilled in the art can make various changes and modifications to the present invention. These equivalents also fall within the scope defined by the appended claims.

1. A compound of formula (I), or the optical isomers (including racemates, single enantiomers, and possible diastereomers), pharmaceutically acceptable salts, prodrugs, deuterated derivatives, hydrates, or solvates thereof: 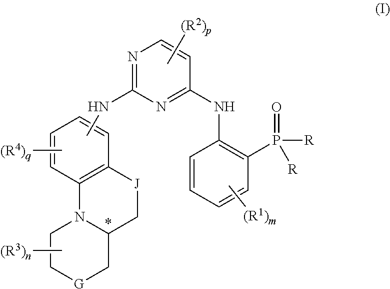
wherein in the formula (I):
“*” indicates a chiral center;
each R is independently C₁₋₄ alkyl;
each R¹ is independently hydrogen, deuterium, halogen, C₁₋₄ alkyl, C₂₋₄ alkenyl, C₂₋₄ alkynyl, C₃₋₆ cycloalkyl, 3- to 8-membered heterocyclic, aryl, heteroaryl, OR^{h}, or CN;
each R² is independently hydrogen, deuterium, halogen, C₁₋₄ alkyl, C₂₋₄ alkenyl, C₂₋₄ alkynyl, C₃₋₆ cycloalkyl, 3- to 8-membered heterocyclic, aryl, heteroaryl or CN;
each R³ is independently hydrogen, deuterium, or C₁₋₄ alkyl; or when two R³ are simultaneously attached to the same carbon atom, the two R³ and the carbon atom to which they are attached may optionally form a carbonyl group (C═O);
each R⁴ is independently hydrogen, deuterium, halogen, C₁₋₄ alkyl, C₁₋₄ haloalkyl, C₂₋₄ alkenyl, C₂₋₄ alkynyl, OR^{h}, SR^{h}, NR^{h}R^{h}, CN, C(O)R^{e}, C(O)OR^{h}, C(O)NR^{h}R^{h}, OC(O)R^{e}, NR^{h}C(O)R^{e}, or S(O)₂R^{e};
J and G are each independently NR^{f}, O, S, S(O), S(O)₂ or CR^{g}R^{g};
m is 0, 1, 2, 3, or 4;
n is 0, 1, 2, or 3;
p is 0, 1, or 2;
q is 0, 1, 2, or 3;
R^{f} is hydrogen, C₁₋₈ alkyl, C₁₋₈ haloalkyl, C₂₋₈ alkenyl, C₂₋₈ alkynyl, C₃₋₈ cycloalkyl, 3- to 12-membered heterocyclic, aryl, heteroaryl, C(O)R^{e}, C(O)OR^{h}, C(O)NR^{h}R^{h} S(O)₂R^{e}, or S(O)₂NR^{h}R^{h}; wherein, each of the above groups is unsubstituted or substituted with 1-3 R^{e};
each R^{e} is independently selected from the group consisting of hydrogen, C₁₋₄ alkyl, C₁₋₄ haloalkyl, C₁₋₄ alkoxy substituted C₁₋₄ alkyl, C₂₋₄ alkenyl, C₂₋₄ haloalkenyl, C₁₋₄ alkoxy substituted C₂₋₄ alkenyl, hydroxyl substituted C₂₋₄ alkenyl, di(C₁₋₄ alkyl)amine substituted C₂₋₄ alkenyl, C₃₋₈ cycloalkyl substituted C₂₋₄ alkenyl, 3- to 8-membered heterocyclic group substituted C₂₋₄ alkenyl, aryl substituted C₂₋₄ alkenyl, heteroaryl substituted C₂₋₄ alkenyl, C₂₋₄ alkynyl, C₂₋₄ haloalkynyl, C₁₋₄ alkoxy substituted C₂₋₄ alkynyl, hydroxyl substituted C₂₋₄ alkynyl, di(C₁₋₄ alkyl)amine substituted C₂₋₄ alkynyl, C₃₋₈ cycloalkyl substituted C₂₋₄ alkynyl, 3- to 8-membered heterocyclic substituted C₂₋₄ alkynyl, aryl substituted C₂₋₄ alkynyl, heteroaryl substituted C₂₋₄ alkynyl, C₃₋₈ cycloalkyl, 3- to 8-membered heterocyclic group, aryl, or heteroaryl;
each R^{g} is independently selected from the group consisting of hydrogen, halogen, or C₁₋₄ alkyl; or two R^{g} together with the carbon atom to which they are attached form a carbonyl group (C═O); or two R^{g} together with the same carbon atom to which they attached form 3- to 8-membered cyclic structure which optionally comprises 0, 1 or 2 heteroatoms selected from N, O, S;
each R^{h} is independently hydrogen or C₁₋₄ alkyl; or two R^{h} together with the nitrogen atom to which they are attached form a 3- to 8-membered cyclic structure, which comprises 1 or 2 N atom and 0 or 1 heteroatom selected from O and S;
wherein each of the above alkyl, alkenyl, alkynyl, cycloalkyl, heterocyclic, aryl and heteroaryl is optionally and independently substituted by 1 to 3 substituents independently selected from the group consisting halogen, C₁₋₄ alkyl, C₁₋₄ haloalkyl, C₂₋₄ alkenyl, C₂₋₄ alkynyl, C₃₋₈ cycloalkyl, 3- to 8-membered heterocyclic, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, or S(O)₂R^{e}, provided that the chemical structure formed is stable and meaningful; wherein R^{e} and R^{h} are defined as above;
unless otherwise specified, the aryl is aromatic groups having 6 to 12 carbon atoms; the heteroaryl is 5- to 15-membered heteroaromatic groups; and the cyclic structure is saturated or unsaturated cyclic groups with or without heteroatoms.

2. A compound of claim 1, or the optical isomers (including racemates, single enantiomers, and possible diastereomers), pharmaceutically acceptable salts, prodrugs, deuterated derivatives, hydrates, or solvates thereof, wherein the formula (I) is: 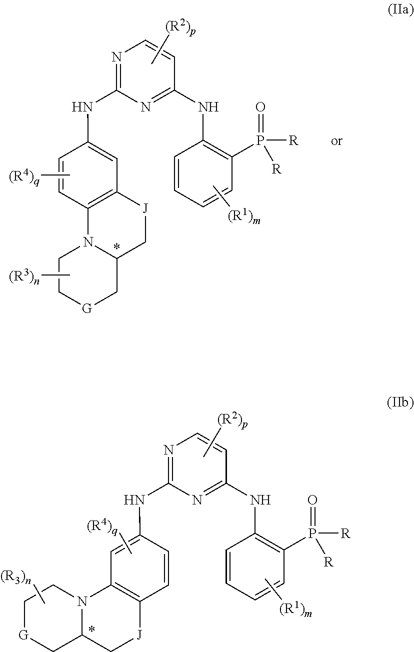
wherein each group is defined as in claim 1.

3. The compound of claim 2, wherein, each R is independently C₁₋₂ alkyl;
each R¹ is independently hydrogen, deuterium, halogen, or C₁₋₂ alkyl;
each R² is independently hydrogen, deuterium, halogen, or C₁₋₂ alkyl;
each R³ is independently hydrogen or C₁₋₄ alkyl; or when two R³ are simultaneously attached to the same carbon atom, the two R³ and the carbon atom to which they are attached form a carbonyl group (C═O);
each R⁴ is independently hydrogen, deuterated, halogen, C₁₋₄ alkyl, NR^{h}R^{h}, or NR^{h}C(O)R^{e};
m is 0, 1, or 2;
n is 0, 1, or 2;
p is 0, 1, or 2;
q is 0, 1, or 2;
wherein R^{e} and R^{h} are defined as in claim 1.

4. The compound of claim 3, wherein the formula (I) is 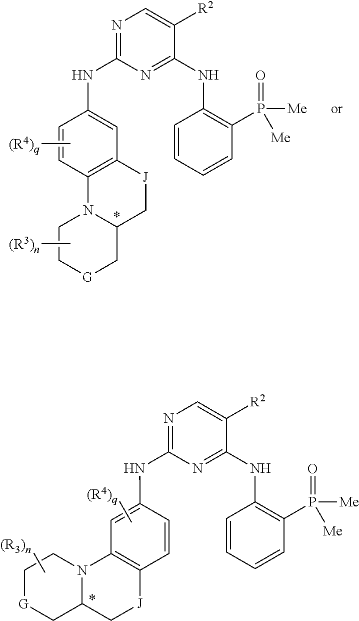
wherein R² is F, Cl or Br; each R³ is independently hydrogen or C₁₋₄ alkyl; or when two R³ are simultaneously connected to the same carbon atom, the two R³ and the carbon atom to which they connected form a carbonyl group (C═O); each R⁴ is independently hydrogen, deuterium, halogen, C₁₋₄ alkyl, NR^{h}R^{h}, or NR^{h}C(O)R^{e}; n is 0, 1 or 2; q is 0, 1 or 2; wherein J, G, R^{e} and R^{h} are as defined in claim 1.

5. The compound of claim 4, wherein the structure fragment 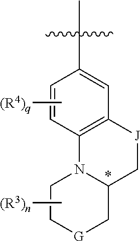
in formula (IIIa) is selected from: 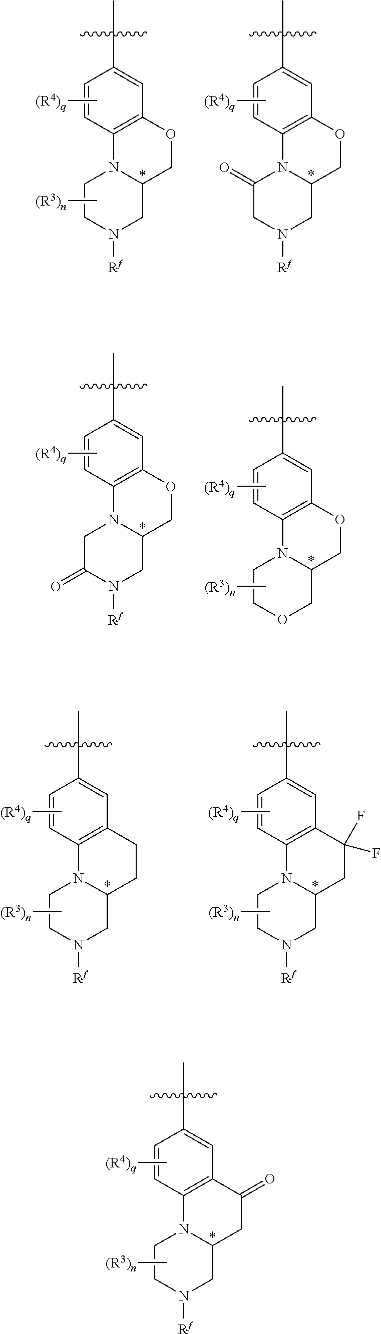
“ [symbol] ” means the connection site of the above structural fragment to other part in formula (IIIa);
wherein, each R³ is independently hydrogen or C₁₋₄ alkyl; when two R³ are simultaneously attached to the same carbon atom, the two R³ and the carbon atom to which they are attached form a carbonyl group (C═O);
each R⁴ is independently hydrogen, deuterium, halogen, C₁₋₂ alkyl, NR^{h}R^{h}, or NR^{h}C(O)R^{e};
n is 0, 1 or 2; q is 0 or 1;
R^{f} is hydrogen, C₁₋₄ alkyl, C₁₋₄ haloalkyl, C₂₋₄ alkenyl, C₂₋₄ alkynyl, C₃₋₆ cycloalkyl, 3- to 9-membered heterocyclic group, aryl, heteroaryl, C(O)R^{e}, C(O)OR^{h}, C(O)NR^{h}R^{h}, S(O)₂R^{e}, or S(O)₂NR^{h}R^{h}; wherein each alkyl, alkenyl, alkynyl, cycloalkyl, heterocyclic group, aryl and heteroaryl is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, S(O)₂R^{e}, or S(O)₂NR^{h}R^{h}; wherein R^{e} and R^{h} are as described in claim 1.

6. The compound of claim 5, wherein the formula (I) is 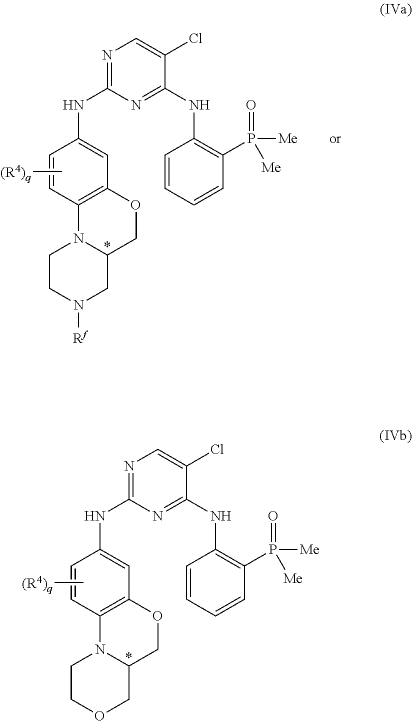
wherein each R⁴ is independently hydrogen, deuterium, halogen, C₁₋₂ alkyl, NR^{h}R^{h}, or NR^{h}C(O)R^{e}; q is 0 or 1;
R^{f} is hydrogen, C₁₋₄ alkyl, C₁₋₄ haloalkyl, C₂₋₄ alkenyl, C₂₋₄ alkynyl, C₃₋₆ cycloalkyl, 3- to 9-membered heterocyclic group, aryl, heteroaryl, C(O)R^{e}, C(O)OR^{h}, C(O)NR^{h}R^{h}, S(O)₂R^{e}, or S(O)₂NR^{h}R^{h}; wherein each alkyl, alkenyl, alkynyl, cycloalkyl, heterocyclic, aryl and heteroaryl group is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, S(O)₂R^{e}, or S(O)₂NR^{h}R^{h}; wherein the definitions of R^{e} and R^{h} are as described in claim 1.

7. The compound of claim 6, wherein R^{f} is selected from the group consisting of hydrogen, C₁₋₄ alkyl, C₁₋₄ haloalkyl, C₂₋₄ alkenyl, C₂₋₄ alkynyl, C₃₋₆ cycloalkyl, 3- to 9-membered heterocyclic group, aryl, heteroaryl, C(O)R^{e}, or S(O)₂R^{e}; wherein each alkyl, alkenyl, alkynyl, cycloalkyl, heterocyclic group, aryl and heteroaryl is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{e}, SR^{e}, NR^{e}R^{e}, C(O)R^{e}, C(O)OR^{e}, C(O)NR^{e}R^{e}, NR^{e}C(O)R^{e}, S(O)₂R^{e}, or S(O)₂NR^{h}R^{h}; wherein the definitions of R^{e} and R^{h} are as described above.

8. The compound of claim 6, wherein the formula (I) is 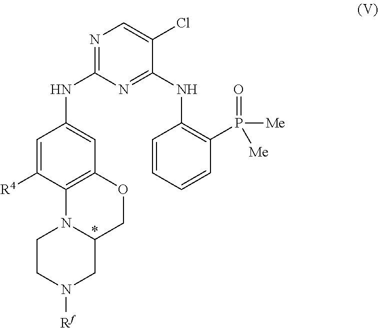
wherein, R⁴ is hydrogen, halogen, C₁₋₂ alkyl, NR^{h}R^{h}, or NR^{h}C(O)R^{e};
R^{f} is hydrogen, C₁₋₄ alkyl, C₁₋₄ haloalkyl, C₃₋₆ cycloalkyl, 3- to 9-membered heterocyclic group, aryl, heteroaryl, C(O)R^{e}, or S(O)R^{e}; wherein each alkyl, cycloalkyl, heterocyclic, aryl and heteroaryl group is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, S(O)₂R^{e}, or S(O)₂NR^{h}R^{h}; wherein the definitions of R^{e} and R^{h} are as described in claim 1.

9. The compound of claim 2, wherein formula (I) is: 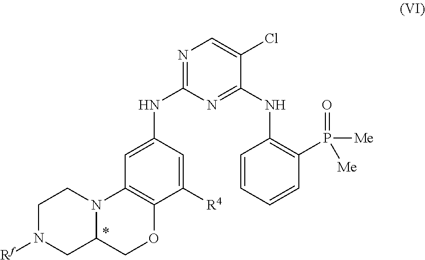
wherein, R⁴ is hydrogen, halogen, C₁₋₂ alkyl, NR^{h}R^{h}, or NR^{h}C(O)R^{e};
R^{f} is hydrogen, C₁₋₄ alkyl, C₁₋₄ haloalkyl, C₃₋₆ cycloalkyl, 3- to 9-membered heterocyclic group, aryl, heteroaryl, C(O)R^{e}, or S(O)R^{e}; wherein each alkyl, cycloalkyl, heterocyclic group, aryl and heteroaryl is optionally substituted by 1-3 groups independently selected from the group consisting of halogen, C₁₋₄ alkyl, C₂₋₄ alkenyl, C₂₋₄ alkynyl, C₃₋₈ cycloalkyl, 3- to 8-membered heterocyclic group, aryl, heteroaryl, CN, NO₂, OR^{h}, SR^{h}, NR^{h}R^{h}, C(O)R^{e}, C(O)OR^{h}, C(O)NR^{h}R^{h}, NR^{h}C(O)R^{e}, S(O)₂R^{e}, or S(O)₂NR^{h}R^{h}; wherein the definitions of R^{e} and R^{h} are as described in claim 1.

10. The compound of claim 8, wherein the formula (I) is 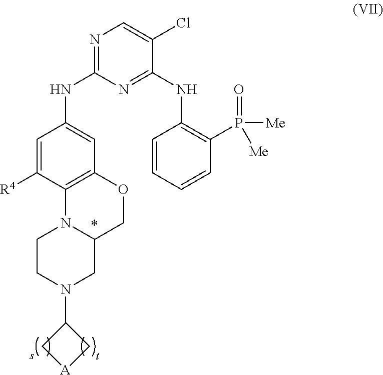
wherein, R⁴ is hydrogen, halogen or C₁₋₂ alkyl;
s and t are each independently 1, 2 or 3;
A is NR^{k}, O, or CR^{g}R^{g}; wherein R^{k} is hydrogen, C₁₋₄ alkyl, C₁₋₄ haloalkyl, hydroxy substituted C₁₋₄ alkyl, C₁₋₄ alkoxy substituted C₁₋₄ alkyl, di(C₁₋₄ alkyl) amine substituted C₁₋₄ alkyl, C₃₋₆ cycloalkyl, 3- to 9-membered heterocyclic group, aryl, heteroaryl, C(O)R^{e}, C(O)OR^{h}, C(O)NR^{h}R^{h}, S(O)₂R^{e}, or S(O)₂NR^{h}R^{h}; wherein each R^{e} is independently selected from the group consisting of hydrogen, C₁₋₄ alkyl, C₁₋₄ haloalkyl, C₂₋₄ alkenyl, C₁₋₄ alkoxy substituted C₂₋₄ alkenyl, di(C₁₋₄ alkyl)amine substituted C₂₋₄ alkenyl, C₂₋₄ alkynyl, C₃₋₆ cycloalkyl, 3- to 8-membered heterocyclic group, aryl, or heteroaryl; the definitions of R^{g} and R^{h} are as described in claim 1.

11. The compound of claim 1, wherein each R^{e} is independently selected from the group consisting of hydrogen, C₁₋₄ alkyl, C₁₋₄ haloalkyl, C₁₋₄ alkoxy substituted C₁₋₄ alkyl, C₂₋₄ alkenyl, C₁₋₄ alkoxy substituted C₂₋₄ alkenyl, hydroxyl substituted C₂₋₄ alkenyl, di(C₁₋₄ alkyl)amine substituted C₂₋₄ alkenyl, 3- to 6-membered heterocyclic substituted C₂₋₄ alkenyl, aryl substituted C₂₋₄ alkenyl, heteroaryl substituted C₂₋₄ alkenyl, C₂₋₄ alkynyl, C₃₋₈ cycloalkyl, 3- to 8-membered heterocyclic group, aryl, or heteroaryl.

12. The compound of claim 1, wherein each R⁴ is independently hydrogen, deuterium, halogen, C₁₋₂ alkyl, or NHC(O)CH═CH₂.

13. The compound of claim 1, wherein the compound is selected from the group consisting of: 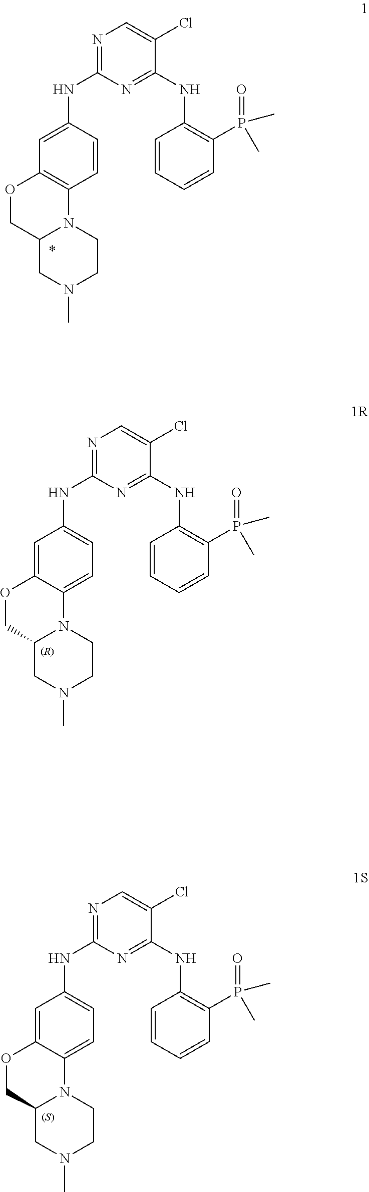 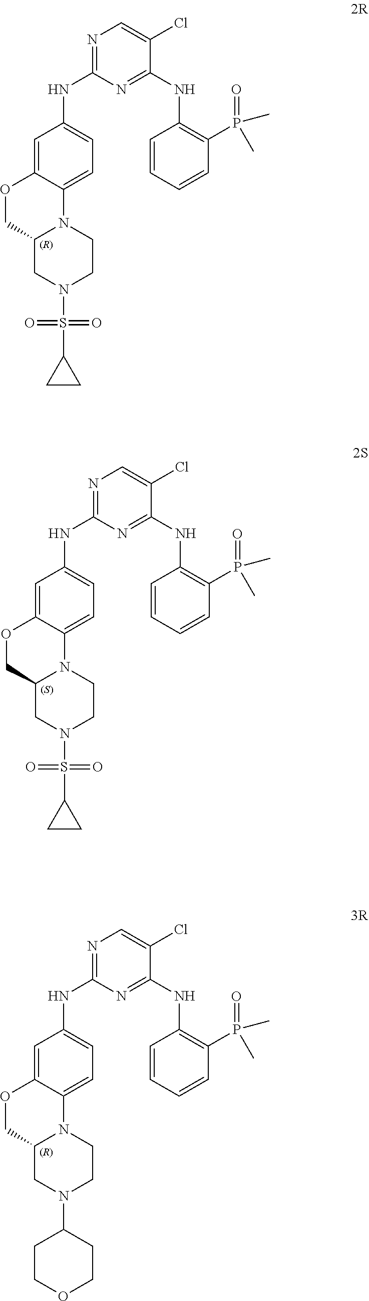 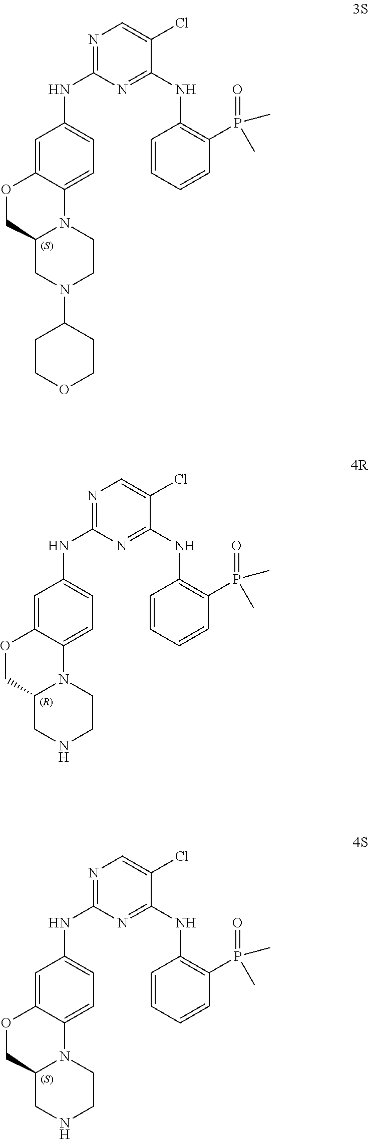 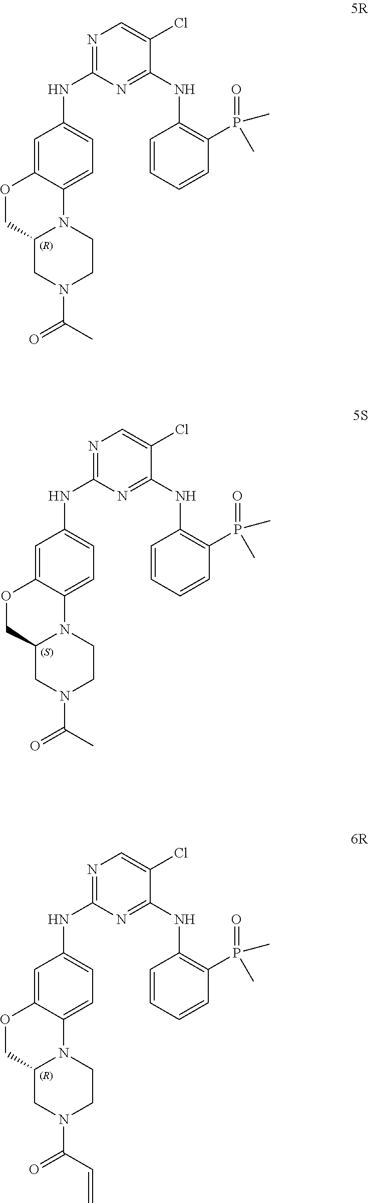 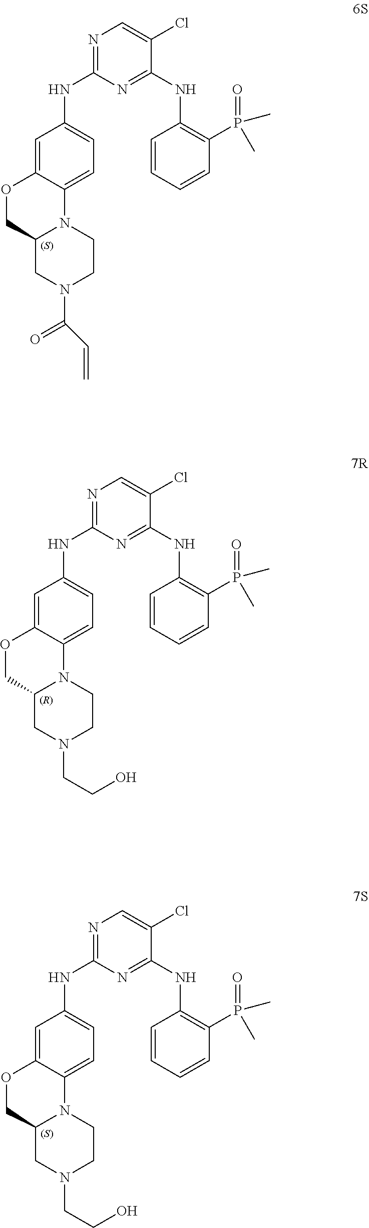 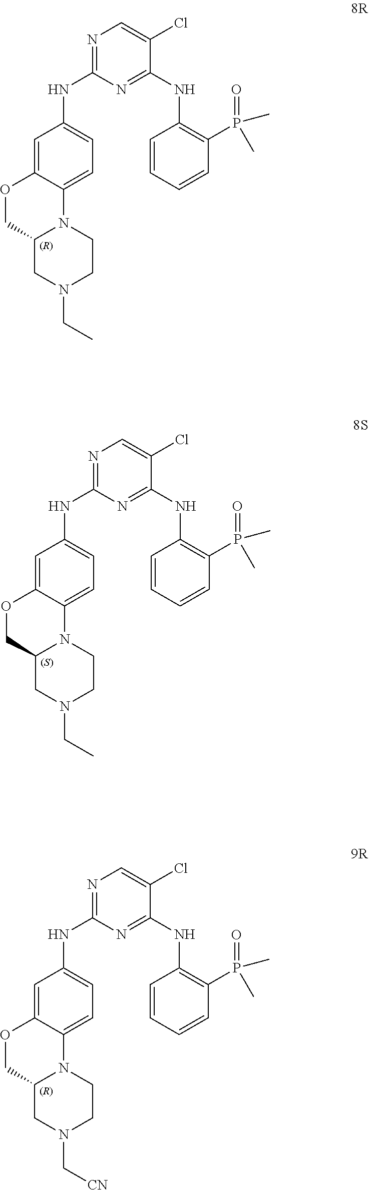 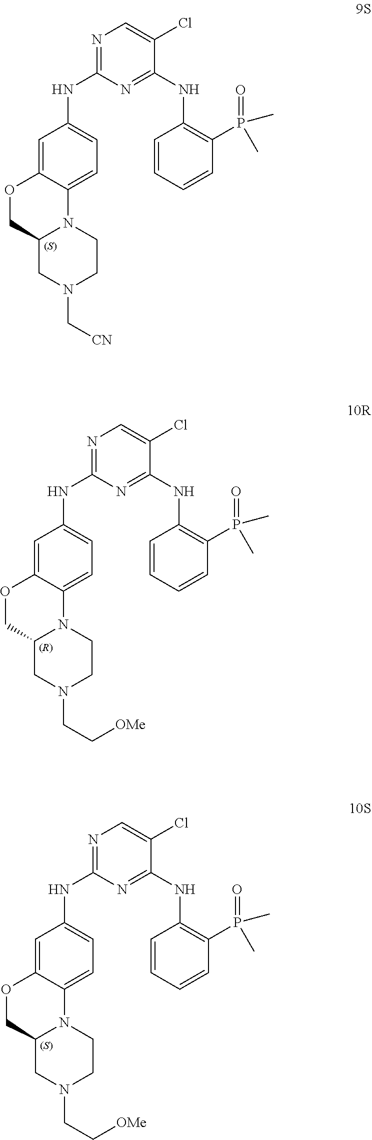 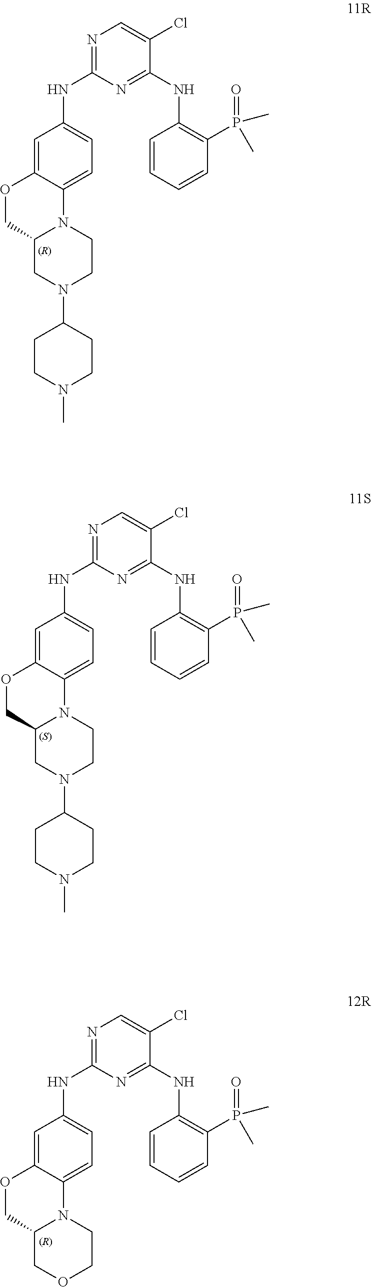 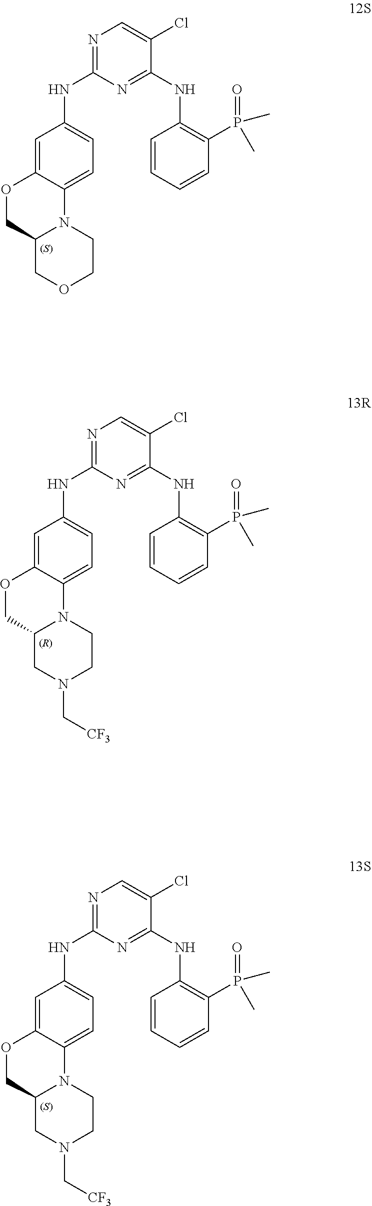 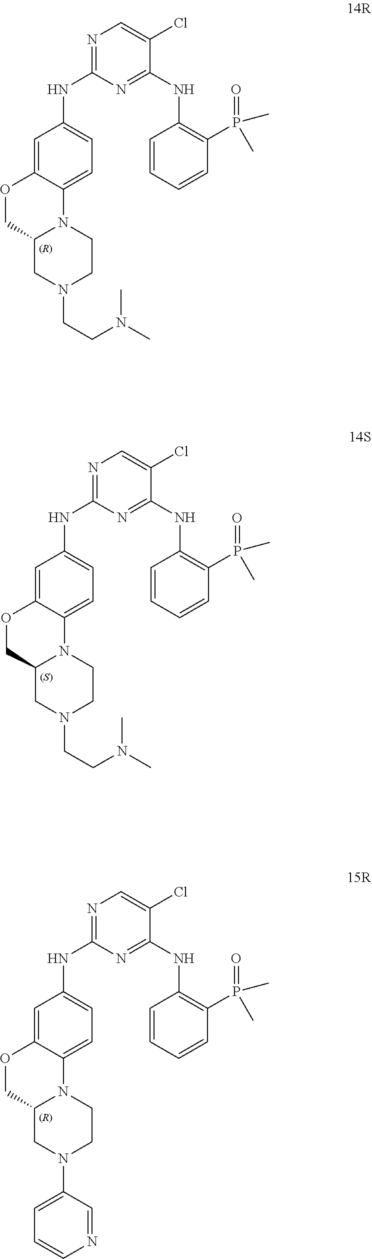 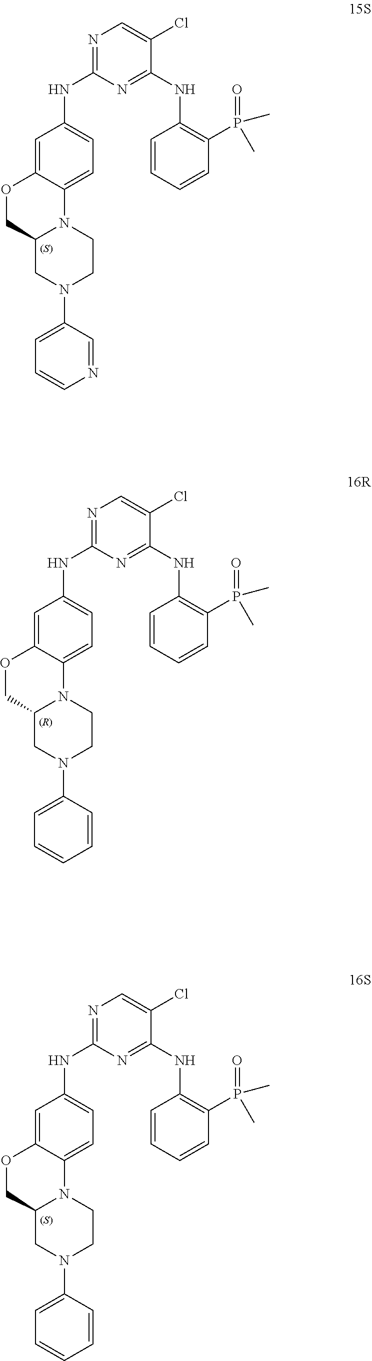 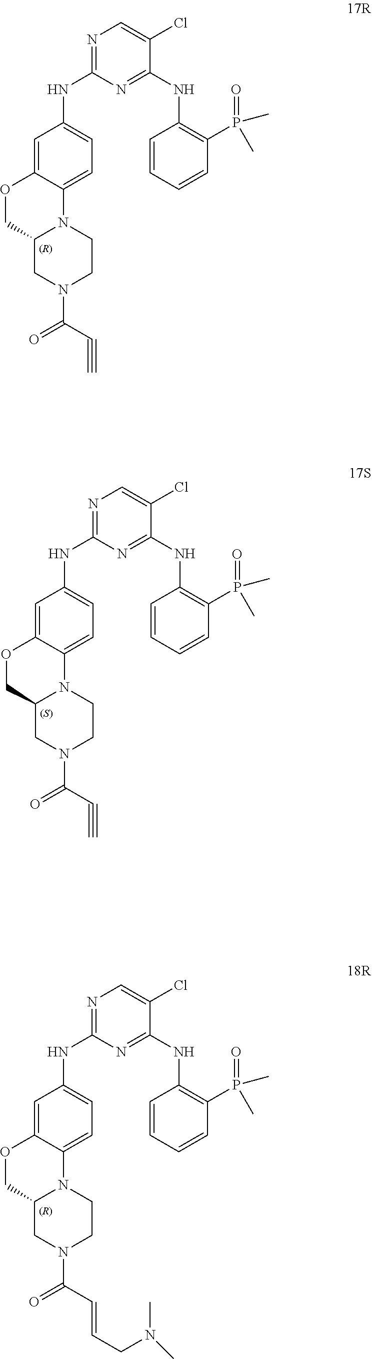 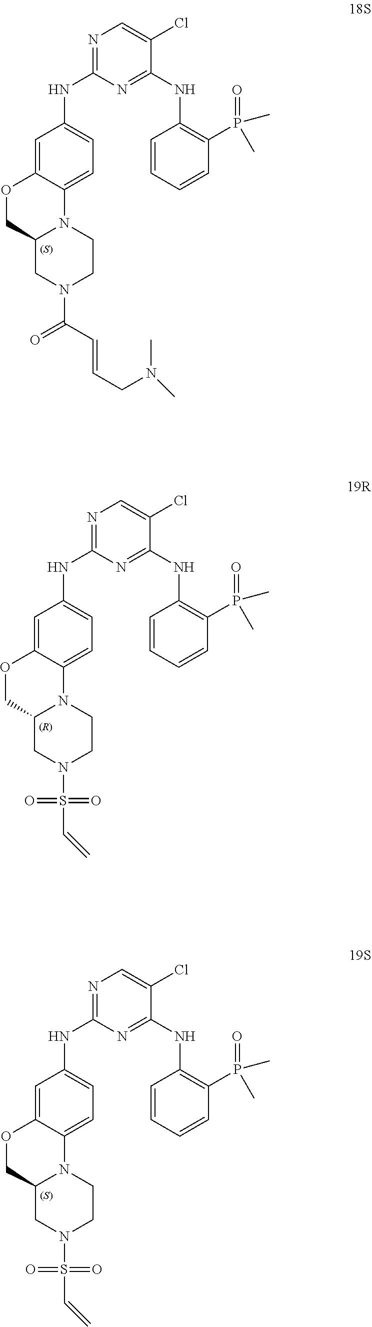 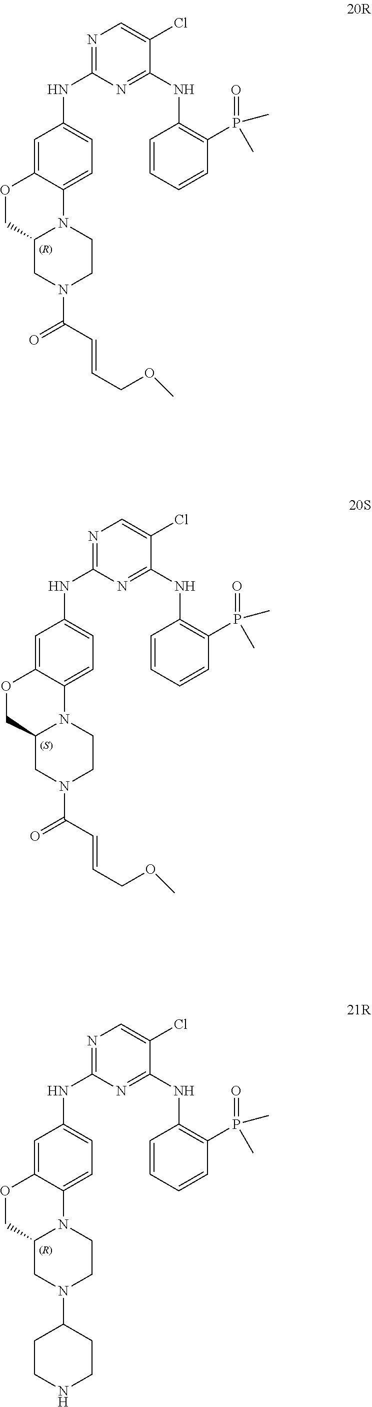 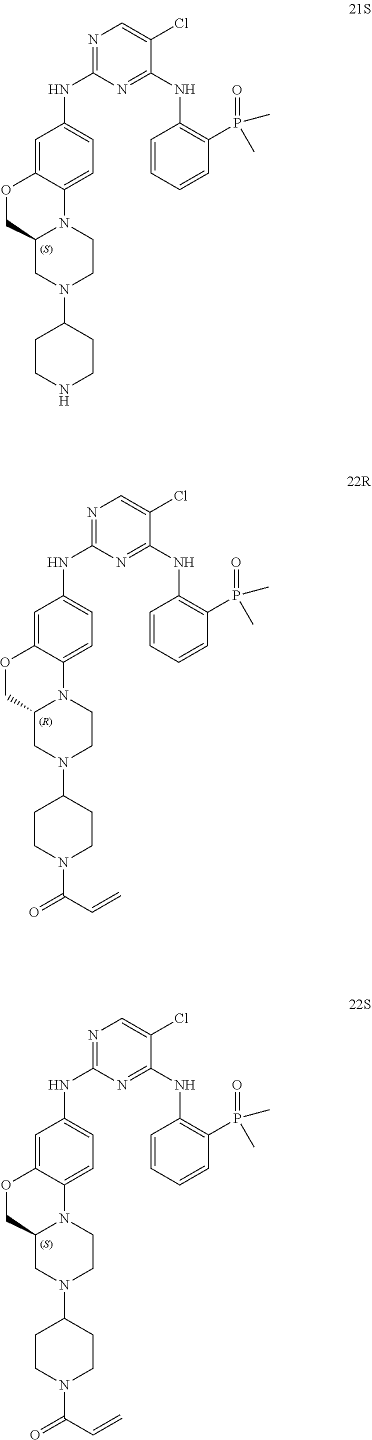 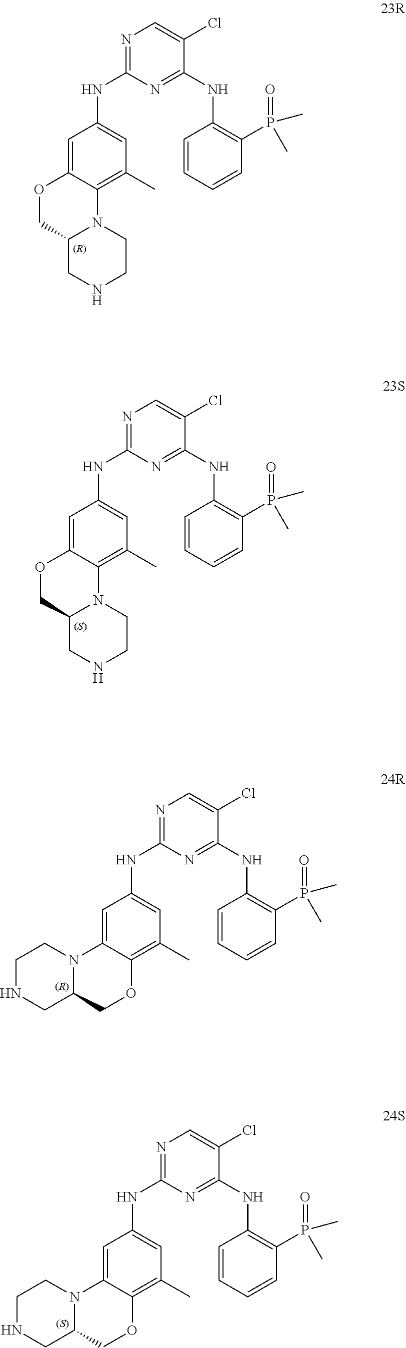 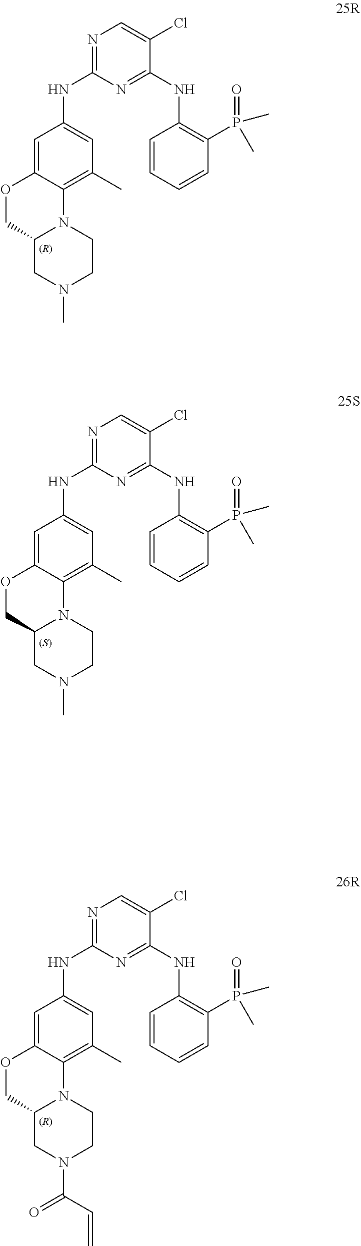 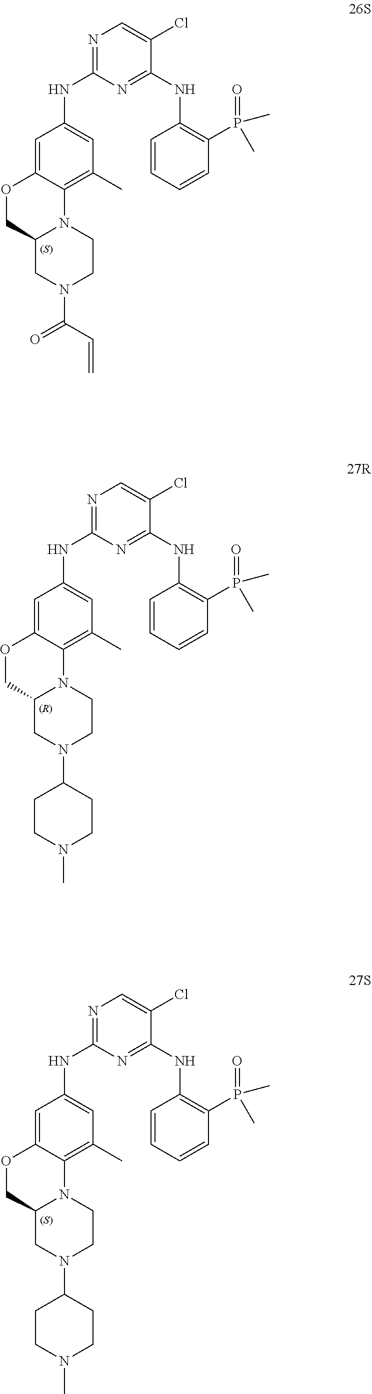 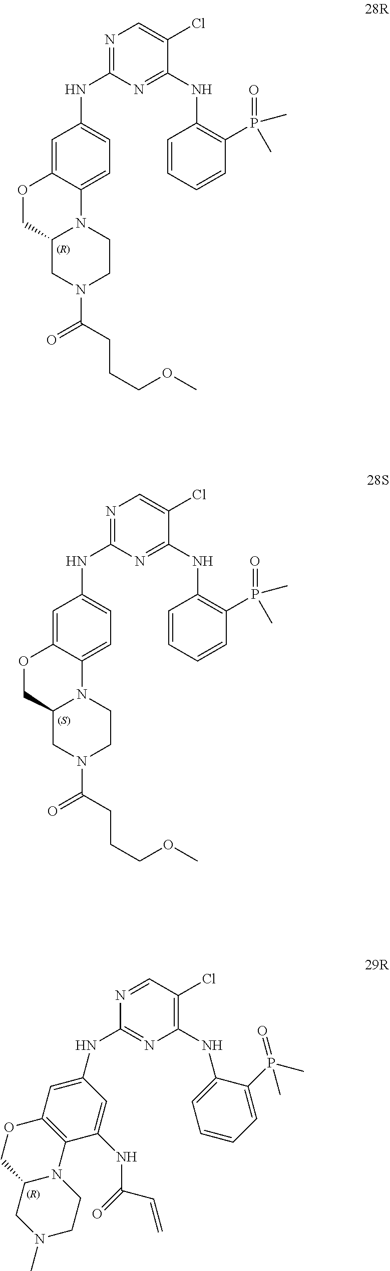 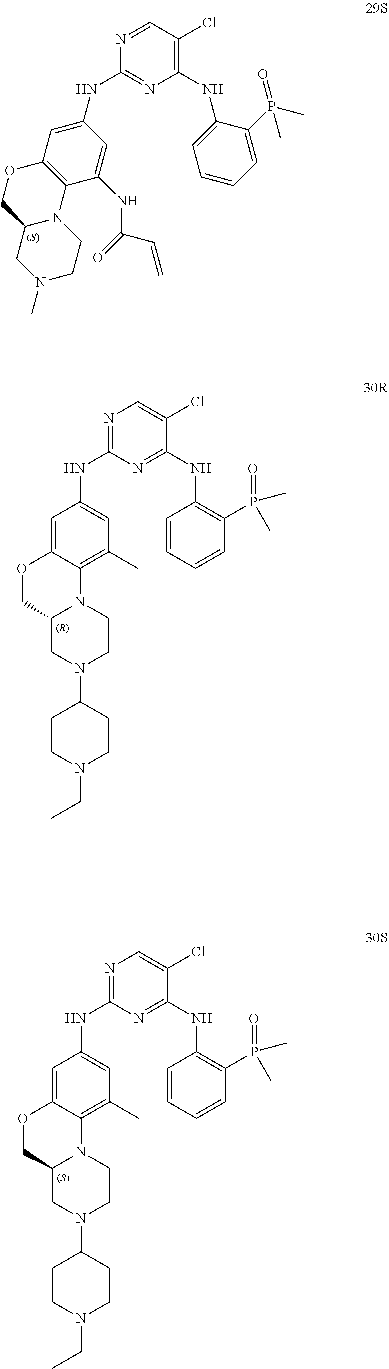 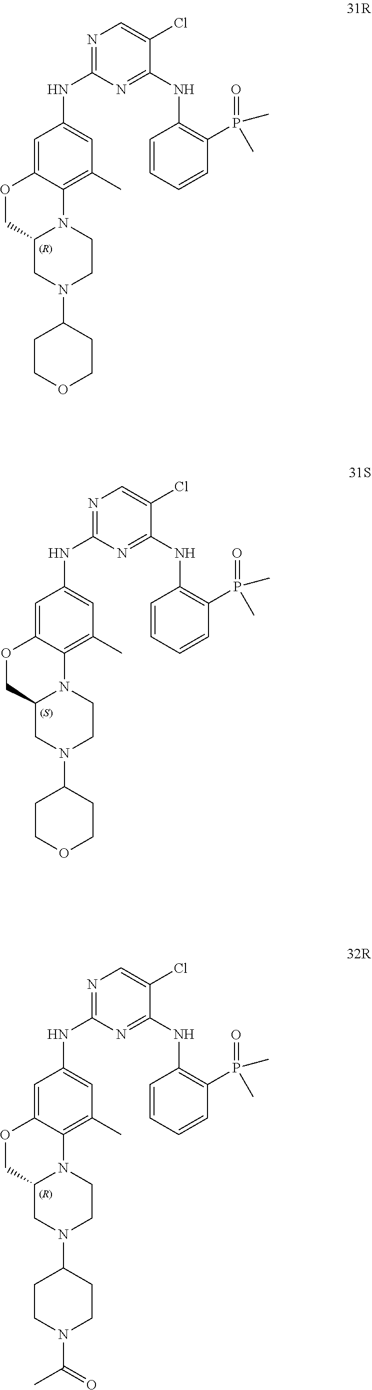 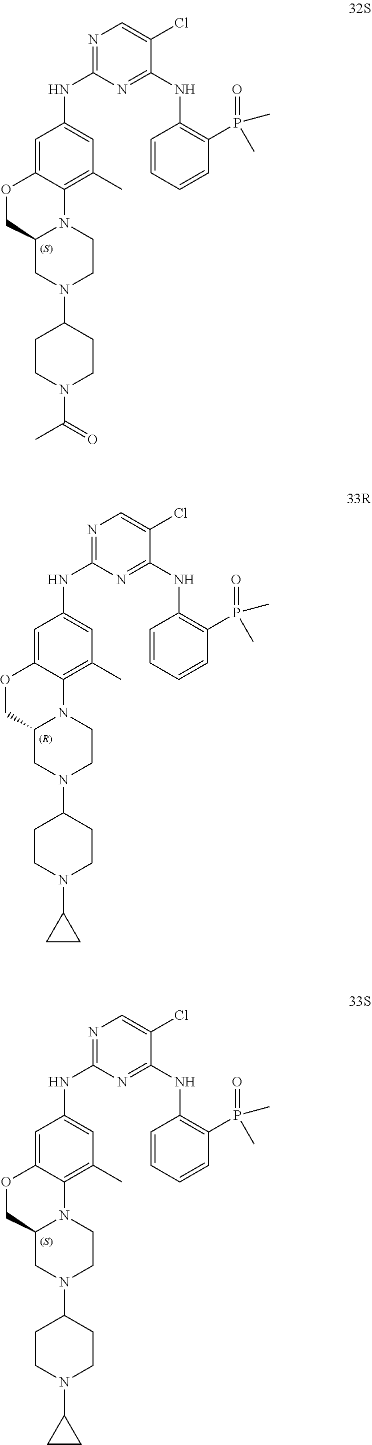 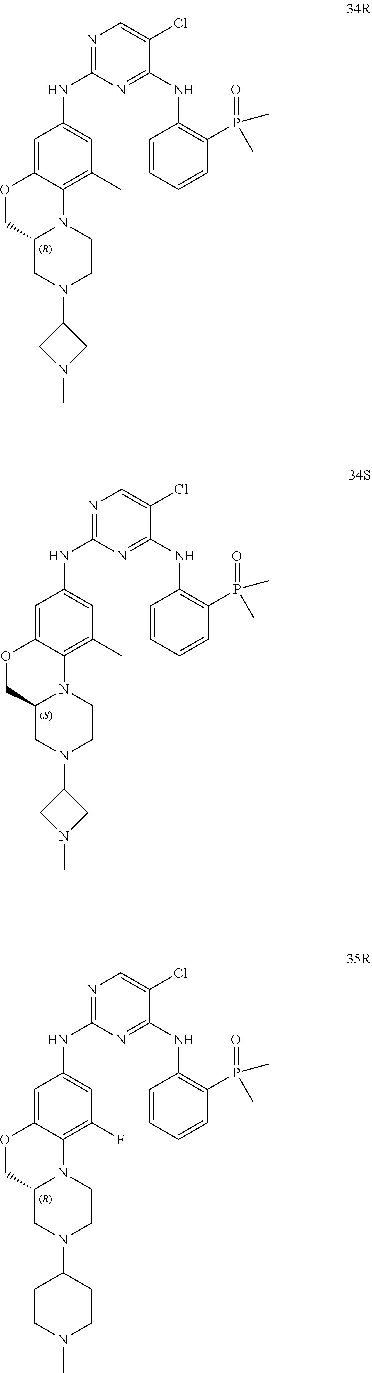 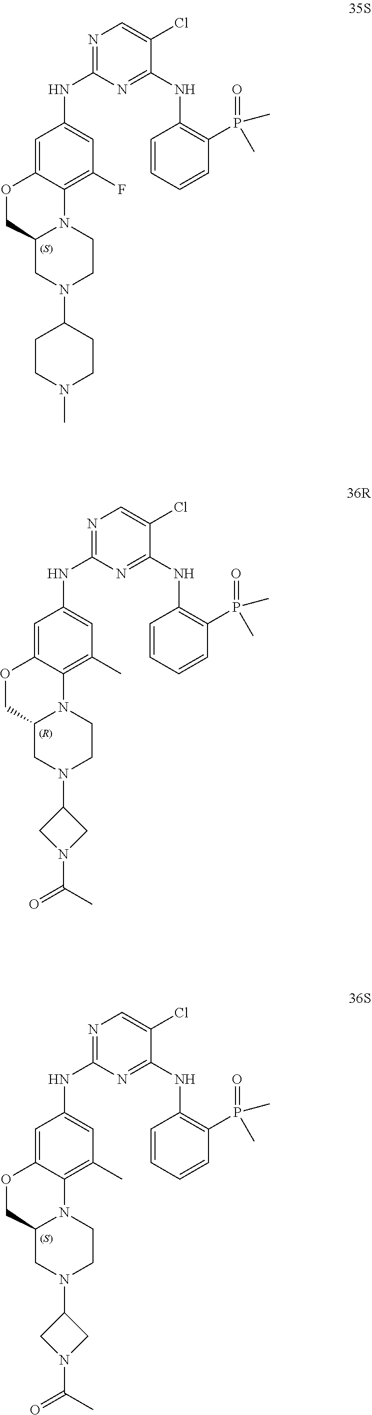 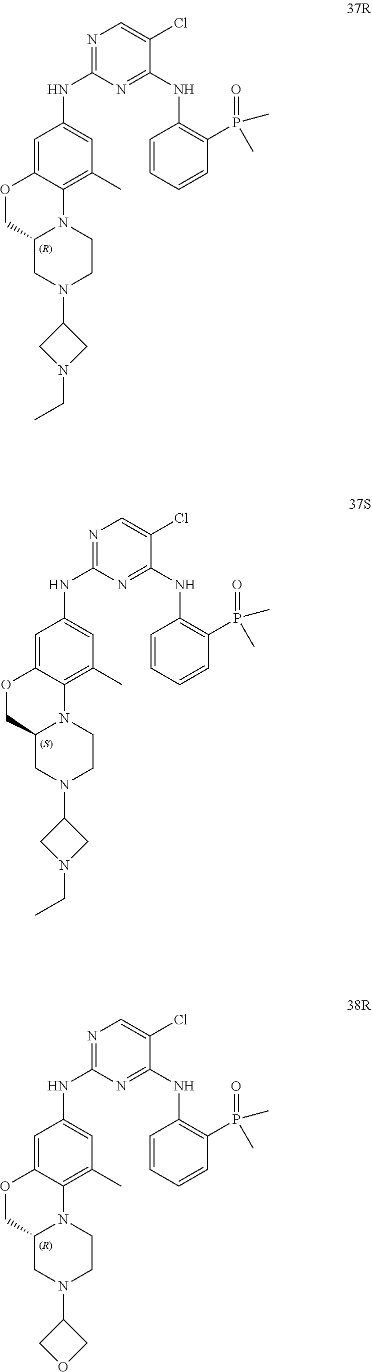 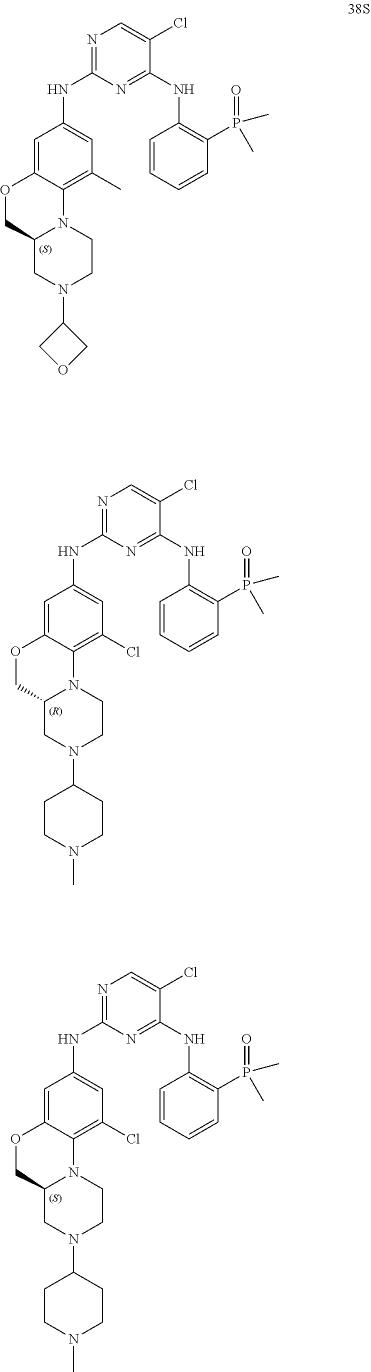 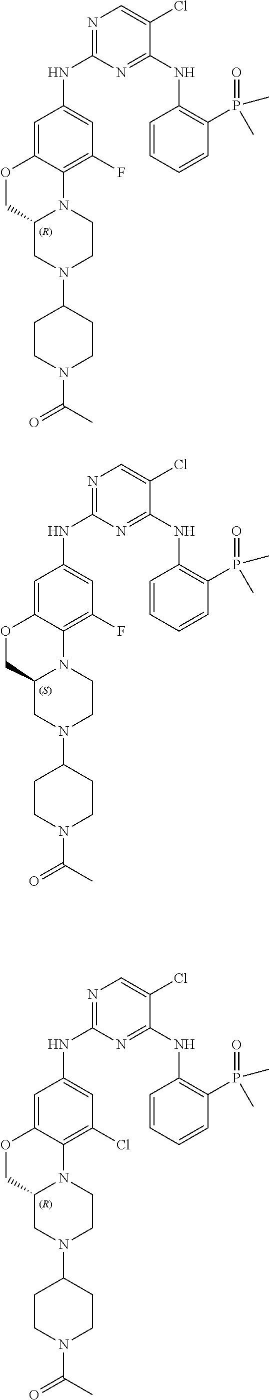 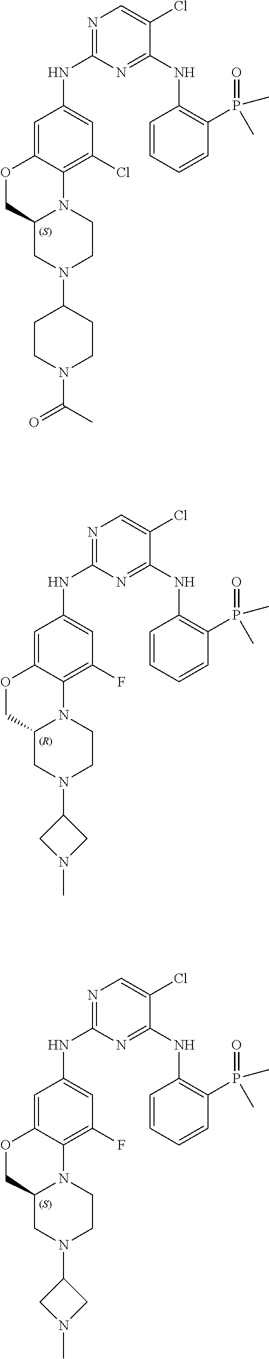 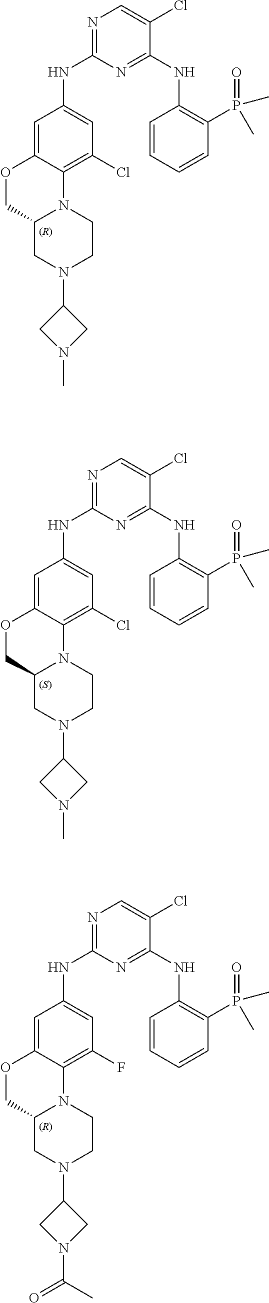 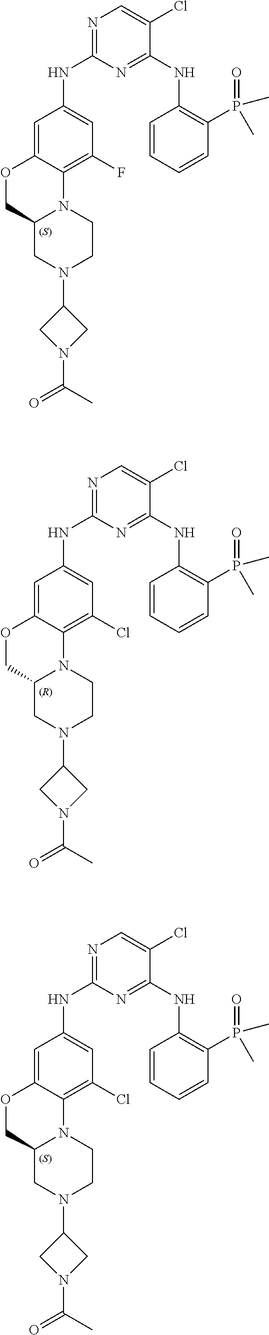 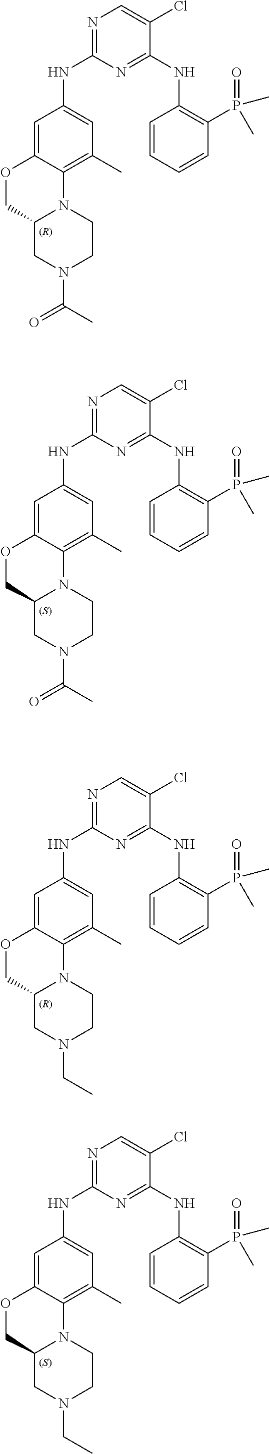 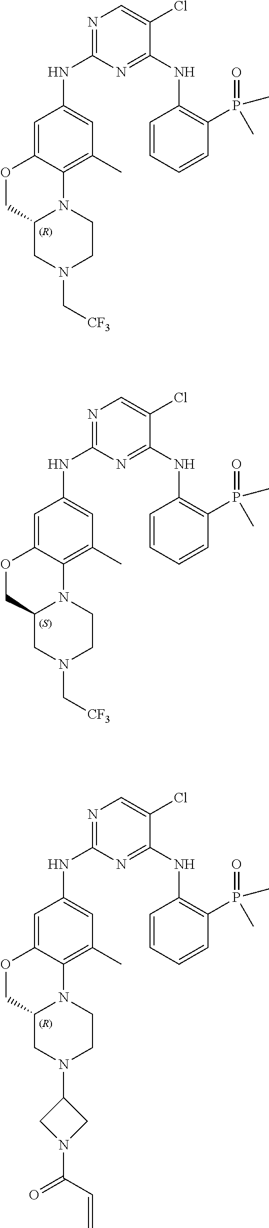 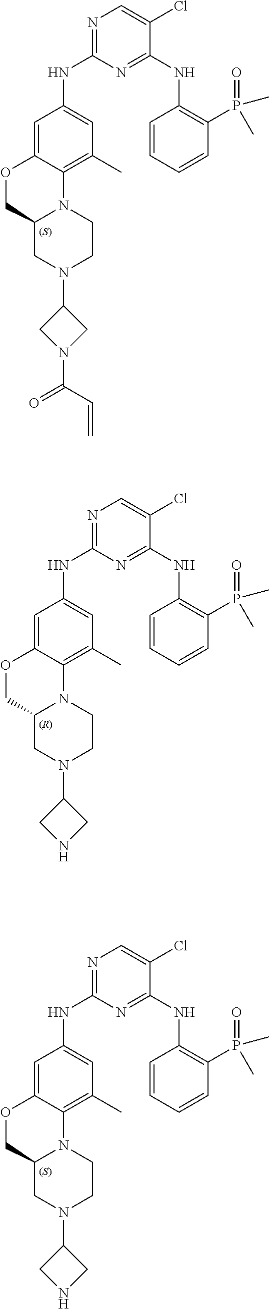 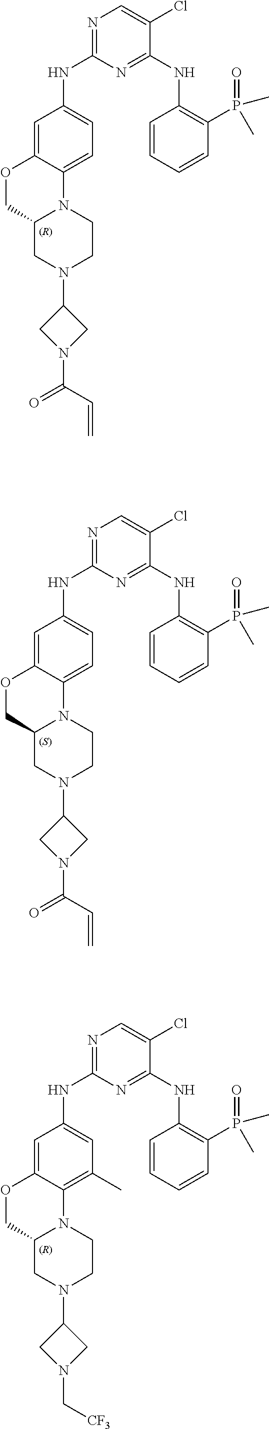 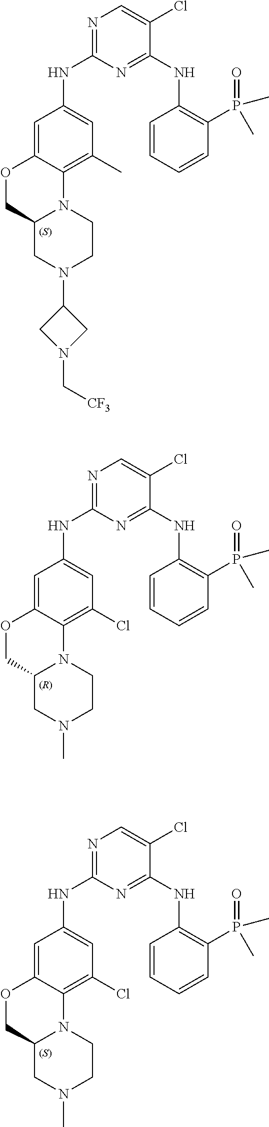 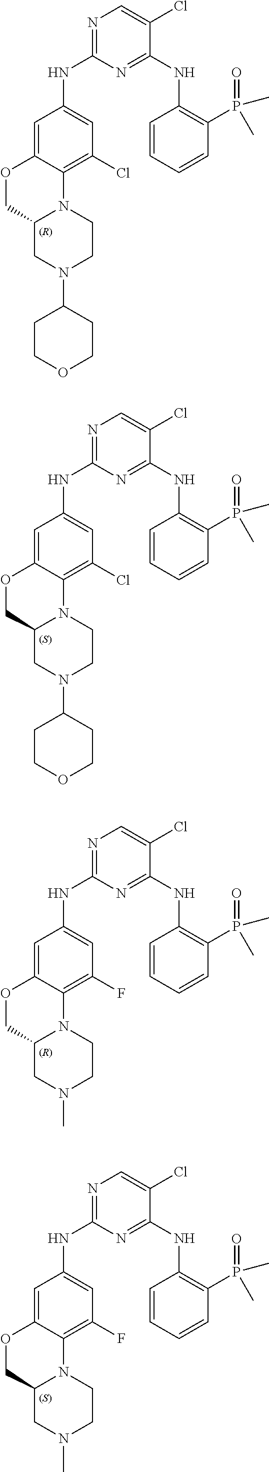 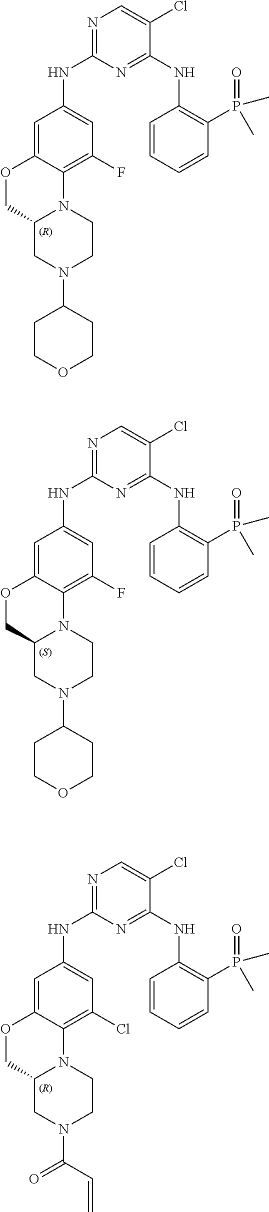 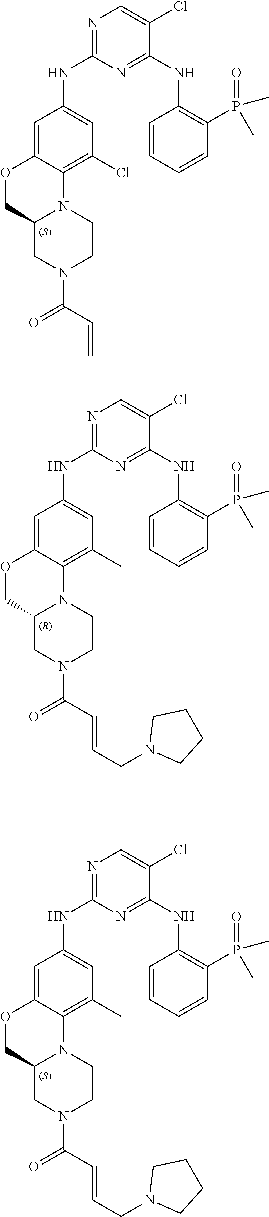 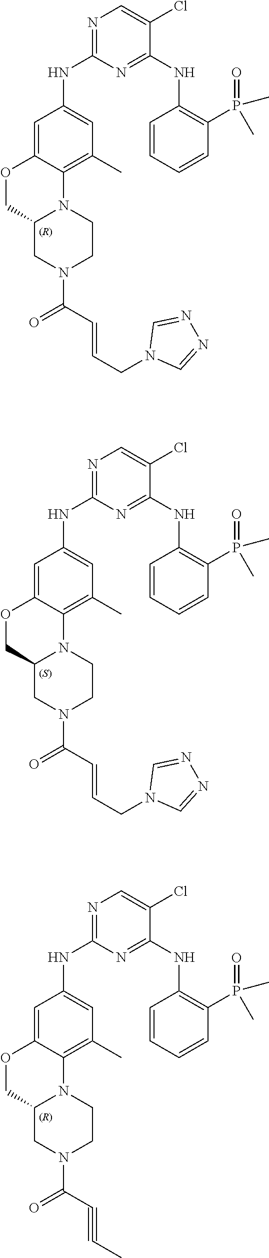 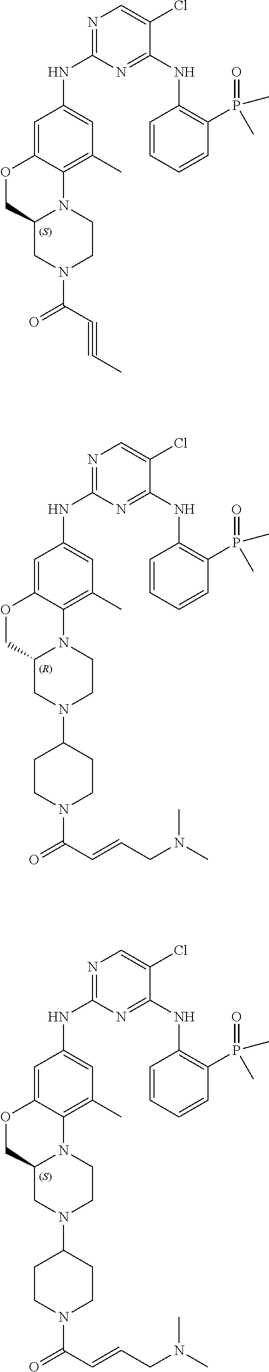 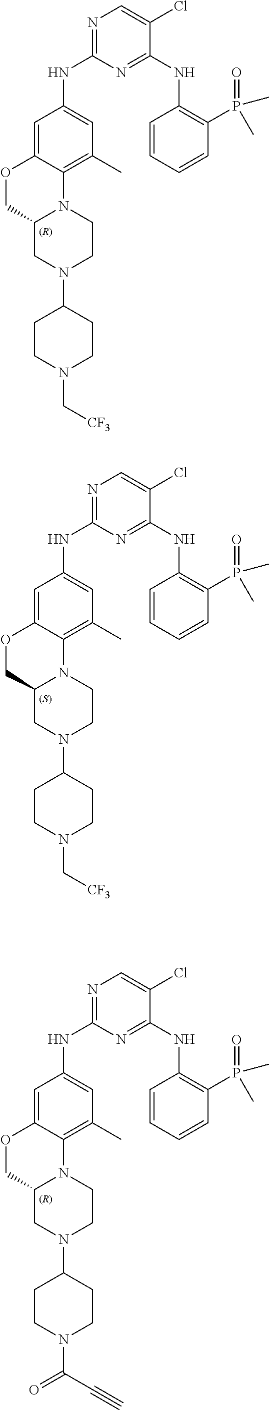 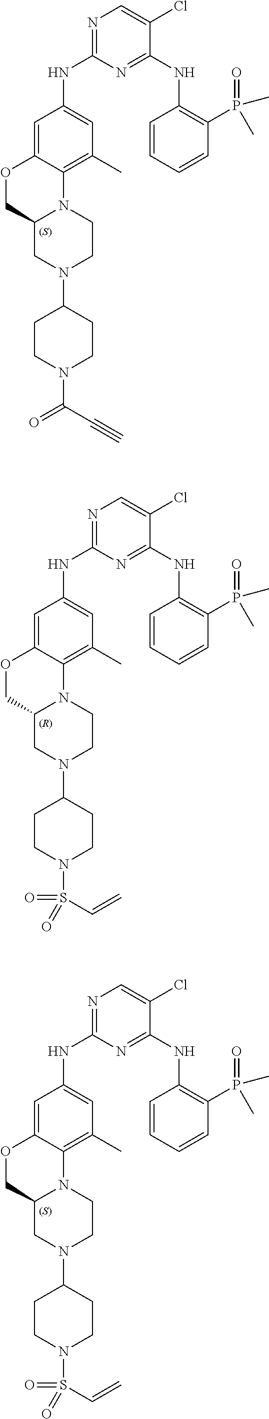 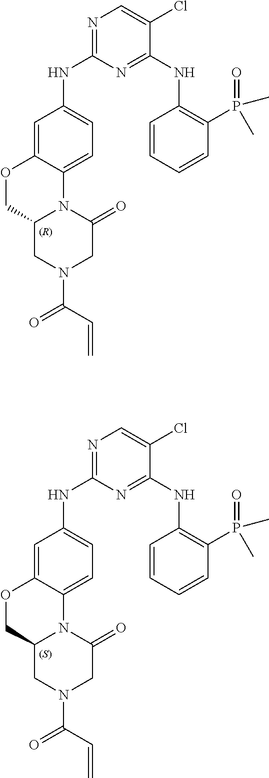
wherein, “*” indicates the chiral center, and when it have not been stated as R or S, the compound with “*” may be racemate, or may be R configuration or S configuration.

14. A method of treating a disease associated with a protein kinase activity or expression level in a subject in need thereof, comprising administering to the subject a therapeutically effective amount of the compound (I) of claim 1, or the optical isomers, pharmaceutically acceptable salts, prodrugs, deuterated derivatives, hydrates or solvates thereof;
wherein the protein kinase is selected from the group consisting of EGFR, EGFR (C797S), ALK, and HPK1, or the combinations thereof.

15. A pharmaceutical composition, comprising: (i) an effective amount of the compound of formula I according to claim 1, or its the optical isomers, pharmaceutically acceptable salts, prodrugs, deuterated derivatives, hydrates or solvates thereof; and (ii) pharmaceutically acceptable carriers.

16. A method of preparing the compound of formula (I) according to claim 1, wherein the method comprises the following steps: 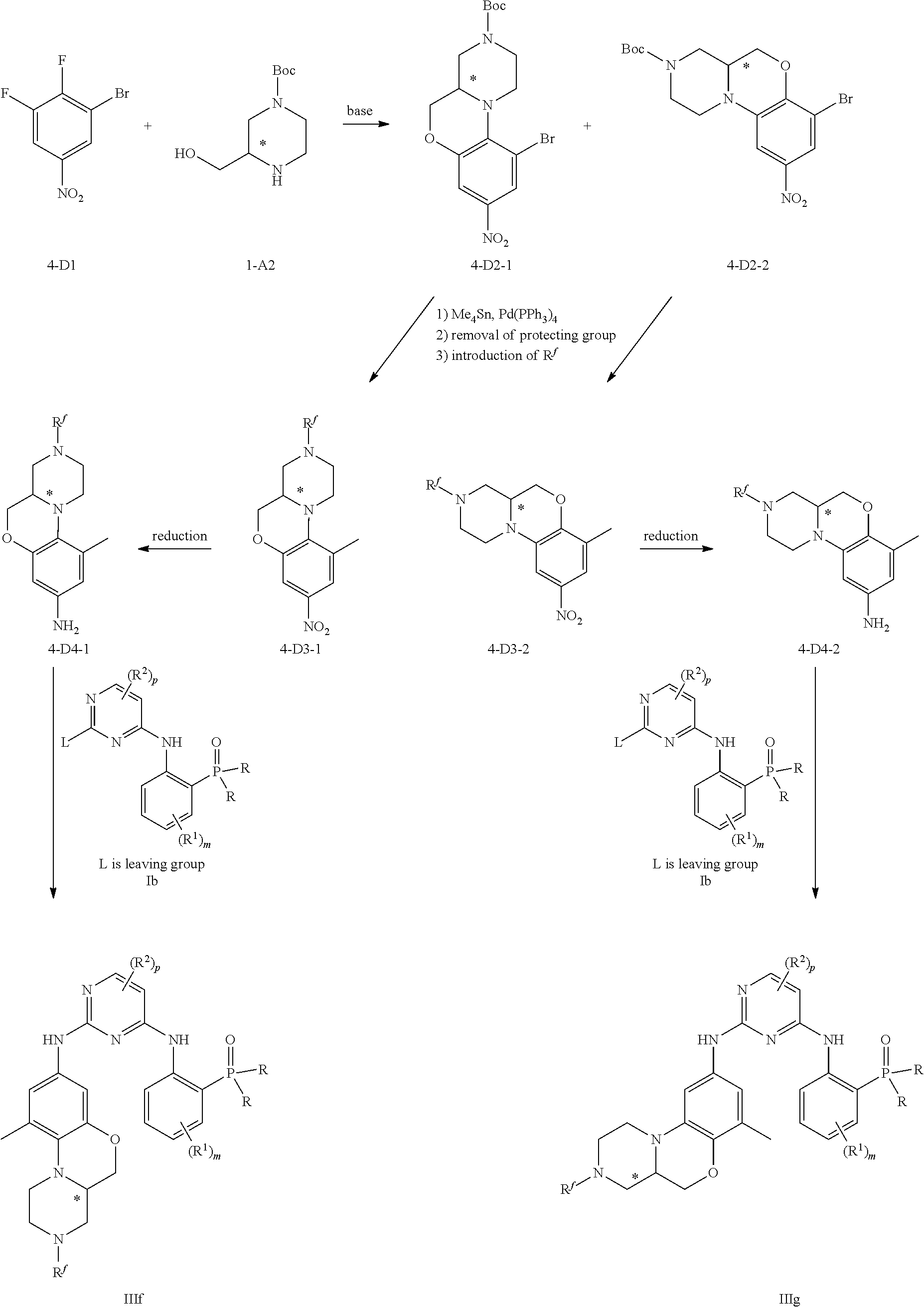
The reaction of the compounds of formula 4-D1 with the compound of formula 1-A2 will produce a compound of formula 4-D2-1 or 4-D2-2;
In the presence of a palladium catalyst, reacting compound of formula 4-D2-1 or formula 4-D2-2 with Me₄Sn will produce the compound of formula 4-D3-1 or 4-D3-2;
The reduction of the compound of formula 4-D3-1 or formula 4-D3-2 will produce compound of formula 4-D4-1 or formula 4-D4-2;
Reacting compound of formula Ib with compound of formula 4-D4-1 or formula 4-D4-2 will produce compound of formula IIIf or IIIg. Compound IIIf or compound IIIg is part of the compounds in formula (I).

17. A method of preparing the compound of formula (I) according to claim 1, wherein the method comprises the following steps: 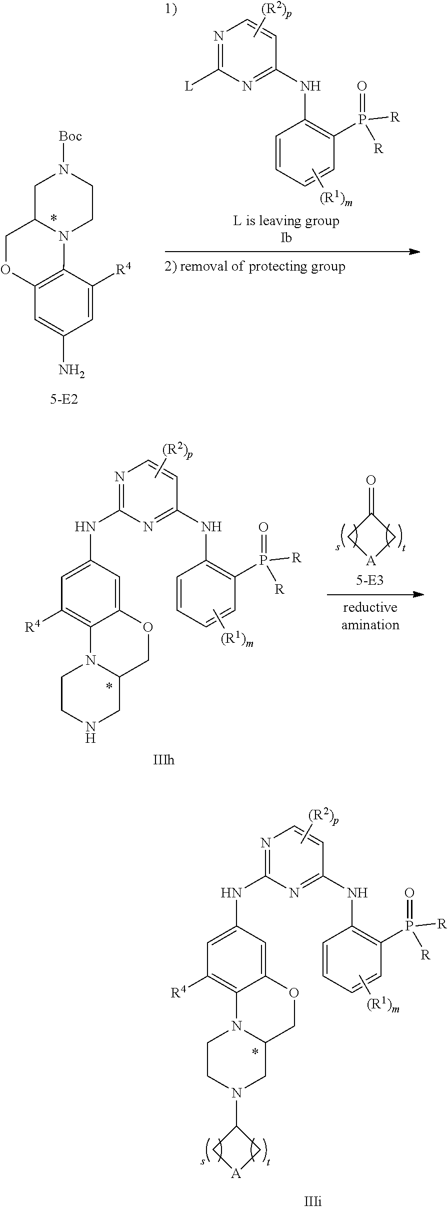
Reaction of compound of formula 5-E2 with compound of formula Tb will produce a compound of formula IIIh;
The reductive amination using compound of formula IIIh and compound of formula 5-E3 will produce compound of formula IIIi. Compound IIIi is part of the compound of formula (I).

18. A method of inhibiting a protein kinase's activity in a subject in need thereof, comprising administering to the subject an effective amount of the compound (I) of claim 1, or the optical isomers, pharmaceutically acceptable salts, prodrugs, deuterated derivatives, hydrates or solvates thereof,
wherein the protein kinase is selected from the group consisting of EGFR, EGFR (C797S), ALK, and HPK1, or the combinations thereof.

19. A method of inhibiting protein kinase activity in a subject in need thereof for the purpose of in vitro non-therapeutics, comprising administering to the subject an effective amount of the compound (I) of claim 1, or the optical isomers, pharmaceutically acceptable salts, prodrugs, deuterated derivatives, hydrates or solvates thereof,
wherein the protein kinase is selected from the group consisting of EGFR, EGFR (C797S), ALK, and HPK1, or the combinations thereof.
